(19) United States
(12) Patent Application Publication
(10) Pub. No.: US 2024/0059759 A1
(43) Pub. Date: Feb. 22, 2024

(54) SARS-COV(-2) SPIKE GLYCOPROTEIN-BINDING DOMAINS AND POLYPEPTIDES COMPRISING THE SAME AND USE OF THE SAME

(71) Applicant: BigHat Biosciences, Inc., San Mateo, CA (US)

(72) Inventors: Mark DEPRISTO, Los Altos, CA (US); Peyton GREENSIDE, San Francisco, CA (US); Randall BREZSKI, San Carlos, CA (US); Ryan HENRICI, Philadelphia, PA (US)

(21) Appl. No.: 18/119,183

(22) Filed: Mar. 8, 2023

Related U.S. Application Data
(63) Continuation of application No. PCT/US2021/049724, filed on Sep. 9, 2021.
(60) Provisional application No. 63/076,885, filed on Sep. 10, 2020.

(51) Int. Cl. C07K 16/10

(52) U.S. Cl. CPC C07K 16/1003; C07K 2317/569; C07K 2317/24; C07K 2317/31; C07K 2317/565; C07K 2317/92

(57) ABSTRACT

Provided herein are antigen-binding domains that specifically bind to a SARS-CoV(-2) spike glycoprotein, polypeptides comprising the same, and uses of the same.

### CROSS-REFERENCE

This application is a continuation of International Patent Application No. PCT/US2021/049724, filed Sep. 9, 2021, which claims the benefit of U.S. Provisional Patent Application No. 63/076,885 filed on Sep. 10, 2020, which are incorporated by reference in their entirety for all purposes.

### SEQUENCE LISTING

The instant application contains a Sequence Listing which has been submitted electronically in XML format and is hereby incorporated by reference in its entirety. Said XML copy, created on Jul. 12, 2023, is named 61301_701_301_SL.xml and is 500,706 bytes in size.

### BACKGROUND OF THE INVENTION

The present disclosure relates generally to polypeptides that include an antigen-binding domain that specifically bind to a spike glycoprotein of SARS-CoV(-2) coronavirus.

Coronaviruses (CoVs) are enveloped RNA viruses that are characterized by club-like spikes that project from their surface, a unique replication strategy, and a large RNA genome (Fehr and Perlman, Coronaviruses 1282:1-23, 2015). Coronaviruses cause a variety of diseases in mammals and birds ranging from enteritis in cows and pigs, and upper respiratory disease in chickens, to lethal respiratory infections in humans.

Severe acute respiratory syndrome (SARS) is a newly emerging infectious disease caused by a coronavirus, which includes SARS-coronavirus (SARS-CoV) and SARS-coronavirus-2 (SARS-CoV-2). The SARS-CoV and SARS-CoV-2 each include a spike (S) protein is composed of two subunits. The S1 subunit contains a receptor-binding domain that engages with the host cell receptor angiotensin-converting enzyme 2 (ACE2) and the S2 subunit mediates fusion between the viral and host cell membranes. The S protein plays a key part in the induction of humoral immunity (e.g., neutralizing-antibody) and cellular immunity (e.g., T-cell responses), as well as protective immunity, during infection with SARS-CoV and SARS-CoV-2. Given the emergence of SARS-CoV-2 (COVID-19), there remains a critical unmet need for a therapies to treat and/or prevent a coronavirus infection.

### SUMMARY

This present disclosure is based on the discovery of antigen-binding domains that selectively bind to SARS-CoV and SARS-CoV-2 spike glycoprotein.

In one aspect, this disclosure features a polypeptide including an antigen-binding domain including: (i) a CDR1 including a sequence of Formula I: G-R-T-F-S-E-Y-A-M-G (SEQ ID NO: 1); (ii) a CDR2 including a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 including a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; Xis is G, E, R, or W; X₁₉ is E or A; X₂₀ is W or A; and X₂₁ is D or A.

In some embodiments of any of the polypeptides described herein, the antigen-binding domain includes: (i) a CDR1 sequence selected from the group consisting of: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), or TISWSGGATYYTRSVKG (SEQ ID NO: 44); and (iii) a CDR3 sequence selected from the group consisting of: AAAGLGTVVSEWDYDYDY (SEQ ID NO: 45), AAAGLGTVVSEWDDYDY (SEQ ID NO: 46), APAGLGTVVSEWDYDYDY (SEQ ID NO: 47), AAADLGTVVSEWDYDYDY (SEQ ID NO: 48), AAAGLGTVVSEADYDYDY (SEQ ID NO: 49), AAAGLRTVVSEWDYDYDY (SEQ ID NO: 50), AYAGLGTVVSEWDYDYDY (SEQ ID NO: 51), AAAGLGTVVSEWDYDYDS (SEQ ID NO: 52), AAAGLWTVVSEWDYDYDY (SEQ ID NO: 53), AAAGLGTVVSAWDYDYDY (SEQ ID NO: 54), AAAGLGTVVSEWDYDDY (SEQ ID NO: 55), AAAGQGTVVSEWDYDYDY (SEQ ID NO: 56), AAAGLETVVSEWDYDYDY (SEQ ID NO: 57), AAARLGTVVSEWDYDYDY (SEQ ID NO: 58), AAAGLETVVSEWDYDYDY (SEQ ID NO: 57); or AAAGLGTVVSEWAYDYDA (SEQ ID NO: 60).

In some embodiments of any of the polypeptides described herein, the antigen-binding domain includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45).

In some embodiments of any of the polypeptides described herein, the antigen-binding domain includes: a framework region 1 (FR1) sequence selected from the group consisting of:

(SEQ ID NO: 61)
MQVQLQESGGGLVQAGGSLRLSCAASG,
and
(SEQ ID NO: 62)
MQVQLQESGGGLVQAGGSLRLSCAASGSG.

In some embodiments of any of the polypeptides described herein, the antigen-binding domain includes a sequence that is at least 80% identical to SEQ ID NO: 63. In some embodiments, the antigen-binding domain includes SEQ ID NO: 63.

In some embodiments of any of the polypeptides described herein, the antigen-binding domain includes a sequence that is at least 80% identical to SEQ ID NO: 68. In some embodiments, the antigen-binding domain includes SEQ ID NO: 68.

In some embodiments of any of the polypeptides described herein, the antigen-binding domain includes a sequence that is at least 80% identical to SEQ ID NO: 71. In some embodiments, the antigen-binding domain includes SEQ ID NO: 71.

In some embodiments of any of the polypeptides described herein, the antigen-binding domain includes a sequence that is at least 80% identical to SEQ ID NO: 72. In some embodiments, the antigen-binding domain includes SEQ ID NO: 72.

In some embodiments of any of the polypeptides described herein, the antigen-binding domain includes a sequence that is at least 80% identical to SEQ ID NO: 73. In some embodiments, the antigen-binding domain includes SEQ ID NO: 73.

In some embodiments of any of the polypeptides described herein, the antigen-binding domain includes a sequence that is at least 80% identical to SEQ ID NO: 75. In some embodiments, the antigen-binding domain includes SEQ ID NO: 75.

In some embodiments of any of the polypeptides described herein, the antigen-binding domain includes a sequence that is at least 80% identical to SEQ ID NO: 76. In some embodiments, the antigen-binding domain includes SEQ ID NO: 76.

In some embodiments of any of the polypeptides described herein, the antigen-binding domain includes a sequence that is at least 80% identical to SEQ ID NO: 77. In some embodiments, the antigen-binding domain includes SEQ ID NO: 77.

In some embodiments of any of the polypeptides described herein, the antigen-binding domain includes a sequence that is at least 80% identical to SEQ ID NO: 79. In some embodiments, the antigen-binding domain includes SEQ ID NO: 79.

In some embodiments of any of the polypeptides described herein, the antigen-binding domain includes a sequence that is at least 80% identical to SEQ ID NO: 80. In some embodiments, the antigen-binding domain includes SEQ ID NO: 80.

In some embodiments of any of the polypeptides described herein, the antigen-binding domain includes a sequence that is at least 80% identical to SEQ ID NO: 81. In some embodiments, the antigen-binding domain includes SEQ ID NO: 81.

In some embodiments of any of the polypeptides described herein, the antigen-binding domain includes a sequence that is at least 80% identical to SEQ ID NO: 82. In some embodiments, the antigen-binding domain includes SEQ ID NO: 82.

In some embodiments of any of the polypeptides described herein, the antigen-binding domain includes a sequence that is at least 80% identical to SEQ ID NO: 84. In some embodiments, the antigen-binding domain includes SEQ ID NO: 84.

In some embodiments of any of the polypeptides described herein, the antigen-binding domain includes a sequence that is at least 80% identical to SEQ ID NO: 85. In some embodiments, the antigen-binding domain includes SEQ ID NO: 85.

In some embodiments of any of the polypeptides described herein, antigen-binding domain includes a sequence that is at least 80% identical to SEQ ID NO: 86. In some embodiments, the antigen-binding domain includes SEQ ID NO: 86.

In some embodiments of any of the polypeptides described herein, the antigen-binding domain includes a sequence that is at least 80% identical to SEQ ID NO: 90. In some embodiments, the antigen-binding domain includes SEQ ID NO: 90.

In some embodiments of any of the polypeptides described herein, the antigen-binding domain includes a sequence that is at least 80% identical to SEQ ID NO: 91. In some embodiments, the antigen-binding domain includes SEQ ID NO: 91.

In some embodiments of any of the polypeptides described herein, the antigen-binding domain includes a sequence that is at least 80% identical to SEQ ID NO: 92. In some embodiments, the antigen-binding domain includes SEQ ID NO: 92.

In some embodiments of any of the polypeptides described herein, the antigen-binding domain includes a sequence that is at least 80% identical to SEQ ID NO: 93. In some embodiments, the antigen-binding domain includes SEQ ID NO: 93.

In some embodiments of any of the polypeptides described herein, the antigen-binding domain includes a sequence that is at least 80% identical to SEQ ID NO: 95. In some embodiments, the antigen-binding domain includes SEQ ID NO: 95.

In some embodiments of any of the polypeptides described herein, the antigen-binding domain includes a sequence that is at least 80% identical to SEQ ID NO: 97. In some embodiments, the antigen-binding domain includes SEQ ID NO: 97.

In some embodiments of any of the polypeptides described herein, the antigen-binding domain includes a sequence that is at least 80% identical to SEQ ID NO: 98. In some embodiments, the antigen-binding domain includes SEQ ID NO: 98.

In some embodiments of any of the polypeptides described herein, the antigen-binding domain includes a sequence that is at least 80% identical to SEQ ID NO: 99. In some embodiments, the antigen-binding domain includes SEQ ID NO: 99.

In some embodiments of any of the polypeptides described herein, the antigen-binding domain includes a sequence that is at least 80% identical to SEQ ID NO: 107. In some embodiments, the antigen-binding domain includes SEQ ID NO: 107.

In some embodiments of any of the polypeptides described herein, the antigen-binding domain includes a sequence that is at least 80% identical to SEQ ID NO: 108. In some embodiments, the antigen-binding domain includes SEQ ID NO: 108.

In some embodiments of any of the polypeptides described herein, the antigen-binding domain includes a sequence that is at least 80% identical to SEQ ID NO: 113. In some embodiments, the antigen-binding domain includes SEQ ID NO: 113.

In some embodiments of any of the polypeptides described herein, the antigen-binding domain includes a sequence that is at least 80% identical to SEQ ID NO: 114. In some embodiments, the antigen-binding domain includes SEQ ID NO: 114.

In some embodiments of any of the polypeptides described herein, the antigen-binding domain includes a sequence that is at least 80% identical to SEQ ID NO: 115. In some embodiments, the antigen-binding domain includes SEQ ID NO: 115.

In some embodiments of any of the polypeptides described herein, the antigen-binding domain includes a sequence that is at least 80% identical to SEQ ID NO: 117. In some embodiments, the antigen-binding domain includes SEQ ID NO: 117.

In some embodiments of any of the polypeptides described herein, the antigen-binding domain includes a sequence that is at least 80% identical to SEQ ID NO: 118. In some embodiments, the antigen-binding domain includes SEQ ID NO: 118.

In some embodiments of any of the polypeptides described herein, the antigen-binding domain includes a sequence that is at least 80% identical to SEQ ID NO: 119. In some embodiments, the antigen-binding domain includes SEQ ID NO: 119.

In some embodiments of any of the polypeptides described herein, the antigen-binding domain includes a sequence that is at least 80% identical to SEQ ID NO: 120. In some embodiments, the antigen-binding domain includes SEQ ID NO: 120.

In some embodiments of any of the polypeptides described herein, the antigen-binding domain includes a sequence that is at least 80% identical to SEQ ID NO: 121. In some embodiments, the antigen-binding domain includes SEQ ID NO: 121.

In some embodiments of any of the polypeptides described herein, the antigen-binding domain includes a sequence that is at least 80% identical to SEQ ID NO: 122. In some embodiments, the antigen-binding domain includes SEQ ID NO: 122.

In some embodiments of any of the polypeptides described herein, the antigen-binding domain includes a sequence that is at least 80% identical to SEQ ID NO: 124. In some embodiments, the antigen-binding domain includes SEQ ID NO: 124.

In some embodiments of any of the polypeptides described herein, the antigen-binding domain includes a sequence that is at least 80% identical to SEQ ID NO: 125. In some embodiments, the antigen-binding domain includes SEQ ID NO: 125.

In some embodiments of any of the polypeptides described herein, the antigen-binding domain includes a sequence that is at least 80% identical to SEQ ID NO: 126. In some embodiments, the antigen-binding domain includes SEQ ID NO: 126.

In some embodiments of any of the polypeptides described herein, the antigen-binding domain includes a sequence that is at least 80% identical to a sequence selected from SEQ ID NOs: 64-67, 69, 70, 74, 78, 83, 87-89, 94, 96, 100-106, 109-111, 112, 116, and 123. In some embodiments, the antigen-binding domain includes a sequence selected from SEQ ID NOs: 64-67, 69, 70, 74, 78, 83, 87-89, 94, 96, 100-106, 109-111, 112, 116 and 123.

In another aspect, this disclosure features a polypeptide including an antigen-binding domain including: (i) a CDR1 including a sequence of Formula IV of: G-F-P-V-Y-S-W-N(SEQ ID NO: 127); (ii) a CDR2 including a sequence of Formula V of: I-E-S-H-X₁-D-S-T (SEQ ID NO: 128), wherein X₁ is G or A; and (iii) a CDR3 including a sequence of Formula VI: Y-V-W-V-X₂-H-T-Y-Y-G-Q (SEQ ID NO: 129), wherein X₂ is G or A.

In some embodiments of any of the polypeptides described herein, the antigen-binding domain includes: (i) a CDR1 sequence selected from the group consisting of: GFPVYSWN (SEQ ID NO: 127); (ii) a CDR2 sequence selected from the group consisting of: IESHGDST (SEQ ID NO: 130), and IESYAHGT (SEQ ID NO: 131); and (iii) a CDR3 sequence selected from the group consisting of: YVWVGHTYYGQ (SEQ ID NO: 132), and YVWVAHTYYGQ (SEQ ID NO: 133).

In some embodiments of any of the polypeptides described herein, the antigen-binding domain includes: GFPVYSWN (SEQ ID NO: 127), IESHGDST (SEQ ID NO: 130), and YVWVAHTYYGQ (SEQ ID NO: 133); GFPVYSWN (SEQ ID NO: 127), IESHADST (SEQ ID NO: 231), and YVWVGHTYYGQ (SEQ ID NO: 132); or GFPVYSWN (SEQ ID NO: 127), IESHADST (SEQ ID NO: 231), and YVWVAHTYYGQ (SEQ ID NO: 133).

In some embodiments of any of the polypeptides described herein, the antigen-binding domain includes a sequence that is at least 80% identical to SEQ ID NO: 134. In some embodiments, the antigen-binding domain includes SEQ ID NO: 134.

In some embodiments of any of the polypeptides described herein, the antigen-binding domain includes a sequence that is at least 80% identical to SEQ ID NO: 135. In some embodiments, the antigen-binding domain includes SEQ ID NO: 135.

In some embodiments of any of the polypeptides described herein, the antigen-binding domain includes a sequence that is at least 80% identical to SEQ ID NO: 136. In some embodiments, the antigen-binding domain includes SEQ ID NO: 136.

In another aspect, this disclosure features a polypeptide including an antigen-binding domain including: (i) a CDR1 including a sequence of Formula VII of: G-X₁-T-X₂-S-T-A-A (SEQ ID NO: 137), wherein X₁ is R or A; X₂ is F, H, or Y; (ii) a CDR2 including a sequence of Formula VIII of: I-R-W-S-X₃-G-S-A (SEQ ID NO: 138), wherein X₃ is G or A; and (iii) a CDR3 including a sequence of Formula IX: A-R-T-E-N-V-R-X₄-X₅-L-S-D-Y-A-T-X₆-P-Y-X₇-Y (SEQ ID NO: 139), wherein X₄ is S or A; X₅ is L or K; X₆ is W or Y, and X₇ is A, Q, or R.

In some embodiments of any of the polypeptides described herein, the antigen-binding domain includes: (i) a CDR1 sequence selected from the group consisting of: GRTFSTAA (SEQ ID NO: 140), GRTFATAA (SEQ ID NO: 141), GATFSTAA (SEQ ID NO: 142), GRTYSTAA (SEQ ID NO: 143), and GRTHSTAA (SEQ ID NO: 144); (ii) a CDR2 sequence selected from the group consisting of: IRWSGGSA (SEQ ID NO: 145), and IRWSAGSA (SEQ ID NO: 146); and (iii) a CDR3 sequence selected from the group consisting of: ARTENVRSLLSDYATWPYDY (SEQ ID NO: 147), ARTENVRSLLSDYATYPYDY (SEQ ID NO: 148), ARTENVRALLSDYATWPYDY (SEQ ID NO: 149), ARTENVRSKLSDYATWPYDY (SEQ ID NO: 150), ARTENVRSLLSDYATWPYRY (SEQ ID NO: 151), ARTENVRSLLSDYATWPYQY (SEQ ID NO: 152), and ARTENVRSLLSDYATWPYAY (SEQ ID NO: 153).

In some embodiments of any of the polypeptides described herein, the antigen-binding domain includes: GRTFSTAA (SEQ ID NO: 140), IRWSGGSA (SEQ ID NO: 145), and ARTENVRSLLSDYATYPYDY (SEQ ID NO: 148); GRTFATAA (SEQ ID NO: 141), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRALLSDYATWPYDY (SEQ ID NO: 149); GATFSTAA (SEQ ID NO: 142), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSKLSDYATWPYDY (SEQ ID NO: 150); GRTFSTAA (SEQ ID NO: 140), IRWSAGSA (SEQ ID NO: 146), and ARTENVRSLLSDYATWPYDY (SEQ ID NO: 147); GRTYSTAA (SEQ ID NO: 143), IRWSGGSA (SEQ ID NO: 145), and ARTENVRSLLSDYATWPYDY (SEQ ID NO: 147); GRTHSTAA (SEQ ID NO: 144), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSLLSDYATWPYRY (SEQ ID NO: 151); GRTFSTAA (SEQ ID NO: 140), IRWSGGSA (SEQ ID NO: 145), and ARTENVRSLLSDYATWPYQY (SEQ ID NO:152); or GRTFSTAA (SEQ ID NO: 140), IRWSGGSA (SEQ ID NO: 145), and ARTENVRSLLSDYATWPYAY (SEQ ID NO: 153).

In some embodiments of any of the polypeptides described herein, the antigen-binding domain includes a sequence that is at least 80% identical to SEQ ID NO: 154. In some embodiments, the antigen-binding domain includes SEQ ID NO: 154.

In some embodiments of any of the polypeptides described herein, the antigen-binding domain includes a sequence that is at least 80% identical to SEQ ID NO: 155. In some embodiments, the antigen-binding domain includes SEQ ID NO: 155.

In some embodiments of any of the polypeptides described herein, the antigen-binding domain includes a sequence that is at least 80% identical to SEQ ID NO: 156. In some embodiments, the antigen-binding domain includes SEQ ID NO: 156.

In some embodiments of any of the polypeptides described herein, the antigen-binding domain includes a sequence that is at least 80% identical to SEQ ID NO: 157. In some embodiments, the antigen-binding domain includes SEQ ID NO: 157.

In some embodiments of any of the polypeptides described herein, the antigen-binding domain includes a sequence that is at least 80% identical to SEQ ID NO: 158. In some embodiments, the antigen-binding domain includes SEQ ID NO: 158.

In some embodiments of any of the polypeptides described herein, the antigen-binding domain includes a sequence that is at least 80% identical to SEQ ID NO: 159. In some embodiments, the antigen-binding domain includes SEQ ID NO: 159.

In some embodiments of any of the polypeptides described herein, the antigen-binding domain includes a sequence that is at least 80% identical to SEQ ID NO: 160. In some embodiments, the antigen-binding domain includes SEQ ID NO: 160.

In some embodiments of any of the polypeptides described herein, the antigen-binding domain includes a sequence that is at least 80% identical to SEQ ID NO: 161. In some embodiments, the antigen-binding domain includes SEQ ID NO: 161.

In some embodiments of any of the polypeptides described herein, the antigen-binding domain includes a sequence that is at least 80% identical to SEQ ID NO: 162. In some embodiments, the antigen-binding domain includes SEQ ID NO: 162.

In some embodiments of any of the polypeptides described herein, the antigen-binding domain includes a sequence that is at least 80% identical to SEQ ID NO: 163. In some embodiments, the antigen-binding domain includes SEQ ID NO: 163.

In some embodiments of any of the polypeptides described herein, the antigen-binding domain includes a sequence that is at least 80% identical to SEQ ID NO: 164. In some embodiments, the antigen-binding domain includes SEQ ID NO: 164.

In some embodiments of any of the polypeptides described herein, the antigen-binding domain includes a sequence that is at least 80% identical to SEQ ID NO: 165. In some embodiments, the antigen-binding domain includes SEQ ID NO: 165.

In another aspect, this disclosure feature a polypeptide including an antigen-binding domain including: (i) a CDR1 including a sequence of Formula X of: G-F-P-V-E-X₁-X₂-X₃ (SEQ ID NO: 166), wherein X₁ is V or A, X₂ is W, Y, or A, and X₃ is R or A; (ii) a CDR2 including a sequence of Formula XI of: I-E-X₄-X₅-G-H-G-X₆ (SEQ ID NO: 167), wherein X₄ is G or A, X₅ is Y or A, and X₆ is T or A; and (iii) a CDR3 including a sequence of Formula XII: N-V-X₇-D-D-X₈-X₉-L-A-Y-H-Y-D-Y (SEQ ID NO: 168), wherein X₇ is G or A, X₈ is G or N, and X₉ is Q or H.

In some embodiments of any of the polypeptides described herein, the antigen-binding domain includes: (i) a CDR1 sequence selected from the group consisting of: GFPVEVWR (SEQ ID NO: 169), GFPVEVYR (SEQ ID NO: 170), GFPVEVAR (SEQ ID NO: 171), GFPVEAWR (SEQ ID NO: 172), and GFPVEVWA (SEQ ID NO: 173); (ii) a CDR2 sequence selected from the group consisting of: IESYGHGT (SEQ ID NO: 174), IESAGHGT (SEQ ID NO: 175), IESYGHGA (SEQ ID NO: 176), and IEAYGHGT (SEQ ID NO: 177); and (iii) a CDR3 sequence selected from the group consisting of: NVKDDGQLAYHYDY (SEQ ID NO: 178), NVYDDGQLAYHYDY (SEQ ID NO: 179), NVYDDGHLAYHYDY (SEQ ID NO: 180), and NVYDDNQLAYHYDY (SEQ ID NO: 181).

In some embodiments of any of the polypeptides described herein, the antigen-binding domain includes: GFPVEVWR (SEQ ID NO: 169), IESAGHGT (SEQ ID NO: 175), and NVYDDGQLAYHYDY (SEQ ID NO: 179); GFPVEVYR (SEQ ID NO: 170), IESYGHGT (SEQ ID NO: 174), and NVYDDGQLAYHYDY (SEQ ID NO: 179); GFPVEVAR (SEQ ID NO: 171), IESYGHGT (SEQ ID NO: 174), and NVYDDGQLAYHYDY (SEQ ID NO: 179); GFPVEAWR (SEQ ID NO: 172), IESYGHGT (SEQ ID NO: 174), and NVYDDGQLAYHYDY (SEQ ID NO: 179); GFPVEVWR (SEQ ID NO: 169), IESYGHGA (SEQ ID NO: 176), and NVYDDGQLAYHYDY (SEQ ID NO: 179); GFPVEVWR (SEQ ID NO: 169), IEAYGHGT (SEQ ID NO: 177), and NVYDDGQLAYHYDY (SEQ ID NO: 179); GFPVEVWA (SEQ ID NO: 173), IESYGHGT (SEQ ID NO: 174), and NVYDDGQLAYHYDY (SEQ ID NO: 179); GFPVEVWR (SEQ ID NO: 169), IESYGHGT (SEQ ID NO: 174), and NVYDDGHLAYHYDY (SEQ ID NO: 180); GFPVEVWR (SEQ ID NO: 169), IESYGHGT (SEQ ID NO: 174), and NVYDDGQLAYHYDY (SEQ ID NO: 179); or GFPVEVWR (SEQ ID NO: 169), IESYGHGT (SEQ ID NO: 174), and NVYDDNQLAYHYDY (SEQ ID NO: 181).

In some embodiments of any of the polypeptides described herein, the first antigen-binding domain includes a framework region 3 (FR3) sequence selected from the group consisting of:

(SEQ ID NO: 182)
RYADSVKGRFTISRDNAKNTVYLQMNSLKPEDTAVYYC,
(SEQ ID NO: 183)
RYADSVRGRFTISRDNAKNTVYLQMNSLKPEDTAVYYC,
or
(SEQ ID NO: 184)
RYADSVAGRFTISRDNAKNTVYLQMNSLKPEDTAVYYC.

In some embodiments of any of the polypeptides described herein, the antigen-binding domain includes a sequence that is at least 80% identical to SEQ ID NO: 185. In some embodiments, the antigen-binding domain includes SEQ ID NO: 185.

In some embodiments of any of the polypeptides described herein, the antigen-binding domain includes a sequence that is at least 80% identical to SEQ ID NO: 186. In some embodiments, the antigen-binding domain includes SEQ ID NO: 186.

In some embodiments of any of the polypeptides described herein, the antigen-binding domain includes a sequence that is at least 80% identical to SEQ ID NO: 187. In some embodiments, the antigen-binding domain includes SEQ ID NO: 187.

In some embodiments of any of the polypeptides described herein, the antigen-binding domain includes a sequence that is at least 80% identical to SEQ ID NO: 188. In some embodiments, the antigen-binding domain includes SEQ ID NO: 188.

In some embodiments of any of the polypeptides described herein, the antigen-binding domain includes a sequence that is at least 80% identical to SEQ ID NO: 189. In some embodiments, the antigen-binding domain includes SEQ ID NO: 189.

In some embodiments of any of the polypeptides described herein, the antigen-binding domain includes a sequence that is at least 80% identical to SEQ ID NO: 190. In some embodiments, the antigen-binding domain includes SEQ ID NO: 190.

In some embodiments of any of the polypeptides described herein, the antigen-binding domain includes a sequence that is at least 80% identical to SEQ ID NO: 191. In some embodiments, the antigen-binding domain includes SEQ ID NO: 191.

In some embodiments of any of the polypeptides described herein, the antigen-binding domain includes a sequence that is at least 80% identical to SEQ ID NO: 192. In some embodiments, the antigen-binding domain includes SEQ ID NO: 192.

In some embodiments of any of the polypeptides described herein, the antigen-binding domain includes a sequence that is at least 80% identical to SEQ ID NO: 193. In some embodiments, the antigen-binding domain includes SEQ ID NO: 193.

In some embodiments of any of the polypeptides described herein, the antigen-binding domain includes a sequence that is at least 80% identical to SEQ ID NO: 194. In some embodiments, the antigen-binding domain includes SEQ ID NO: 194.

In some embodiments of any of the polypeptides described herein, the antigen-binding domain includes a sequence that is at least 80% identical to SEQ ID NO: 195. In some embodiments, the antigen-binding domain includes SEQ ID NO: 195.

In some embodiments of any of the polypeptides described herein, the antigen-binding domain includes a sequence that is at least 80% identical to SEQ ID NO: 196. In some embodiments, the antigen-binding domain includes SEQ ID NO: 196.

In some embodiments of any of the polypeptides described herein, the antigen-binding domain includes a sequence that is at least 80% identical to SEQ ID NO: 197. In some embodiments, the antigen-binding domain includes SEQ ID NO: 197.

In some embodiments of any of the polypeptides described herein, the antigen-binding domain is a single domain antibody. In some embodiments of any of the polypeptides described herein, the polypeptide further includes one or more additional antigen-binding domains. In some embodiments of any of the polypeptides described herein, the polypeptide is a single-chain polypeptide. In some embodiments of any of the polypeptides described herein, the polypeptide is a multi-chain polypeptide. In some embodiments of any of the polypeptides described herein, the antigen-binding domain is humanized.

In another aspect, this disclosure features a pharmaceutical composition including any of the polypeptides described herein and a pharmaceutically acceptable carrier. In some embodiments, the pharmaceutical composition is formulated for intravenous administration.

In another aspect, this disclosure features a method of treating a subject having or suspected of having a coronavirus infection, wherein the method includes administering a therapeutically effective amount of any of the pharmaceutical compositions described herein. In some embodiments of any of the methods described herein, the coronavirus infection is a SARS-CoV-2 infection.

In another aspect, this disclosure features a kit including any of the pharmaceutical compositions described herein.

In another aspect, this disclosure features a nucleic acid encoding any of the polypeptides described herein.

In another aspect, this disclosure features a vector including any of the nucleic acids described herein.

In another aspect, this disclosure features a host cell that includes any of the nucleic acids described herein or any of the vectors described herein.

In another aspect, this disclosure features a method of producing a polypeptide including: (a) culturing any of the host cells described herein in a culture medium under conditions sufficient to allow for the production of the polypeptide; and (b) harvesting the polypeptide from the host cell or the culture medium. In some embodiments, the method further includes isolating the polypeptide. In some embodiments, the method further includes formulating the isolated polypeptide.

In another aspect, this disclosure features a polypeptide including: (a) a first antigen-binding domain including: (i) a CDR1 including a sequence of Formula I: G-R-T-F-S-E-Y-A-M-G (SEQ ID NO: 1); (ii) a CDR2 including a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 including a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain including: (i) a CDR1 including a sequence of Formula IV of: G-F-P-V-Y-S-W-N (SEQ ID NO: 127); (ii) a CDR2 including a sequence of Formula V of: I-E-S-H-X₁-D-S-T (SEQ ID NO: 128), wherein X₁ is G or A; and (iii) a CDR3 including a sequence of Formula VI: Y-V-W-V-X₂-H-T-Y-Y-G-Q (SEQ ID NO: 129), wherein X₂ is G or A.

In some embodiments of any of the polypeptides described herein, the first antigen-binding domain includes: (i) a CDR1 sequence selected from the group consisting of: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), TISWSGGATYYTRSVKG (SEQ ID NO: 44), TISWSGGSTYYTASVKG (SEQ ID NO: 228), TISWSGGATYYTASVKG (SEQ ID NO: 229), or TISWSGGGTYYTDSVKG (SEQ ID NO: 230); and (iii) a CDR3 sequence selected from the group consisting of:

(SEQ ID NO: 45)
AAAGLGTVVSEWDYDYDY,
(SEQ ID NO: 46)
AAAGLGTVVSEWDDYDY,
(SEQ ID NO: 47)
APAGLGTVVSEWDYDYDY,
(SEQ ID NO: 48)
AAADLGTVVSEWDYDYDY,
(SEQ ID NO: 49)
AAAGLGTVVSEADYDYDY,
(SEQ ID NO: 50)
AAAGLRTVVSEWDYDYDY,
(SEQ ID NO: 51)
AYAGLGTVVSEWDYDYDY,
(SEQ ID NO: 52)
AAAGLGTVVSEWDYDYDS,
(SEQ ID NO: 53)
AAAGLWTVVSEWDYDYDY,
(SEQ ID NO: 54)
AAAGLGTVVSAWDYDYDY,
(SEQ ID NO: 55)
AAAGLGTVVSEWDYDDY,
(SEQ ID NO: 56)
AAAGQGTVVSEWDYDYDY,
(SEQ ID NO: 57)
AAAGLETVVSEWDYDYDY,
(SEQ ID NO: 58)
AAARLGTVVSEWDYDYDY,
(SEQ ID NO: 57)
AAAGLETVVSEWDYDYDY;
or,
(SEQ ID NO: 60)
AAAGLGTVVSEWAYDYDA.

In some embodiments of any of the polypeptides described herein, the first antigen-binding domain includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45).

In some embodiments of any of the polypeptides described herein, the first antigen-binding domain includes a framework region 1 (FR1) sequence selected from the group consisting of:

(SEQ ID NO: 61)
MQVQLQESGGGLVQAGGSLRLSCAASG,
and
(SEQ ID NO: 62)
MQVQLQESGGGLVQAGGSLRLSCAASGSG.

In some embodiments of any of the polypeptides described herein, the first antigen-binding domain includes a sequence that is at least 80% identical to a sequence selected from SEQ ID NOs: 63-126 or 216-226. In some embodiments, the first antigen-binding domain includes a sequence that is selected from SEQ ID NOs: 63-126 or 216-226.

In some embodiments of any of the polypeptides described herein, the second antigen-binding domain includes: (i) a CDR1 sequence selected from the group consisting of: GFPVYSWN (SEQ ID NO: 127); (ii) a CDR2 sequence selected from the group consisting of: IESHGDST (SEQ ID NO: 130), and IESHADST (SEQ ID NO: 231); and (iii) a CDR3 sequence selected from the group consisting of: YVWVGHTYYGQ (SEQ ID NO: 132), and YVWVAHTYYGQ (SEQ ID NO: 133).

In some embodiments of any of the polypeptides described herein, the second antigen-binding domain includes: GFPVYSWN (SEQ ID NO: 127), IESHGDST (SEQ ID NO: 130), and YVWVAHTYYGQ (SEQ ID NO: 133); GFPVYSWN (SEQ ID NO: 127), IESHADST (SEQ ID NO: 231), and YVWVGHTYYGQ (SEQ ID NO: 132); or GFPVYSWN (SEQ ID NO: 127), IESHADST (SEQ ID NO: 231), and YVWVAHTYYGQ (SEQ ID NO: 133).

In some embodiments of any of the polypeptides described herein, the second antigen-binding domain includes a sequence that is at least 80% identical to a sequence selected from SEQ ID NOs: 134-136. In some embodiments, the second antigen-binding domain includes a sequence that is selected from SEQ ID NOs: 134-136.

In some embodiments of any of the polypeptides described herein, the first antigen-binding domain includes a CDR1 including SEQ ID NO: 1, a CDR2 including SEQ ID NO: 5, and a CDR3 including SEQ ID NO: 45, and the second antigen-binding domain includes a CDR1 including SEQ ID NO: 140, a CDR2 including SEQ ID NO: 145, and a CDR3 including SEQ ID NO: 151.

In some embodiments of any of the polypeptides described herein, the first antigen-binding domain includes a CDR1 including SEQ ID NO: 1, a CDR2 including SEQ ID NO: 27, and a CDR3 including SEQ ID NO: 45, and the second antigen-binding domain includes a CDR1 including SEQ ID NO: 127, a CDR2 including SEQ ID NO: 130, and a CDR3 including SEQ ID NO: 133.

In some embodiments of any of the polypeptides described herein, the first antigen-binding domain includes a CDR1 including SEQ ID NO: 1, a CDR2 including SEQ ID NO: 5, and a CDR3 including SEQ ID NO: 45, and the second antigen-binding domain includes a CDR1 including SEQ ID NO: 127, a CDR2 including SEQ ID NO: 130, and a CDR3 including SEQ ID NO: 132.

In some embodiments of any of the polypeptides described herein, the first antigen-binding domain includes a CDR1 including SEQ ID NO: 1, a CDR2 including SEQ ID NO: 35, and a CDR3 including SEQ ID NO: 57, and the second antigen-binding domain includes a CDR1 including SEQ ID NO: 127, a CDR2 including SEQ ID NO: 130, and a CDR3 including SEQ ID NO: 133.

In some embodiments of any of the polypeptides described herein, the first antigen-binding domain includes a CDR1 including SEQ ID NO: 1, a CDR2 including SEQ ID NO: 13, and a CDR3 including SEQ ID NO: 57, and the second antigen-binding domain includes a CDR1 including SEQ ID NO: 127, a CDR2 including SEQ ID NO: 130, and a CDR3 including SEQ ID NO: 133.

In some embodiments of any of the polypeptides described herein, the polypeptide includes a sequence that is at least 80% identical to SEQ ID NO: 203. In some embodiments, the polypeptide includes SEQ ID NO: 203.

In another aspect, this disclosure features a polypeptide including: (a) a first antigen-binding domain including: (i) a CDR1 including a sequence of Formula I: G-R-T-F-S-E-Y-A-M-G (SEQ ID NO: 1); (ii) a CDR2 including a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 including a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain including: (i) a CDR1 including a sequence of Formula VII of: G-X₁-T-X₂-S-T-A-A (SEQ ID NO: 137), wherein X₁ is R or A; X₂ is F, H, or Y; (ii) a CDR2 including a sequence of Formula VIII of: I-R-W-S-X₃-G-S-A (SEQ ID NO: 138), wherein X₃ is G or A; and (iii) a CDR3 including a sequence of Formula IX: A-R-T-E-N-V-R-X₄-X₅-L-S-D-Y-A-T-X₆-P-Y-X₇-Y (SEQ ID NO: 139), wherein X₄ is S or A; X₅ is L or K; X₆ is W or Y, and X₇ is A, Q, or R.

In some embodiments of any of the polypeptides described herein, the first antigen-binding domain includes: (i) a CDR1 sequence selected from the group consisting of: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), TISWSGGATYYTRSVKG (SEQ ID NO: 44), TISWSGGSTYYTASVKG (SEQ ID NO: 228), TISWSGGATYYTASVKG (SEQ ID NO: 229), or TISWSGGGTYYTDSVKG (SEQ ID NO: 230); and (iii) a CDR3 sequence selected from the group consisting of:

(SEQ ID NO: 45)
AAAGLGTVVSEWDYDYDY,
(SEQ ID NO: 46)
AAAGLGTVVSEWDDYDY,
(SEQ ID NO: 47)
APAGLGTVVSEWDYDYDY,
(SEQ ID NO: 48)
AAADLGTVVSEWDYDYDY,
(SEQ ID NO: 49)
AAAGLGTVVSEADYDYDY,
(SEQ ID NO: 50)
AAAGLRTVVSEWDYDYDY,
(SEQ ID NO: 51)
AYAGLGTVVSEWDYDYDY,
(SEQ ID NO: 52)
AAAGLGTVVSEWDYDYDS,
(SEQ ID NO: 53)
AAAGLWTVVSEWDYDYDY,
(SEQ ID NO: 54)
AAAGLGTVVSAWDYDYDY,
(SEQ ID NO: 55)
AAAGLGTVVSEWDYDDY,
(SEQ ID NO: 56)
AAAGQGTVVSEWDYDYDY,
(SEQ ID NO: 57)
AAAGLETVVSEWDYDYDY,
(SEQ ID NO: 58)
AAARLGTVVSEWDYDYDY,
(SEQ ID NO: 57)
AAAGLETVVSEWDYDYDY;
or,
(SEQ ID NO: 60)
AAAGLGTVVSEWAYDYDA.

In some embodiments of any of the polypeptides described herein, the first antigen-binding domain includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45).

In some embodiments of any of the polypeptides described herein, the first antigen-binding domain includes a framework region 1 (FR1) sequence selected from the group consisting of:

(SEQ ID NO: 61)
MQVQLQESGGGLVQAGGSLRLSCAASG,
and
(SEQ ID NO: 62)
MQVQLQESGGGLVQAGGSLRLSCAASGSG.

In some embodiments of any of the polypeptides described herein, the first antigen-binding domain includes a sequence that is at least 80% identical to a sequence selected from SEQ ID NOs: 63-126 or 216-226. In some embodiments, the first antigen-binding domain includes a sequence that is selected from SEQ ID NOs: 63-126 or 216-226.

In some embodiments of any of the polypeptides described herein, the second antigen-binding domain includes: (i) a CDR1 sequence selected from the group consisting of: GRTFSTAA (SEQ ID NO: 140), GRTFATAA (SEQ ID NO: 141), GATFSTAA (SEQ ID NO: 142), GRTYSTAA (SEQ ID NO: 143), and GRTHSTAA (SEQ ID NO: 144); (ii) a CDR2 sequence selected from the group consisting of: IRWSGGSA (SEQ ID NO: 145), and IRWSAGSA (SEQ ID NO: 146); and iii a CDR3 sequence selected from the group consisting of:

(SEQ ID NO: 147)
ARTENVRSLLSDYATWPYDY,
(SEQ ID NO: 148)
ARTENVRSLLSDYATYPYDY,
(SEQ ID NO: 149)
ARTENVRALLSDYATWPYDY,
(SEQ ID NO: 150)
ARTENVRSKLSDYATWPYDY,
(SEQ ID NO: 151)
ARTENVRSLLSDYATWPYRY,
(SEQ ID NO: 152)
ARTENVRSLLSDYATWPYQY,
and
(SEQ ID NO: 153)
ARTENVRSLLSDYATWPYAY.

In some embodiments of any of the polypeptides described herein, the second antigen-binding domain includes: GRTFSTAA (SEQ ID NO: 140), IRWSGGSA (SEQ ID NO: 145), and ARTENVRSLLSDYATYPYDY (SEQ ID NO: 148); GRTFATAA (SEQ ID NO: 141), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRALLSDYATWPYDY (SEQ ID NO: 149); GATFSTAA (SEQ ID NO: 142), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSKLSDYATWPYDY (SEQ ID NO: 150); GRTFSTAA (SEQ ID NO: 140), IRWSAGSA (SEQ ID NO: 146), and ARTENVRSLLSDYATWPYDY (SEQ ID NO: 147); GRTYSTAA (SEQ ID NO: 143), IRWSGGSA (SEQ ID NO: 145), and ARTENVRSLLSDYATWPYDY (SEQ ID NO: 147); GRTHSTAA (SEQ ID NO: 144), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSLLSDYATWPYRY (SEQ ID NO: 151); GRTFSTAA (SEQ ID NO: 140), IRWSGGSA (SEQ ID NO: 145), and ARTENVRSLLSDYATWPYQY (SEQ ID NO: 152); or GRTFSTAA (SEQ ID NO: 140), IRWSGGSA (SEQ ID NO: 145), and ARTENVRSLLSDYATWPYAY (SEQ ID NO: 153).

In some embodiments of any of the polypeptides described herein, the antigen-binding domain includes a sequence that is at least 80% identical to a sequence selected from SEQ ID NOs: 154-165. In some embodiments, the antigen-binding domain includes a sequence that is selected from SEQ ID NOs: 154-165.

In some embodiments of any of the polypeptides described herein, the first antigen-binding domain includes a CDR1 including SEQ ID NO: 1, a CDR2 including SEQ ID NO: 5, and a CDR3 including SEQ ID NO: 45, and the second antigen-binding domain includes a CDR1 including SEQ ID NO: 140, a CDR2 including SEQ ID NO: 145, and a CDR3 including SEQ ID NO: 151.

In some embodiments of any of the polypeptides described herein, the first antigen-binding domain includes a CDR1 including SEQ ID NO: 1, a CDR2 including SEQ ID NO: 27, and a CDR3 including SEQ ID NO: 45, and the second antigen-binding domain includes a CDR1 including SEQ ID NO: 140, a CDR2 including SEQ ID NO: 145, and a CDR3 including SEQ ID NO: 151.

In some embodiments of any of the polypeptides described herein, the first antigen-binding domain includes a CDR1 including SEQ ID NO: 1, a CDR2 including SEQ ID NO: 5, and a CDR3 including SEQ ID NO: 45, and the second antigen-binding domain includes a CDR1 including SEQ ID NO: 140, a CDR2 including SEQ ID NO: 145, and a CDR3 including SEQ ID NO: 153.

In some embodiments of any of the polypeptides described herein, the first antigen-binding domain includes a CDR1 including SEQ ID NO: 1, a CDR2 including SEQ ID NO: 35, and a CDR3 including SEQ ID NO: 57, and the second antigen-binding domain includes a CDR1 including SEQ ID NO: 140, a CDR2 including SEQ ID NO: 145, and a CDR3 including SEQ ID NO: 151.

In some embodiments of any of the polypeptides described herein, the first antigen-binding domain includes a CDR1 including SEQ ID NO: 1, a CDR2 including SEQ ID NO: 13, and a CDR3 including SEQ ID NO: 57, and the second antigen-binding domain includes a CDR1 including SEQ ID NO: 140, a CDR2 including SEQ ID NO: 145, and a CDR3 including SEQ ID NO: 151.

In some embodiments of any of the polypeptides described herein, the polypeptide includes a sequence that is at least 80% identical to SEQ ID NO: 201. In some embodiments, the polypeptide includes SEQ ID NO: 201.

In some embodiments of any of the polypeptides described herein, the polypeptide includes a sequence that is at least 80% identical to SEQ ID NO: 202. In some embodiments, the polypeptide includes SEQ ID NO: 202.

In another aspect, this disclosure features a polypeptide including: (a) a first antigen-binding domain including: (i) a CDR1 including a sequence of Formula I: G-R-T-F-S-E-Y-A-M-G (SEQ ID NO: 1); (ii) a CDR2 including a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 including a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain including: (i) a CDR1 including a sequence of Formula IV of: G-F-P-V-E-X₁-X₂-X₃ (SEQ ID NO: 166), wherein X₁ is V or A, X₂ is W, Y, or A, and X₃ is R or A; (ii) a CDR2 including a sequence of Formula V of: I-E-X₄-X₅-G-H-G-X₆ (SEQ ID NO: 167), wherein X₄ is G or A, X₅ is Y or A, and X₆ is T or A; and (iii) a CDR3 including a sequence of Formula VI: N-V-X₇-D-D-X₈-X₉-L-A-Y-H-Y-D-Y (SEQ ID NO: 168), wherein X₇ is G or A, X₈ is G or N, and X₉ is Q or H.

In some embodiments of any of the polypeptides described herein, the first antigen-binding domain includes: (i) a CDR1 sequence selected from the group consisting of: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), TISWSGGATYYTRSVKG (SEQ ID NO: 44), TISWSGGSTYYTASVKG (SEQ ID NO: 228), TISWSGGATYYTASVKG (SEQ ID NO: 229), or TISWSGGGTYYTDSVKG (SEQ ID NO: 230; and iii a CDR3 se uence selected from the group consisting of:

(SEQ ID NO: 45)
AAAGLGTVVSEWDYDYDY,
(SEQ ID NO: 46)
AAAGLGTVVSEWDDYDY,
(SEQ ID NO: 47)
APAGLGTVVSEWDYDYDY,
(SEQ ID NO: 48)
AAADLGTVVSEWDYDYDY,
(SEQ ID NO: 49)
AAAGLGTVVSEADYDYDY,
(SEQ ID NO: 50)
AAAGLRTVVSEWDYDYDY,
(SEQ ID NO: 51)
AYAGLGTVVSEWDYDYDY,
(SEQ ID NO: 52)
AAAGLGTVVSEWDYDYDS,
(SEQ ID NO: 53)
AAAGLWTVVSEWDYDYDY,
(SEQ ID NO: 54)
AAAGLGTVVSAWDYDYDY,
(SEQ ID NO: 55)
AAAGLGTVVSEWDYDDY,
(SEQ ID NO: 56)
AAAGQGTVVSEWDYDYDY,
(SEQ ID NO: 57)
AAAGLETVVSEWDYDYDY,
(SEQ ID NO: 58)
AAARLGTVVSEWDYDYDY,
(SEQ ID NO: 57)
AAAGLETVVSEWDYDYDY;
or,
(SEQ ID NO: 60)
AAAGLGTVVSEWAYDYDA.

In some embodiments of any of the polypeptides described herein, the first antigen-binding domain includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45).

In some embodiments of any of the polypeptides described herein, the first antigen-binding domain includes a framework region 1 (FR1) sequence selected from the group consisting of:

(SEQ ID NO: 61)
MQVQLQESGGGLVQAGGSLRLSCAASG,
and
(SEQ ID NO: 62)
MQVQLQESGGGLVQAGGSLRLSCAASGSG.

In some embodiments of any of the polypeptides described herein, the first antigen-binding domain includes a sequence that is at least 80% identical to a sequence selected from SEQ ID NOs: 63-126 or 216-226. In some embodiments, the first antigen-binding domain includes a sequence that is selected from SEQ ID NOs: 63-126 or 216-226.

In some embodiments of any of the polypeptides described herein, the second antigen-binding domain includes: (i) a CDR1 sequence selected from the group consisting of: GFPVEVWR (SEQ ID NO: 169), GFPVEVYR (SEQ ID NO: 170), GFPVEVAR (SEQ ID NO: 171), GFPVEAWR (SEQ ID NO: 172), and GFPVEVWA (SEQ ID NO: 173); (ii) a CDR2 sequence selected from the group consisting of: IESYGHGT (SEQ ID NO: 174), IESAGHGT (SEQ ID NO: 175), IESYGHGA (SEQ ID NO: 176), and IEAYGHGT (SEQ ID NO: 177); and (iii) a CDR3 sequence selected from the group consisting of: NVKDDGQLAYHYDY (SEQ ID NO: 178), NVYDDGQLAYHYDY (SEQ ID NO: 179), NVYDDGHLAYHYDY (SEQ ID NO: 180), and NVYDDNQLAYHYDY (SEQ ID NO: 181).

In some embodiments of any of the polypeptides described herein, the second antigen-binding domain includes: GFPVEVWR (SEQ ID NO: 169), IESAGHGT (SEQ ID NO: 175), and NVYDDGQLAYHYDY (SEQ ID NO: 179); GFPVEVYR (SEQ ID NO: 170), IESYGHGT (SEQ ID NO: 174), and NVYDDGQLAYHYDY (SEQ ID NO: 179); GFPVEVAR (SEQ ID NO: 171), IESYGHGT (SEQ ID NO: 174), and NVYDDGQLAYHYDY (SEQ ID NO: 179); GFPVEAWR (SEQ ID NO: 172), IESYGHGT (SEQ ID NO: 174), and NVYDDGQLAYHYDY (SEQ ID NO: 179); GFPVEVWR (SEQ ID NO: 169), IESYGHGA (SEQ ID NO: 176), and NVYDDGQLAYHYDY (SEQ ID NO: 179); GFPVEVWR (SEQ ID NO: 169), IEAYGHGT (SEQ ID NO: 177), and NVYDDGQLAYHYDY (SEQ ID NO: 179); GFPVEVWA (SEQ ID NO: 173), IESYGHGT (SEQ ID NO: 174), and NVYDDGQLAYHYDY (SEQ ID NO: 179); GFPVEVWR (SEQ ID NO: 169), IESYGHGT (SEQ ID NO: 174), and NVYDDGHLAYHYDY (SEQ ID NO: 180); GFPVEVWR (SEQ ID NO: 169), IESYGHGT (SEQ ID NO: 174), and NVYDDGQLAYHYDY (SEQ ID NO: 179); or GFPVEVWR (SEQ ID NO: 169), IESYGHGT (SEQ ID NO: 174), and NVYDDNQLAYHYDY (SEQ ID NO: 181).

In some embodiments of any of the polypeptides described herein, the second antigen-binding domain includes a framework region 3 (FR3) sequence selected from the group consisting of: RYADSVKGRFTISRDNAKNTVYLQMNSLKPEDTAVYYC (SEQ ID NO: 182), RYADSVRGRFTISRDNAKNTVYLQMNSLKPEDTAVYYC (SEQ ID NO: 183), or RYADSVAGRFTISRDNAKNTVYLQMNSLKPEDTAVYYC (SEQ ID NO: 184).

In some embodiments of any of the polypeptides described herein, the antigen-binding domain includes a sequence that is at least 80% identical to a sequence selected from SEQ ID NOs: 185-197. In some embodiments, the antigen-binding domain includes a sequence that is selected from SEQ ID NOs: 185-197.

In some embodiments of any of the polypeptides described herein, the first antigen-binding domain includes a CDR1 including SEQ ID NO: 1, a CDR2 including SEQ ID NO: 5, and a CDR3 including SEQ ID NO: 45, and the second antigen-binding domain includes a CDR1 including SEQ ID NO: 169, a CDR2 including SEQ ID NO: 174, and a CDR3 including SEQ ID NO: 179.

In some embodiments of any of the polypeptides described herein, the first antigen-binding domain includes a CDR1 including SEQ ID NO: 1, a CDR2 including SEQ ID NO: 27, and a CDR3 including SEQ ID NO: 45, and the second antigen-binding domain includes a CDR1 including SEQ ID NO: 169, a CDR2 including SEQ ID NO: 174, and a CDR3 including SEQ ID NO: 179.

In some embodiments of any of the polypeptides described herein, the first antigen-binding domain includes a CDR1 including SEQ ID NO: 1, a CDR2 including SEQ ID NO: 5, and a CDR3 including SEQ ID NO: 45, and the second antigen-binding domain includes a CDR1 including SEQ ID NO: 169, a CDR2 including SEQ ID NO: 174, and a CDR3 including SEQ ID NO: 178.

In some embodiments of any of the polypeptides described herein, the first antigen-binding domain includes a CDR1 including SEQ ID NO: 1, a CDR2 including SEQ ID NO: 35, and a CDR3 including SEQ ID NO: 57, and the second antigen-binding domain includes a CDR1 including SEQ ID NO: 169, a CDR2 including SEQ ID NO: 174, and a CDR3 including SEQ ID NO: 179.

In some embodiments of any of the polypeptides described herein, the first antigen-binding domain includes a CDR1 including SEQ ID NO: 1, a CDR2 including SEQ ID NO: 13, and a CDR3 including SEQ ID NO: 57, and the second antigen-binding domain includes a CDR1 including SEQ ID NO: 169, a CDR2 including SEQ ID NO: 174, and a CDR3 including SEQ ID NO: 179.

In some embodiments of any of the polypeptides described herein, the polypeptide includes a sequence that is at least 80% identical to SEQ ID NO: 198. In some embodiments, the polypeptide includes SEQ ID NO: 198.

In some embodiments of any of the polypeptides described herein, the polypeptide includes a sequence that is at least 80% identical to SEQ ID NO: 199. In some embodiments, the polypeptide includes SEQ ID NO: 199.

In some embodiments of any of the polypeptides described herein, the polypeptide includes a sequence that is at least 80% identical to SEQ ID NO: 200. In some embodiments, the polypeptide includes a sequence of SEQ ID NO: 200.

In some embodiments of any of the polypeptides described herein, the polypeptide includes a sequence that is at least 80% identical to SEQ ID NO: 206. In some embodiments, the polypeptide includes a sequence of SEQ ID NO: 206.

In some embodiments of any of the polypeptides the first antigen-binding domain and/or the second antigen-binding domain is a single domain antibody. In some embodiments of any of the polypeptides described herein, the polypeptide further includes one or more additional antigen-binding domains.

In some embodiments of any of the polypeptides described herein, the polypeptide is a single-chain polypeptide.

In some embodiments of any of the polypeptides described herein, the polypeptide includes a linker disposed between the first antigen-binding domain and the second antigen-binding domain. In some embodiments, the linker includes a total of about 1 amino acid to about 25 amino acids. In some embodiments, the linker includes a sequence selected from the group consisting of: GS, GGGGS (SEQ ID NO: 207), GGGGSGGGGS (SEQ ID NO: 208), GGGGSGGGGSGGGGS (SEQ ID NO: 209), and GGGGSGGGGSGGGGSGGGGS (SEQ ID NO: 210).

In some embodiments of any of the polypeptides described herein, the polypeptide is a multi-chain polypeptide. In some embodiments, the antigen-binding domain is humanized.

In another aspect, this disclosure features a pharmaceutical composition including any of polypeptide described herein and a pharmaceutically acceptable carrier. In some embodiments, the pharmaceutical composition is formulated for intravenous administration.

In another aspect, this disclosure features a method of treating a subject having or suspected of having a coronavirus infection, wherein the method includes administering a therapeutically effective amount of any of the pharmaceutical compositions described herein. In some embodiments, the coronavirus infection is a SARS-CoV-2 infection.

In another aspect, this disclosure features a kit including any of the pharmaceutical compositions described herein.

In another aspect, this disclosure features a nucleic acid encoding any of the polypeptides described herein.

In another aspect, this disclosure features a vector including any of the nucleic acids described herein.

In another aspect, this disclosure features a host cell that includes any of the nucleic acids described herein or any of the vectors described herein.

In another aspect, this disclosure features a method of producing a polypeptide including: (a) culturing any of the host cells described herein in a culture medium under conditions sufficient to allow for the production of the polypeptide; and (b) harvesting the polypeptide from the host cell or the culture medium. In some embodiments, the method further includes isolating the polypeptide. In some embodiments, the method further includes formulating the isolated polypeptide.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Described herein, in certain embodiments, are antibodies that specifically binds SARS-CoV-2 receptor binding domain (RBD) comprising: a) a heavy chain complementarity determining region 1 (VH-CDR1) comprising an amino acid sequence GRTFSEYA (SEQ ID NO: 227), or an amino acid sequence that has 1, 2, or 3 amino acid substitutions, additions, or deletions relative to SEQ ID NO: 227; b) a heavy chain complementarity determining region 2 (VH-CDR2) comprising an amino acid sequence TISWSGGATYYTDSVKG (SEQ ID NO: 38), an amino acid sequence TISWSGGSTYYTASVKG (SEQ ID NO: 228), an amino acid sequence TISWSGGATYYTASVKG (SEQ ID NO: 229); or an amino acid sequence TISWSGGGTYYTDSVKG (SEQ ID NO: 230); and c) a heavy chain complementarity determining region 3 (VH-CDR3) comprising an amino acid sequence AAAGLGTVVSEWDYDYDY (SEQ ID NO: 45), or an amino acid sequence that has 1, 2, or 3 amino acid substitutions, additions, or deletions relative to SEQ ID NO: 45, wherein the recombinant antibody specifically binds to the SARS-CoV-2 receptor binding domain (RBD). In some embodiments, the VH-CDR1 comprises an amino acid sequence GX₁TX₂X₃X₄X₅A (SEQ ID NO: 337), wherein X₁ is R or A, X₂ is F, Y or H, X₃ is S or A, X₄ is T or E, and X₅ is Y or A. In some embodiments, the VH-CDR1 comprises an amino acid sequence GRTFSTAA (SEQ ID NO: 140), GRTFATAA (SEQ ID NO: 141), GATFSTAA (SEQ ID NO: 142), GRTYSTAA (SEQ ID NO: 143), or GRTHSTAA (SEQ ID NO: 144). In some embodiments, the VH-CDR1 comprises an amino acid sequence GRTFSEYA (SEQ ID NO: 227). In some embodiments, the VH-CDR2 comprises an amino acid sequence TISWSGGATYYTDSVKG (SEQ ID NO: 38). In some embodiments, the VH-CDR2 comprises an amino acid sequence TISWSGGSTYYTASVKG (SEQ ID NO: 228). In some embodiments, the VH-CDR3 comprises an amino acid sequence AX₆AX₇X₈X₉TVVSX₁₀X₁₁X₁₂YDYDX₁₃ (SEQ ID NO: 338), wherein X₆ is Y, P or A, X₇ is G, D, or R, X₈ is L or Q, X₉ is G, R, W, or E, X₁₀ is E or A, X₁₁ is W or A, X₁₂ is D or A, and X₁₃ is Y, S, or A. In some embodiments, the VH-CDR3 comprises an amino acid sequence AAAGLGTVVSEWDYDYDY (SEQ ID NO: 45), APAGLGTVVSEWDYDYDY (SEQ ID NO: 47), AAADLGTVVSEWDYDYDY (SEQ ID NO: 48), AAAGLGTVVSEADYDYDY (SEQ ID NO: 49), AAAGLRTVVSEWDYDYDY (SEQ ID NO: 50), AYAGLGTVVSEWDYDYDY (SEQ ID NO: 51), AAAGLGTVVSEWDYDYDS (SEQ ID NO: 52), AAAGLWTVVSEWDYDYDY (SEQ ID NO: 53), AAAGLGTVVSAWDYDYDY (SEQ ID NO: 54), AAAGQGTVVSEWDYDYDY (SEQ ID NO: 56), AAAGLETVVSEWDYDYDY (SEQ ID NO: 57), AAARLGTVVSEWDYDYDY (SEQ ID NO: 58), AAAGLETVVSEWDYDYDY (SEQ ID NO: 57); or AAAGLGTVVSEWAYDYDA (SEQ ID NO: 60). In some embodiments, the VH-CDR3 comprises an amino acid sequence AAAGLGTVVSEWDYDYDY (SEQ ID NO: 45). In some embodiments, the VH-CDR1 comprises an amino acid sequence GRTFSEYA (SEQ ID NO: 227), wherein the VH-CDR2 comprises an amino acid sequence TISWSGGATYYTDSVKG (SEQ ID NO: 38), and wherein the VH-CDR3 comprises an amino acid sequence AAAGLGTVVSEWDYDYDY (SEQ ID NO: 45). In some embodiments, the VH-CDR1 comprises an amino acid sequence GRTFSEYA (SEQ ID NO: 227), wherein the VH-CDR2 comprises an amino acid sequence TISWSGGSTYYTASVKG (SEQ ID NO: 228), and wherein the VH-CDR3 comprises an amino acid sequence AAAGLGTVVSEWDYDYDY (SEQ ID NO: 45). In some embodiments, the VH-CDR1 comprises an amino acid sequence GRTFSEYA (SEQ ID NO: 227), wherein the VH-CDR2 comprises an amino acid sequence TISWSGGATYYTASVKG (SEQ ID NO: 229), and wherein the VH-CDR3 comprises an amino acid sequence AAAGLGTVVSEWDYDYDY (SEQ ID NO: 45). In some embodiments, the VH-CDR1 comprises an amino acid sequence GRTFSEYA (SEQ ID NO: 227), wherein the VH-CDR2 comprises an amino acid sequence TISWSGGATYYTDSVKG (SEQ ID NO: 38), and wherein the VH-CDR3 comprises an amino acid sequence AAAGQGTVVSEWDYDYDY (SEQ ID NO: 56). In some embodiments, the VH-CDR1 comprises an amino acid sequence GRTFSEYA (SEQ ID NO: 227), wherein the VH-CDR2 comprises an amino acid sequence TISWSGGGTYYTDSVKG (SEQ ID NO: 230), and wherein the VH-CDR3 comprises an amino acid sequence AAAGQGTVVSEWDYDYDY (SEQ ID NO: 56). In some embodiments, the antibody is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the antibody is a bispecific antibody. In some embodiments, the antibody is a VHH antibody. In some embodiments, the antibody is humanized. In some embodiments, the antibody specifically binds to the SARS-CoV-2 receptor binding domain (RBD) with a dissociation constant (K_{D}) of less than about 50 nanomolar. In some embodiments, the antibody specifically binds to the SARS-CoV-2 receptor binding domain (RBD) with a dissociation constant (K_{D}) of less than about 10 nanomolar. In some embodiments, the antibody specifically binds to the SARS-CoV-2 receptor binding domain (RBD) with a dissociation constant (K_{D}) of less than about 200 picomolar.

Described herein, in certain embodiments, are pharmaceutical compositions comprising the antibody described herein and pharmaceutically acceptable excipient. In some embodiments, the pharmaceutical composition is formulated for inhalation. In some embodiments, the pharmaceutical composition is formulated for subcutaneous administration.

Described herein, in certain embodiments, are methods of immunizing a subject against SARS-CoV-2 comprising administering the antibody described herein. Described herein, in certain embodiments, are methods of treating a subject with SARS-CoV-2 comprising administering the antibody described herein. Described herein, in certain embodiments, are methods of preventing SARS-CoV-2 in a subject comprising administering the antibody described herein. In some embodiments, the subject is a human. In some embodiments, the subject is a cat, a dog, a goat, a human, a non-human primate, a rodent, a pig, or a sheep.

Described herein, in certain embodiments, are methods of treating a coronavirus comprising administering the antibody of described herein. In some embodiments, the coronavirus is SARS-CoV, MERS-CoV, CoV-229E, HCoV-NL63, HCoV-OC43, or HCoV-HKU1.

Described herein, in certain embodiments, are methods of immunizing an individual against SARS-CoV-2 comprising administering the antibody described herein.

Described herein, in certain embodiments, are methods of treating an individual with SARS-CoV-2 comprising administering the antibody described herein.

Other features and advantages of the invention will be apparent from the following detailed description and figures, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows binding activity to SARS-CoV spike glycoprotein, SARS-CoV-2 spike glycoprotein and/or ACE2-competitive binding activity for each construct tested. The “xmid improve from ctrl (log 10)” value indicates the estimated EC₅₀ in log 10 space for each construct relative to the EC₅₀ for a control. The data shown the columns to the right of “xmid improve from ctlr (log 10)” represent the data from different binding/competitive binding assays described in the “Antigen descriptor” column with the xmid/EC50 estimate with variance+95% confidence intervals for each construct shown.

FIG. 2 shows a plot of apparent affinities of 115 variants of VHH-72 for the SARS-CoV-2 RBD as measured by ELISA. Round 0 reflects the monovalent wildtype VHH-72, followed by three sequential rounds of human-driven affinity optimization and one round of sequence pattern-driven optimization (Markov). An affinity of 10 μg/ml or greater cannot be accurately measured and thus was assigned a value of 10 μg/ml.

FIG. 3 shows the effects of neutral amino acid mutagenesis of CDR3 residues on RBD binding affinity by ELISA. In the structural models, residues are labeled reflecting the change in EC50 caused by the neutral mutant relative to the wildtype, where equivalent binding equals 1 and loss of binding equals 0.

FIG. 4 shows molecular visualization of position 57 in VHH-72 with the wildtype structure (left) and model of the high-affinity mutant VHH-72(S57G) (right) juxtaposed.

### DETAILED DESCRIPTION OF THE INVENTION

Provided herein are antigen-binding domains that bind specifically to coronavirus spike glycoprotein, polypeptides including one or more of the same, pharmaceutical compositions including these polypeptides, and methods of making and uses these polypeptides and pharmaceutical compositions. Described herein, in some embodiments, are polypeptides (e.g., antibodies) for SARS-CoV, MERS-CoV, CoV-229E, HCoV-NL63, HCoV-OC43, or HCoV-HKU1. Described herein, in some embodiments, are polypeptides (e.g., antibodies) for SARS-CoV-2.

Provided herein are polypeptides that include an antigen-binding domain that binds specifically to a coronavirus spike glycoprotein, wherein the polypeptide includes: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A.

Also provided herein are polypeptides that include an antigen-binding domain that binds specifically to a coronavirus spike glycoprotein, wherein the single polypeptide includes: (i) a CDR1 comprising a sequence of Formula IV of: (i) a CDR1 comprising a sequence of Formula IV of: G-F-P-V-Y-S-W-N(SEQ ID NO: 127); (ii) a CDR2 comprising a sequence of Formula V of: I-E-S-H-X₁-D-S-T (SEQ ID NO: 128), wherein X₁ is G or A; and (iii) a CDR3 comprising a sequence of Formula VI: Y-V-W-V-X₂-H-T-Y-Y-G-Q (SEQ ID NO: 129), wherein X₂ is G or A.

Also provided herein are polypeptides that include an antigen-binding domain that binds specifically to a coronavirus spike glycoprotein, wherein the single polypeptide includes: (i) a CDR1 comprising a sequence of Formula VII of: G-X₁-T-X₂-S-T-A-A (SEQ ID NO: 137), wherein X₁ is R or A; X₂ is F, H, or Y; (ii) a CDR2 comprising a sequence of Formula VIII of: I-R-W-S-X₃-G-S-A (SEQ ID NO: 138), wherein X₃ is G or A; and (iii) a CDR3 comprising a sequence of Formula IX: A-R-T-E-N-V-R-X₄-X₅-L-S-D-Y-A-T-X₆-P-Y-X₇-Y (SEQ ID NO: 139), wherein X₄ is S or A; X₅ is L or K; X₆ is W or Y, and X₇ is A, Q, or R.

Also provided herein are polypeptides that include an antigen-binding domain that binds specifically to a coronavirus spike glycoprotein, wherein the single polypeptide includes: (i) a CDR1 comprising a sequence of Formula X of: G-F-P-V-E-X₁-X₂-X₃ (SEQ ID NO: 166), wherein X₁ is V or A, X₂ is W, Y, or A, and X₃ is R or A; (ii) a CDR2 comprising a sequence of Formula XI of: I-E-X₄-X₅-G-H-G-X₆ (SEQ ID NO: 167), wherein X₄ is G or A, X₅ is Y or A, and X₆ is T or A; and (iii) a CDR3 comprising a sequence of Formula XII: N-V-X₇-D-D-X₈-X₉-L-A-Y-H-Y-D-Y (SEQ ID NO: 168), wherein X₇ is G or A, X₈ is G or N, and X₉ is Q or H.

Provided herein are polypeptides that includes (a) a first antigen-binding domain that binds specifically to a coronavirus spike glycoprotein that includes: (a) a first antigen-binding domain comprising: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain comprising: (i) a CDR1 comprising a sequence of Formula IV of: G-F-P-V-Y-S-W-N(SEQ ID NO: 127); (ii) a CDR2 comprising a sequence of Formula V of: I-E-S-H-X₁-D-S-T (SEQ ID NO: 128), wherein X₁ is G or A; and (iii) a CDR3 comprising a sequence of Formula VI: Y-V-W-V-X₂-H-T-Y-Y-G-Q (SEQ ID NO: 129), wherein X₂ is G or A.

Provided herein are polypeptides that includes (a) a first antigen-binding domain that binds specifically to a coronavirus spike glycoprotein that includes: (a) a first antigen-binding domain comprising: (a) a first antigen-binding domain comprising: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain comprising: (i) a CDR1 comprising a sequence of Formula VII of: G-X₁-T-X₂-S-T-A-A (SEQ ID NO: 137), wherein X₁ is R or A; X₂ is F, H, or Y; (ii) a CDR2 comprising a sequence of Formula VIII of: I-R-W-S-X₃-G-S-A (SEQ ID NO: 138), wherein X₃ is G or A; and (iii) a CDR3 comprising a sequence of Formula IX: A-R-T-E-N-V-R-X₄-X₅-L-S-D-Y-A-T-X₆-P-Y-X₇-Y (SEQ ID NO: 139), wherein X₄ is S or A; X₅ is L or K; X₆ is W or Y, and X₇ is A, Q, or R.

Provided herein are polypeptides that includes (a) a first antigen-binding domain that binds specifically to a coronavirus spike glycoprotein that includes: (a) a first antigen-binding domain comprising: (a) a first antigen-binding domain comprising: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain comprising: (i) a CDR1 comprising a sequence of Formula IV of: G-F-P-V-E-X₁-X₂-X₃ (SEQ ID NO: 166), wherein X₁ is V or A, X₂ is W, Y, or A, and X₃ is R or A; (ii) a CDR2 comprising a sequence of Formula V of: I-E-X₄-X₅-G-H-G-X₆ (SEQ ID NO: 167), wherein X₄ is G or A, X₅ is Y or A, and X₆ is T or A; and (iii) a CDR3 comprising a sequence of Formula VI: N-V-X₇-D-D-X₈-X₉-L-A-Y-H-Y-D-Y (SEQ ID NO: 168), wherein X₇ is G or A, X₈ is G or N, and X₉ is Q or H.

In some embodiments described herein, the antigen-binding domain or one or both of the first or second antigen-binding domain described herein binds to a SARS-CoV glycoprotein or SARS-CoV-2 glycoprotein with a K_{D} of about 1 pM to about 1 mM, about 1 pM to about 800 uM, about 1 pM to about 600 uM, about 1 pM to about 400 uM, about 1 pM to about 200 uM, about 1 pM to about 100 uM, about 1 pM to about 50 uM, about 1 pM to about 10 uM, about 1 pM to about 1 uM, about 1 pM to about 800 nM, about 1 pM to about 600 nM, about 1 pM to about 400 nM, about 1 pM to about 200 nM, about 1 pM to about 100 nM, about 1 pM to about 50 nM, about 1 pM to about 10 nM, about 1 pM to about 1 nM, about 1 pM to about 800 pM, about 1 pM to about 600 pM, about 1 pM to about 400 pM, about 1 pM to about 200 pM, about 200 pM to about 1 mM, about 200 pM to about 800 uM, about 200 pM to about 600 uM, about 200 pM to about 400 uM, about 200 pM to about 200 uM, about 200 pM to about 100 uM, about 200 pM to about 50 uM, about 200 pM to about 10 uM, about 200 pM to about 1 uM, about 200 pM to about 800 nM, about 200 pM to about 600 nM, about 200 pM to about 400 nM, about 200 pM to about 200 nM, about 200 pM to about 100 nM, about 200 pM to about 50 nM, about 200 pM to about 10 nM, about 200 pM to about 1 nM, about 200 pM to about 800 pM, about 200 pM to about 600 pM, about 200 pM to about 400 pM, about 400 pM to about 1 mM, about 400 pM to about 800 uM, about 400 pM to about 600 uM, about 400 pM to about 400 uM, about 400 pM to about 200 uM, about 400 pM to about 100 uM, about 400 pM to about 50 uM, about 400 pM to about 10 uM, about 400 pM to about 1 uM, about 400 pM to about 800 nM, about 400 pM to about 600 nM, about 400 pM to about 400 nM, about 400 pM to about 200 nM, about 400 pM to about 100 nM, about 400 pM to about 50 nM, about 400 pM to about 10 nM, about 400 pM to about 1 nM, about 400 pM to about 800 pM, about 400 pM to about 600 pM, about 600 pM to about 1 mM, about 600 pM to about 800 uM, about 600 pM to about 600 uM, about 600 pM to about 400 uM, about 600 pM to about 200 uM, about 600 pM to about 100 uM, about 600 pM to about 50 uM, about 600 pM to about 10 uM, about 600 pM to about 1 uM, about 600 pM to about 800 nM, about 600 pM to about 600 nM, about 600 pM to about 400 nM, about 600 pM to about 200 nM, about 600 pM to about 100 nM, about 600 pM to about 50 nM, about 400 pM to about 10 nM, about 600 pM to about 1 nM, about 600 pM to about 800 pM, about 800 pM to about 1 mM, about 800 pM to about 800 uM, about 800 pM to about 600 uM, about 800 pM to about 400 uM, about 800 pM to about 200 uM, about 800 pM to about 100 uM, about 800 pM to about 50 uM, about 800 pM to about 10 uM, about 800 pM to about 1 uM, about 800 pM to about 800 nM, about 800 pM to about 600 nM, about 800 pM to about 400 nM, about 800 pM to about 200 nM, about 800 pM to about 100 nM, about 800 pM to about 50 nM, about 800 pM to about 10 nM, about 800 pM to about 1 nM, about 1 nM to about 1 mM, about 1 nM to about 800 uM, about 1 nM to about 600 uM, about 1 nM to about 400 uM, about 1 nM to about 200 uM, about 1 nM to about 100 uM, about 1 nM to about 50 uM, about 1 nM to about 10 uM, about 1 nM to about 1 uM, about 1 nM to about 800 nM, about 1 nM to about 600 nM, about 1 nM to about 400 nM, about 1 nM to about 200 nM, about 1 nM to about 100 nM, about 1 nM to about 50 nM, about 1 nM to about 10 nM, about 10 nM to about 1 mM, about 10 nM to about 800 uM, about 10 nM to about 600 uM, about 10 nM to about 400 uM, about 10 nM to about 200 uM, about 10 nM to about 100 uM, about 10 nM to about 50 uM, about 10 nM to about 10 uM, about 10 nM to about 1 uM, about 10 nM to about 800 nM, about 10 nM to about 600 nM, about 10 nM to about 400 nM, about 10 nM to about 200 nM, about 10 nM to about 100 nM, about 10 nM to about 50 nM, about 50 nM to about 1 mM, about 50 nM to about 800 uM, about 50 nM to about 600 uM, about 50 nM to about 400 uM, about 50 nM to about 200 uM, about 50 nM to about 100 uM, about 50 nM to about 50 uM, about 50 nM to about 10 uM, about 50 nM to about 1 uM, about 50 nM to about 800 nM, about 50 nM to about 600 nM, about 50 nM to about 400 nM, about 50 nM to about 200 nM, about 50 nM to about 100 nM, about 100 nM to about 1 mM, about 100 nM to about 800 uM, about 100 nM to about 600 uM, about 100 nM to about 400 uM, about 100 nM to about 200 uM, about 100 nM to about 100 uM, about 100 nM to about 50 uM, about 100 nM to about 10 uM, about 100 nM to about 1 uM, about 100 nM to about 800 nM, about 100 nM to about 600 nM, about 100 nM to about 400 nM, about 100 nM to about 200 nM, about 200 nM to about 1 mM, about 200 nM to about 800 uM, about 200 nM to about 600 uM, about 200 nM to about 400 uM, about 200 nM to about 200 uM, about 200 nM to about 100 uM, about 200 nM to about 50 uM, about 200 nM to about 10 uM, about 200 nM to about 1 uM, about 200 nM to about 800 nM, about 200 nM to about 600 nM, about 200 nM to about 400 nM, about 400 nM to about 1 mM, about 400 nM to about 800 uM, about 400 nM to about 600 uM, about 400 nM to about 400 uM, about 400 nM to about 200 uM, about 400 nM to about 100 uM, about 400 nM to about 50 uM, about 400 nM to about 10 uM, about 400 nM to about 1 uM, about 400 nM to about 800 nM, about 400 nM to about 600 nM, about 600 nM to about 1 mM, about 600 nM to about 800 uM, about 600 nM to about 600 uM, about 600 nM to about 400 uM, about 600 nM to about 200 uM, about 600 nM to about 100 uM, about 600 nM to about 50 uM, about 600 nM to about 10 uM, about 600 nM to about 1 uM, about 600 nM to about 800 nM, about 800 nM to about 1 mM, about 800 nM to about 800 uM, about 800 nM to about 600 uM, about 800 nM to about 400 uM, about 800 nM to about 200 uM, about 800 nM to about 100 uM, about 800 nM to about 50 uM, about 800 nM to about 10 uM, about 800 nM to about 1 uM, about 1 uM to about 1 mM, about 1 uM to about 800 uM, about 1 uM to about 600 uM, about 1 uM to about 400 uM, about 1 uM to about 200 uM, about 1 uM to about 100 uM, about 1 uM to about 50 uM, about 1 uM to about 10 uM, about 10 uM to about 1 mM, about 10 uM to about 800 uM, about 10 uM to about 600 uM, about 10 uM to about 400 uM, about 10 uM to about 200 uM, about 10 uM to about 100 uM, about 10 uM to about 50 uM, about 50 uM to about 1 mM, about 50 uM to about 800 uM, about 50 uM to about 600 uM, about 50 uM to about 400 uM, about 50 uM to about 200 uM, about 50 uM to about 100 uM, about 100 uM to about 1 mM, about 100 uM to about 800 uM, about 100 uM to about 600 uM, about 100 uM to about 400 uM, about 100 uM to about 200 uM, about 200 uM to about 1 mM, about 200 uM to about 800 uM, about 200 uM to about 600 uM, about 200 uM to about 400 uM, about 400 uM to about 1 mM, about 400 uM to about 800 uM, about 400 uM to about 600 uM, about 600 uM to about 1 mM, about 600 uM to about 800 uM, or about 600 uM to about 1 mM (e.g., as measured in phosphate buffered saline using surface plasmon resonance).

In some embodiments, any of the polypeptides described herein can have an EC₅₀ value for competitive binding to ACE2 of about 0.001 ug/mL to about 0.1 ug/mL, about 0.001 ug/mL to about 0.8 ug/mL, about 0.001 ug/mL to about 0.08 ug/mL, about 0.001 ug/mL to about 0.06 ug/mL, about 0.001 ug/mL to about 0.04 ug/mL, about 0.001 ug/mL to about 0.02 ug/mL, about 0.001 ug/mL to about 0.01 ug/mL, about 0.001 ug/mL to about 0.008 ug/mL, about 0.001 ug/mL to about 0.006 ug/mL, about 0.001 ug/mL to about 0.004 ug/mL, about 0.002 ug/mL to about 0.1 ug/mL, about 0.002 ug/mL to about 0.08 ug/mL, about 0.002 ug/mL to about 0.06 ug/mL, about 0.002 ug/mL to about 0.04 ug/mL, about 0.002 ug/mL to about 0.02 ug/mL, about 0.002 ug/mL to about 0.01 ug/mL, about 0.002 ug/mL to about 0.008 ug/mL, about 0.002 ug/mL to about 0.006 ug/mL, about 0.002 ug/mL to about 0.004 ug/mL, about 0.004 ug/mL to about 0.1 ug/mL, about 0.004 ug/mL to about 0.08 ug/mL, about 0.004 ug/mL to about 0.06 ug/mL, about 0.004 ug/mL to about 0.04 ug/mL, about 0.004 ug/mL to about 0.02 ug/mL, about 0.004 ug/mL to about 0.01 ug/mL, about 0.004 ug/mL to about 0.008 ug/mL, about 0.004 ug/mL to about 0.006 ug/mL, about 0.006 ug/mL to about 0.1 ug/mL, about 0.006 ug/mL to about 0.08 ug/mL, about 0.006 ug/mL to about 0.06 ug/mL, about 0.006 ug/mL to about 0.04 ug/mL, about 0.006 ug/mL to about 0.02 ug/mL, about 0.006 ug/mL to about 0.01 ug/mL, about 0.006 ug/mL to about 0.008 ug/mL, about 0.008 ug/mL to about 0.1 ug/mL, about 0.008 ug/mL to about 0.08 ug/mL, about 0.008 ug/mL to about 0.06 ug/mL, about 0.008 ug/mL to about 0.04 ug/mL, about 0.008 ug/mL to about 0.02 ug/mL, about 0.008 ug/mL to about 0.01 ug/mL, about 0.01 ug/mL to about 0.1 ug/mL, about 0.01 ug/mL to about 0.08 ug/mL, about 0.01 ug/mL to about 0.06 ug/mL, about 0.01 ug/mL to about 0.04 ug/mL, about 0.01 ug/mL to about 0.02 ug/mL, about 0.02 ug/mL to about 0.1 ug/mL, about 0.02 ug/mL to about 0.08 ug/mL, about 0.02 ug/mL to about 0.06 ug/mL, about 0.02 ug/mL to about 0.04 ug/mL, about 0.04 ug/mL to about 0.1 ug/mL, about 0.04 ug/mL to about 0.08 ug/mL, about 0.04 ug/mL to about 0.06 ug/mL, about 0.06 ug/mL to about 0.1 ug/mL, about 0.06 ug/mL to about 0.8 ug/mL, or about 0.08 ug/mL to about 0.1 ug/mL.

### Definitions

The term “a” and “an” refers to one or more (i.e., at least one) of the grammatical object of the article. By way of example, “a polypeptide” encompasses one or more polypeptides.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

Unless otherwise specified, a “nucleotide sequence encoding a protein” includes all nucleotide sequences that are degenerate versions of each other and thus encode the same amino acid sequence.

The term “exogenous” refers to any material introduced from or originating from outside a cell, a tissue or an organism that is not produced by or does not originate from the same cell, tissue, or organism in which it is being introduced.

The term “transduced”, “transfected”, or “transformed” refers to a process by which exogenous nucleic acid is introduced or transferred into a cell. A “transduced,” “transfected,” or “transformed” cell (e.g., a mammalian cell, a hepatocyte) is one that has been transduced, transfected or transformed with exogenous nucleic acid (e.g., an expression vector) that includes an exogenous nucleic acid encoding a polypeptide).

The term “subject” is intended to include any mammal. In some embodiments, the subject is a cat, a dog, a goat, a human, a non-human primate, a rodent (e.g., a mouse or a rat), a pig, or a sheep. In some embodiments, the subject has an increased risk of mortality due to coronavirus infection. In some embodiments, the subject is determined to be at increased risk of infection of coronavirus infection. In some embodiments, the subject has a profession that exposes him or her to persons having a coronavirus infection.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

The term “antigen-binding domain” is used to refer to one or more antibody variable domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

Unless otherwise specified, a “nucleotide sequence encoding a protein” includes all nucleotide sequences that are degenerate versions of each other and thus encode the same amino acid sequence.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

The term “exogenous” refers to any material introduced from or originating from outside a cell, a tissue, or an organism that is not produced by or does not originate from the same cell, tissue, or organism in which it is being introduced.

The term “antibody” refers to a protein with an immunoglobulin fold that specifically binds to an antigen via its variable region or regions. The term “antibody” is used herein in the broadest sense and encompasses monoclonal antibodies, polyclonal antibodies, dimers, multimers, multispecific antibodies (e.g., bispecific antibodies), and antibody fragments, so long as they exhibit the desired biological activity (Miller et al., J. Immunol. 170:4854-4861, 2003). Antibodies may be murine, human, humanized, chimeric, or derived from other species. (Janeway, C., Travers, P., Walport, M., Shlomchik (2001) Immunol. Biology, 5th Ed., Garland Publishing, New York). A target antigen generally has numerous binding sites, also called epitopes, recognized by CDRs (complementarity determining regions) on multiple antibodies. Each antibody that specifically binds to a different epitope has a different structure. Thus, one antigen may have more than one corresponding antibody. An antibody can, e.g., include a full-length immunoglobulin molecule or an immunologically active portion of a full-length immunoglobulin molecule, i.e., a molecule that contains an antigen binding site that immunospecifically binds an antigen of a target of interest or part thereof. The immunoglobulin disclosed herein can be of any type (e.g., IgG, IgE, IgM, IgD, and IgA), class (e.g., IgG₁, IgG₂, IgG₃, IgG₄, IgA₁ and IgA₂) or subclass of immunoglobulin molecule. The immunoglobulins can be derived from any species. In one aspect, however, the immunoglobulin is of human, murine, or rabbit origin.

Antibodies bound to various types of molecules, such as polyethylene glycols (PEGs), may be used as modified antibodies. Methods for modifying antibodies are already established in the art.

The term “antibody fragments” refers to a portion of a full-length antibody or a polypeptide that includes a portion of a full-length antibody, that retains antigen-binding activity via its variable region or regions. Examples of antibody fragments include Fab, Fab′, F(ab′)₂, and Fv fragments; diabodies; linear antibodies; minibodies (Olafsen et al., Protein Eng. Design & Sel. 17(4):315-323, 2004), fragments produced by a Fab expression library, single-chain antibody molecules; and multispecific antibodies formed from antibody fragments.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDRH1, CDRH2, CDRH3) and three CDRs in each light chain variable region (CDRL1, CDRL2, CDRL3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The “framework regions” or “FRs” of different light immunoglobulin chains and different heavy immunoglobulin chains are relatively conserved within different antibodies produced by a mammal. The framework regions of light and heavy immunoglobulin chains serve to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well-known definitions in the art, e.g., Kabat.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

The term “treating” means a reduction in the number, frequency, severity, and/or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

The term “administer” refers to a method of polypeptides or compositions to the desired site of biological action. These methods include, but are not limited to, topical delivery, parenteral delivery, intravenous delivery, intradermal delivery, intramuscular delivery, colonic delivery, rectal delivery, or intraperitoneal delivery. In one embodiment, the compositions described herein are administered intravenously. In some embodiments, the compositions described herein are administered through inhalation. In some embodiments, the compositions described herein are administered subcutaneously.

The term “transduced”, “transfected”, or “transformed” refers to a process by which an exogenous nucleic acid is introduced or transferred into a cell. A “transduced”, “transfected,” or “transformed” cell (e.g., mammalian cell) is one that has been transduced, transfected, or transformed with exogenous nucleic acid (e.g., a vector) that includes an exogenous nucleic acid encoding any of the polypeptides described herein.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), alphatic family (e.g., alanine, valine, leucine and isoleucine), aromatic family (e.g., phenylalanine, tryptophan, and tyrosine).

The term “promoter” means a DNA sequence recognized by enzymes/proteins in a cell (e.g., a mammalian cell, a hepatocyte) required to initiate the transcription of an operably linked coding sequence (e.g., a nucleic acid encoding a polypeptide (e.g., any of the exemplary polypeptides described herein). A promoter typically refers, to e.g. a nucleotide sequence to which an RNA polymerase and/or any associated factor binds and at which transcription is initiated. The promoter can be constitutive, inducible, or tissue-specific (e.g., a liver-specific promoter).

The term “enhancer” refers to a nucleotide sequence that can increase the transcription of an operably linked nucleic acid (e.g., a nucleic acid encoding a polypeptide (e.g., any of the exemplary polypeptides described herein). An enhancer can increase the level of transcription by providing additional binding sites for transcription-associated proteins (e.g., transcription factors). Unlike promoters, enhancers can act at distances further away from the transcription start site (e.g., as compared to a promoter).

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 or Megalign. The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The term “affinity” refers to the strength of the sum of all non-covalent interactions between an antigen-binding site and its antigen. Unless otherwise indicated, “affinity” refers to intrinsic binding affinity, which reflects a 1:1 interaction between an antigen-binding domain and an antigen. Affinity can be measured, e.g., using surface plasmon resonance (SPR) technology (e.g., BIACORE®) or biolayer interferometry (e.g., FORTEBIO®). Additional methods for determining the affinity of an antigen-binding domain and its antigen are known in the art.

The term “single-chain polypeptide” means a polypeptide comprising a single polypeptide chain.

The term “multi-chain polypeptide” means a complex of two or more (e.g., 2, 3, 4, 5, 6, 7, or 8) polypeptide chains (e.g., the same or different polypeptide chains) that covalently and/or non-covalently associate with each other. For example, two or more polypeptide chains of a multi-chain polypeptide can associate through the use of two domains that associate with each other (e.g., two Fc domains or IL-15 and the sushi domain of IL-15 receptor alpha).

### Polypeptides Including a Minimum of One Antigen-Binding Domain

Provided herein are polypeptides that include an antigen-binding domain that binds specifically to a coronavirus spike glycoprotein, wherein the polypeptide includes: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A.

In some embodiments, the polypeptides include an antigen-binding domain that binds specifically to a coronavirus spike glycoprotein where the polypeptide includes: (i) a CDR1 sequence selected from the group consisting of: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), TISWSGGATYYTRSVKG (SEQ ID NO: 44), TISWSGGSTYYTASVKG (SEQ ID NO: 228), TISWSGGATYYTASVKG (SEQ ID NO: 229), or TISWSGGGTYYTDSVKG (SEQ ID NO: 230); and (iii) a CDR3 sequence selected from the group consisting of: AAAGLGTVVSEWDYDYDY (SEQ ID NO: 45), AAAGLGTVVSEWDDYDY (SEQ ID NO: 46), APAGLGTVVSEWDYDYDY (SEQ ID NO: 47), AAADLGTVVSEWDYDYDY (SEQ ID NO: 48), AAAGLGTVVSEADYDYDY (SEQ ID NO: 49), AAAGLRTVVSEWDYDYDY (SEQ ID NO: 50), AYAGLGTVVSEWDYDYDY (SEQ ID NO: 51), AAAGLGTVVSEWDYDYDS (SEQ ID NO: 52), AAAGLWTVVSEWDYDYDY (SEQ ID NO: 53), AAAGLGTVVSAWDYDYDY (SEQ ID NO: 54), AAAGLGTVVSEWDYDDY (SEQ ID NO: 55), AAAGQGTVVSEWDYDYDY (SEQ ID NO: 56), AAAGLETVVSEWDYDYDY (SEQ ID NO: 57), AAARLGTVVSEWDYDYDY (SEQ ID NO: 58), AAAGLETVVSEWDYDYDY (SEQ ID NO: 57); or AAAGLGTVVSEWAYDYDA (SEQ ID NO: 60).

In some embodiments, the polypeptides include an antigen-binding domain that binds specifically to a coronavirus spike glycoprotein where the polypeptide includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45). In some embodiments, the polypeptides do not include an antigen-binding domain that includes the three CDRs of: GRTFSEYAMG (SEQ ID NO: 1), TISWSGGATYTTDSVKG (SEQ ID NO: 43), and AAAGLGTVVSEWDYDYDY (SEQ ID NO: 45).

In some embodiments, the polypeptides include an antigen-binding domain that binds specifically to a coronavirus spike glycoprotein where the polypeptide includes: a framework region 1 (FR1) sequence selected from the group consisting of:

(SEQ ID NO: 61)
MQVQLQESGGGLVQAGGSLRLSCAASG,
and
(SEQ ID NO: 62)
MQVQLQESGGGLVQAGGSLRLSCAASGSG.

In some embodiments, the polypeptides include an antigen-binding domain that binds specifically to a coronavirus spike glycoprotein, where the antigen-binding domain includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 63-127.

Provided herein are polypeptides that include an antigen-binding domain that binds specifically to a coronavirus spike glycoprotein, wherein the single polypeptide includes: (i) a CDR1 comprising a sequence of Formula IV of: G-F-P-V-Y-S-W-N(SEQ ID NO: 127); (ii) a CDR2 comprising a sequence of Formula V of: I-E-S-H-X₁-D-S-T (SEQ ID NO: 128), wherein X₁ is G or A; and (iii) a CDR3 comprising a sequence of Formula VI: Y-V-W-V-X₂-H-T-Y-Y-G-Q (SEQ ID NO: 129), wherein X₂ is G or A.

In some embodiments, the polypeptides include an antigen-binding domain that binds specifically to a coronavirus spike glycoprotein where the polypeptide includes: (i) a CDR1 sequence selected from the group consisting of: (i) a CDR1 sequence selected from the group consisting of: GFPVYSWN (SEQ ID NO: 127); (ii) a CDR2 sequence selected from the group consisting of: IESHGDST (SEQ ID NO: 130), and IESYAHGT (SEQ ID NO: 131); and (iii) a CDR3 sequence selected from the group consisting of: YVWVGHTYYGQ (SEQ ID NO: 132), and YVWVAHTYYGQ (SEQ ID NO: 133).

In some embodiments, the polypeptides include an antigen-binding domain that binds specifically to a coronavirus spike glycoprotein where the polypeptide includes: GFPVYSWN (SEQ ID NO: 127), IESHGDST (SEQ ID NO: 130), and YVWVAHTYYGQ (SEQ ID NO: 133); GFPVYSWN (SEQ ID NO: 127), IESHADST (SEQ ID NO: 231), and YVWVGHTYYGQ (SEQ ID NO: 132); or GFPVYSWN (SEQ ID NO: 127), IESHADST (SEQ ID NO: 231), and YVWVAHTYYGQ (SEQ ID NO: 133). In some embodiments, the polypeptides does not include an antigen-binding domain comprising the three CDRs of: GFPVYSWN (SEQ ID NO: 127), IESHGDST (SEQ ID NO: 130), and YVWVGHTYYGQ (SEQ ID NO: 132).

In some embodiments, the polypeptides include an antigen-binding domain that binds specifically to a coronavirus spike glycoprotein, where the antigen-binding domain includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 134-136.

Provided herein are polypeptides that include an antigen-binding domain that binds specifically to a coronavirus spike glycoprotein, wherein the single polypeptide includes: (i) a CDR1 comprising a sequence of Formula VII of: G-X₁-T-X₂-S-T-A-A (SEQ ID NO: 137), wherein X₁ is R or A; X₂ is F, H, or Y; (ii) a CDR2 comprising a sequence of Formula VIII of: I-R-W-S-X₃-G-S-A (SEQ ID NO: 138), wherein X₃ is G or A; and (iii) a CDR3 comprising a sequence of Formula IX: A-R-T-E-N-V-R-X₄-X₅-L-S-D-Y-A-T-X₆-P-Y-X₇-Y (SEQ ID NO: 139), wherein X₄ is S or A; X₅ is L or K; X₆ is W or Y, and X₇ is A, Q, or R.

In some embodiments, the polypeptides include an antigen-binding domain that binds specifically to a coronavirus spike glycoprotein where the polypeptide includes: (i) a CDR1 sequence selected from the group consisting of: (i) a CDR1 sequence selected from the group consisting of: GRTFSTAA (SEQ ID NO: 140), GRTFATAA (SEQ ID NO: 141), GATFSTAA (SEQ ID NO: 142), GRTYSTAA (SEQ ID NO: 143), and GRTHSTAA (SEQ ID NO: 144); (ii) a CDR2 sequence selected from the group consisting of: IRWSGGSA (SEQ ID NO: 145), and IRWSAGSA (SEQ ID NO: 146); and (iii) a CDR3 sequence selected from the group consisting of: ARTENVRSLLSDYATWPYDY (SEQ ID NO: 147), ARTENVRSLLSDYATYPYDY (SEQ ID NO: 148), ARTENVRALLSDYATWPYDY (SEQ ID NO: 149), ARTENVRSKLSDYATWPYDY (SEQ ID NO: 150), ARTENVRSLLSDYATWPYRY (SEQ ID NO: 151), ARTENVRSLLSDYATWPYQY (SEQ ID NO: 152), and ARTENVRSLLSDYATWPYAY (SEQ ID NO: 153).

In some embodiments, the polypeptides include an antigen-binding domain that binds specifically to a coronavirus spike glycoprotein where the polypeptide includes: GRTFSTAA (SEQ ID NO: 140), IRWSGGSA (SEQ ID NO: 145), and ARTENVRSLLSDYATYPYDY (SEQ ID NO: 148); GRTFATAA (SEQ ID NO: 141), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRALLSDYATWPYDY (SEQ ID NO: 149); GATFSTAA (SEQ ID NO: 142), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSKLSDYATWPYDY (SEQ ID NO: 150); GRTFSTAA (SEQ ID NO: 140), IRWSAGSA (SEQ ID NO: 146), and ARTENVRSLLSDYATWPYDY (SEQ ID NO: 147); GRTYSTAA (SEQ ID NO: 143), IRWSGGSA (SEQ ID NO: 145), and ARTENVRSLLSDYATWPYDY (SEQ ID NO: 147); GRTHSTAA (SEQ ID NO: 144), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSLLSDYATWPYRY (SEQ ID NO: 151); GRTFSTAA (SEQ ID NO: 140), IRWSGGSA (SEQ ID NO: 145), and ARTENVRSLLSDYATWPYQY (SEQ ID NO:152); or GRTFSTAA (SEQ ID NO: 140), IRWSGGSA (SEQ ID NO: 145), and ARTENVRSLLSDYATWPYAY (SEQ ID NO: 153). In some embodiments, the polypeptides do not include an antigen-binding domain that includes the three CDRs of: GRTFSTAA (SEQ ID NO: 140), IRWSGGSA (SEQ ID NO: 145), and ARTENVRSLLSDYATWPYDY (SEQ ID NO: 147).

In some embodiments, the polypeptides include an antigen-binding domain that binds specifically to a coronavirus spike glycoprotein, where the antigen-binding domain comprises a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 154-165.

Provided herein are polypeptides that include an antigen-binding domain that binds specifically to a coronavirus spike glycoprotein, wherein the single polypeptide includes: (i) a CDR1 comprising a sequence of Formula X of: G-F-P-V-E-X₁-X₂-X₃ (SEQ ID NO: 166), wherein X₁ is V or A, X₂ is W, Y, or A, and X₃ is R or A; (ii) a CDR2 comprising a sequence of Formula XI of: I-E-X₄-X₅-G-H-G-X₆ (SEQ ID NO: 167), wherein X₄ is G or A, X₅ is Y or A, and X₆ is T or A; and (iii) a CDR3 comprising a sequence of Formula XII: N-V-X₇-D-D-X₈-X₉-L-A-Y-H-Y-D-Y (SEQ ID NO: 168), wherein X₇ is G or A, X₈ is G or N, and X₉ is Q or H.

In some embodiments, the polypeptides include an antigen-binding domain that binds specifically to a coronavirus spike glycoprotein where the polypeptide includes: (i) a CDR1 sequence selected from the group consisting of: GFPVEVWR (SEQ ID NO: 169), GFPVEVYR (SEQ ID NO: 170), GFPVEVAR (SEQ ID NO: 171), GFPVEAWR (SEQ ID NO: 172), and GFPVEVWA (SEQ ID NO: 173); (ii) a CDR2 sequence selected from the group consisting of: IESYGHGT (SEQ ID NO: 174), IESAGHGT (SEQ ID NO: 175), IESYGHGA (SEQ ID NO: 176), and IEAYGHGT (SEQ ID NO: 177); and (iii) a CDR3 sequence selected from the group consisting of: NVKDDGQLAYHYDY (SEQ ID NO: 178), NVYDDGQLAYHYDY (SEQ ID NO: 179), NVYDDGHLAYHYDY (SEQ ID NO: 180), and NVYDDNQLAYHYDY (SEQ ID NO: 181).

In some embodiments, the polypeptides include an antigen-binding domain that binds specifically to a coronavirus spike glycoprotein where the polypeptide includes: GFPVEVWR (SEQ ID NO: 169), IESAGHGT (SEQ ID NO: 175), and NVYDDGQLAYHYDY (SEQ ID NO: 179); GFPVEVYR (SEQ ID NO: 170), IESYGHGT (SEQ ID NO: 174), and NVYDDGQLAYHYDY (SEQ ID NO: 179); GFPVEVAR (SEQ ID NO: 171), IESYGHGT (SEQ ID NO: 174), and NVYDDGQLAYHYDY (SEQ ID NO: 179); GFPVEAWR (SEQ ID NO: 172), IESYGHGT (SEQ ID NO: 174), and NVYDDGQLAYHYDY (SEQ ID NO: 179); GFPVEVWR (SEQ ID NO: 169), IESYGHGA (SEQ ID NO: 176), and NVYDDGQLAYHYDY (SEQ ID NO: 179); GFPVEVWR (SEQ ID NO: 169), IEAYGHGT (SEQ ID NO: 177), and NVYDDGQLAYHYDY (SEQ ID NO: 179); GFPVEVWA (SEQ ID NO: 173), IESYGHGT (SEQ ID NO: 174), and NVYDDGQLAYHYDY (SEQ ID NO: 179); GFPVEVWR (SEQ ID NO: 169), IESYGHGT (SEQ ID NO: 174), and NVYDDGHLAYHYDY (SEQ ID NO: 180); GFPVEVWR (SEQ ID NO: 169), IESYGHGT (SEQ ID NO: 174), and NVYDDGQLAYHYDY (SEQ ID NO: 179); or GFPVEVWR (SEQ ID NO: 169), IESYGHGT (SEQ ID NO: 174), and NVYDDNQLAYHYDY (SEQ ID NO: 181). In some embodiments, the polypeptides do not include an antigen-binding domain that includes the three CDRs of: GFPVEVWR (SEQ ID NO: 169), IESYGHGT (SEQ ID NO: 174), and NVKDDGQLAYHYDY (SEQ ID NO: 178).

In some embodiments, the polypeptides include an antigen-binding domain that binds specifically to a coronavirus spike glycoprotein where the antigen-binding domain includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 185-197.

In some embodiments, the antigen-binding domain is a single domain antibody (e.g., VHH or VNAR). In some embodiments, the polypeptide further includes one or more additional antigen-binding domains (e.g., any of the exemplary antigen-binding domains described herein) (optionally separated by any of the exemplary linker sequences described herein). In some embodiments, a linker sequence comprises a total of about 1 amino acid to about 25 amino acids. Non-limiting examples of a linker sequence include: GS, GGGGS (SEQ ID NO: 207), GGGGSGGGGS (SEQ ID NO: 208), GGGGSGGGGSGGGGS (SEQ ID NO: 209), and GGGGSGGGGSGGGGSGGGGS (SEQ ID NO: 210). Additional examples of linker sequences are described herein and are known in the art.

In some embodiments, the polypeptide is a single-chain polypeptide. In some embodiments of any of the single-chain polypeptides described herein, the single-chain polypeptide can be or include a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the polypeptide is a multi-chain polypeptide. In some embodiments, the multi-chain polypeptide can be or can include an antibody, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KX-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv2.

In some embodiments, a multi-chain polypeptide can include a first polypeptide and a second polypeptide. In some embodiments, the first polypeptide is an antibody heavy chain. In some embodiments, the first polypeptide is an IgG1 antibody heavy chain (e.g., a human IgG1 antibody heavy chain), an IgG2 antibody heavy chain (e.g., a human IgG2 antibody heavy chain), an IgG3 antibody heavy chain (e.g., a human IgG3 antibody heavy chain), or an IgG4 antibody heavy chain (e.g., a human IgG4 antibody heavy chain).

In some embodiments, the second polypeptide is an antibody light chain (e.g., a kappa antibody light chain (e.g., a human kappa antibody light chain) or a lambda antibody light chain (e.g., a human lambda antibody light chain).

In some embodiments, the first polypeptide and the second polypeptide form a human or a humanized antibody (e.g., IgG1, IgG2, IgG3, and IgG4).

Non-limiting examples of multi-chain polypeptides include an Fv fragment, a Fab fragment, a F(ab′)₂ fragment, and a Fab′ fragment. Additional examples of protein constructs include an antigen-binding fragment of an IgG (e.g., an antigen-binding fragment of IgG1 (e.g., human IgG1), an antigen-binding fragment of IgG2 (e.g., human IgG2), an antigen-binding fragment of IgG3 (e.g., human IgG3), or an antigen-binding fragment of IgG4 (e.g., human IgG4)).

In some embodiments of any of the multi-chain polypeptides described herein, the protein complex is a human or a humanized IgG (e.g., a human or humanized IgG1, a human or humanized IgG2, a human or humanized IgG3, or a human or humanized IgG4), a human or a humanized IgGA (e.g., IgGA1 or IgGA2), a human or a humanized IgD, a human or a humanized IgE, or a human or a humanized IgM.

In some embodiments of any of the multi-chain polypeptides described herein, the multi-chain polypeptide is an antigen-binding fragment of an IgA (e.g., an antigen-binding fragment of IgA1 or IgA2) (e.g., a human or humanized antigen-binding fragment of IgA1 or IgA2).

In some embodiments of any of the multi-chain polypeptides described herein, the multi-chain polypeptide is an antigen-binding fragment of an IgD (e.g., a human or humanized antigen-binding fragment of IgD).

In some embodiments of any of the multi-chain polypeptides described herein, the multi-chain polypeptide is an antigen-binding fragment of an IgE (e.g., a human or humanized antigen-binding fragment of IgE).

In some embodiments of any of the multi-chain polypeptides described herein, the multi-chain polypeptide is an antigen-binding fragment of an IgM (e.g., a human or humanized antigen-binding fragment of IgM).

In some embodiments, the antigen-binding domain is humanized or human.

### Polypeptides Including at a Minimum Two Antigen-Binding Domains

Provided herein are polypeptides that include: (a) a first antigen-binding domain that specifically binds to a coronavirus spike glycoprotein including: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain that specifically binds to a coronavirus spike glycoprotein including: (i) a CDR1 comprising a sequence of Formula IV of: G-F-P-V-Y-S-W-N(SEQ ID NO: 127); (ii) a CDR2 comprising a sequence of Formula V of: I-E-S-H-X₁-D-S-T (SEQ ID NO: 128), wherein X₁ is G or A; and (iii) a CDR3 comprising a sequence of Formula VI: Y-V-W-V-X₂-H-T-Y-Y-G-Q (SEQ ID NO: 129), wherein X₂ is G or A.

In some embodiments of any of the polypeptides described herein, the first antigen-binding domain that binds specifically to a coronavirus spike glycoprotein includes: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), TISWSGGATYYTRSVKG (SEQ ID NO: 44), TISWSGGSTYYTASVKG (SEQ ID NO: 228), TISWSGGATYYTASVKG (SEQ ID NO: 229), or TISWSGGGTYYTDSVKG (SEQ ID NO: 230); and (iii) a CDR3 sequence selected from the group consisting of

(SEQ ID NO: 45)
AAAGLGTVVSEWDYDYDY,
(SEQ ID NO: 46)
AAAGLGTVVSEWDDYDY,
(SEQ ID NO: 47)
APAGLGTVVSEWDYDYDY,
(SEQ ID NO: 48)
AAADLGTVVSEWDYDYDY,
(SEQ ID NO: 49)
AAAGLGTVVSEADYDYDY,
(SEQ ID NO: 50)
AAAGLRTVVSEWDYDYDY,
(SEQ ID NO: 51)
AYAGLGTVVSEWDYDYDY,
(SEQ ID NO: 52)
AAAGLGTVVSEWDYDYDS,
(SEQ ID NO: 53)
AAAGLWTVVSEWDYDYDY,
(SEQ ID NO: 54)
AAAGLGTVVSAWDYDYDY,
(SEQ ID NO: 55)
AAAGLGTVVSEWDYDDY,
(SEQ ID NO: 56)
AAAGQGTVVSEWDYDYDY,
(SEQ ID NO: 57)
AAAGLETVVSEWDYDYDY,
(SEQ ID NO: 58)
AAARLGTVVSEWDYDYDY,
(SEQ ID NO: 57)
AAAGLETVVSEWDYDYDY;
or,
(SEQ ID NO: 60)
AAAGLGTVVSEWAYDYDA.

In some embodiments, the first antigen-binding domain that binds specifically to a coronavirus spike glycoprotein includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45). In some embodiments, the polypeptides do not include an antigen-binding domain comprising the three CDRs of: GRTFSEYAMG (SEQ ID NO: 1), TISWSGGATYTTDSVKG (SEQ ID NO: 43), and AAAGLGTVVSEWDYDYDY (SEQ ID NO: 45).

In some embodiments, the first antigen-binding domain that binds specifically to a coronavirus spike glycoprotein includes a framework region 1 (FR1) sequence selected from the group consisting of: MQVQLQESGGGLVQAGGSLRLSCAASG (SEQ ID NO: 61), and MQVQLQESGGGLVQAGGSLRLSCAASGSG (SEQ ID NO: 62).

In some embodiments, the first antigen-binding domain that binds specifically to a coronavirus spike glycoprotein includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 63-126 or 216-226.

In some embodiments, the polypeptides described herein comprises a CDR1 sequence present in any one of SEQ ID NOs: 63-126, 134-136, 154-165, 185-206, and 213-226 according to Kabat Numbering. In some embodiments, the polypeptides described herein comprises a CDR2 sequence present in any one of SEQ ID NOs: 63-126, 134-136, 154-165, 185-206, and 213-226 according to Kabat Numbering. In some embodiments, the polypeptides described herein comprises a CDR3 sequence present in any one of SEQ ID NOs: 63-126, 134-136, 154-165, 185-206, and 213-226 according to Kabat Numbering.

In some embodiments, the polypeptides described herein comprises a CDR1 sequence present in any one of SEQ ID NOs: 63-126, 134-136, 154-165, 185-206, and 213-226 according to Chothia Numbering. In some embodiments, the polypeptides described herein comprises a CDR2 sequence present in any one of SEQ ID NOs: 63-126, 134-136, 154-165, 185-206, and 213-226 according to Chothia Numbering. In some embodiments, the polypeptides described herein comprises a CDR3 sequence present in any one of SEQ ID NOs: 63-126, 134-136, 154-165, 185-206, and 213-226 according to Chothia Numbering.

In some embodiments, the polypeptides described herein comprises a CDR1 sequence present in any one of SEQ ID NOs: 63-126, 134-136, 154-165, 185-206, and 213-226 according to IMGT Numbering. In some embodiments, the polypeptides described herein comprises a CDR2 sequence present in any one of SEQ ID NOs: 63-126, 134-136, 154-165, 185-206, and 213-226 according to IMGT Numbering. In some embodiments, the polypeptides described herein comprises a CDR3 sequence present in any one of SEQ ID NOs: 63-126, 134-136, 154-165, 185-206, and 213-226 according to IMGT Numbering.

In some embodiments, the polypeptides described herein comprises a CDR1 sequence present in any one of SEQ ID NOs: 63-126, 134-136, 154-165, 185-206, and 213-226 according to Aho Numbering. In some embodiments, the polypeptides described herein comprises a CDR2 sequence present in any one of SEQ ID NOs: 63-126, 134-136, 154-165, 185-206, and 213-226 according to Aho Numbering. In some embodiments, the polypeptides described herein comprises a CDR3 sequence present in any one of SEQ ID NOs: 63-126, 134-136, 154-165, 185-206, and 213-226 according to Aho Numbering.

In some embodiments, the polypeptides described herein comprises a CDR1 sequence present in any one of SEQ ID NOs: 63-126, 134-136, 154-165, 185-206, and 213-226 according to AbM Numbering. In some embodiments, the polypeptides described herein comprises a CDR2 sequence present in any one of SEQ ID NOs: 63-126, 134-136, 154-165, 185-206, and 213-226 according to AbM Numbering. In some embodiments, the polypeptides described herein comprises a CDR3 sequence present in any one of SEQ ID NOs: 63-126, 134-136, 154-165, 185-206, and 213-226 according to AbM Numbering.

In some embodiments of any of the polypeptides described herein, the second antigen-binding domain that binds specifically to a coronavirus spike glycoprotein includes: (i) a CDR1 sequence selected from the group consisting of: GFPVYSWN (SEQ ID NO: 127); (ii) a CDR2 sequence selected from the group consisting of: IESHGDST (SEQ ID NO: 130), and IESYAHGT (SEQ ID NO: 131); and (iii) a CDR3 sequence selected from the group consisting of: YVWVGHTYYGQ (SEQ ID NO: 132), and YVWVAHTYYGQ (SEQ ID NO: 133).

In some embodiments, the second antigen-binding domain that binds specifically to a coronavirus spike glycoprotein includes: GFPVYSWN (SEQ ID NO: 127), IESHGDST (SEQ ID NO: 130), and YVWVAHTYYGQ (SEQ ID NO: 133); GFPVYSWN (SEQ ID NO: 127), IESHADST (SEQ ID NO: 231), and YVWVGHTYYGQ (SEQ ID NO: 132); or GFPVYSWN (SEQ ID NO: 127), IESHADST (SEQ ID NO: 231), and YVWVAHTYYGQ (SEQ ID NO: 133). In some embodiments, the polypeptides do not include an antigen-binding domain that includes the three CDRs of: GFPVYSWN (SEQ ID NO: 127), IESHGDST (SEQ ID NO: 130), and YVWVGHTYYGQ (SEQ ID NO: 132).

In some embodiments, the second antigen-binding domain that binds specifically to a coronavirus spike glycoprotein includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 134-136.

In some embodiments, the first antigen-binding domain comprises a CDR1 of SEQ ID NO: 1, a CDR2 selected from SEQ ID NOs: 2 or 4-44, and a CDR3 selected from SEQ ID NOs: 3 or 45-60, and the second antigen-binding domain comprises a CDR1 of SEQ ID NO: 127, a CDR2 selected from SEQ ID NO: 130, 131, or 231, and a CDR3 comprising SEQ ID NO: 132 or 133.

In some embodiments, the polypeptide includes a sequence that is at least 80% (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) identical to SEQ ID NO: 203.

Provided herein are polypeptides that includes: (a) a first antigen-binding domain that binds specifically to a coronavirus spike glycoprotein that includes: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain that binds specifically to a coronavirus spike glycoprotein that includes: (i) a CDR1 comprising a sequence of Formula VII of: G-X₁-T-X₂-S-T-A-A (SEQ ID NO: 137), wherein X₁ is R or A; X₂ is F, H, or Y; (ii) a CDR2 comprising a sequence of Formula VIII of: I-R-W-S-X₃-G-S-A (SEQ ID NO: 138), wherein X₃ is G or A; and (iii) a CDR3 comprising a sequence of Formula IX: A-R-T-E-N-V-R-X₄-X₅-L-S-D-Y-A-T-X₆-P-Y-X₇-Y (SEQ ID NO: 139), wherein X₄ is S or A; X₅ is L or K; X₆ is W or Y, and X₇ is A, Q, or R.

In some embodiments of any of the polypeptides described herein, the first antigen-binding domain that binds specifically to a coronavirus spike glycoprotein includes: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), TISWSGGATYYTRSVKG (SEQ ID NO: 44), TISWSGGSTYYTASVKG (SEQ ID NO: 228), TISWSGGATYYTASVKG (SEQ ID NO: 229), or TISWSGGGTYYTDSVKG (SEQ ID NO: 230); and (iii) a CDR3 sequence selected from the group consisting of:

(SEQ ID NO: 45)
AAAGLGTVVSEWDYDYDY,
(SEQ ID NO: 46)
AAAGLGTVVSEWDDYDY,
(SEQ ID NO: 47)
APAGLGTVVSEWDYDYDY,
(SEQ ID NO: 48)
AAADLGTVVSEWDYDYDY,
(SEQ ID NO: 49)
AAAGLGTVVSEADYDYDY,
(SEQ ID NO: 50)
AAAGLRTVVSEWDYDYDY,
(SEQ ID NO: 51)
AYAGLGTVVSEWDYDYDY,
(SEQ ID NO: 52)
AAAGLGTVVSEWDYDYDS,
(SEQ ID NO: 53)
AAAGLWTVVSEWDYDYDY,
(SEQ ID NO: 54)
AAAGLGTVVSAWDYDYDY,
(SEQ ID NO: 55)
AAAGLGTVVSEWDYDDY,
(SEQ ID NO: 56)
AAAGQGTVVSEWDYDYDY,
(SEQ ID NO: 57)
AAAGLETVVSEWDYDYDY,
(SEQ ID NO: 58)
AAARLGTVVSEWDYDYDY,
(SEQ ID NO: 57)
AAAGLETVVSEWDYDYDY;
or,
(SEQ ID NO: 60)
AAAGLGTVVSEWAYDYDA.

In some embodiments, the first antigen-binding domain that binds specifically to a coronavirus spike glycoprotein includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45). In some embodiments, the polypeptides do not include an antigen-binding domain that includes the three CDRs of: GRTFSEYAMG (SEQ ID NO: 1), TISWSGGATYTTDSVKG (SEQ ID NO: 43), and AAAGLGTVVSEWDYDYDY (SEQ ID NO: 45).

In some embodiments, the first antigen-binding domain that binds specifically to a coronavirus spike glycoprotein includes a framework region 1 (FR1) sequence selected from the group consisting of: MQVQLQESGGGLVQAGGSLRLSCAASG (SEQ ID NO: 61), and MQVQLQESGGGLVQAGGSLRLSCAASGSG (SEQ ID NO: 62).

In some embodiments, the first antigen-binding domain that binds specifically to a coronavirus spike glycoprotein includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 63-126 or 216-226.

In some embodiments of any of the polypeptides described herein, the second antigen-binding domain that binds specifically to a coronavirus spike glycoprotein includes: (i) a CDR1 sequence selected from the group consisting of: GRTFSTAA (SEQ ID NO: 140), GRTFATAA (SEQ ID NO: 141), GATFSTAA (SEQ ID NO: 142), GRTYSTAA (SEQ ID NO: 143), and GRTHSTAA (SEQ ID NO: 144); (ii) a CDR2 sequence selected from the group consisting of: IRWSGGSA (SEQ ID NO: 145), and IRWSAGSA (SEQ ID NO: 146); and (iii) a CDR3 sequence selected from the group consisting of: ARTENVRSLLSDYATWPYDY (SEQ ID NO: 147), ARTENVRSLLSDYATYPYDY (SEQ ID NO: 148), ARTENVRALLSDYATWPYDY (SEQ ID NO: 149), ARTENVRSKLSDYATWPYDY (SEQ ID NO: 150), ARTENVRSLLSDYATWPYRY (SEQ ID NO: 151), ARTENVRSLLSDYATWPYQY (SEQ ID NO: 152), and ARTENVRSLLSDYATWPYAY (SEQ ID NO: 153).

In some embodiments, the second antigen-binding domain that binds specifically to a coronavirus spike glycoprotein includes: GRTFSTAA (SEQ ID NO: 140), IRWSGGSA (SEQ ID NO: 145), and ARTENVRSLLSDYATYPYDY (SEQ ID NO: 148); GRTFATAA (SEQ ID NO: 141), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRALLSDYATWPYDY (SEQ ID NO: 149); GATFSTAA (SEQ ID NO: 142), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSKLSDYATWPYDY (SEQ ID NO: 150); GRTFSTAA (SEQ ID NO: 140), IRWSAGSA (SEQ ID NO: 146), and ARTENVRSLLSDYATWPYDY (SEQ ID NO: 147); GRTYSTAA (SEQ ID NO: 143), IRWSGGSA (SEQ ID NO: 145), and ARTENVRSLLSDYATWPYDY (SEQ ID NO: 147); GRTHSTAA (SEQ ID NO: 144), IRWSGGSA (SEQ ID NO: 145), and ARTENVRSLLSDYATWPYDY (SEQ ID NO: 147); GRTFSTAA (SEQ ID NO: 140), IRWSGGSA (SEQ ID NO: 145), and ARTENVRSLLSDYATWPYRY (SEQ ID NO: 151); GRTFSTAA (SEQ ID NO: 140), IRWSGGSA (SEQ ID NO: 145), and ARTENVRSLLSDYATWPYQY (SEQ ID NO:152); or GRTFSTAA (SEQ ID NO: 140), IRWSGGSA (SEQ ID NO: 145), and ARTENVRSLLSDYATWPYAY (SEQ ID NO: 153). In some embodiments, the polypeptides do not include an antigen-binding domain comprising the three CDRs of: GRTFSTAA (SEQ ID NO: 140), IRWSGGSA (SEQ ID NO: 145), and ARTENVRSLLSDYATWPYDY (SEQ ID NO: 147).

In some embodiments, the second antigen-binding domain that binds specifically to a coronavirus spike glycoprotein includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 154-165.

In some embodiments of the first antigen-binding domain comprises a CDR1 of SEQ ID NO: 1, a CDR2 selected from SEQ ID NOs: 2 or 4-44, and a CDR3 selected from SEQ ID NOs: 3 or 45-60, and the second antigen-binding domain comprises a CDR1 selected from SEQ ID NOs: 137 or 140-144, a CDR2 selected from SEQ ID NOs: 138 or 145-146, and a CDR3 selected from SEQ ID NOs: 139 or 147-153.

In some embodiments, the polypeptide includes a sequence that is at least 80% (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) identical to SEQ ID NO: 201 or 202.

Provided herein are polypeptides that includes: (a) a first antigen-binding domain that binds specifically to a coronavirus spike glycoprotein that includes: (i) a CDR1 comprising a sequence of Formula I: G-R-T-F-S-E-Y-A-M-G (SEQ ID NO: 1); (ii) a CDR2 comprising a sequence of Formula II: A-X₁-X₂-S-X₃-X₄-G-X₅-X₆-X₇-X₈-X₉-X₁₀-X₁₁-X₁₂-V-X₁₃-X₁₄ (SEQ ID NO: 2), wherein X₁ is T or S; X₂ is I, E, or S; X₃ is W, L, P, GW, GL, GP, GGW, GGL, or GGP; X₄ is S, G, or I; X₅ is G or W; X₆ is S, A, G, or S; and X₇ is T, A, G, or S; X₈ is Y or G; X₉ is Y, E, H, Q, S, T, or Y; X₁₀ is T or Y; X₁₁ is D, A, Q, R, T, or W; X₁₂ is S, T, or A; X₁₃ is K or D; and X₁₄ is G or A; and (iii) a CDR3 comprising a sequence of Formula III: A-X₁₅-A-X₁₆-X₁₇-X₁₈-T-V-V-S-X₁₉-X₂₀-X₂₁-Y-D-Y-D-Y (SEQ ID NO: 3), wherein X₁₅ is A, P, or Y; X₁₆ is G, D, E, or R; X₁₇ is L or Q; X₁₈ is G, E, R, or W; X₁₉ is E or A; X₂₀ is W or A; and X₂₁ is D or A; and (b) a second antigen-binding domain that binds specifically to a coronavirus spike glycoprotein that includes: (i) a CDR1 comprising a sequence of Formula IV of: G-F-P-V-E-X₁-X₂-X₃ (SEQ ID NO: 166), wherein X₁ is V or A, X₂ is W, Y, or A, and X₃ is R or A; (ii) a CDR2 comprising a sequence of Formula V of: I-E-X₄-X₅-G-H-G-X₆ (SEQ ID NO: 167), wherein X₄ is G or A, X₅ is Y or A, and X₆ is T or A; and (iii) a CDR3 comprising a sequence of Formula VI: N-V-X₇-D-D-X₈-X₉-L-A-Y-H-Y-D-Y (SEQ ID NO: 168), wherein X₇ is G or A, X₈ is G or N, and X₉ is Q or H.

In some embodiments of any of the polypeptides described herein, the first antigen-binding domain that binds specifically to a coronavirus spike glycoprotein includes: GRTFSEYAMG (SEQ ID NO: 1); (ii) a CDR2 sequence selected from the group consisting of: TISWSGGATYHTDTVKG (SEQ ID NO: 4), TISWSGGATYHTDSVKG (SEQ ID NO: 5), SISWSGGATYHTDSVKG (SEQ ID NO: 6), TISWSGGATYHTWSVKG (SEQ ID NO: 7), TISWSGGATGHTDSVKG (SEQ ID NO: 8), TESWSGGATYHTDSVKG (SEQ ID NO: 9), TISWSGGATYHTDSVDG (SEQ ID NO: 10), TISWSGWATYHTDSVKG (SEQ ID NO: 11), TISWIGGATYHTDSVKG (SEQ ID NO: 12), TISWSGGGTYHTDSVKG (SEQ ID NO: 13), TISWSGGASYHTDSVKG (SEQ ID NO: 14), TISWSGGAAYHTDSVKG (SEQ ID NO: 15), TISPSGGATYHTDSVKG (SEQ ID NO: 16), TISWSGGATYHTTSVKG (SEQ ID NO: 17), TISWSGGATYHTDSVKA (SEQ ID NO: 18), TISWGGGATYHTDSVKG (SEQ ID NO: 19), TSSWSGGATYHTDSVKG (SEQ ID NO: 20), TISLSGGATYHTDSVKG (SEQ ID NO: 21), TISWSGGATYHTDAVKG (SEQ ID NO: 22), TISWSGGATYHTASVKG (SEQ ID NO: 23); TISWSGGATYHYDSVKG (SEQ ID NO: 24), TISWSGGAGYHTDSVKG (SEQ ID NO: 25), TISGGWSGGATYHTDSVKG (SEQ ID NO: 26), TISGWSGGATYHTDSVKG (SEQ ID NO: 27), TISWSGGSTYYTRSVKG (SEQ ID NO: 28), TISWSGGSTYTTRSVKG (SEQ ID NO: 29), TISWSGGSTYTTDSVKG (SEQ ID NO: 30), TISWSGGSTYHTRSVKG (SEQ ID NO: 31), TISWSGGSTYHTDSVKG (SEQ ID NO: 32), TISWSGGGTYYTWSVKG (SEQ ID NO: 33), TISWSGGGTYYTRSVKG (SEQ ID NO: 34), TISWSGGGTYTTRSVKG (SEQ ID NO: 35), TISWSGGGTYHTRSVKG (SEQ ID NO: 36), TISWSGGATYYTQSVKG (SEQ ID NO: 37), TISWSGGATYYTDSVKG (SEQ ID NO: 38), TISWSGGGTYTTDSVKG (SEQ ID NO: 39), TISWSGGATYQTDSVKG (SEQ ID NO: 40), TISWSGGATYSTDSVKG (SEQ ID NO: 41), TISWSGGATYETDSVKG (SEQ ID NO: 42), TISWSGGATYTTDSVKG (SEQ ID NO: 43), TISWSGGATYYTRSVKG (SEQ ID NO: 44), TISWSGGSTYYTASVKG (SEQ ID NO: 228), TISWSGGATYYTASVKG (SEQ ID NO: 229), or TISWSGGGTYYTDSVKG (SEQ ID NO: 230); and (iii) a CDR3 sequence selected from the group consisting of:

(SEQ ID NO: 45)
AAAGLGTVVSEWDYDYDY,
(SEQ ID NO: 46)
AAAGLGTVVSEWDDYDY,
(SEQ ID NO: 47)
APAGLGTVVSEWDYDYDY,
(SEQ ID NO: 48)
AAADLGTVVSEWDYDYDY,
(SEQ ID NO: 49)
AAAGLGTVVSEADYDYDY,
(SEQ ID NO: 50)
AAAGLRTVVSEWDYDYDY,
(SEQ ID NO: 51)
AYAGLGTVVSEWDYDYDY,
(SEQ ID NO: 52)
AAAGLGTVVSEWDYDYDS,
(SEQ ID NO: 53)
AAAGLWTVVSEWDYDYDY,
(SEQ ID NO: 54)
AAAGLGTVVSAWDYDYDY,
(SEQ ID NO: 55)
AAAGLGTVVSEWDYDDY,
(SEQ ID NO: 56)
AAAGQGTVVSEWDYDYDY,
(SEQ ID NO: 57)
AAAGLETVVSEWDYDYDY,
(SEQ ID NO: 58)
AAARLGTVVSEWDYDYDY,
(SEQ ID NO: 57)
AAAGLETVVSEWDYDYDY;
or,
(SEQ ID NO: 60)
AAAGLGTVVSEWAYDYDA.

In some embodiments, the first antigen-binding domain that binds specifically to a coronavirus spike glycoprotein includes: GRTFSEYAMG (SEQ ID NO: 1), TISWSGGATYHTDTVKG (SEQ ID NO: 4), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDDYDY (SEQ ID NO: 46); GRTFSEYAMG (SEQ ID NO: 1), TISWSGGATYHTDSVKG (SEQ ID NO: 5), and APAGLGTVVSEWDYDYDY (SEQ ID NO: 47); GRTFSEYAMG (SEQ ID NO: 1), TISWSGGATYHTDSVKG (SEQ ID NO: 5), and AAADLGTVVSEWDYDYDY (SEQ ID NO: 48); GRTFSEYAMG (SEQ ID NO: 1), SISWSGGATYHTDSVKG (SEQ ID NO: 6), and AAAGLGTVVSEWDYDYDY (SEQ ID NO: 45); GRTFSEYAMG (SEQ ID NO: 1), TISWSGGATYHTWSVKG (SEQ ID NO: 7), and AAAGLGTVVSEWDYDYDY (SEQ ID NO: 45); GRTFSEYAMG (SEQ ID NO: 1), TISWSGGATGHTDSVKG (SEQ ID NO: 8), and AAAGLGTVVSEWDYDYDY (SEQ ID NO: 45); GRTFSEYAMG (SEQ ID NO: 1), TESWSGGATYHTDSVKG (SEQ ID NO: 9), and AAAGLGTVVSEWDYDYDY (SEQ ID NO: 45); GRTFSEYAMG (SEQ ID NO: 1), TISWSGGATYHTDSVDG (SEQ ID NO: 10), and AAAGLGTVVSEWDYDYDY (SEQ ID NO: 45); GRTFSEYAMG (SEQ ID NO: 1), TISWSGWATYHTDSVKG (SEQ ID NO: 11), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEADYDYDY (SEQ ID NO: 49); GRTFSEYAMG (SEQ ID NO: 1), TISWIGGATYHTDSVKG (SEQ ID NO: 12), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLRTVVSEWDYDYDY (SEQ ID NO: 50); GRTFSEYAMG (SEQ ID NO: 1), TISWSGGASYHTDSVKG (SEQ ID NO: 14), and AAAGLGTVVSEWDYDYDY (SEQ ID NO: 45); GRTFSEYAMG (SEQ ID NO: 1), TISWSGGAAYHTDSVKG (SEQ ID NO: 15), and AAAGLGTVVSEWDYDYDY (SEQ ID NO: 45); GRTFSEYAMG (SEQ ID NO: 1), TISPSGGATYHTDSVKG (SEQ ID NO: 16), and AAAGLGTVVSEWDYDYDY (SEQ ID NO: 45); GRTFSEYAMG (SEQ ID NO: 1), TISWSGGATYHTTSVKG (SEQ ID NO: 17), and AAAGLGTVVSEWDYDYDY (SEQ ID NO: 45); GRTFSEYAMG (SEQ ID NO: 1), TISWSGGATYHTDSVKA (SEQ ID NO: 18), and AAAGLGTVVSEWDYDYDY (SEQ ID NO: 45); GRTFSEYAMG (SEQ ID NO: 1), TISWSGGATYHTDSVKG (SEQ ID NO: 5), and AYAGLGTVVSEWDYDYDY (SEQ ID NO: 51); GRTFSEYAMG (SEQ ID NO: 1), TISWGGGATYHTDSVKG (SEQ ID NO: 19), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDS (SEQ ID NO: 52); GRTFSEYAMG (SEQ ID NO: 1), TISWSGGATYHTDSVKG (SEQ ID NO: 5), and AAAGLWTVVSEWDYDYDY (SEQ ID NO: 53); GRTFSEYAMG (SEQ ID NO: 1), TISWSGGATYHTDSVKG (SEQ ID NO: 5), and AAAGLGTVVSAWDYDYDY (SEQ ID NO: 54); GRTFSEYAMG (SEQ ID NO: 1), TISWSGGATYHTDSVKG (SEQ ID NO: 5), and AAAGLGTVVSEWDYDDY (SEQ ID NO: 55); GRTFSEYAMG (SEQ ID NO: 1), TSSWSGGATYHTDSVKG (SEQ ID NO: 20), and AAAGLGTVVSEWDYDYDY (SEQ ID NO: 45); GRTFSEYAMG (SEQ ID NO: 1), TISLSGGATYHTDSVKG (SEQ ID NO: 21), and AAAGLGTVVSEWDYDYDY (SEQ ID NO: 45); GRTFSEYAMG (SEQ ID NO: 1), TISWSGGATYHTDAVKG (SEQ ID NO: 22), and AAAGLGTVVSEWDYDYDY (SEQ ID NO: 45); GRTFSEYAMG (SEQ ID NO: 1), TISWSGGATYHTDSVKG (SEQ ID NO: 5), and AAAGQGTVVSEWDYDYDY (SEQ ID NO: 56); GRTFSEYAMG (SEQ ID NO: 1), TISWSGGATYHTASVKG (SEQ ID NO: 23), and AAAGLGTVVSEWDYDYDY (SEQ ID NO: 45); GRTFSEYAMG (SEQ ID NO: 1), TISWSGGATYHYDSVKG (SEQ ID NO: 24), and AAAGLGTVVSEWDYDYDY (SEQ ID NO: 45); GRTFSEYAMG (SEQ ID NO: 1), TISWSGGAGYHTDSVKG (SEQ ID NO: 25), and AAAGLGTVVSEWDYDYDY (SEQ ID NO: 45); GRTFSEYAMG (SEQ ID NO: 1), TISWSGGATYHTDSVKG (SEQ ID NO: 5), and AAAGLGTVVSEWDYDYDY (SEQ ID NO: 45); GRTFSEYAMG (SEQ ID NO: 1), TISGGWSGGATYHTDSVKG (SEQ ID NO: 26), and AAAGLGTVVSEWDYDYDY (SEQ ID NO: 45); GRTFSEYAMG (SEQ ID NO: 1), TISGWSGGATYHTDSVKG (SEQ ID NO: 27), and AAAGLGTVVSEWDYDYDY (SEQ ID NO: 45); GRTFSEYAMG (SEQ ID NO: 1), TISWSGGSTYYTRSVKG (SEQ ID NO: 28), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLETVVSEWDYDYDY (SEQ ID NO: 57); GRTFSEYAMG (SEQ ID NO: 1), TISWSGGSTYTTRSVKG (SEQ ID NO: 29), and AAAGLGTVVSEWDYDYDY (SEQ ID NO: 45); GRTFSEYAMG (SEQ ID NO: 1), TISWSGGSTYTTDSVKG (SEQ ID NO: 30), and AAAGLETVVSEWDYDYDY (SEQ ID NO: 57); GRTFSEYAMG (SEQ ID NO: 1), TISWSGGSTYHTRSVKG (SEQ ID NO: 31), and AAAGLGTVVSEWDYDYDY (SEQ ID NO: 45); GRTFSEYAMG (SEQ ID NO: 1), TISWSGGSTYHTRSVKG (SEQ ID NO: 31), and AAAGLETVVSEWDYDYDY (SEQ ID NO: 57); GRTFSEYAMG (SEQ ID NO: 1), TISWSGGSTYHTDSVKG (SEQ ID NO: 32), and AAAGLETVVSEWDYDYDY (SEQ ID NO: 57); GRTFSEYAMG (SEQ ID NO: 1), TISWSGGGTYYTWSVKG (SEQ ID NO: 33), and AAAGLETVVSEWDYDYDY (SEQ ID NO: 57); GRTFSEYAMG (SEQ ID NO: 1), TISWSGGATYYTQSVKG (SEQ ID NO: 37), and AAAGLGTVVSEWDYDYDY (SEQ ID NO: 45); GRTFSEYAMG (SEQ ID NO: 1), TISWSGGGTYYTRSVKG (SEQ ID NO: 34), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLETVVSEWDYDYDY (SEQ ID NO: 57); GRTFSEYAMG (SEQ ID NO: 1), TISWSGGGTYTTRSVKG (SEQ ID NO: 35), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLGTVVSEWDYDYDY (SEQ ID NO: 45); GRTFSEYAMG (SEQ ID NO: 1), TISWSGGGTYTTDSVKG (SEQ ID NO: 39), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLETVVSEWDYDYDY (SEQ ID NO: 57); GRTFSEYAMG (SEQ ID NO: 1), TISWSGGGTYHTRSVKG (SEQ ID NO: 36), and AAAGLGTVVSEWDYDYDY (SEQ ID NO: 45); GRTFSEYAMG (SEQ ID NO: 1), TISWSGGGTYHTDSVKG (SEQ ID NO: 13), and AAAGLETVVSEWDYDYDY (SEQ ID NO: 57); GRTFSEYAMG (SEQ ID NO: 1), TISWSGGATYQTDSVKG (SEQ ID NO: 40), and AAAGLGTVVSEWDYDYDY (SEQ ID NO: 45); GRTFSEYAMG (SEQ ID NO: 1), TISWSGGATYSTDSVKG (SEQ ID NO: 41), and AAAGLGTVVSEWDYDYDY (SEQ ID NO: 45); GRTFSEYAMG (SEQ ID NO: 1), TISWSGGATYETDSVKG (SEQ ID NO: 42), and AAAGLGTVVSEWDYDYDY (SEQ ID NO: 45); GRTFSEYAMG (SEQ ID NO: 1), TISWSGGATYYTRSVKG (SEQ ID NO: 44), and AAAGLGTVVSEWDYDYDY (SEQ ID NO: 45); GRTFSEYAMG (SEQ ID NO: 1), TISWSGGATYYTDSVKG (SEQ ID NO: 38), and AAARLGTVVSEWDYDYDY (SEQ ID NO: 58); GRTFSEYAMG (SEQ ID NO: 1), TISWSGGATYYTDSVKG (SEQ ID NO: 38), and AAAELGTVVSEWDYDYDY (SEQ ID NO: 59); GRTFSEYAMG (SEQ ID NO: 1), TISWSGGATYYTDSVKG (SEQ ID NO: 38), and AAAGLETVVSEWDYDYDY (SEQ ID NO: 57); GRTFSEYAMG (SEQ ID NO: 1), TISWSGGATYYTDSVKG (SEQ ID NO: 38), and AAAGLGTVVSEWAYDYDA (SEQ ID NO: 60); or GRTFSEYAMG (SEQ ID NO: 1), TISWSGGATYYTDSVKG (SEQ ID NO: 38), and AAAGLGTVVSEWDYDYDY (SEQ ID NO: 45). In some embodiments, the polypeptides do not include an antigen-binding domain that includes the three CDRs of: GRTFSEYAMG (SEQ ID NO: 1), TISWSGGATYTTDSVKG (SEQ ID NO: 43), and AAAGLGTVVSEWDYDYDY (SEQ ID NO: 45).

In some embodiments, the first antigen-binding domain that binds specifically to a coronavirus spike glycoprotein includes a framework region 1 (FR1) sequence selected from the group consisting of: MQVQLQESGGGLVQAGGSLRLSCAASG (SEQ ID NO: 61), and MQVQLQESGGGLVQAGGSLRLSCAASGSG (SEQ ID NO: 62).

In some embodiments, the first antigen-binding domain that binds specifically to a coronavirus spike glycoprotein includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 63-126 or 216-226.

In some embodiments, the second antigen-binding domain that binds specifically to a coronavirus spike glycoprotein includes: (i) a CDR1 sequence selected from the group consisting of: GFPVEVWR (SEQ ID NO: 169), GFPVEVYR (SEQ ID NO: 170), GFPVEVAR (SEQ ID NO: 171), GFPVEAWR (SEQ ID NO: 172), and GFPVEVWA (SEQ ID NO: 173); (ii) a CDR2 sequence selected from the group consisting of: IESYGHGT (SEQ ID NO: 174), IESAGHGT (SEQ ID NO: 175), IESYGHGA (SEQ ID NO: 176), and IEAYGHGT (SEQ ID NO: 177); and (iii) a CDR3 sequence selected from the group consisting of: NVKDDGQLAYHYDY (SEQ ID NO: 178), NVYDDGQLAYHYDY (SEQ ID NO: 179), NVYDDGHLAYHYDY (SEQ ID NO: 180), and NVYDDNQLAYHYDY (SEQ ID NO: 181).

In some embodiments, the second antigen-binding domain that binds specifically to a coronavirus spike glycoprotein includes: GFPVEVWR (SEQ ID NO: 169), IESAGHGT (SEQ ID NO: 175), and NVYDDGQLAYHYDY (SEQ ID NO: 179); GFPVEVYR (SEQ ID NO: 170), IESYGHGT (SEQ ID NO: 174), and NVYDDGQLAYHYDY (SEQ ID NO: 179); GFPVEVAR (SEQ ID NO: 171), IESYGHGT (SEQ ID NO: 174), and NVYDDGQLAYHYDY (SEQ ID NO: 179); GFPVEAWR (SEQ ID NO: 172), IESYGHGT (SEQ ID NO: 174), and NVYDDGQLAYHYDY (SEQ ID NO: 179); GFPVEVWR (SEQ ID NO: 169), IESYGHGA (SEQ ID NO: 176), and NVYDDGQLAYHYDY (SEQ ID NO: 179); GFPVEVWR (SEQ ID NO: 169), IEAYGHGT (SEQ ID NO: 177), and NVYDDGQLAYHYDY (SEQ ID NO: 179); GFPVEVWA (SEQ ID NO: 173), IESYGHGT (SEQ ID NO: 174), and NVYDDGQLAYHYDY (SEQ ID NO: 179); GFPVEVWR (SEQ ID NO: 169), IESYGHGT (SEQ ID NO: 174), and NVYDDGHLAYHYDY (SEQ ID NO: 180); GFPVEVWR (SEQ ID NO: 169), IESYGHGT (SEQ ID NO: 174), and NVYDDGQLAYHYDY (SEQ ID NO: 179); or GFPVEVWR (SEQ ID NO: 169), IESYGHGT (SEQ ID NO: 174), and NVYDDNQLAYHYDY (SEQ ID NO: 181). In some embodiments, the polypeptides do not include an antigen-binding domain that includes the three CDRs of: GFPVEVWR (SEQ ID NO: 169), IESYGHGT (SEQ ID NO: 174), and NVKDDGQLAYHYDY (SEQ ID NO: 178).

In some embodiments, the second antigen-binding domain that binds specifically to a coronavirus spike glycoprotein includes: a framework region 3 (FR3) sequence selected from the group consisting of: RYADSVKGRFTISRDNAKNTVYLQMNSLKPEDTAVYYC (SEQ ID NO: 182), RYADSVRGRFTISRDNAKNTVYLQMNSLKPEDTAVYYC (SEQ ID NO: 183), or RYADSVAGRFTISRDNAKNTVYLQMNSLKPEDTAVYYC (SEQ ID NO: 184).

In some embodiments, the second antigen-binding domain that binds specifically to a coronavirus spike glycoprotein includes a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 185-197.

In some embodiments of the first antigen-binding domain comprises a CDR1 of SEQ ID NO: 1, a CDR2 selected from SEQ ID NOs: 2 or 4-44, and a CDR3 selected from SEQ ID NO: 3 or 45-60, and the second antigen-binding domain comprises a CDR1 selected from SEQ ID NOs: 166 or 169-173, a CDR2 selected from SEQ ID NOs: 167 or 174-177, and a CDR3 selected from SEQ ID NOs: 168 or 178-181.

In some embodiments, the polypeptide includes a sequence that is at least 80% (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) identical to SEQ ID NO: 198, 199, 200, or 206.

Provided herein are polypeptides that include a sequence that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to a sequence selected from SEQ ID NOs: 198-207.

Provided herein are polypeptides that include a first antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 213; and a second antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 134.

Provided herein are polypeptides that include a first antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 213; and a second antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 154.

Provided herein are polypeptides that include a first antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 213; and a second antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 185.

Provided herein are polypeptides that include a first antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 213; and a second antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 186.

Provided herein are polypeptides that include a first antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 213; and a second antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 214.

Provided herein are polypeptides that include a first antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NOs: 213; and a second antigen-binding domain that is at least 80% identical (e.g., at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 215.

In some embodiments, the first and/or the second antigen-binding domain is a single domain antibody (e.g., VHH or VNAR). In some embodiments, the polypeptide further includes one or more additional antigen-binding domains (e.g., any of the exemplary antigen-binding domains described herein) (optionally separated by any of the exemplary linker sequences described herein).

In some embodiments, the polypeptide is a single-chain polypeptide. In some embodiments of any of the single-chain polypeptides described herein, the single-chain polypeptide can be or include a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv. In some embodiments, a single-chain polypeptide further comprises a linker sequence disposed between the first and the second antigen-binding domains. In some embodiments, the first antigen-binding domain is N-terminally positioned relative to the second antigen-binding domain in the single-chain polypeptide. In some embodiments, the first antigen-binding domain is C-terminally positioned relative to the second antigen-binding domain in the single-chain polypeptide.

In some embodiments, the polypeptide is a multi-chain polypeptide. In some embodiments, the multi-chain polypeptide can be or can include an antibody, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KX-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv₂, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv₂.

In some embodiments, a multi-chain polypeptide can include a first polypeptide and a second polypeptide. In some embodiments, the first polypeptide comprises the first antigen-binding domain and the second polypeptide comprises the second antigen-binding domain. In some embodiments, the first polypeptide is an antibody heavy chain. In some embodiments, the first polypeptide is an IgG1 antibody heavy chain (e.g., a human IgG1 antibody heavy chain), an IgG2 antibody heavy chain (e.g., a human IgG2 antibody heavy chain), an IgG3 antibody heavy chain (e.g., a human IgG3 antibody heavy chain), or an IgG4 antibody heavy chain (e.g., a human IgG4 antibody heavy chain).

In some embodiments, the second polypeptide is an antibody light chain (e.g., a kappa antibody light chain (e.g., a human kappa antibody light chain) or a lambda antibody light chain (e.g., a human lambda antibody light chain).

In some embodiments, the first polypeptide and the second polypeptide form a human or a humanized antibody (e.g., IgG1, IgG2, IgG3, and IgG4).

Non-limiting examples of multi-chain polypeptides include an Fv fragment, a Fab fragment, a F(ab′)₂ fragment, and a Fab′ fragment. Additional examples of protein constructs include an antigen-binding fragment of an IgG (e.g., an antigen-binding fragment of IgG1 (e.g., human IgG1), an antigen-binding fragment of IgG2 (e.g., human IgG2), an antigen-binding fragment of IgG3 (e.g., human IgG3), or an antigen-binding fragment of IgG4 (e.g., human IgG4)).

In some embodiments of any of the multi-chain polypeptides described herein, the protein complex is a human or a humanized IgG (e.g., a human or humanized IgG1, a human or humanized IgG2, a human or humanized IgG3, or a human or humanized IgG4), a human or a humanized IgGA (e.g., IgGA1 or IgGA2), a human or a humanized IgD, a human or a humanized IgE, or a human or a humanized IgM.

In some embodiments of any of the multi-chain polypeptides described herein, the multi-chain polypeptide is an antigen-binding fragment of an IgA (e.g., an antigen-binding fragment of IgA1 or IgA2) (e.g., a human or humanized antigen-binding fragment of IgA1 or IgA2).

In some embodiments of any of the multi-chain polypeptides described herein, the multi-chain polypeptide is an antigen-binding fragment of an IgD (e.g., a human or humanized antigen-binding fragment of IgD).

In some embodiments of any of the multi-chain polypeptides described herein, the multi-chain polypeptide is an antigen-binding fragment of an IgE (e.g., a human or humanized antigen-binding fragment of IgE).

In some embodiments of any of the multi-chain polypeptides described herein, the multi-chain polypeptide is an antigen-binding fragment of an IgM (e.g., a human or humanized antigen-binding fragment of IgM).

In some embodiments, the first or the second polypeptides can include two antigen-binding domains (e.g., the first and second antigen-binding domains, the first antigen-binding domain and an additional antigen-binding domain, or the second antigen-binding domain and an additional antigen-binding domain), where the two antigen-binding domains are separated by a linker sequence.

In some embodiments, a linker sequence comprises a total of about 1 amino acid to about 25 amino acids. Non-limiting examples of a linker sequence include: GS, GGGGS (SEQ ID NO: 207), GGGGSGGGGS (SEQ ID NO: 208), GGGGSGGGGSGGGGS (SEQ ID NO: 209), and GGGGSGGGGSGGGGSGGGGS (SEQ ID NO: 210). Additional examples of linker sequences are described herein and are known in the art.

In some embodiments, the antigen-binding domain is humanized or human.

### Linker-Sequences

In some embodiments, the linker sequence can be a flexible linker sequence. Non-limiting examples of linker sequences that can be used are described in Klein et al., Protein Engineering, Design & Selection 27(10):325-330, 2014; Priyanka et al., Protein Sci. 22(2):153-167, 2013. In some examples, the linker sequence is a synthetic linker sequence.

In some embodiments of any of the polypeptides described herein can include one, two, three, four, five, six, seven, eight, nine, or ten linker sequence(s) (e.g., the same or different linker sequences, e.g., any of the exemplary linker sequences described herein or known in the art). In some embodiments of any of the single-chain chimeric polypeptides described herein can include one, two, three, four, five, six, seven, eight, nine, or ten linker sequence(s) (e.g., the same or different linker sequences, e.g., any of the exemplary linker sequences described herein or known in the art).

In some embodiments, the linker sequence includes a total of about 1 amino acid to about 25 amino acids (e.g., about 1 amino acid to about 24 amino acids, about 1 amino acid to about 22 amino acids, about 1 amino acid to about 20 amino acids, about 1 amino acid to about 18 amino acids, about 1 amino acid to about 16 amino acids, about 1 amino acid to about 15 amino acids, about 1 amino acid to about 14 amino acids, about 1 amino acid to about 12 amino acids, about 1 amino acid to about 10 amino acids, about 1 amino acid to about 8 amino acids, about 1 amino acid to about 6 amino acids, about 1 amino acid to about 5 amino acids, about 1 amino acid to about 4 amino acids, about 1 amino acid to about 3 amino acids, about 1 amino acid to about 2 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 5 amino acids, about 2 amino acids to about 4 amino acids, about 2 amino acids to about 3 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 4 amino acids to about 5 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 15 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 15 amino acids, about 8 amino acids to about 14 amino acids, about 8 amino acids to about 12 amino acids, about 8 amino acids to about 10 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 15 amino acids, about 10 amino acids to about 14 amino acids, about 10 amino acids to about 12 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 15 amino acids, about 12 amino acids to about 14 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 14 amino acids to about 15 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 25 amino acids, about 18 amino acids to about 24 amino acids, about 18 amino acids to about 22 amino acids, about 18 amino acids to about 20 amino acids, about 20 amino acids to about 25 amino acids, about 20 amino acids to about 24 amino acids, about 20 amino acids to about 22 amino acids, about 22 amino acid to about 25 amino acids, about 22 amino acid to about 24 amino acids, or about 24 amino acid to about 25 amino acids).

In some embodiments, the linker sequence includes a total of about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, or about 25 amino acids in length.

In some embodiments, the linker sequence is rich in glycine (Gly or G) residues. In some embodiments, the linker sequence is rich in serine (Ser or S) residues. In some embodiments, the linker sequence is rich in glycine and serine residues. In some embodiments, the linker sequence has one or more glycine-serine residue pairs (GS), e.g., 1, 2, 3, 4, 5, 6, 7, 8, 9, or 10 or more GS pairs. In some embodiments, the linker sequence has one or more Gly-Gly-Gly-Ser (GGGS) (SEQ ID NO: 211) sequences, e.g., 1, 2, 3, 4, 5, 6, 7, 8, 9, or 10 or more GGGS (SEQ ID NO: 211) sequences. In some embodiments, the linker sequence has one or more Gly-Gly-Gly-Gly-Ser (GGGGS) (SEQ ID NO: 207) sequences, e.g., 1, 2, 3, 4, 5, 6, 7, 8, 9, or 10 or more GGGGS (SEQ ID NO: 207) sequences. In some embodiments, the linker sequence has one or more Gly-Gly-Ser-Gly (GGSG) (SEQ ID NO: 212) sequences, e.g., 1, 2, 3, 4, 5, 6, 7, 8, 9, or 10 or more GGSG (SEQ ID NO: 212) sequences.

In some embodiments, the linker sequence can comprise or consist of GGGGSGGGGS (SEQ ID NO: 208). In some embodiments, the linker sequence can comprise or consist of GGGGSGGGGSGGGGS (SEQ ID NO: 209). In some embodiments, the linker sequence can comprise or consist of GGGGSGGGGSGGGGSGGGGS (SEQ ID NO: 210).

Non-limiting examples of linker sequences can include a sequence that is at least 70% identical (e.g., at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, at least 99%, or 100% identical) to:

(SEQ ID NO: 208)
GGGGSGGGGS,
(SEQ ID NO: 209)
GGGGSGGGGSGGGGS,
and
(SEQ ID NO: 210)
GGGGSGGGGSGGGGSGGGGS.

In some embodiments of any of the polypeptides described herein, a linker sequence can be disposed between the first antigen-binding domain and the second antigen binding domain. For example, in certain embodiments, a polypeptide includes the following formula (wherein the formula below represents an amino acid sequence in either N- to C-terminal direction or C- to N-terminal direction): a first antigen-binding domain-linker-a second antigen-binding domain.

### Nucleic Acids

Also provided herein are nucleic acid(s) including sequence(s) that encode the polypeptide (e.g., any of the polypeptides described herein). Also provided herein is a set of nucleic acids that together encode the polypeptide.

### Vectors

As used herein, the term “vector” refers to a polynucleotide capable of inducing the expression of a protein (e.g., any of the polypeptides described herein) in a cell (e.g., any of the cells described herein). A “vector” is able to deliver nucleic acids and fragments thereof into a host cell, and includes regulatory sequences (e.g., promoter, enhancer, poly(A) signal). Exogenous polynucleotides may be inserted into the expression vector in order to be expressed. The term “vector” also includes artificial chromosomes, plasmids, retroviruses, and baculovirus vectors.

Methods for constructing suitable vectors that include any of the nucleic acids described herein, and suitable for transforming cells (e.g., mammalian cells) are well-known in the art. See, e.g., Sambrook et al., Eds. “Molecular Cloning: A Laboratory Manual,” 2^{nd} Ed., Cold Spring Harbor Press, 1989 and Ausubel et al., Eds. “Current Protocols in Molecular Biology,” Current Protocols, 1993.

Non-limiting examples of expression vectors include plasmids and viral vectors. In some embodiments, the expression vectors are plasmids, adeno-associated viral (AAV) vectors, lentiviral vectors, sindbis virus vectors, alphavirus-based vectors, or adenoviral vectors. AAV vectors are generally described in, e.g., Asokan et al., Mol. Ther. 20: 699-708, 2012, and B. J. Carter, in “Handbook of Parvoviruses”, Ed., P. Tijsser, CRC Press, pp. 155-168, 1990. Adenoviral vectors are generally described in, e.g., Wold and Toth, Curr. Gene Ther. 13(6):421-433, 2013; Baron et al., Curr. Opin. Virol. 29:1-7, 2018; and Barry, Expert Rev. Vaccines 17(2): 163-173, 2018. Lentiviral vectors are generally described in, e.g., Milone and O'Doherty, Leukemia 32(7): 1529-1541, 2018, Zheng et al., Anat. Rec. 301(5): 825-836, 2018; and Cai et al., Curr. Gene Ther. 16(3): 194-206, 2016.

Some embodiments of any of the expression vectors described herein, can include a promoter and/or enhancer operably linked to a nucleic acid encoding a polypeptide (e.g., any of the exemplary polypeptides described herein).

In some embodiments, the nucleic acid further includes a promoter and/or enhancer operably linked to the first sequence or the second sequence. In some embodiments, the promoter is constitutive. In some embodiments, the promoter is inducible. In some embodiments, the promoter is a tissue-specific promoter. Exemplary promoters that are constitutive, inducible, and/or tissue-specific are known in the art. A non-limiting example of a promoter is a CMV promoter. A non-limiting examples of enhancers is an apolipoprotein E (ApoE) enhancer.

In some embodiments, the expression vectors described herein include one or more (e.g., two, three, four, five, or six) of a promoter (e.g., any of the promoters described herein or known in the art), an enhancer (e.g., any of the enhancers described herein or known in the art), a Kozak sequence (e.g., any of the Kozak sequences described herein or known in the art), a polyadenylation (poly(A)) signal sequence (e.g., any of the poly(A) signals described herein), and an internal ribosome entry site (IRES) sequence (e.g., any of the IRES sequences described herein or known in the art).

### Poly(A) Signal

In some embodiments, the expression vector (e.g., any of the exemplary expression vectors described herein) can include a polyadenylation (poly(A)) signal sequence. Poly(A) tails are added to most nascent eukaryotic messenger RNAs (mRNAs) at their 3′ end during a complex process that includes cleavage of the primary transcript and a coupled polyadenylation reaction driven by the poly(A) signal sequence. In some embodiments of any of the expression vectors described herein, the expression vector can include a poly(A) signal sequence at the 3′ end of the nucleic acid encoding a polypeptide (e.g., any of the polypeptides described herein), an antibody fragment, an antibody, or a first polypeptide, a second polypeptide, or both (e.g., any of the first polypeptides described herein or any of the second polypeptides described herein).

The term “polyadenylation” refers to the covalent linkage of a polyadenylyl moiety, or its modified variant, to the 3′ end of a mRNA molecule. A poly(A) tail is a long sequence of adenine nucleotides (e.g., 40, 50, 100, 200, 500, 1000) added to the pre-mRNA by a polyadenylate polymerase.

The term “poly(A) signal sequence” or “poly(A) signal” is a sequence that triggers the endonuclease cleavage of a mRNA and the addition of a sequence of adenosine to the 3′end of the cleaved mRNA. Non-limiting examples of poly(A) signals include: bovine growth hormone (bGH) poly(A) signal, human growth hormone (hGH) poly(A) signal. In some embodiments of any of the AAV vectors described herein, the AAV vector can include a poly(A) signal sequence that includes the sequence AATAAA or variations thereof. Additional examples of poly(A) signal sequences are known in the art.

### Cells

Also provided herein is a cell that includes any of the vectors or nucleic acids described herein. Skilled practitioners will appreciate that the expression vectors and nucleic acids described herein can be introduced into any cell (e.g., any mammalian cell) and that a variety of technologies can be utilized for modifying the genome of cells (e.g., mammalian cells). Non-limiting examples of expression vectors and methods for introducing expression vectors and nucleic acids into cells (e.g., any mammalian cell) are described herein.

In some embodiments, the cell is a mammalian cell. In some embodiments, the mammalian cell is a human cell, a rodent cell (e.g., a rat cell or a mouse cell), a rabbit cell, a dog cell, a cat cell, a porcine cell, or a non-human primate cell.

### Methods of Producing a Polypeptide

Also provided herein are methods of producing a polypeptide that include: (a) culturing a cell (e.g., any of the cells described herein) including any of the nucleic acids encoding any of the polypeptides described herein, or any of the expression vectors described herein that include nucleic acid encoding any of the polypeptides described herein, in a culture medium under conditions sufficient to allow for the production of the polypeptide; and (b) harvesting the polypeptide from the host cell or the culture medium. In some embodiments of any of the methods described herein, the method further includes isolating the polypeptide (e.g., through performance of one or more column chromatography steps, ultrafiltration/diafiltration, and/or viral inactivation). In some embodiments of any of the methods described herein, the method further includes formulating the isolated polypeptide into a composition (e.g., a pharmaceutical composition).

Any of the polypeptides described herein can be produced by any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell. For example, a host cell can be a CHO cell or a HEK cell.

Methods of culturing cells are well known in the art. Cells can be maintained in vitro under conditions that favor cell proliferation, cell growth, and/or cell differentiation. For example, cells can be cultured by contacting a cell (e.g., any of the cells described herein) with a cell culture medium that includes supplemental growth factors to support cell viability and cell growth.

Methods of introducing nucleic acids (e.g., any of the exemplary nucleic acids described herein) and/or expression vectors (e.g., any of the exemplary expression vectors described herein (e.g., an AAV vector)) into cells (e.g., mammalian cells) are known in the art. Non-limiting examples of methods that can be used to introduce a nucleic acid (e.g., any of the exemplary nucleic acids described herein) and/or an expression vector (e.g., any of the exemplary expression vectors described herein (e.g., an AAV vector)) include: electroporation, lipofection, transfection, microinjection, calcium phosphate transfection, dendrimer-based transfection, anionic polymer transfection, cationic polymer transfection, transfection using highly branched organic compounds, cell-squeezing, sonoporation, optical transfection, magnetofection, particle-based transfection (e.g., nanoparticle transfection), transfection using liposomes (e.g., cationic liposomes), and viral transduction (e.g., lentiviral transduction, adenoviral transduction).

Some methods described herein further include isolating the polypeptide from cell culture medium or from a cell (e.g., a mammalian cell) using techniques well-known in the art (e.g., ion exchange chromatography (anionic or cation), metal-affinity chromatography, ligand-affinity chromatography, size exclusion chromatography, hydrophobic interaction chromatography, and precipitation (e.g., ammonium sulfate precipitation, polyethylene glycol precipitation).

### Methods of Treatment

Also provided herein are methods of treating a subject having or suspected of having a coronavirus infection (e.g., a SARS-CoV-2 infection) that includes administering to the subject a therapeutically effective amount of any of the polypeptides described herein, any of the vectors described herein, or any of the pharmaceutical compositions described herein.

Also provided herein are methods of decreasing the rate of infection of a coronavirus in a subject that include administering to the subject a therapeutically effective amount of any of the polypeptides described herein, any of the vectors described herein, or any of the pharmaceutical compositions described herein.

In some embodiments of any of these methods described herein, the subject has been identified as not having previously had a coronavirus infection. In some embodiments of any of these methods described herein, prior to administration of any of the polypeptides or pharmaceutical compositions, the subject has been identified as not having a significant titer of antibodies that bind specifically to the SARS-CoV-2 S glycoprotein.

Described herein, in some embodiments, the methods described herein, the coronavirus is SARS-CoV, MERS-CoV, CoV-229E, HCoV-NL63, HCoV-OC43, or HCoV-HKU1. In some embodiments of any of the methods described herein, the coronavirus is a 229E (alpha coronavirus), NL63 (alpha coronavirus), OC43 (beta coronavirus), HKU1 (beta coronavirus), MERS-CoV (beta coronavirus that causes Middle East Respiratory Syndrome (MERS)), SARS-CoV (beta coronavirus that causes severe acute respiratory syndrome (SARS)), or SARS-CoV-2 (novel coronavirus that causes coronavirus disease 2019 (COVID-19)). In some embodiments, the coronavirus is SARS-CoV-2.

In some embodiments of any of the methods described herein, the subject has previously been identified as having one or more medical conditions selected from the group consisting of: chronic lung disease, moderate asthma, severe asthma, heart conditions, diabetes, obesity, liver disease, chronic kidney disease, and a weakened or suppressed immune system. In some embodiments, wherein the subject having a weakened or suppressed immune system is a subject receiving a cancer treatment, a smoker, a subject who is a transplant recipient, a subject having HIV or AIDS, or a subject receiving a corticosteroid or any other immunosuppressant drug. In some embodiments, the subject having a weakened or suppressed immune system is an elderly subject.

In some embodiments, the subject has a profession that puts the subject at increased risk of exposure to persons infected with a coronavirus (e.g., a doctor or other medical professional).

Methods of detecting the presence of a coronavirus (e.g., SARS-CoV-2) in a subject are known in the art. In some embodiments, the presence of a coronavirus can be detected by viral RNA (e.g., detecting viral RNA using any of the exemplary techniques described herein or know in the art). Non-limiting examples of techniques that can be used to detect the presence of coronavirus (e.g., a SARS-CoV-2 coronavirus) include: ELISA, qRT-PCR, next generation sequence, antibody profiling, real-time PCR, Western blotting, immunoprecipitation, immunohistochemistry, nucleic acid detection using loop-mediated isothermal amplification (LAMP), RT-LAMP and LAMP-sequencing (see e.g., Thi et al., Sci, Transl. Med., 12: DOI: 10.1126/scitranslmed.abc7075 (2020)), nucleic acid detection using Cas13 (see e.g., Ackerman et al., Nat., 582: 277-282 (2020), which is herein incorporated by reference in its entirety), nucleic acid detection using CRISPR-Cas12 (see, e.g., Broughton et al., Nat. Biotech., 38: 870-874 (2020), which is herein incorporated by reference in its entirety), any nucleic acid kit that uses recombinase polymerase amplification (RPA) or immunofluorescence. In some embodiments, the method of detecting the presence of a coronavirus includes detecting viral shedding (e.g., as measured by RT-qPCR).

In some embodiments, the method of detecting the presence of a coronavirus (e.g., SARS-CoV-2) in a subject includes taking a sample (e.g., any of the exemplary samples described herein or known in the art) from a subject. Non-limiting examples of the types of samples taken from a subject can include a nasopharyngeal (NP) specimen, oropharyngeal (OP) specimen, nasal mid-turbinate swab, anterior nares (nasal swab) specimen, nasopharyngeal wash/aspirate or nasal wash/aspirate (NW) specimen, blood specimen, saliva specimen, and a fecal specimen.

In some embodiments, these methods can result in a reduction in the number, severity, or frequency of one or more symptoms of the coronavirus in the subject (e.g., as compared to the number, severity, or frequency of the one or more symptoms of the coronavirus in the subject prior to treatment). In some embodiments, these methods can result in a reduction (e.g., about 1% reduction to about 99% reduction, about 1% reduction to about 95% reduction, about 1% reduction to about 90% reduction, about 1% reduction to about 85% reduction, about 1% reduction to about 80% reduction, about 1% reduction to about 75% reduction, about 1% reduction to about 70% reduction, about 1% reduction to about 65% reduction, about 1% reduction to about 60% reduction, about 1% reduction to about 55% reduction, about 1% reduction to about 50% reduction, about 1% reduction to about 45% reduction, about 1% reduction to about 40% reduction, about 1% reduction to about 35% reduction, about 1% reduction to about 30% reduction, about 1% reduction to about 25% reduction, about 1% reduction to about 20% reduction, about 1% reduction to about 15% reduction, about 1% reduction to about 10% reduction, about 1% reduction to about 5% reduction, about 5% reduction to about 99% reduction, about 5% reduction to about 95% reduction, about 5% reduction to about 90% reduction, about 5% reduction to about 85% reduction, about 5% reduction to about 80% reduction, about 5% reduction to about 75% reduction, about 5% reduction to about 70% reduction, about 5% reduction to about 65% reduction, about 5% reduction to about 60% reduction, about 5% reduction to about 55% reduction, about 5% reduction to about 50% reduction, about 5% reduction to about 45% reduction, about 5% reduction to about 40% reduction, about 5% reduction to about 35% reduction, about 5% reduction to about 30% reduction, about 5% reduction to about 25% reduction, about 5% reduction to about 20% reduction, about 5% reduction to about 15% reduction, about 5% reduction to about 10% reduction, about 10% reduction to about 99% reduction, about 10% reduction to about 95% reduction, about 10% reduction to about 90% reduction, about 10% reduction to about 85% reduction, about 10% reduction to about 80% reduction, about 10% reduction to about 75% reduction, about 10% reduction to about 70% reduction, about 10% reduction to about 65% reduction, about 10% reduction to about 60% reduction, about 10% reduction to about 55% reduction, about 10% reduction to about 50% reduction, about 10% reduction to about 45% reduction, about 10% reduction to about 40% reduction, about 10% reduction to about 35% reduction, about 10% reduction to about 30% reduction, about 10% reduction to about 25% reduction, about 10% reduction to about 20% reduction, about 10% reduction to about 15% reduction, about 15% reduction to about 99% reduction, about 15% reduction to about 95% reduction, about 15% reduction to about 90% reduction, about 15% reduction to about 85% reduction, about 15% reduction to about 80% reduction, about 15% reduction to about 75% reduction, about 15% reduction to about 70% reduction, about 15% reduction to about 65% reduction, about 15% reduction to about 60% reduction, about 15% reduction to about 55% reduction, about 15% reduction to about 50% reduction, about 15% reduction to about 45% reduction, about 15% reduction to about 40% reduction, about 15% reduction to about 35% reduction, about 15% reduction to about 30% reduction, about 15% reduction to about 25% reduction, about 15% reduction to about 20% reduction, about 20% reduction to about 99% reduction, about 20% reduction to about 95% reduction, about 20% reduction to about 90% reduction, about 20% reduction to about 85% reduction, about 20% reduction to about 80% reduction, about 20% reduction to about 75% reduction, about 20% reduction to about 70% reduction, about 20% reduction to about 65% reduction, about 20% reduction to about 60% reduction, about 20% reduction to about 55% reduction, about 20% reduction to about 50% reduction, about 20% reduction to about 45% reduction, about 20% reduction to about 40% reduction, about 20% reduction to about 35% reduction, about 20% reduction to about 30% reduction, about 20% reduction to about 25% reduction, about 25% reduction to about 99% reduction, about 25% reduction to about 95% reduction, about 25% reduction to about 90% reduction, about 25% reduction to about 85% reduction, about 25% reduction to about 80% reduction, about 25% reduction to about 75% reduction, about 25% reduction to about 70% reduction, about 25% reduction to about 65% reduction, about 25% reduction to about 60% reduction, about 25% reduction to about 55% reduction, about 25% reduction to about 50% reduction, about 25% reduction to about 45% reduction, about 25% reduction to about 40% reduction, about 25% reduction to about 35% reduction, about 25% reduction to about 30% reduction, about 30% reduction to about 99% reduction, about 30% reduction to about 95% reduction, about 30% reduction to about 90% reduction, about 30% reduction to about 85% reduction, about 30% reduction to about 80% reduction, about 30% reduction to about 75% reduction, about 30% reduction to about 70% reduction, about 30% reduction to about 65% reduction, about 30% reduction to about 60% reduction, about 30% reduction to about 55% reduction, about 30% reduction to about 50% reduction, about 30% reduction to about 45% reduction, about 30% reduction to about 40% reduction, about 30% reduction to about 35% reduction, about 35% reduction to about 99% reduction, about 35% reduction to about 95% reduction, about 35% reduction to about 90% reduction, about 35% reduction to about 85% reduction, about 35% reduction to about 80% reduction, about 35% reduction to about 75% reduction, about 35% reduction to about 70% reduction, about 35% reduction to about 65% reduction, about 35% reduction to about 60% reduction, about 35% reduction to about 55% reduction, about 35% reduction to about 50% reduction, about 35% reduction to about 45% reduction, about 35% reduction to about 40% reduction, about 40% reduction to about 99% reduction, about 40% reduction to about 95% reduction, about 40% reduction to about 90% reduction, about 40% reduction to about 85% reduction, about 40% reduction to about 80% reduction, about 40% reduction to about 75% reduction, about 40% reduction to about 70% reduction, about 40% reduction to about 65% reduction, about 40% reduction to about 60% reduction, about 40% reduction to about 55% reduction, about 40% reduction to about 50% reduction, about 40% reduction to about 45% reduction, about 45% reduction to about 99% reduction, about 45% reduction to about 95% reduction, about 45% reduction to about 90% reduction, about 45% reduction to about 85% reduction, about 45% reduction to about 80% reduction, about 45% reduction to about 75% reduction, about 45% reduction to about 70% reduction, about 45% reduction to about 65% reduction, about 45% reduction to about 60% reduction, about 45% reduction to about 55% reduction, about 45% reduction to about 50% reduction, about 50% reduction to about 99% reduction, about 50% reduction to about 95% reduction, about 50% reduction to about 90% reduction, about 50% reduction to about 85% reduction, about 50% reduction to about 80% reduction, about 50% reduction to about 75% reduction, about 50% reduction to about 70% reduction, about 50% reduction to about 65% reduction, about 50% reduction to about 60% reduction, about 50% reduction to about 55% reduction, about 55% reduction to about 99% reduction, about 55% reduction to about 95% reduction, about 55% reduction to about 90% reduction, about 55% reduction to about 85% reduction, about 55% reduction to about 80% reduction, about 55% reduction to about 75% reduction, about 55% reduction to about 70% reduction, about 55% reduction to about 65% reduction, about 55% reduction to about 60% reduction, about 60% reduction to about 99% reduction, about 60% reduction to about 95% reduction, about 60% reduction to about 90% reduction, about 60% reduction to about 85% reduction, about 60% reduction to about 80% reduction, about 60% reduction to about 75% reduction, about 60% reduction to about 70% reduction, about 60% reduction to about 65% reduction, about 65% reduction to about 99% reduction, about 65% reduction to about 95% reduction, about 65% reduction to about 90% reduction, about 65% reduction to about 85% reduction, about 65% reduction to about 80% reduction, about 65% reduction to about 75% reduction, about 65% reduction to about 70% reduction, about 70% reduction to about 99% reduction, about 70% reduction to about 95% reduction, about 70% reduction to about 90% reduction, about 70% reduction to about 85% reduction, about 70% reduction to about 80% reduction, about 70% reduction to about 75% reduction, about 75% reduction to about 99% reduction, about 75% reduction to about 95% reduction, about 75% reduction to about 90% reduction, about 75% reduction to about 85% reduction, about 75% reduction to about 80% reduction, about 80% reduction to about 99% reduction, about 80% reduction to about 95% reduction, about 80% reduction to about 90% reduction, about 80% reduction to about 85% reduction, about 85% reduction to about 99% reduction, about 85% reduction to about 95% reduction, about 85% reduction to about 90% reduction, about 90% reduction to about 99% reduction, about 90% reduction to about 95% reduction, or about 95% reduction to about 99% reduction) in the requirement for supplemental oxygen in the subject (e.g., as compared to the requirement for supplemental oxygen prior to treatment or at the start of treatment).

In some embodiments, the methods can reduce (e.g., about 1% reduction to about 99% reduction, or any of the subranges of this range described herein) the requirement for high-intensity oxygen therapy in a subject (e.g., as compared to the requirement for high-intensity oxygen therapy in a subject prior to treatment or in a similar subject or a population of subjects administered a different treatment).

In some embodiments, the methods can reduce (e.g., about 1% reduction to about 99% reduction or any of the subranges of this range described herein) the requirement for mechanical ventilation in a subject (e.g., as compared to the requirement for high-intensity oxygen therapy in a subject prior to treatment or in a similar subject or a population of subjects administered a different treatment).

In some embodiments, the methods can result in an increase in the oxygen level and/or oxygen saturation levels (SpO₂) in the subject (e.g., as compared to the oxygen level and/or oxygen saturation levels (SpO₂) in the subject prior to treatment). A non-limiting example of measuring oxygen level and/or oxygen saturation levels (SpO₂) can include using a pulse oximeter.

In some embodiments, the methods can result in a decrease (e.g., about 1% decrease to about 99% decrease, or any of the subranges of this range described herein) in the level or concentration of coronavirus RNA in the subject (e.g., a decrease in the amount of coronavirus RNA in the sample taken from the subject), e.g., as compared to the level of coronavirus RNA in the subject prior to treatment.

### Additional Therapeutic Agents

Also provided herein are methods of treating a subject having or suspected of having a coronavirus (e.g., SARS-CoV-2) infection that includes administering to the subject (i) a therapeutically effective amount of any of the polypeptides described herein, any of the vectors described herein, and/or any of the pharmaceutical compositions described herein, and (ii) a therapeutically effective amount of an additional therapeutic agent used to treat a subject having or suspected of having a coronavirus infection. In some embodiments, the additional therapeutic agent is not used specifically to treat a subject having or suspected of having a coronavirus infection.

Some embodiments of any of the methods described herein can further include administering to a subject (e.g., any of the subjects described herein) a therapeutically effective amount of one or more additional therapeutic agents. The one or more additional therapeutic agents can be administered to the subject at substantially the same time as a polypeptide (e.g., any of the exemplary polypeptides described herein) or a pharmaceutical composition (e.g., any of the exemplary pharmaceutical compositions described herein). In some embodiments, one or more additional therapeutic agents can be administered to the subject prior to administration of a polypeptide (e.g., any of the exemplary polypeptides described herein) or pharmaceutical composition (e.g., any of the exemplary pharmaceutical compositions described herein). In some embodiments, one or more additional therapeutic agents can be administered to the subject after administration of a polypeptide (e.g., any of the exemplary polypeptides described herein) or a pharmaceutical composition (e.g., any of the exemplary pharmaceutical compositions described herein) to the subject.

Non-limiting examples of additional therapeutic agents include: anti-viral drugs, convalescent plasma (e.g., convalescent plasma collected from subjects who have survived a coronavirus infection by producing protective antibodies), spike glycoprotein-angiotensin converting enzyme 2 receptor (ACE2) blockers, chloroquine and hydroxychloroquine, antibodies, JAK inhibitors, cell therapies (e.g., mesenchymal stem cells, and NK cells), immunoenhancers, corticosterioids, vitamins, anticoagulants, non-sterodial anti-inflammatory drugs, a vaccine, anti-parasitic drugs and nutritional supplements (e.g., zinc).

Non-limiting example of an anti-viral drug includes: remdesivir (interferes with virus RNA polymerases to inhibit viral replication), lopinavir/ritonavir (e.g., viral protease inhibitors), favipiravir (e.g., drug that inhibits viral RNA polymerase), EIDD-2801 and EIDD-1931 (EIDD-2801 and EIDD-1931 are both ribonucleotide analog incorporated into viral RNA during RNA synthesis to drive mutagenesis thereby inhibiting viral replication).

In some embodiments, the methods further include administering to a subject (e.g., any of the subjects described herein) a therapeutically effective amount of one or more additional therapeutic agents include administering an agent that blocks the spike glycoprotein from binding to the ACE2 receptor thereby blocking entry into the cell. Non-limiting examples of agents that can block the spike glycoprotein from binding to an ACE2 receptor include: recombinant human ACE2 (rhACE2), rhACE fused to an Fc domain, ACE inhibitors, angiotensin receptor blockers, and JAK inhibitor (e.g., baricitinib).

Some embodiments of any of the methods described herein that further include administering to a subject (e.g., any of the subject described herein) a therapeutically effective amount of one or more antibodies. The antibodies can target proteins (e.g., without limitation receptors and cytokines) that are involved in cytokine release. For example, the methods provided herein include administering used to treat cytokine release syndrome (CRS). In some embodiments where the additional therapeutic agent is an antibody, the antibody can be directed against interleukin-6 (IL-6), IL-6 receptors, IL-1, IL-2, IL-7, IL-10, granulocyte stimulating factor, tumor necrosis factor alpha (TNF-α), interferon-7 inducible protein 10, macrophage inflammatory protein-1 alpha, monocyte chemoattractant protein 1, and vascular endothelial growth factor (VEGF). Non-limiting examples of antibodies that can be used as an additional therapeutic agent in the methods described herein include: tocilizumab, sarilumab, siltuximab, clazakizumab, and bevacizumab. In some embodiments, the methods described herein include an additional therapeutic agent that includes a small molecule inhibitor directed against interleukin-6 (IL-6), IL-6 receptors, IL-1, IL-2, IL-7, IL-10, granulocyte stimulating factor, tumor necrosis factor alpha (TNF-α), interferon-7 inducible protein 10, macrophage inflammatory protein-1 alpha, monocyte chemoattractant protein 1, and vascular endothelial growth factor (VEGF).

### Methods of Detecting a Coronavirus

Also provided herein are methods of detecting a coronavirus infection in a subject using a polypeptide (e.g., any of the exemplary polypeptides described herein). In some embodiments, a sample (e.g., a blood sample, a fluid sample, or a tissue sample) taken from a subject (e.g., a subject either previously identified as having a coronavirus infection or not previously identified as having a coronavirus infection) can be contacted with a polypeptide (e.g., any of the exemplary polypeptides described herein). In some embodiments, any of the polypeptides described herein can be labeled with a detectable label. As used herein, the terms “detectable label” and “label” are used interchangeably herein to refer to a directly or indirectly detectable moiety that is associated with (e.g., conjugated to) a molecule to be detected (e.g., a polypeptide, an antibody, or an antibody fragment). The detectable label can be directly detectable by itself (e.g., radioisotope labels or fluorescent labels) or, in the case of an enzymatic label, can be indirectly detectable, for example, by catalyzing chemical alterations of a chemical substrate compound or composition, which chemical substrate compound or composition is directly detectable. In some embodiments, the detectable labels can be suitable for small scale detection and/or suitable for high-throughput screening. Non-limiting examples of suitable detectable labels include, but are not limited to, metals, radioisotopes, fluorophores, bioluminescent compounds, chemiluminescent compounds, and dyes.

In some embodiments where a polypeptide (e.g., any of the exemplary polypeptides described herein) is used to detect a coronavirus infection in a subject, the polypeptide can be tagged with (e.g., attached to) metals, radioisotopes, fluorophores, bioluminescent compounds, chemiluminescent compounds, and/or dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties.

In some embodiments, the detectable label is a metal ion. Non-limiting examples of metal ions include: lanthanides (e.g., lanthanum, cerium, praseodymium, neodymium, promethium, samarium, europium, gadolinium, terbium, dysprosium, holmium, erbium, thulium, ytterbium, and lutetium), indium, yttrium, palladium, and bismuth. Methods for tagging any of the exemplary polypeptides, antibodies, or antibody fragments are provided by Han et al. (Nat. Protocols, 13: 2121-2148 (2018), which is herein incorporated by reference in its entirety).

In some embodiments, the detectable label is a fluorophore. Non-limiting examples of fluorophores include: Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™ SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate chemical substrates (e.g., an oxidizing reagent plus a chemiluminescent compound). A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. Other non-limiting examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters.

Methods of detecting tagged polypeptides are well-known in the art and include but are not limited to enzyme linked immunosorbent assay (ELISA), fluorescent activated cell sorting (FACS), Western blotting, immunoprecipitation, immunofluorescence, mass spectrometry (LC-MS, Inductively Coupled Plasma Mass Spectrometry (ICP-MS), MS based methods (LC-MS/MS), electrochemicalluminecence immunoassay, and homogenous mobile shift assays. Additional methods of detecting the detectable labels are known in the art.

### Methods of Neutralizing a Coronavirus

Also provided herein are methods of neutralizing a coronavirus (e.g., SARS-CoV-2) in a subject in need thereof (e.g., any of the exemplary subjects described herein or known in the art) that include administering to the subject a therapeutically effective amount of any of the polypeptides described herein or any of the compositions (e.g., pharmaceutical compositions) described herein. The polypeptide works to neutralize the coronavirus by binding to the coronavirus and blocking entry into a subject's cells

Also provided herein are methods of neutralizing a coronavirus (e.g., SARS-CoV-2) present on the surface of a PPE or other surfaces or present in water or in the air. In some cases, neutralization of a coronavirus can occur in a subject (e.g., any of the exemplary subjects described herein).

### Compositions and Kits

Also provided herein are compositions (e.g., pharmaceutical compositions) that include any of the polypeptides, vectors, or nucleic acids described herein. Any of the pharmaceutical compositions can include any of the polypeptides, vectors, or nucleic acids described herein and one or more (e.g., 1, 2, 3, 4, or 5) pharmaceutically or physiologically acceptable carriers, diluents, or excipients. In some embodiments, any of the pharmaceutical compositions described herein can include one or more buffers (e.g., a neutral-buffered saline, a phosphate-buffered saline (PBS)), one or more carbohydrates (e.g., glucose, mannose, sucrose, dextran, or mannitol), one or more proteins, polypeptides, or amino acids (e.g., glycine), one or more antioxidants, one or more chelating agents (e.g., glutathione or EDTA), one or more preservatives, and/or a pharmaceutically acceptable carrier (e.g., PBS, saline, or bacteriostatic water).

In some embodiments, any of the pharmaceutical compositions described herein can further include one or more (e.g., 1, 2, 3, 4, or 5) agents that promote the entry of any of the vectors or nucleic acids described herein into a cell (e.g., a mammalian cell) (e.g., a liposome or cationic lipid).

In some embodiments, any of the vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers and poly(lactic-co-glycolic acid) (PLGA) polymers.

In some embodiments of any of the pharmaceutical compositions described herein, a single dose of a pharmaceutical composition can include a total sum amount of at least 1 ng (e.g., at least 2 ng, at least 4 ng, at least 5 ng, at least 6 ng, at least 8 ng, at least 10 ng, at least 15 ng, at least 20 ng, at least 30 ng, at least 40 ng, at least 50 ng, at least 60 ng, at least 80 ng, at least 100 ng, at least 120 ng, at least 200 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μg, at least 20 μg, at least 24 μg, at least 25 μg, at least 30 μg, at least 40 μg, at least 50 μg, at least 60 μg, at least 80 μg, at least 100 μg, at least 120 μg, at least 140 μg, at least 150 μg, at least 160 μg, at least 180 μg, or at least 200 μg) of any of the polypeptides, nucleic acids, or vectors described herein, e.g., in a buffered solution.

The pharmaceutical compositions provided herein can be, e.g., formulated to be compatible with their intended route of administration. In some embodiments, the compositions are formulated for subcutaneous, intramuscular, or intravenous administration. In some examples, the compositions include a therapeutically effective amount of any of the polypeptides, vectors, or nucleic acids described herein. Single or multiple administrations of any of the pharmaceutical compositions described herein can be given (e.g., administered) to a subject depending on, for example, the frequency and the dosage required and tolerated by the subject. A dosage of the pharmaceutical composition including any of the polypeptides described herein, any of the vectors described herein, or any of the nucleic acids described herein should provide a sufficient quantity to effectively ameliorate or treat symptoms, conditions or diseases.

Also provided are kits that include any of the compositions (e.g., pharmaceutical compositions) described herein that include any of the nucleic acids, any of the polypeptides, any, or any of the vectors described herein. In some embodiments, a kit can include a solid composition (e.g., a lyophilized composition including any of the vectors, polypeptides, or nucleic acids described herein) and a liquid for solubilizing the lyophilized composition.

In some embodiments, a kit can include at least one dose of any of the compositions (e.g., any of the pharmaceutical compositions) described herein.

In some embodiments, a kit can include a pre-loaded syringe including any of the pharmaceutical compositions described herein.

In some embodiments, the kit includes a vial including any of the pharmaceutical compositions described herein (e.g., formulated as an aqueous pharmaceutical composition).

In some embodiments, the kit can include instructions for performing any of the methods described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1. Binding Affinity Measurements

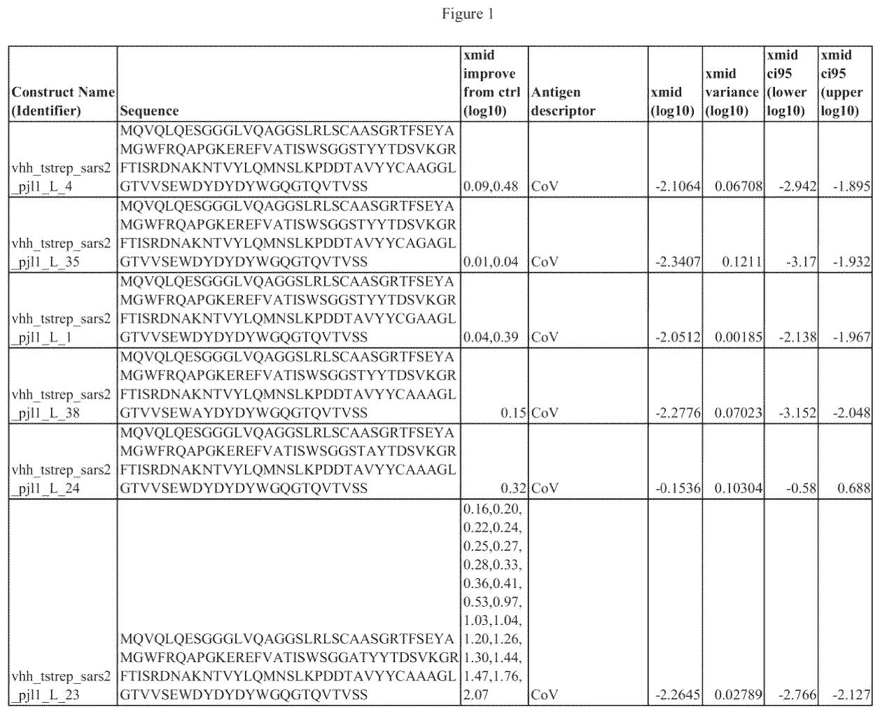

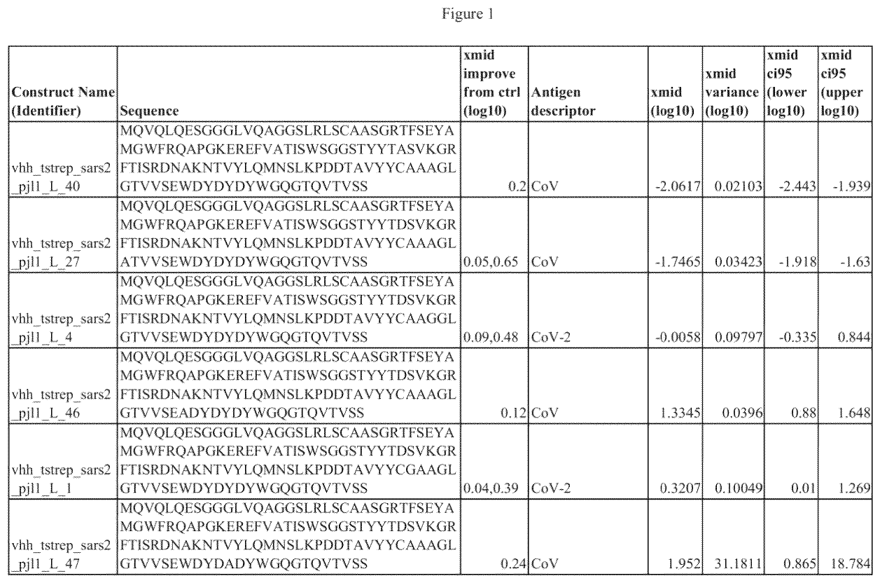

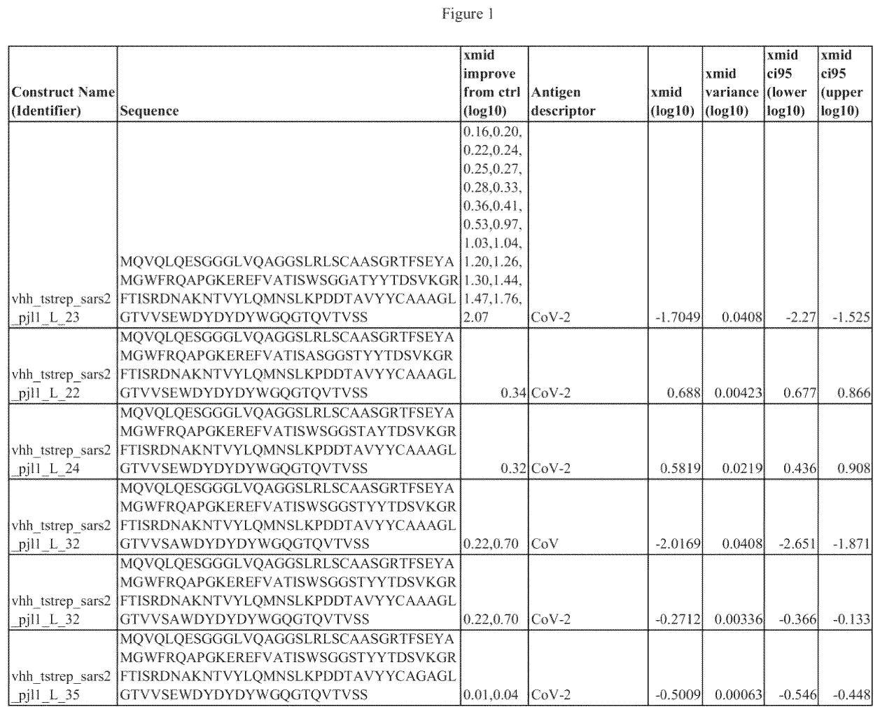

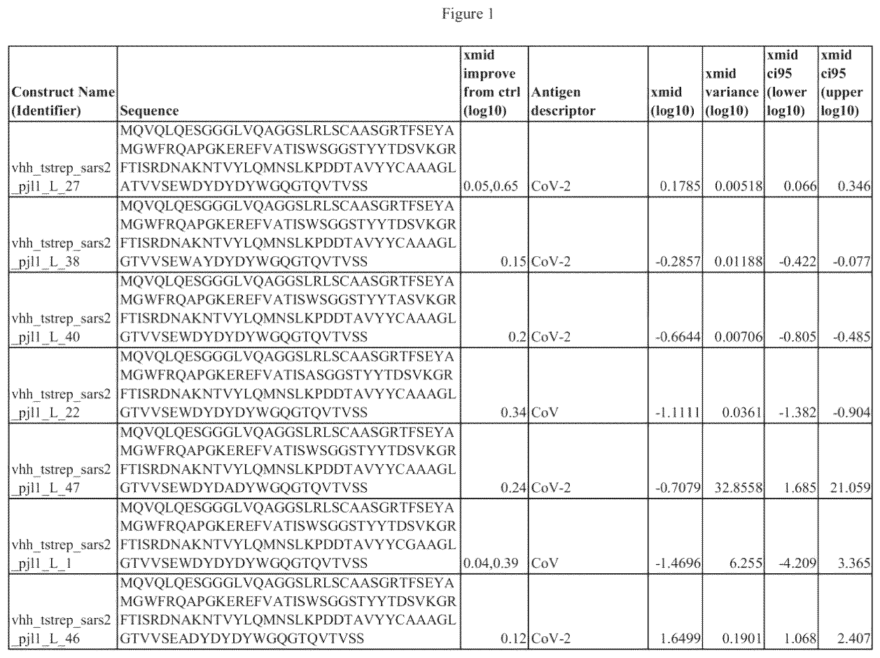

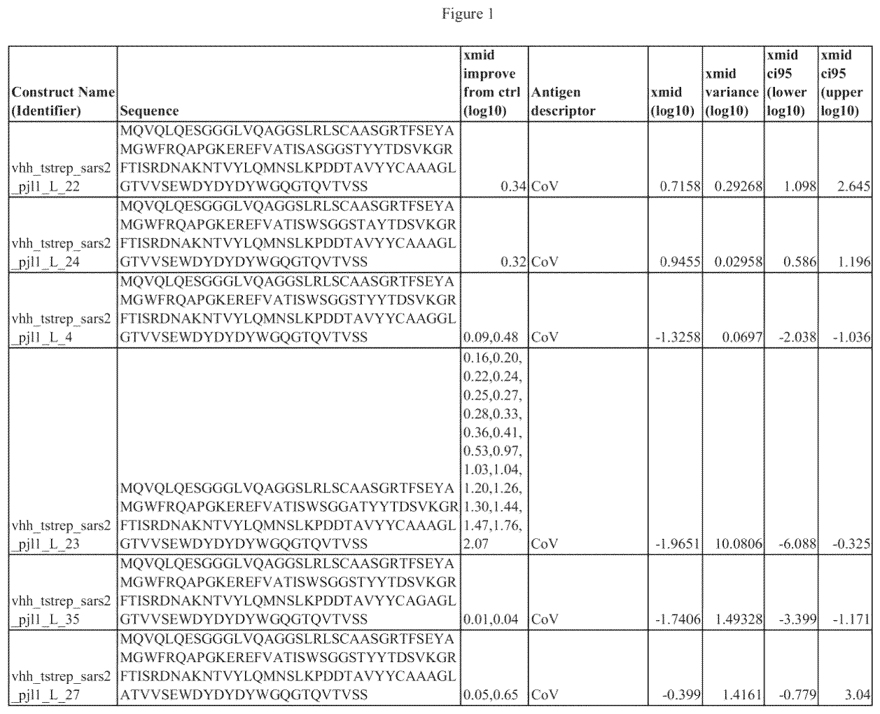

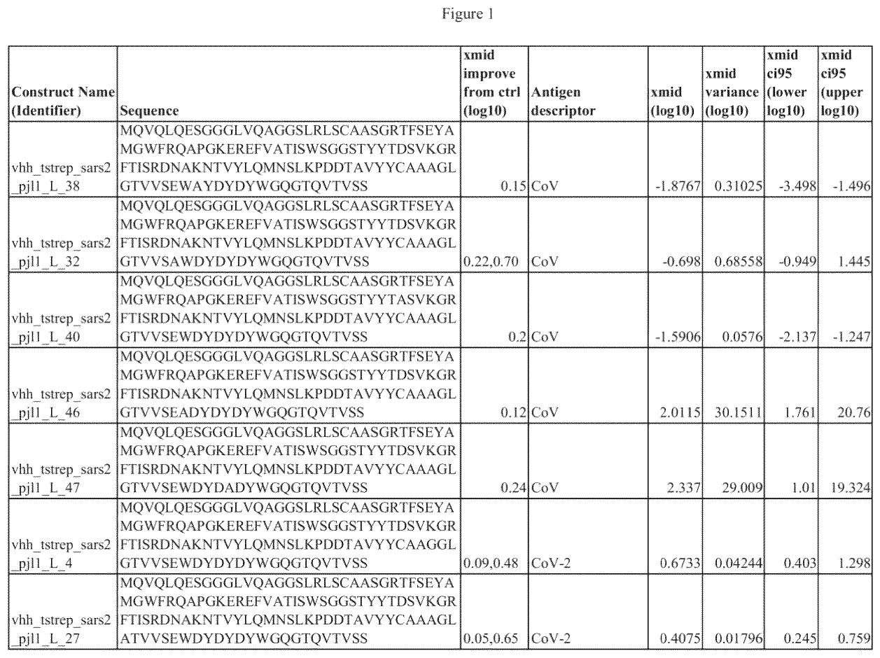

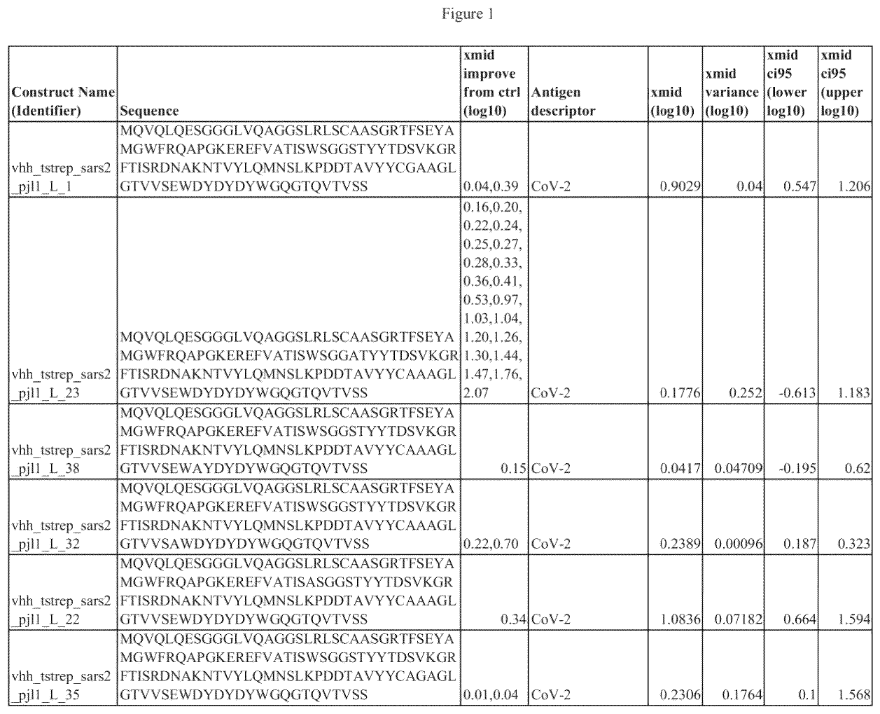

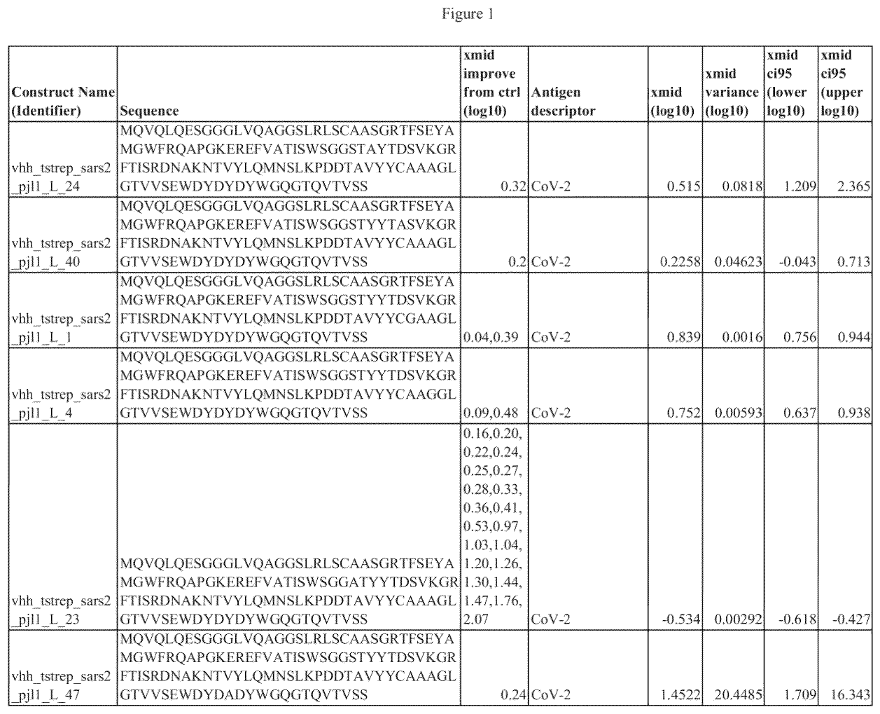

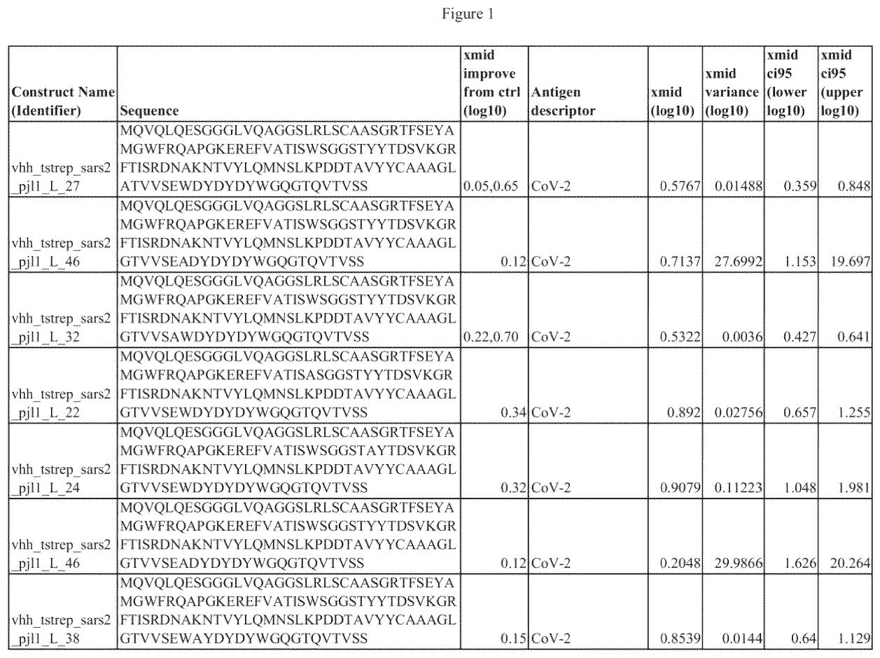

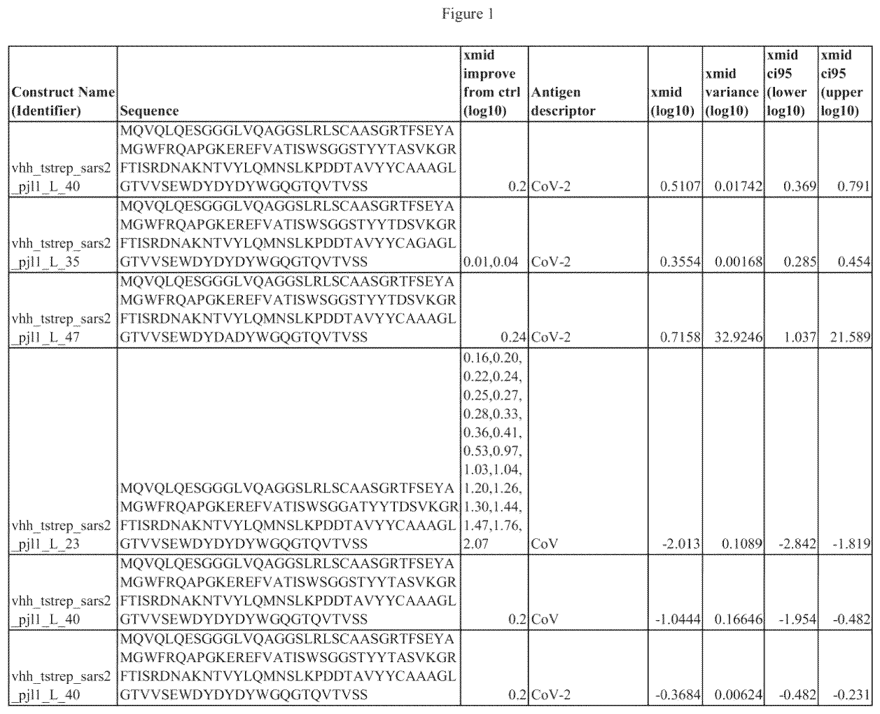

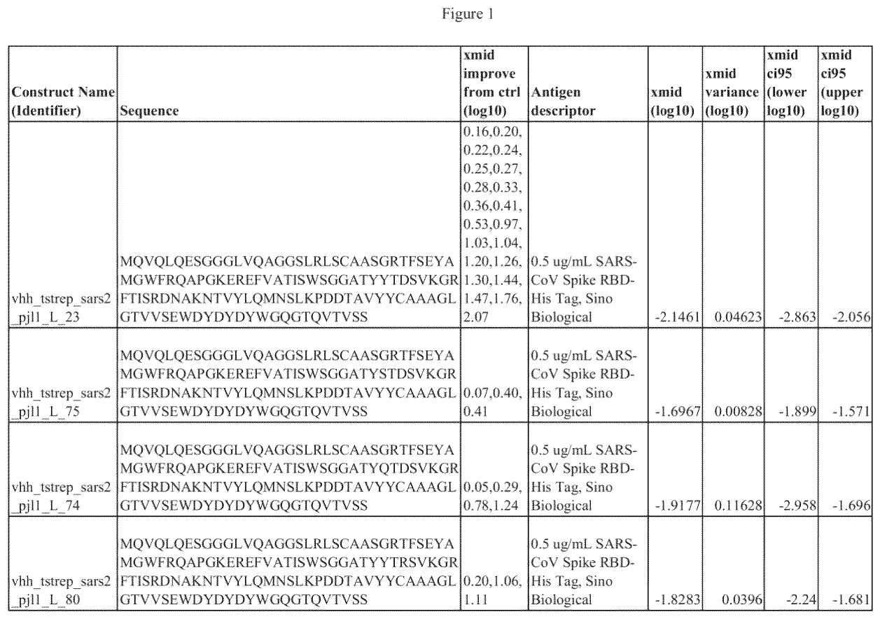

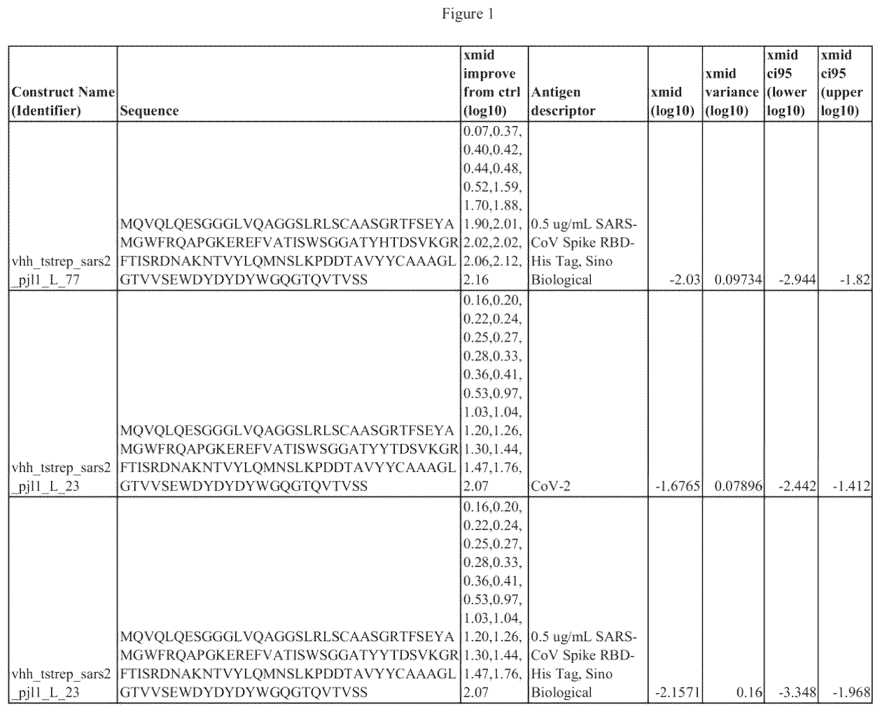

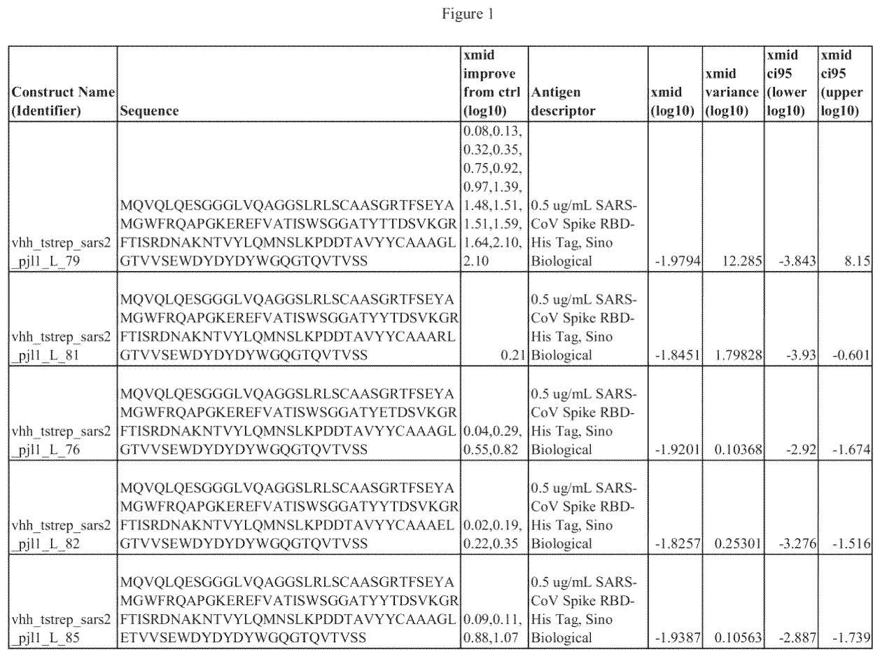

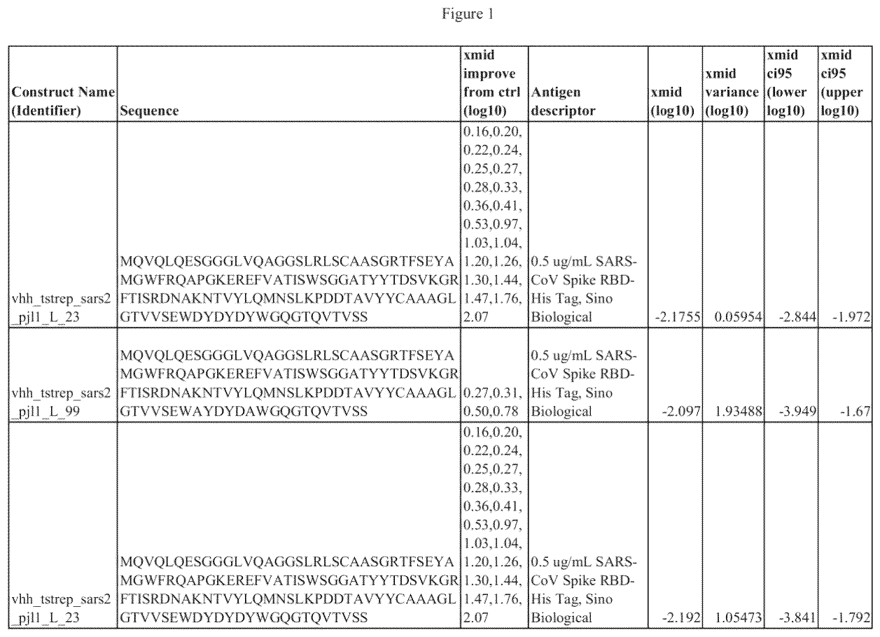

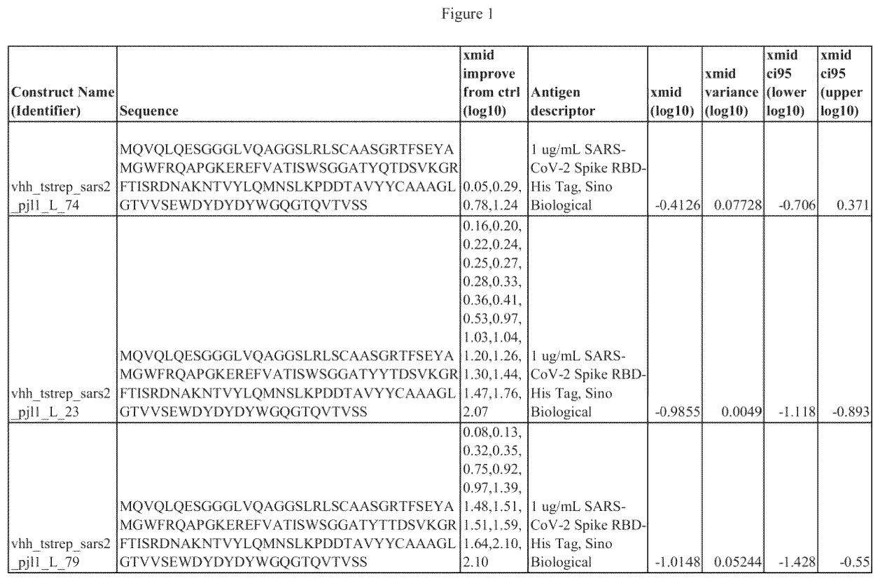

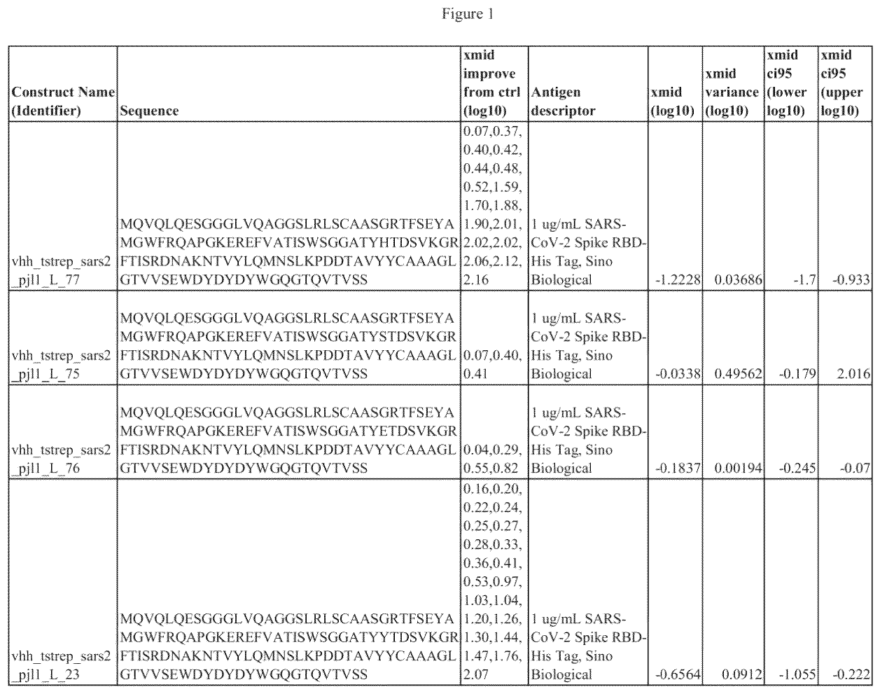

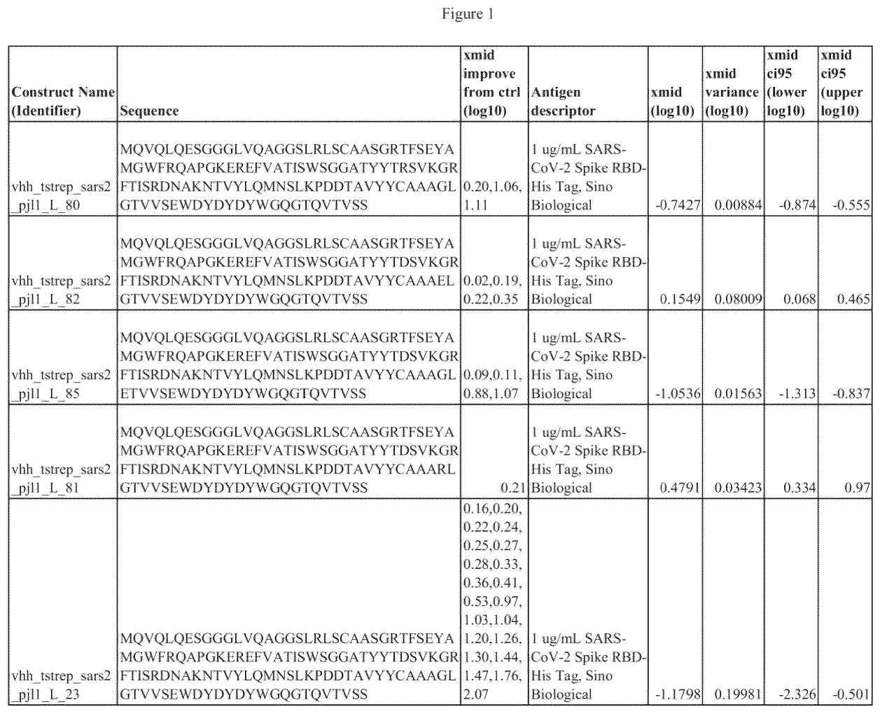

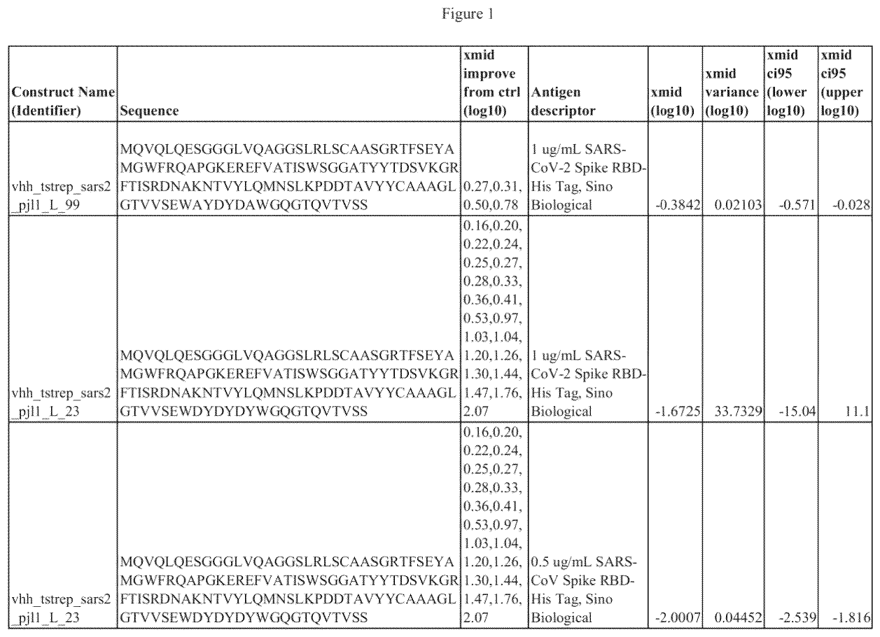

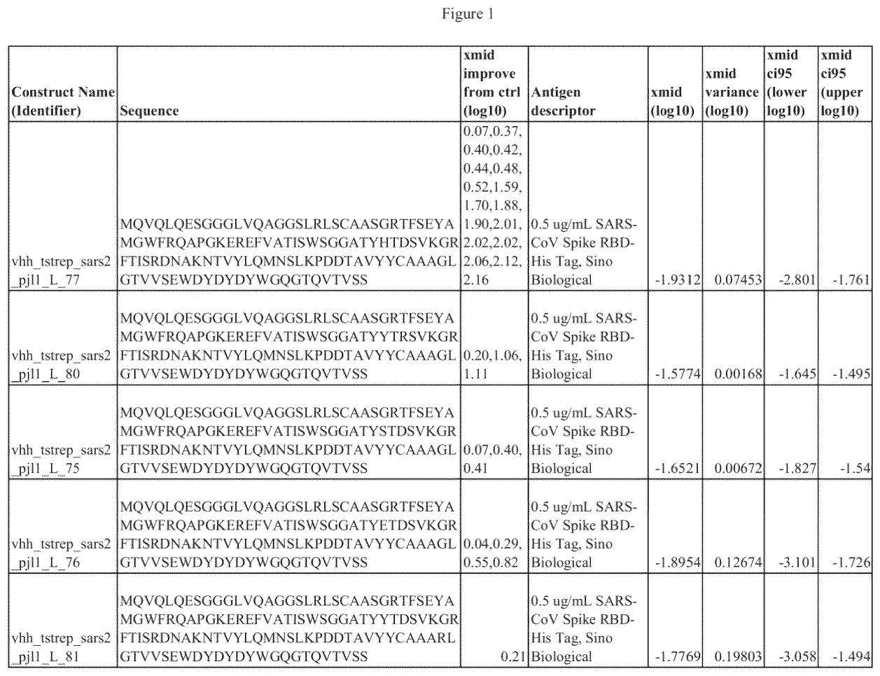

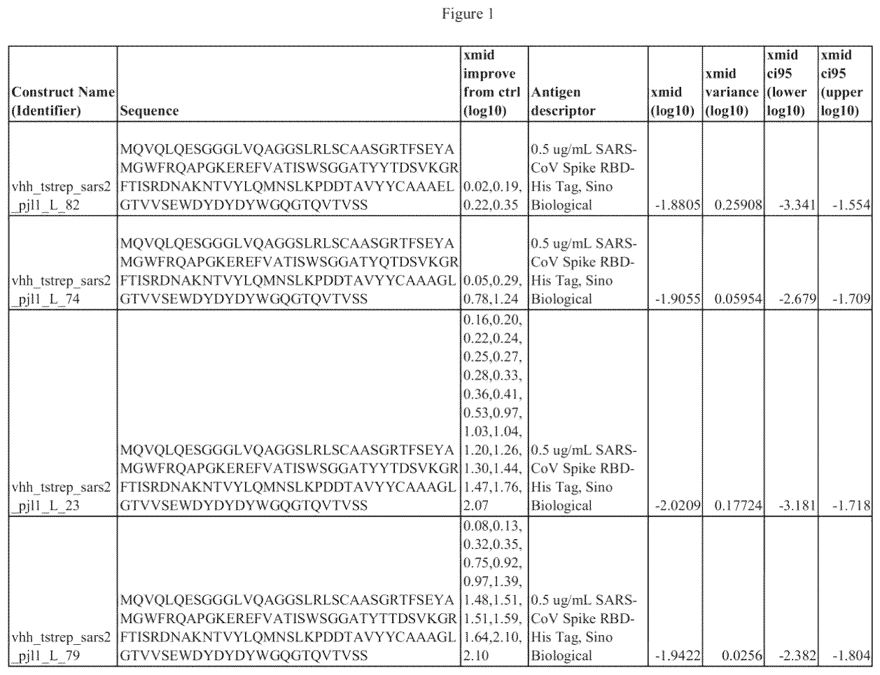

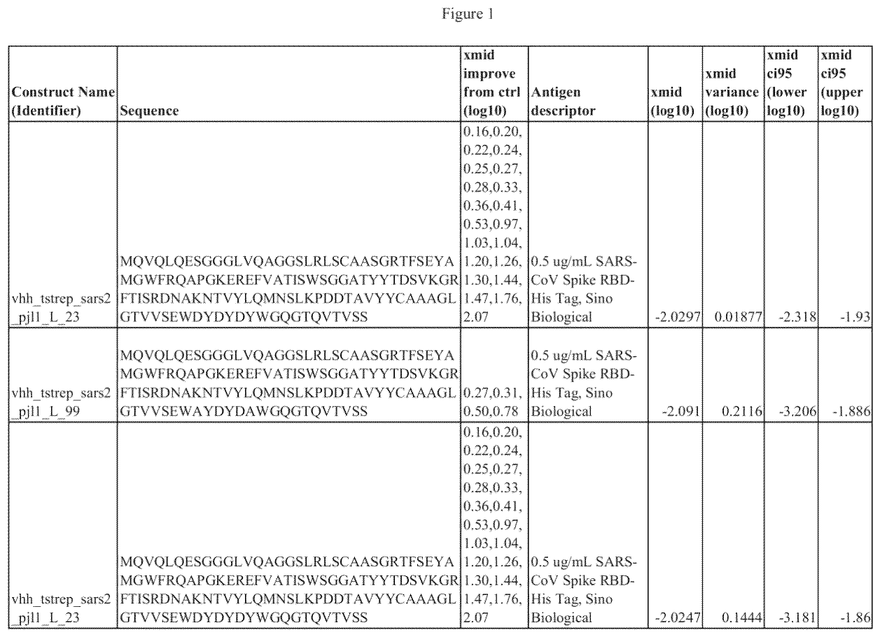

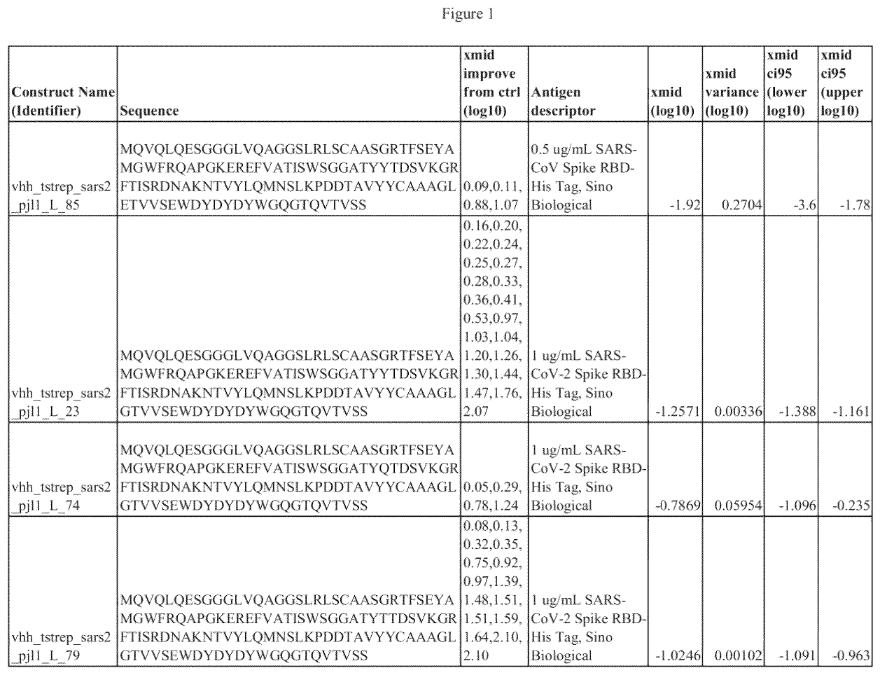

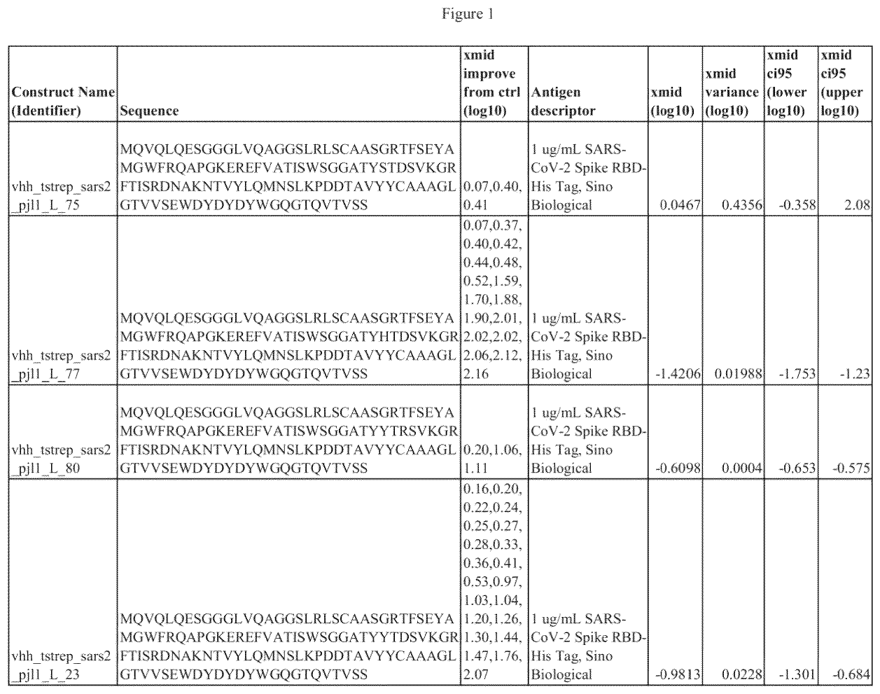

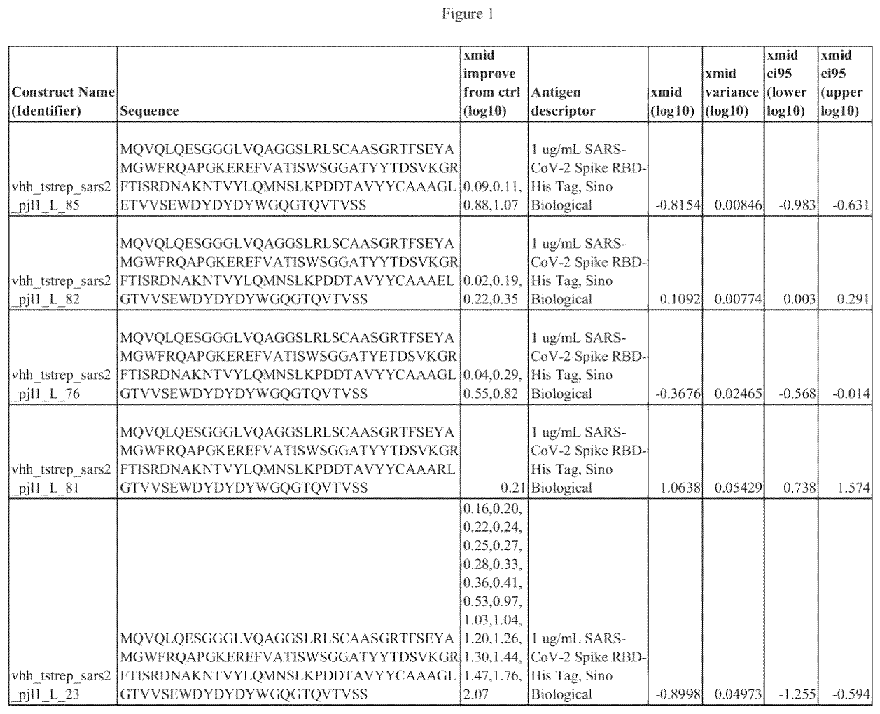

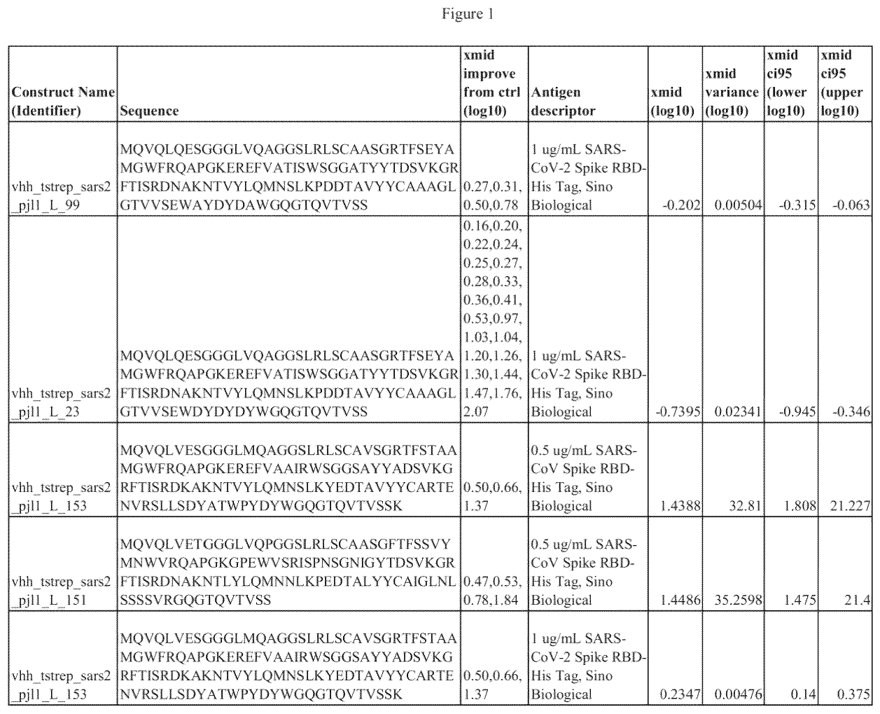

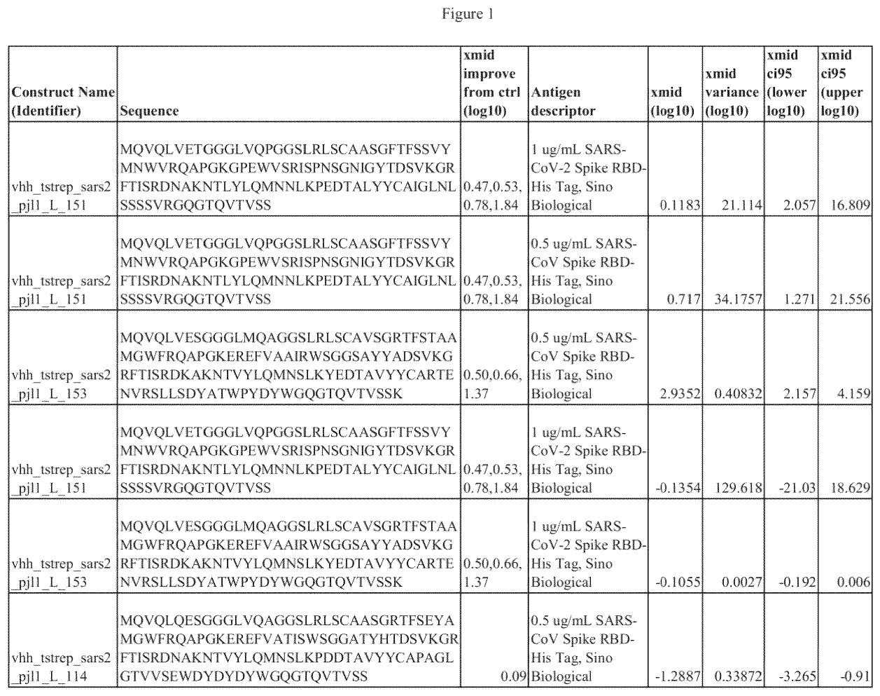

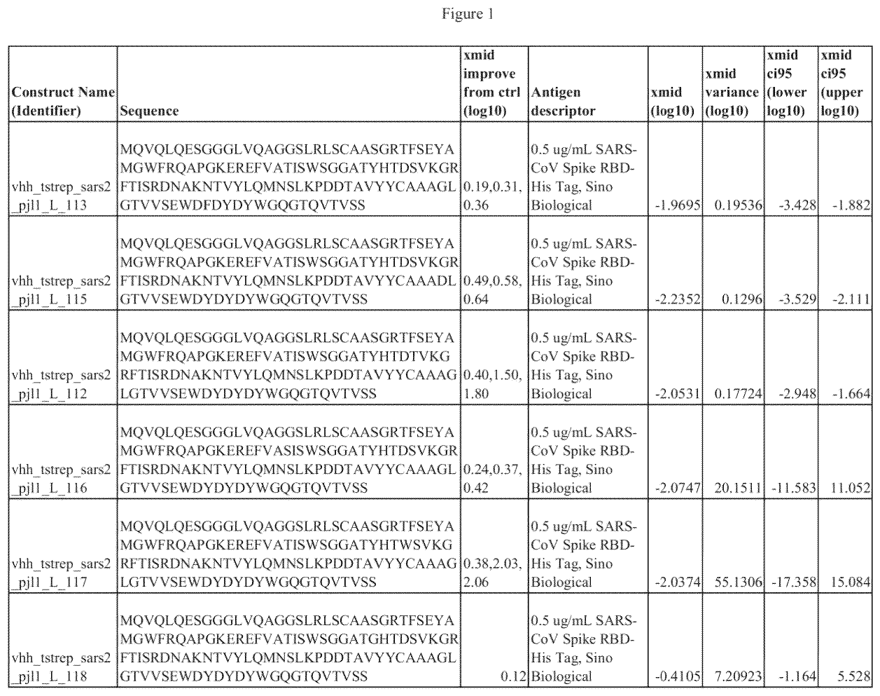

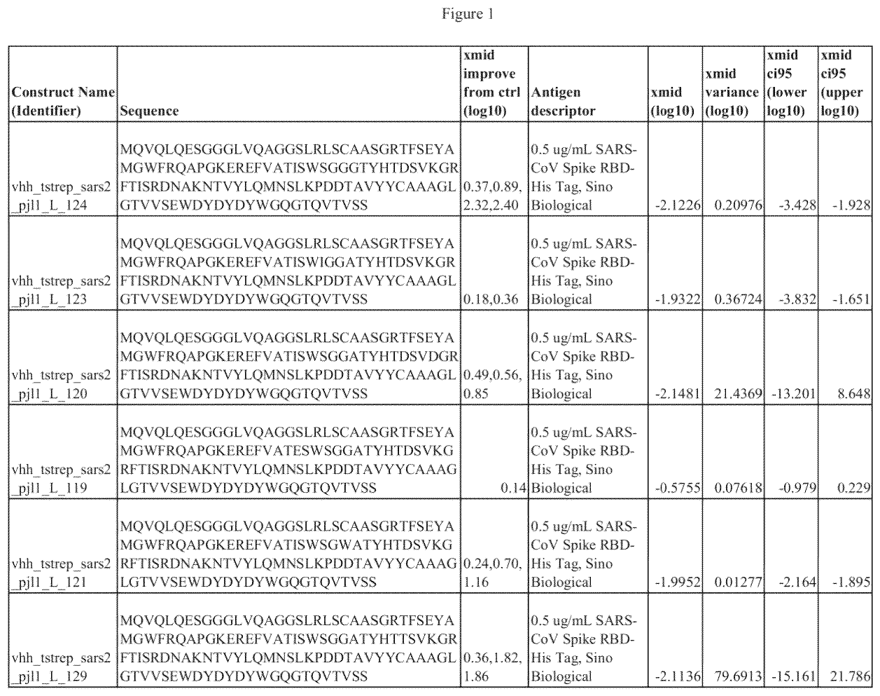

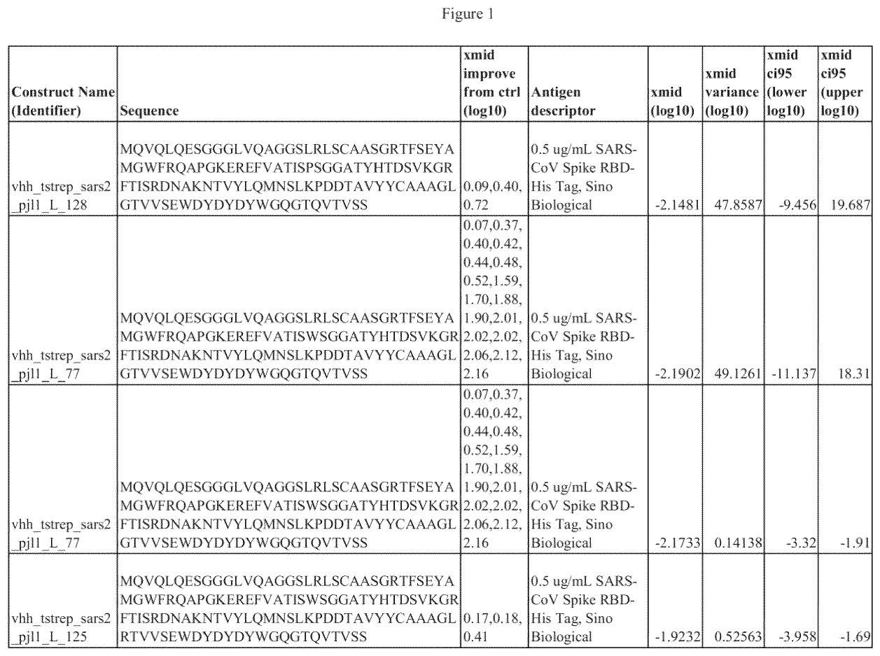

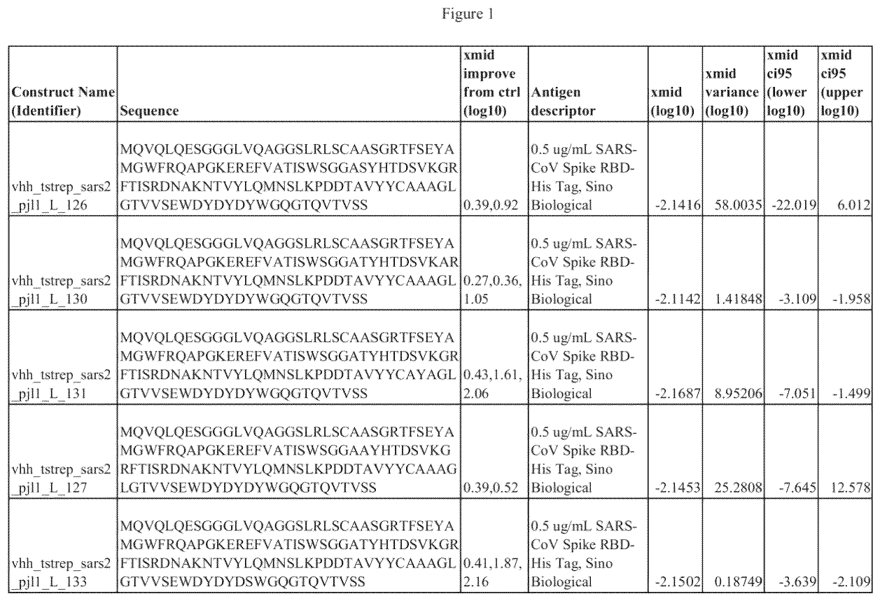

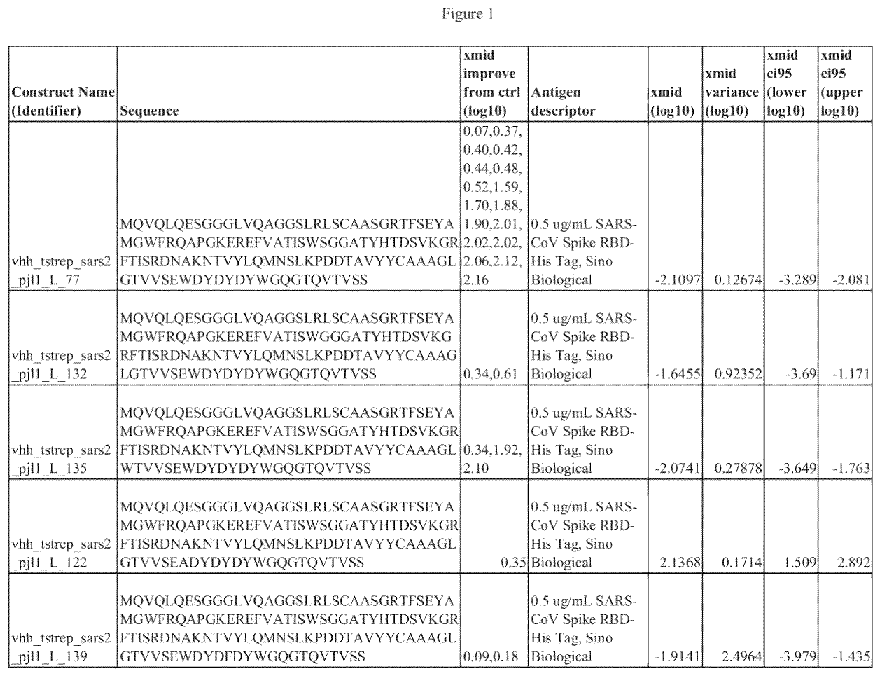

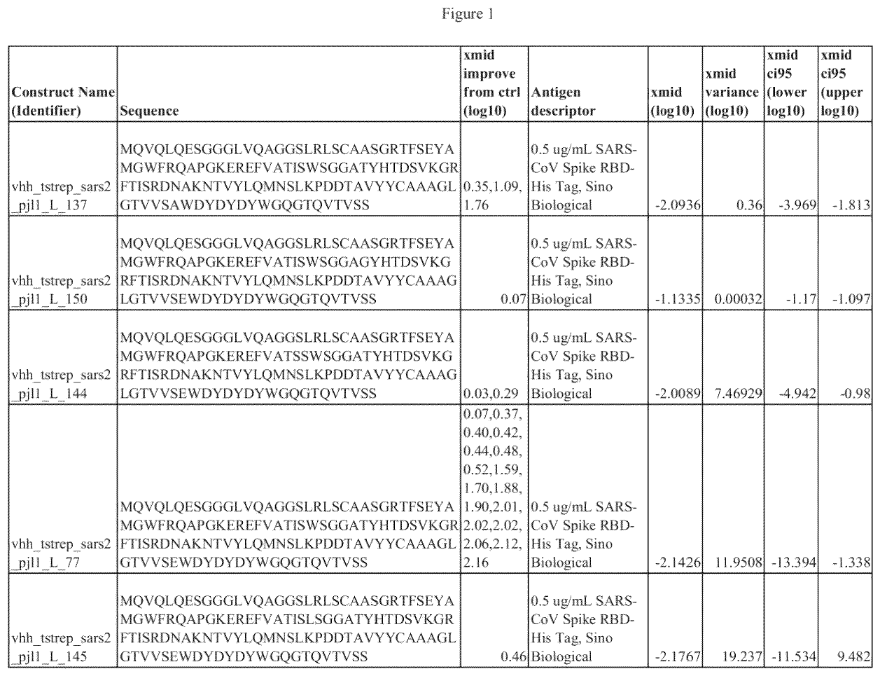

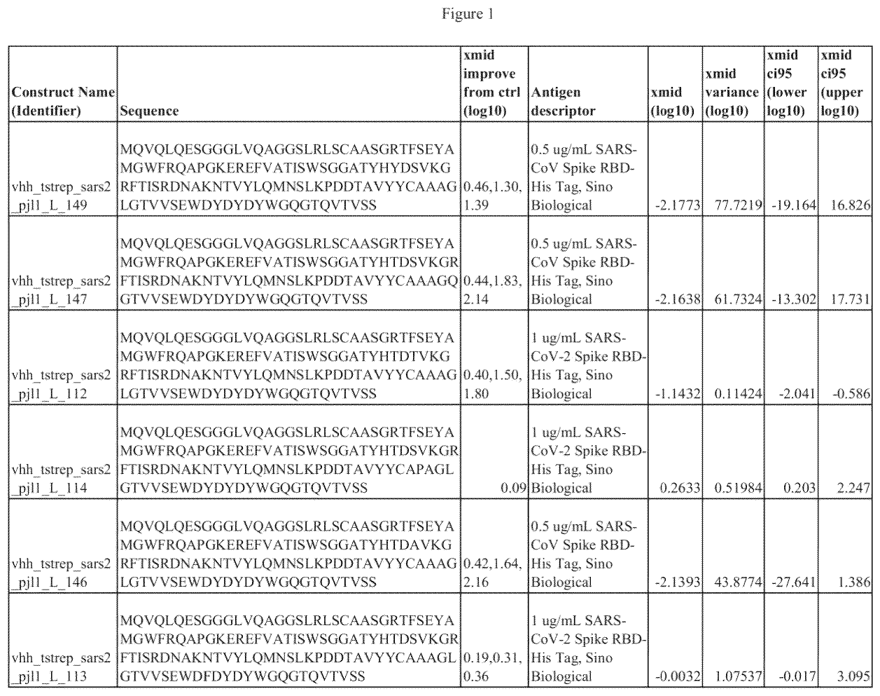

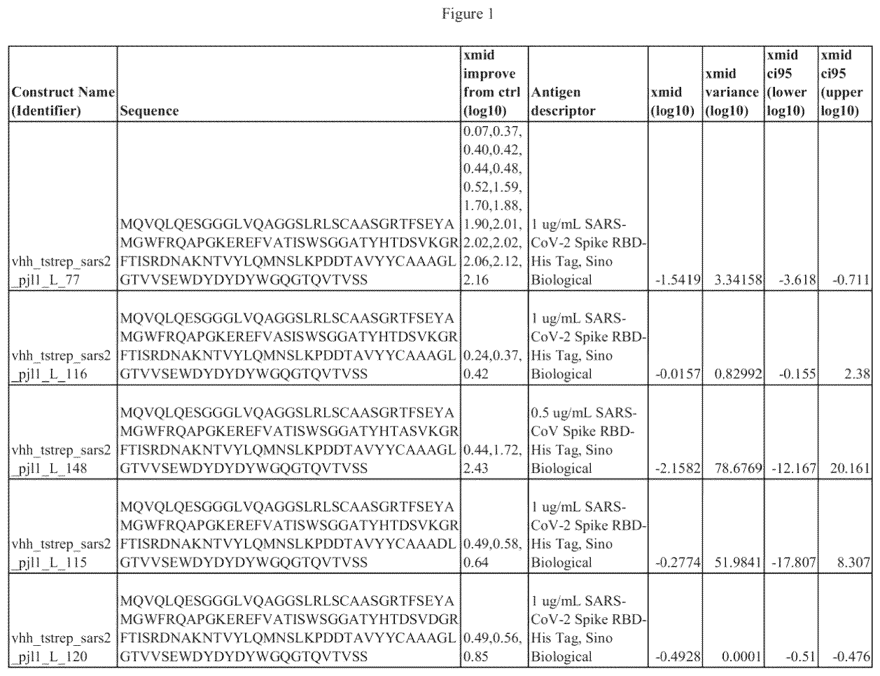

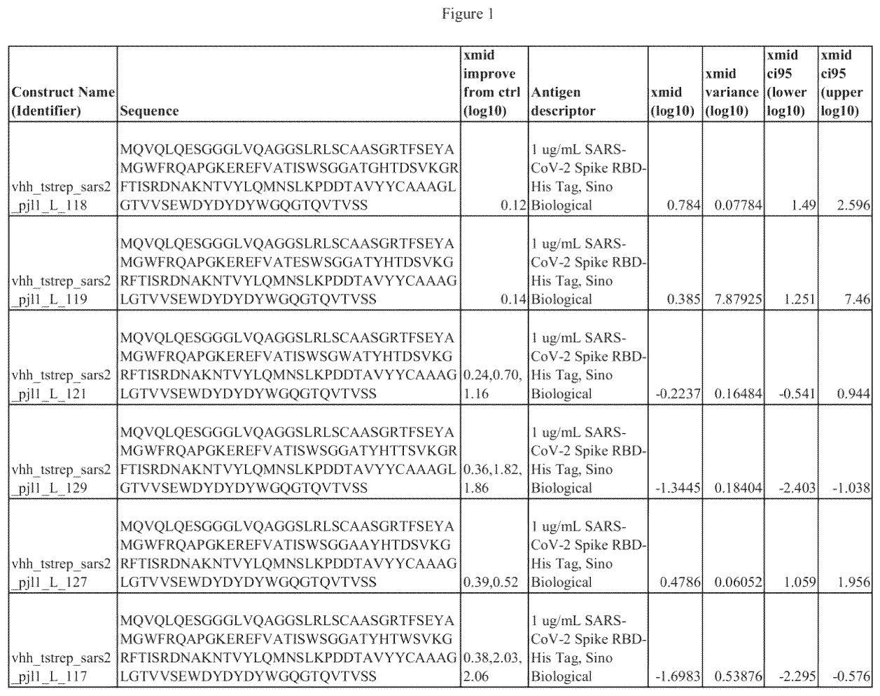

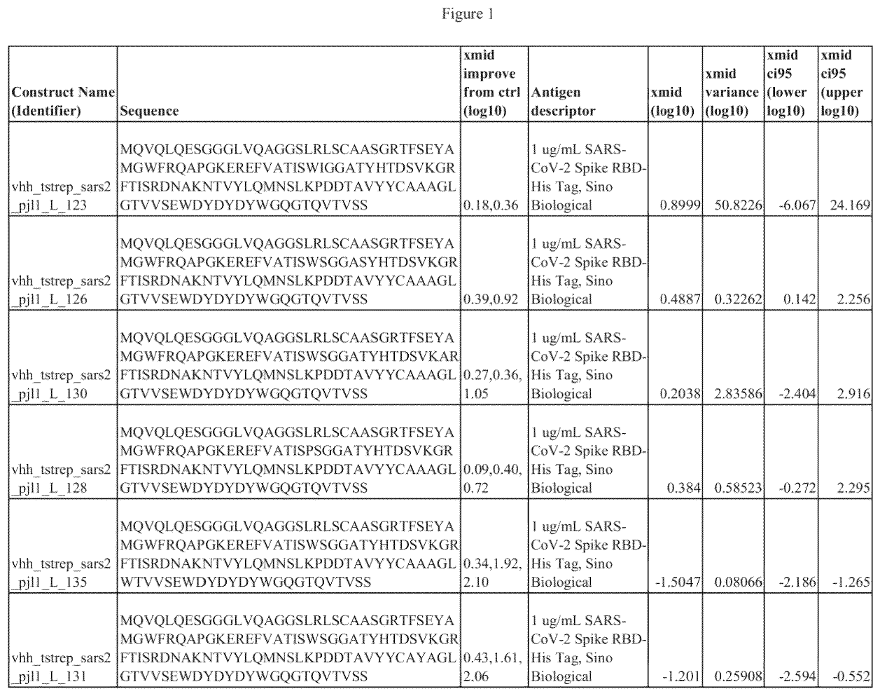

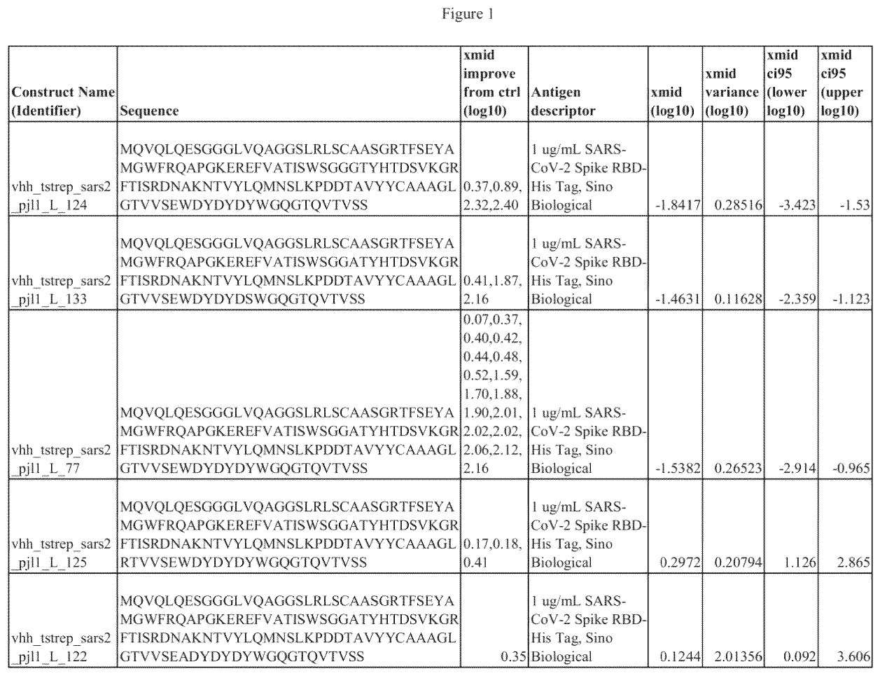

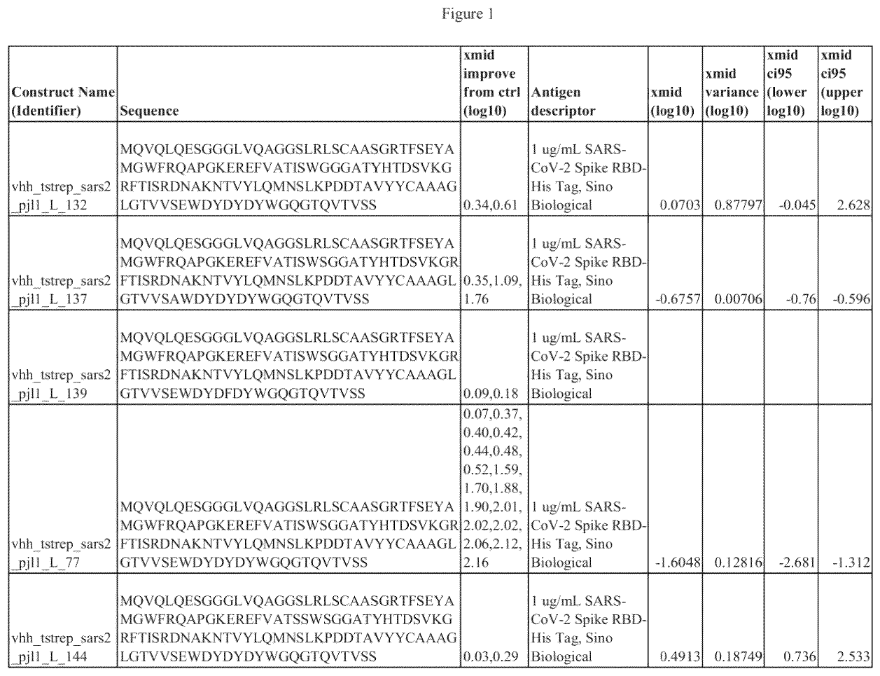

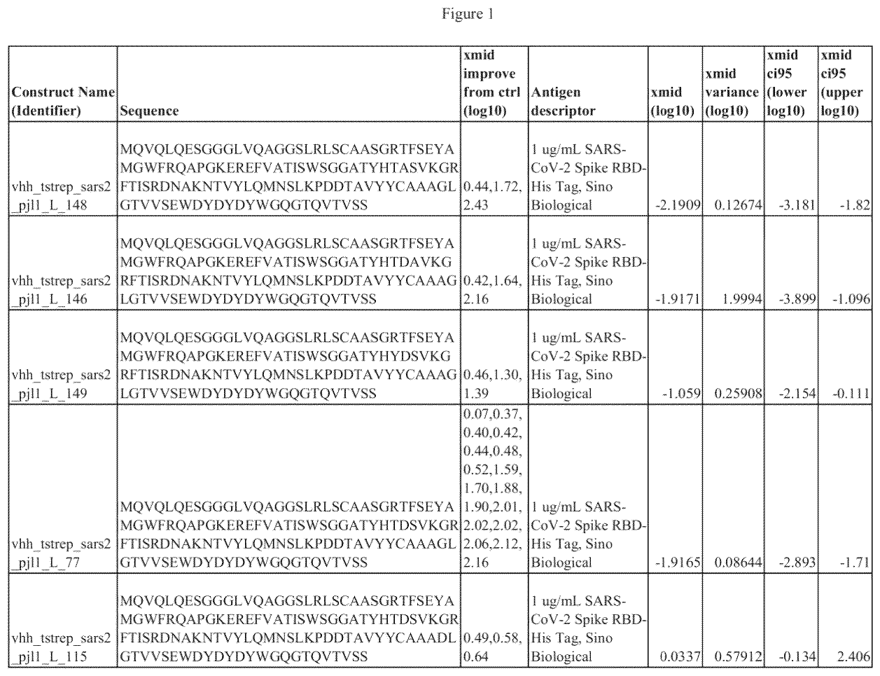

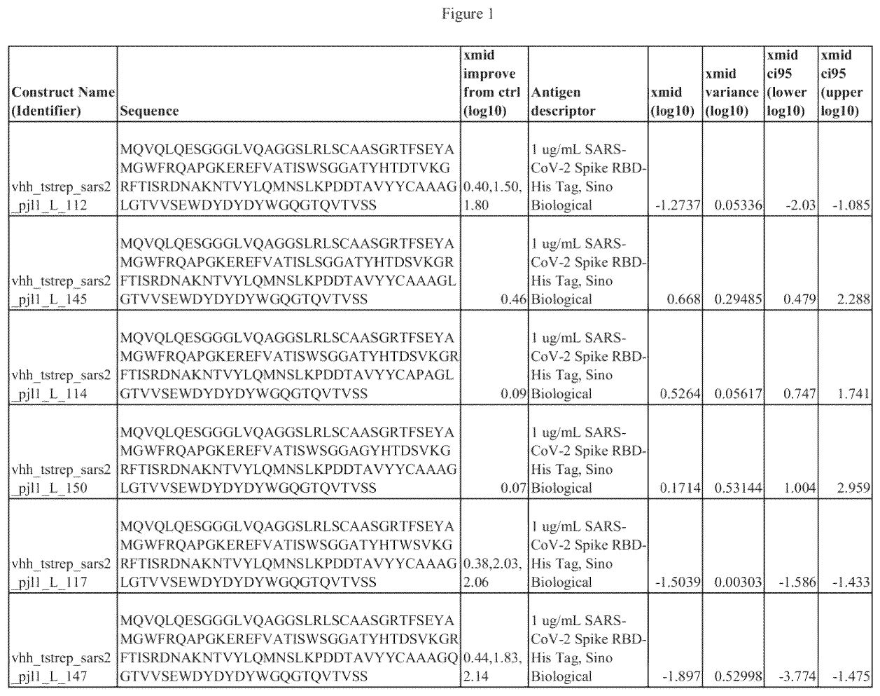

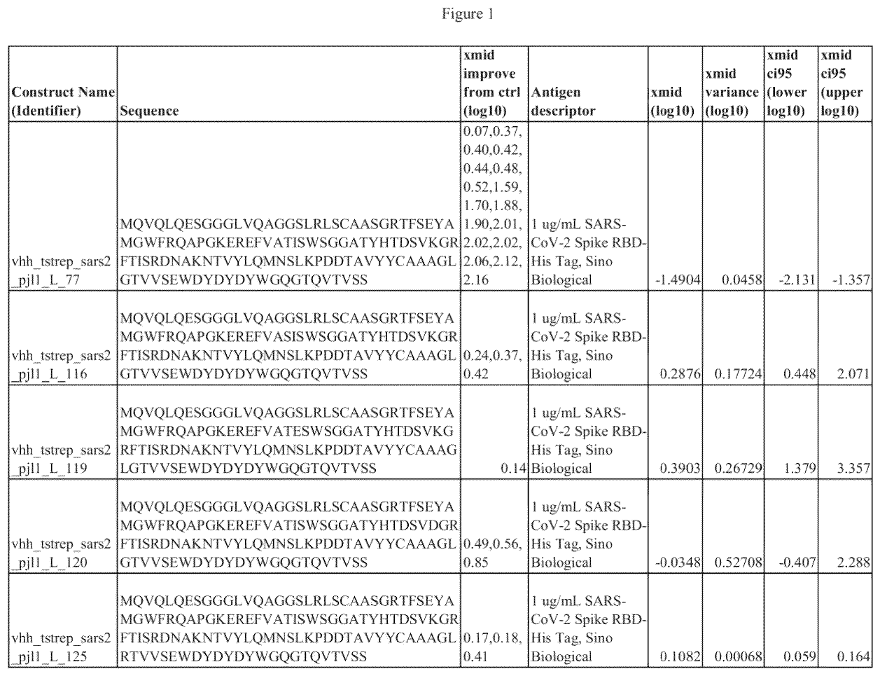

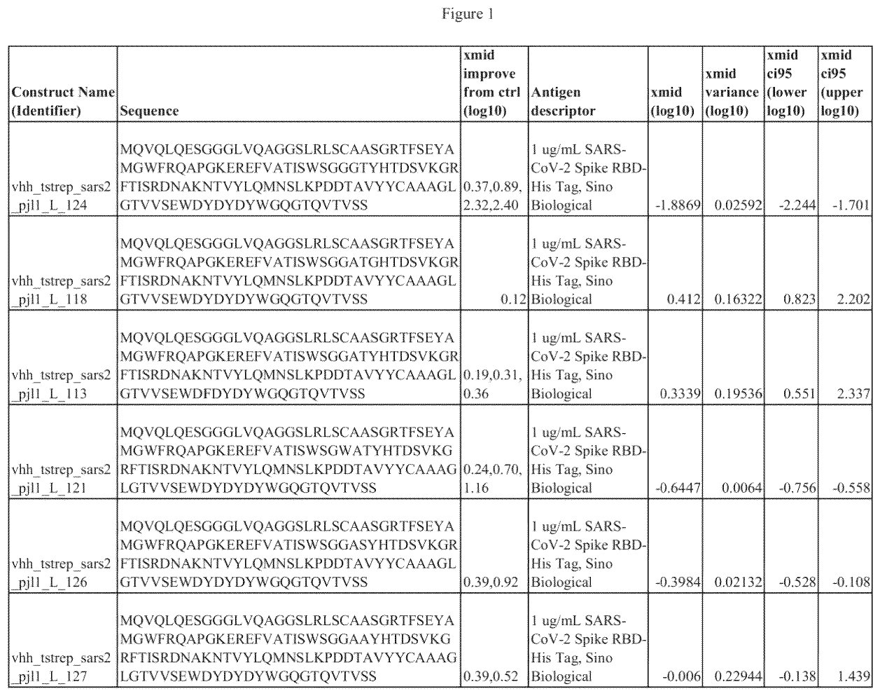

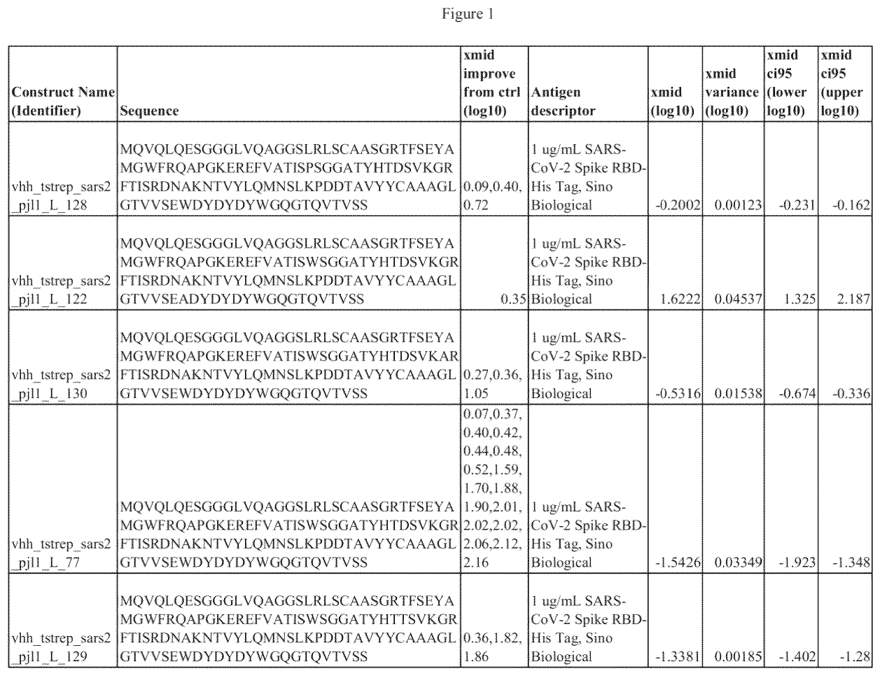

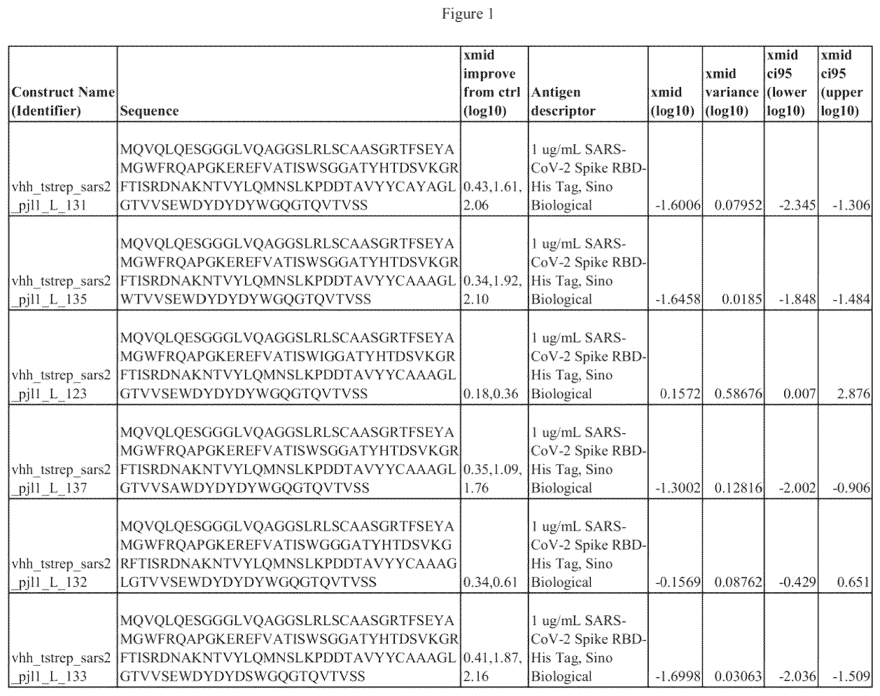

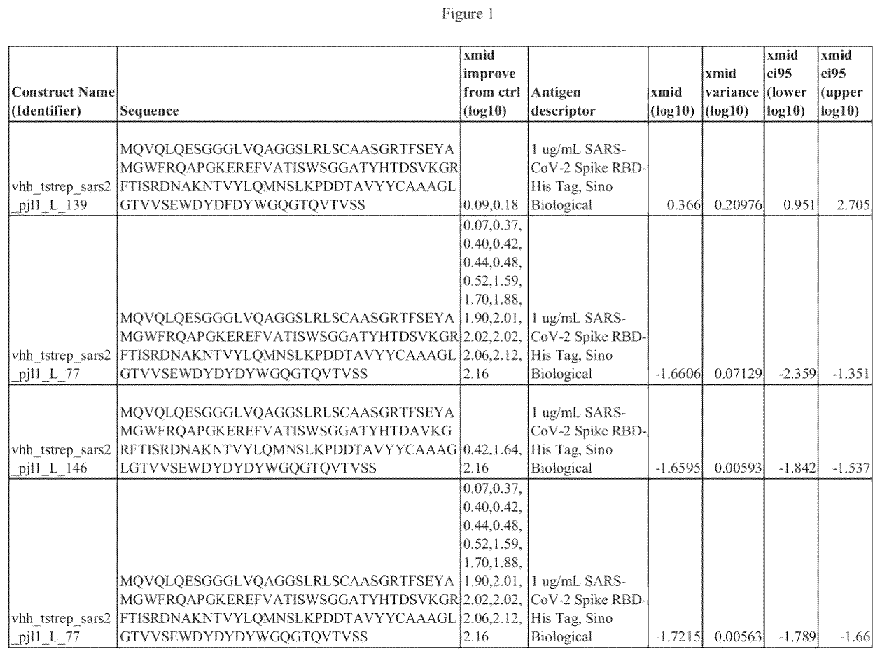

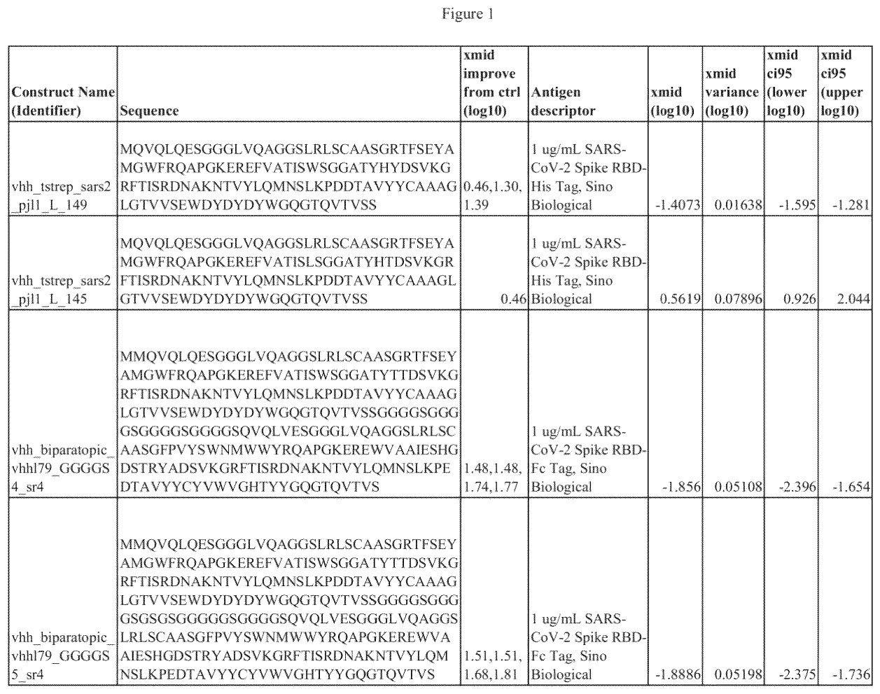

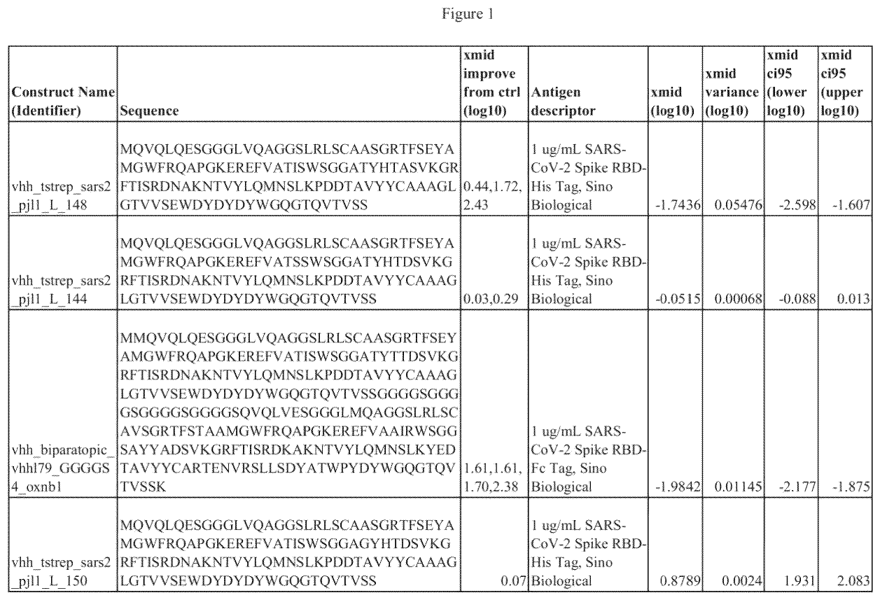

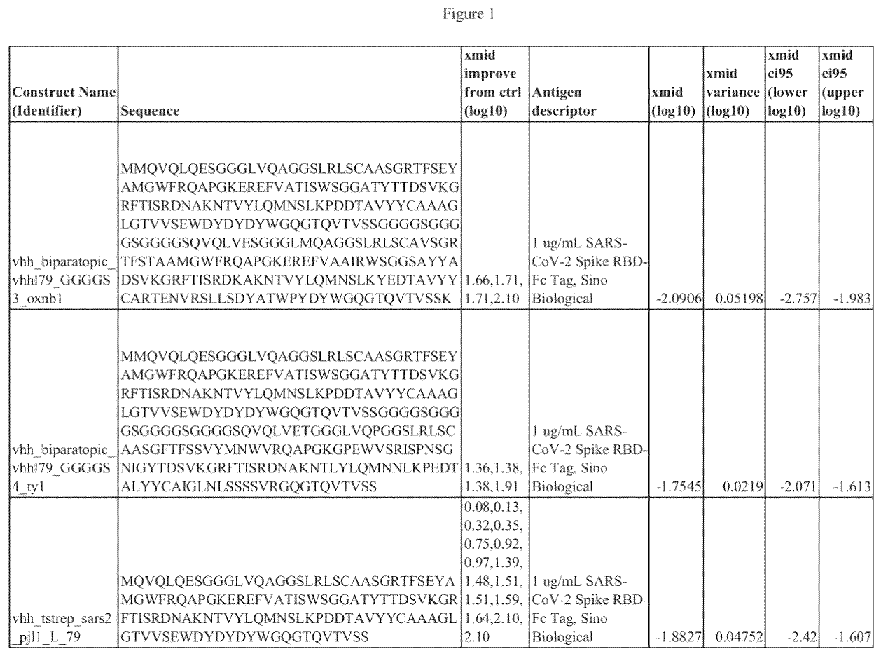

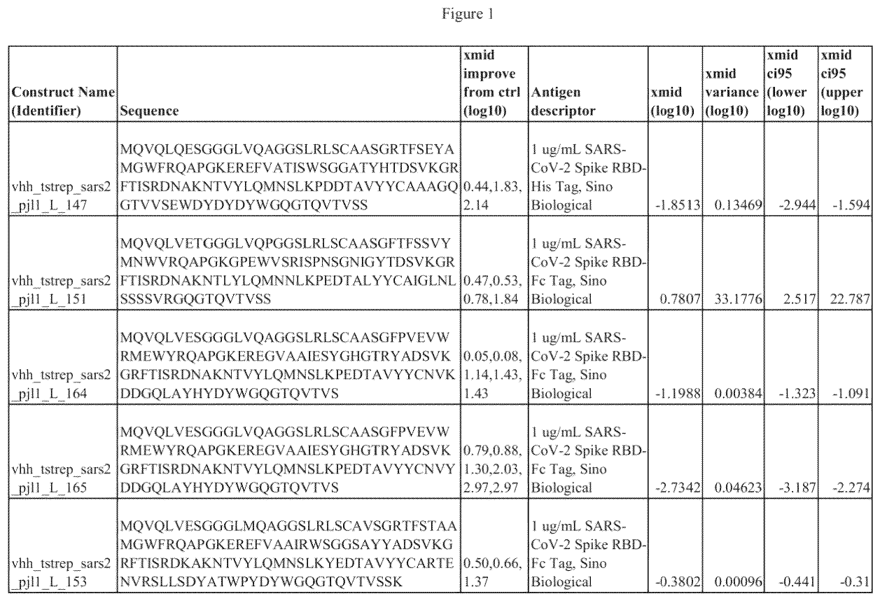

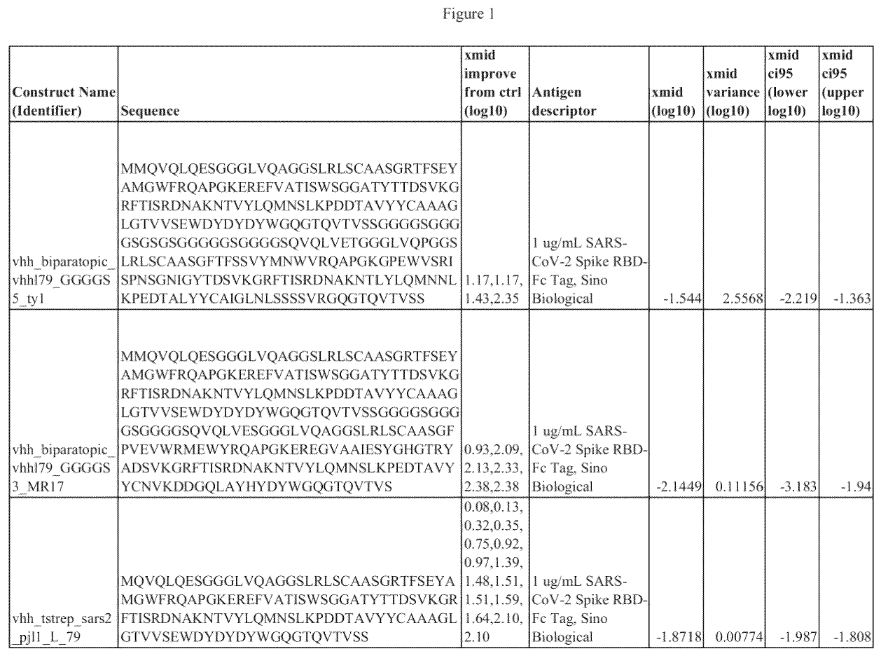

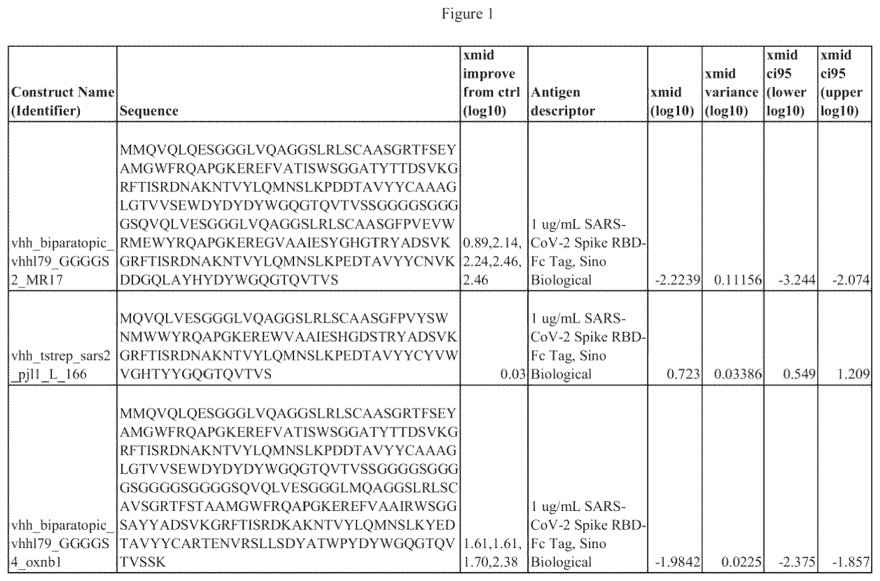

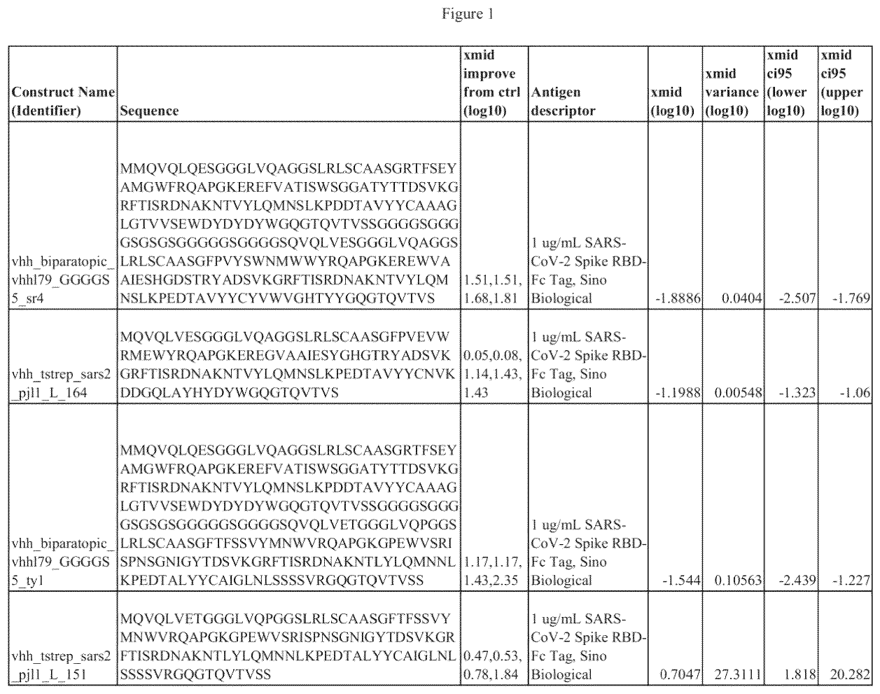

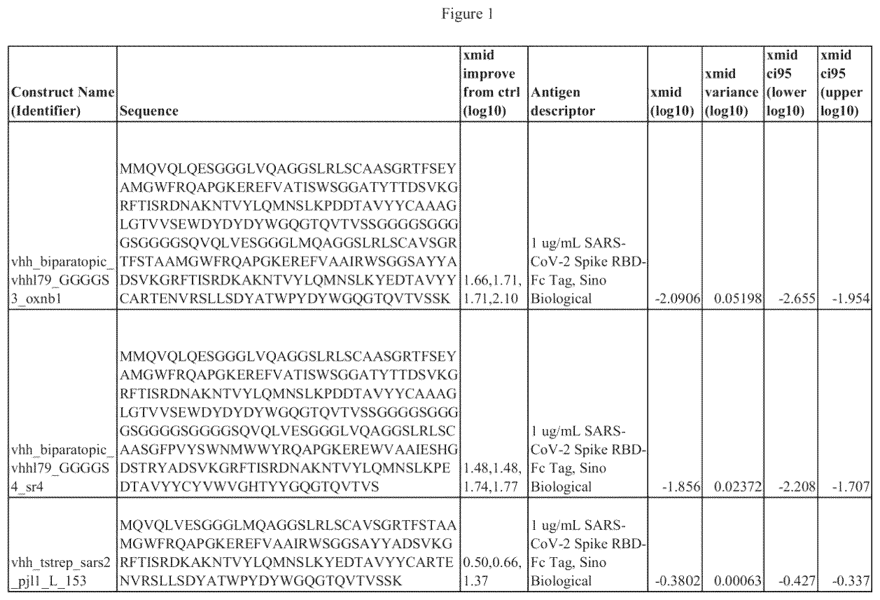

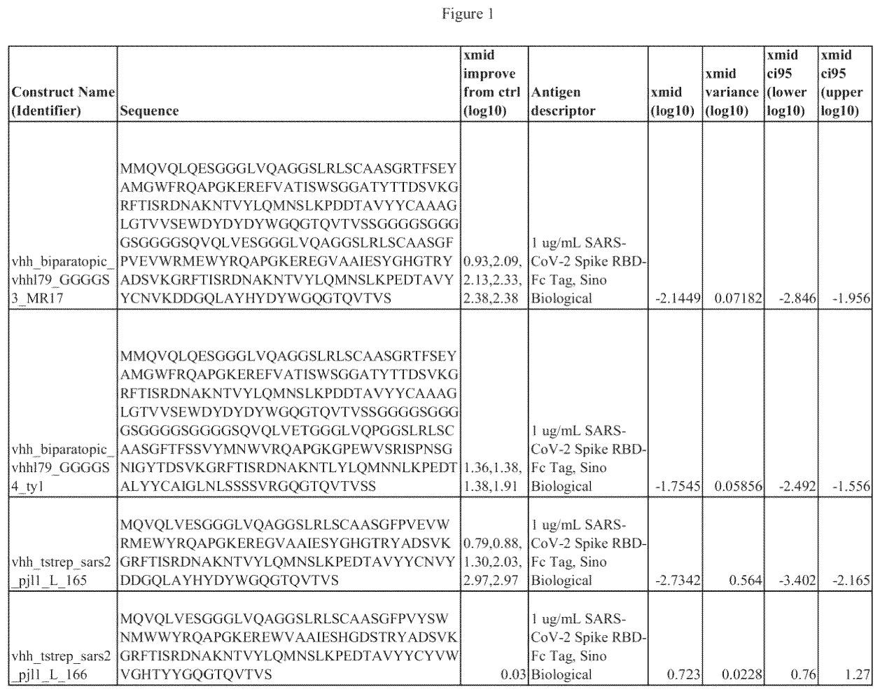

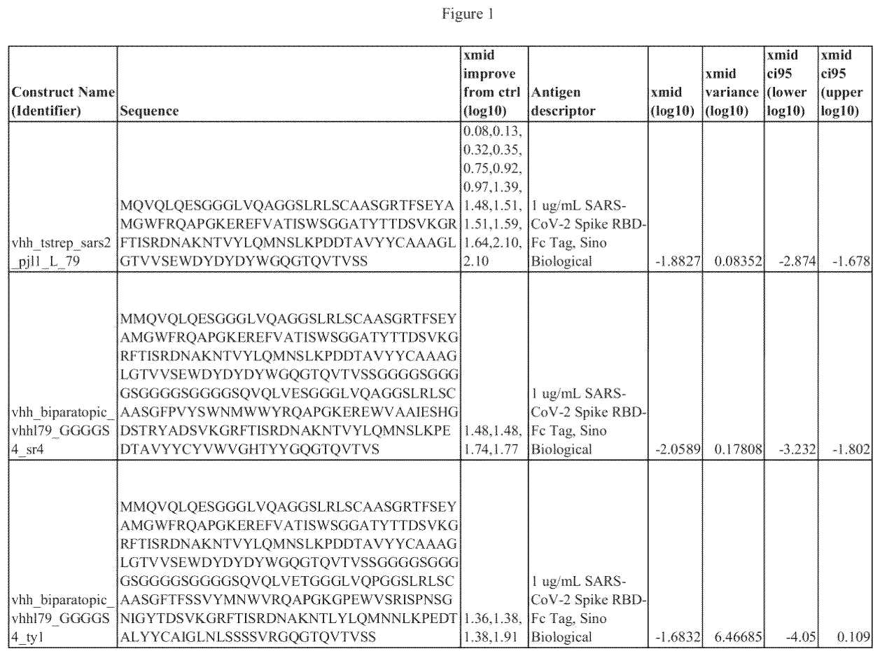

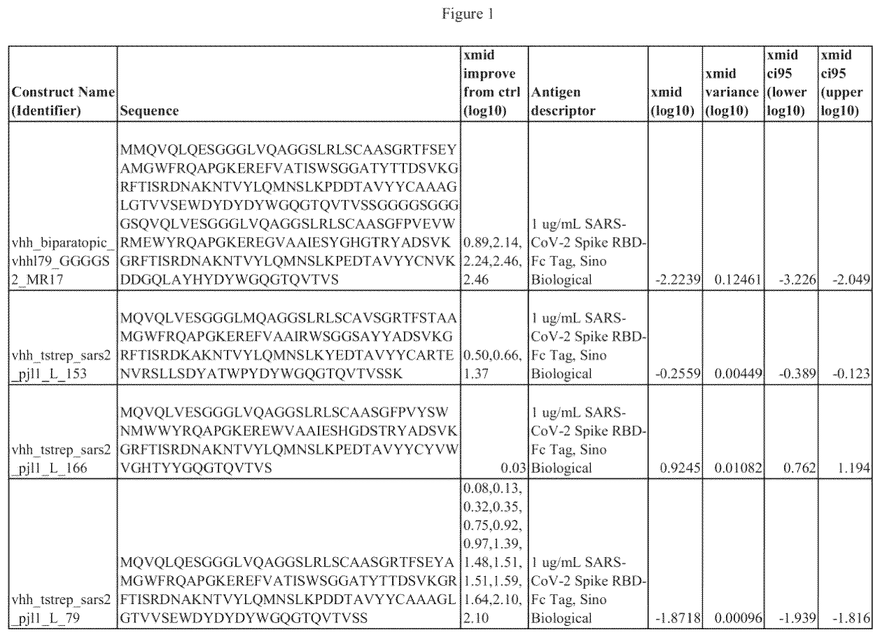

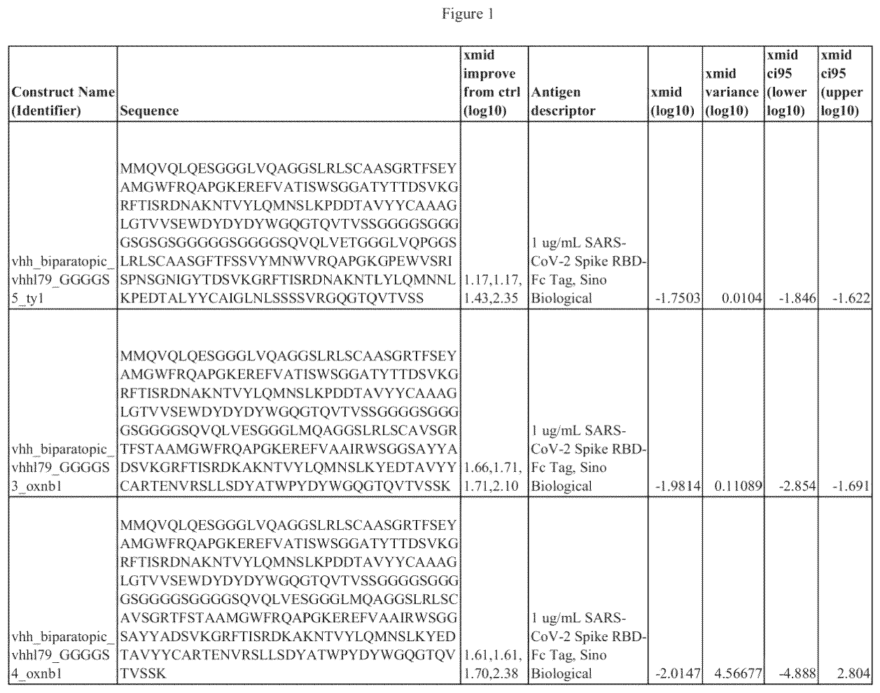

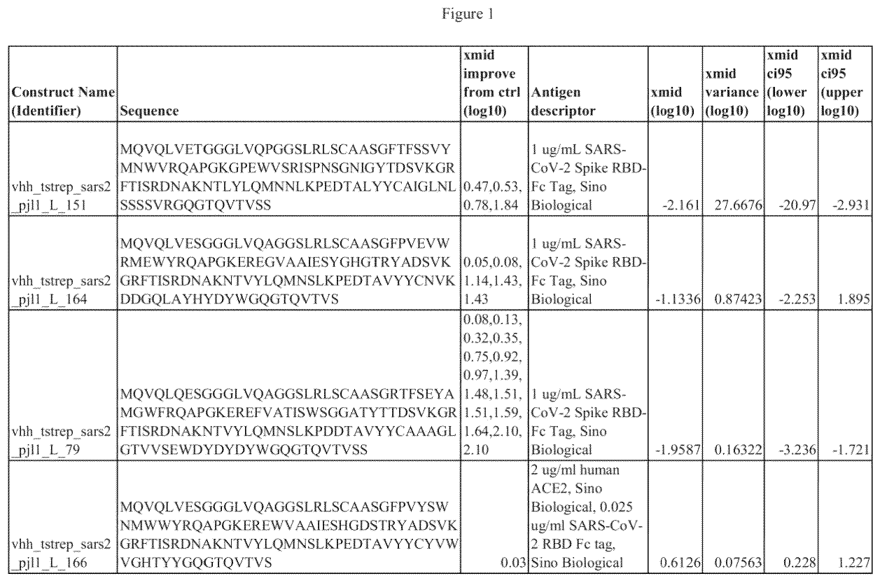

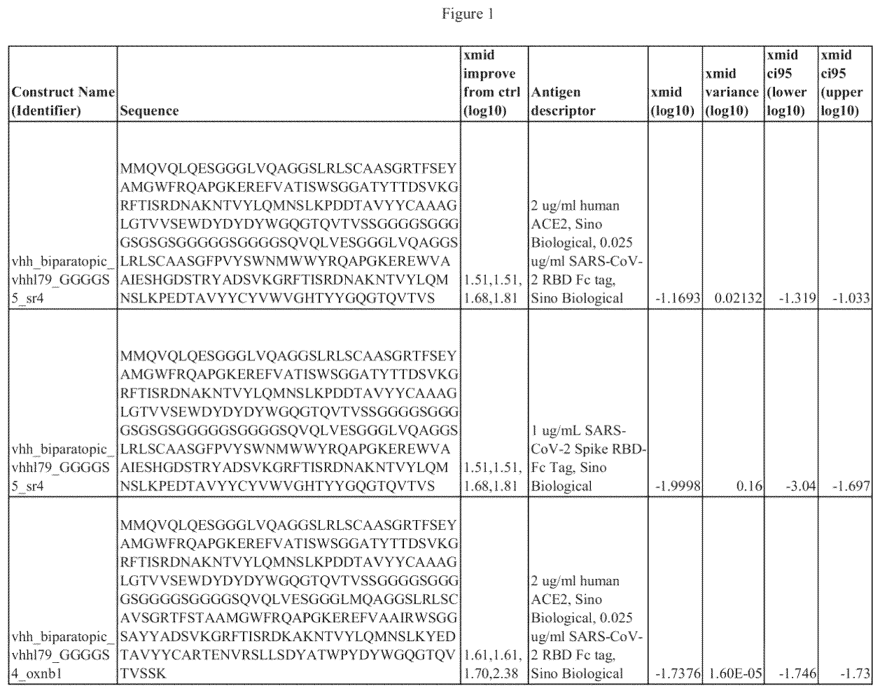

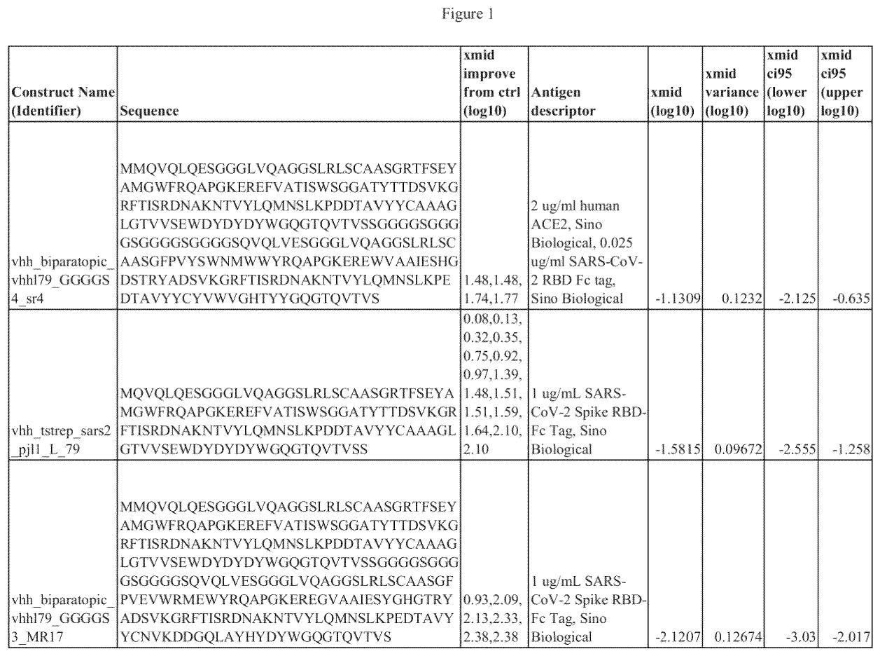

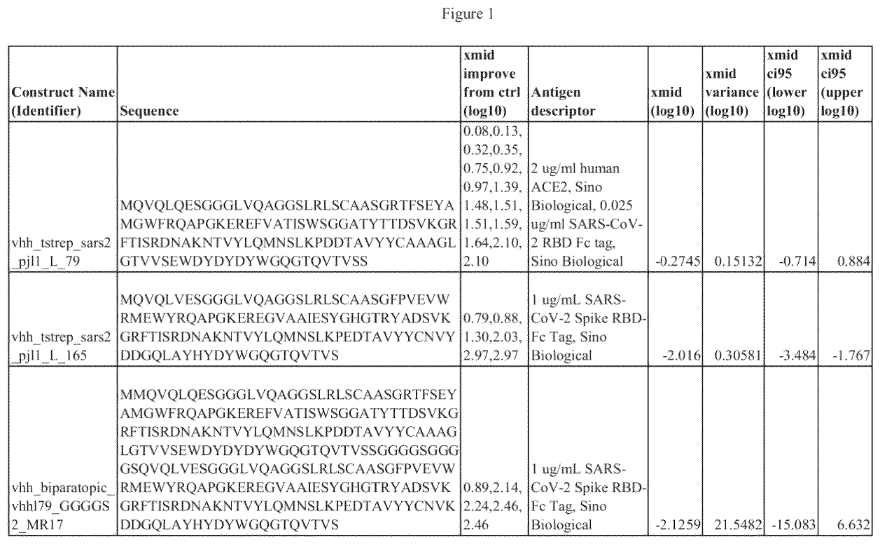

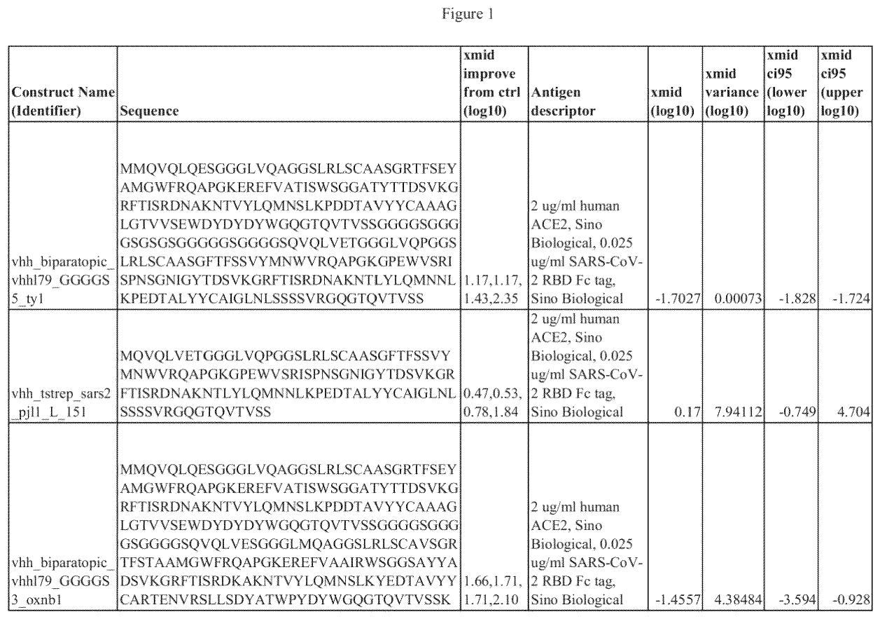

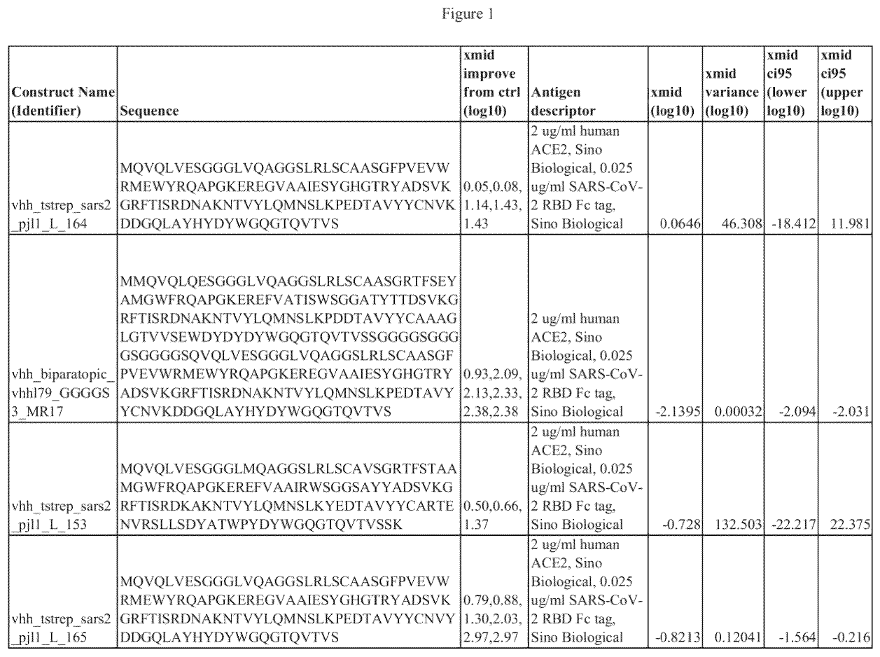

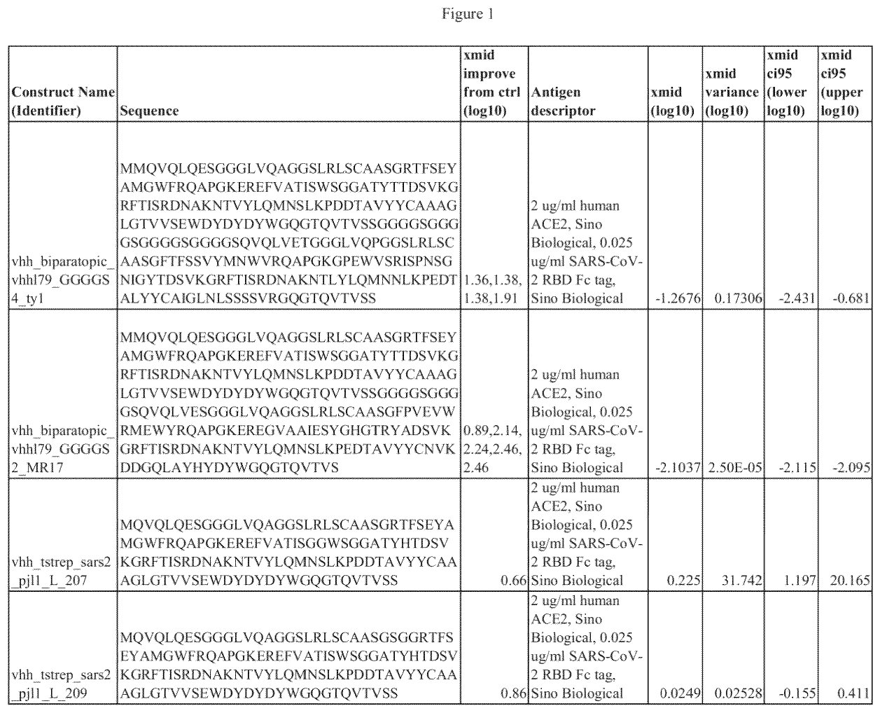

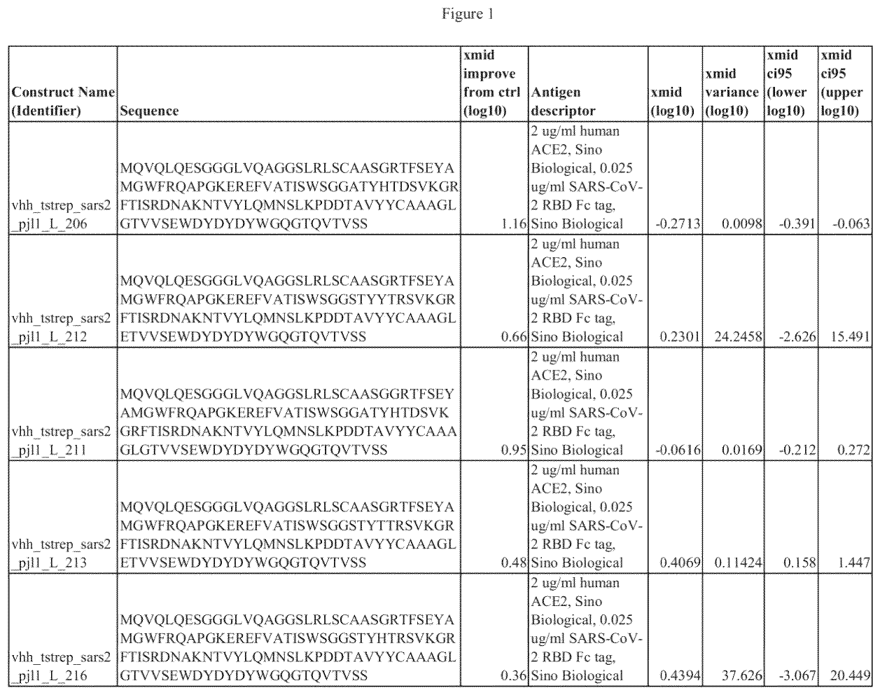

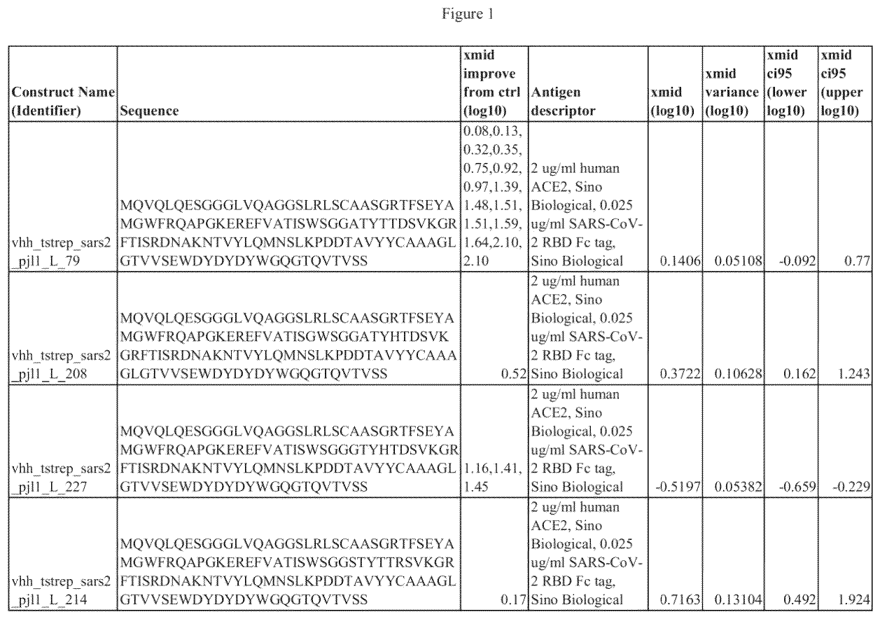

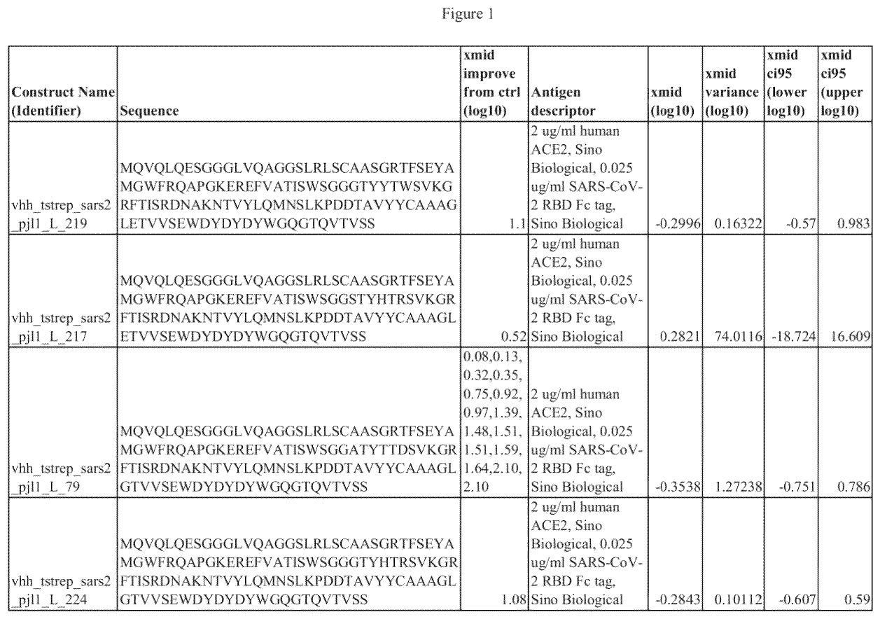

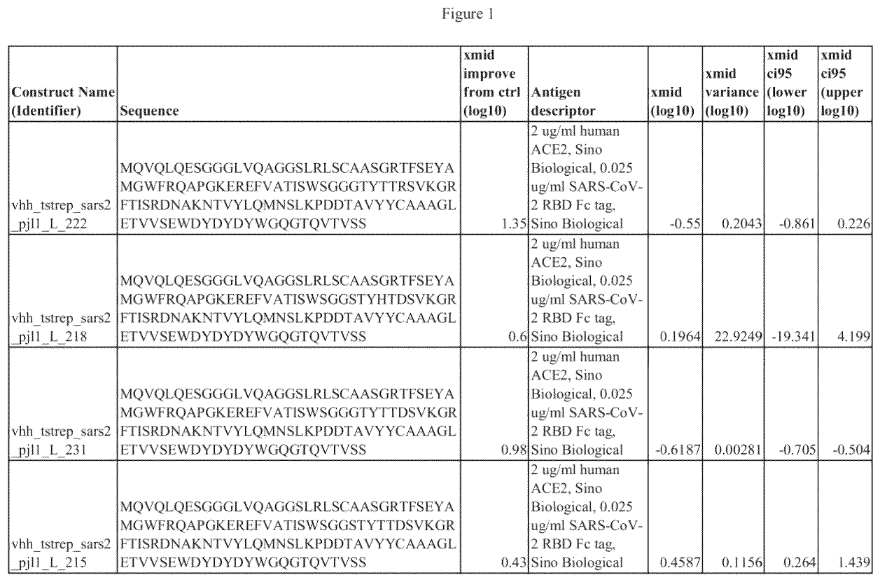

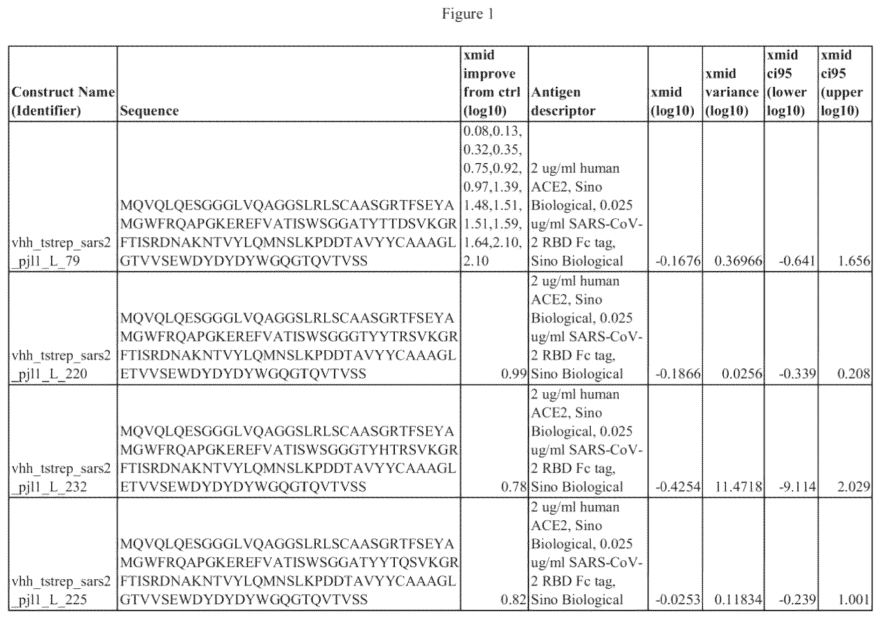

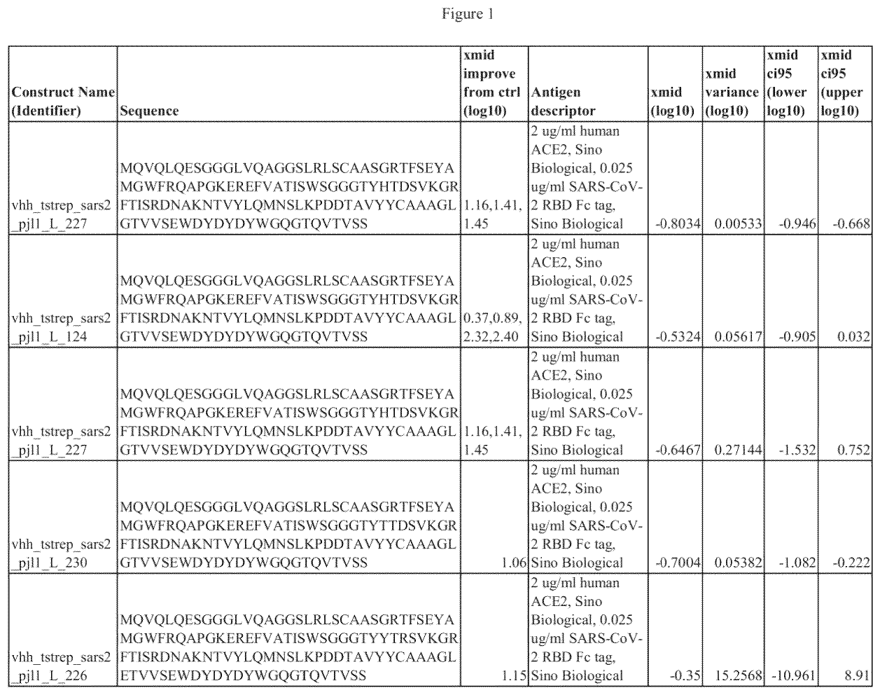

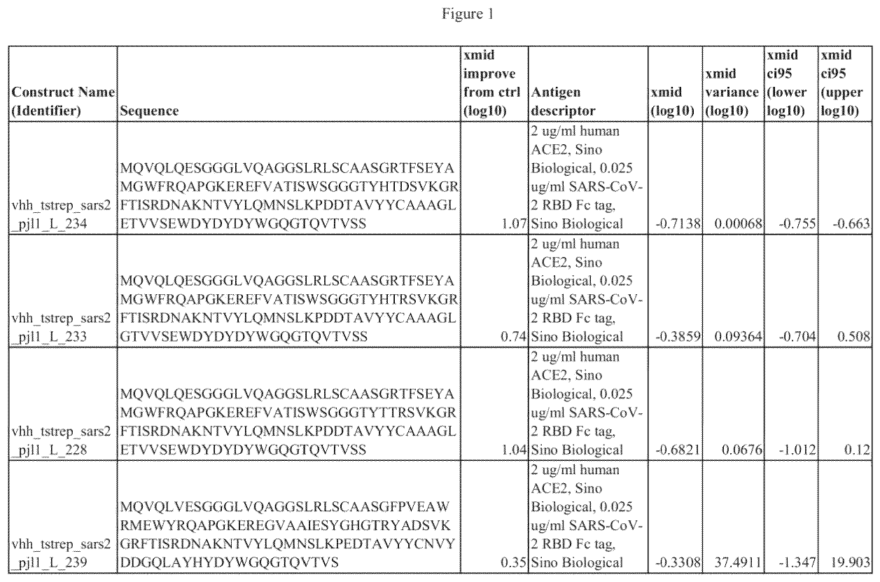

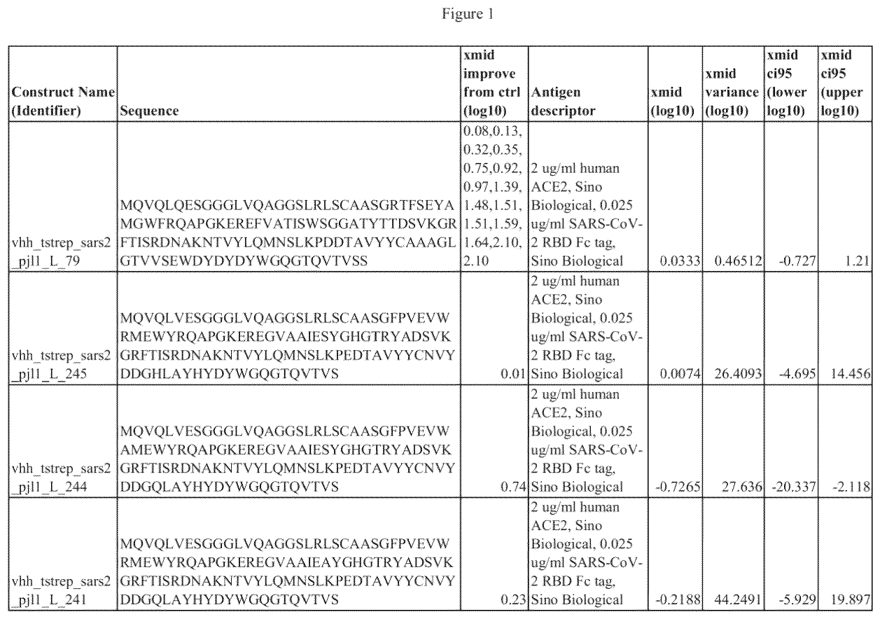

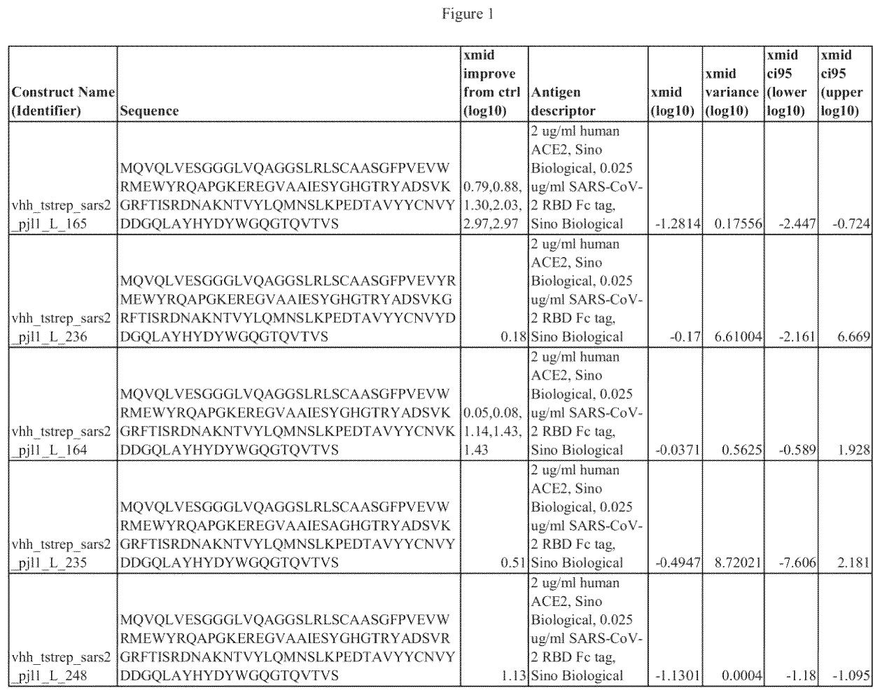

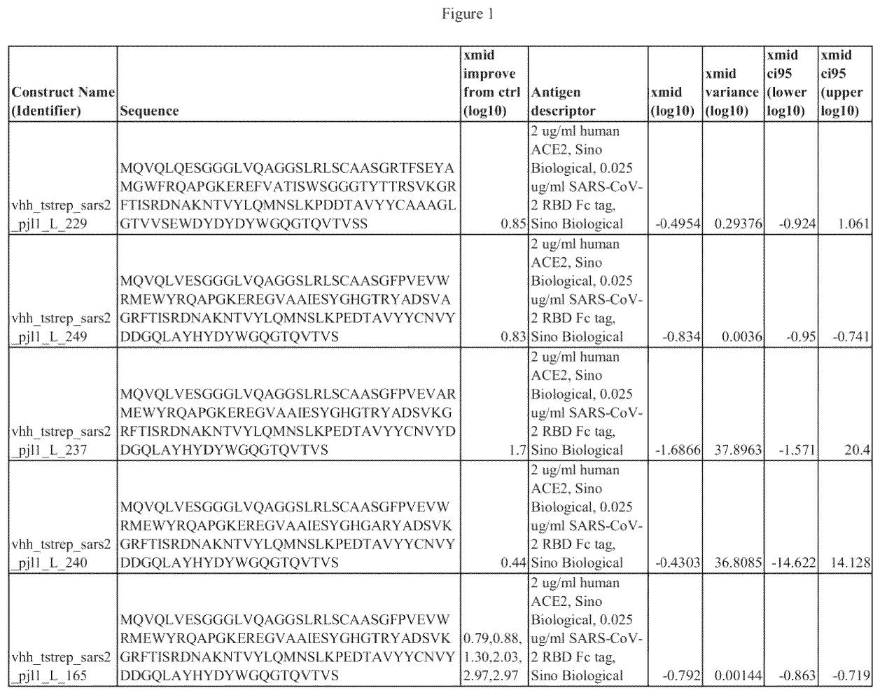

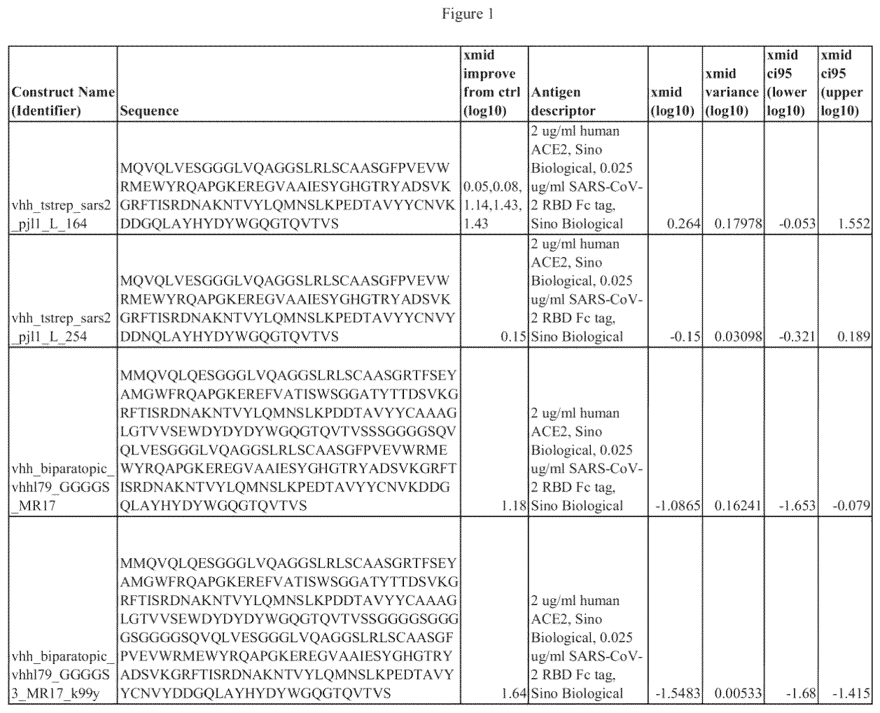

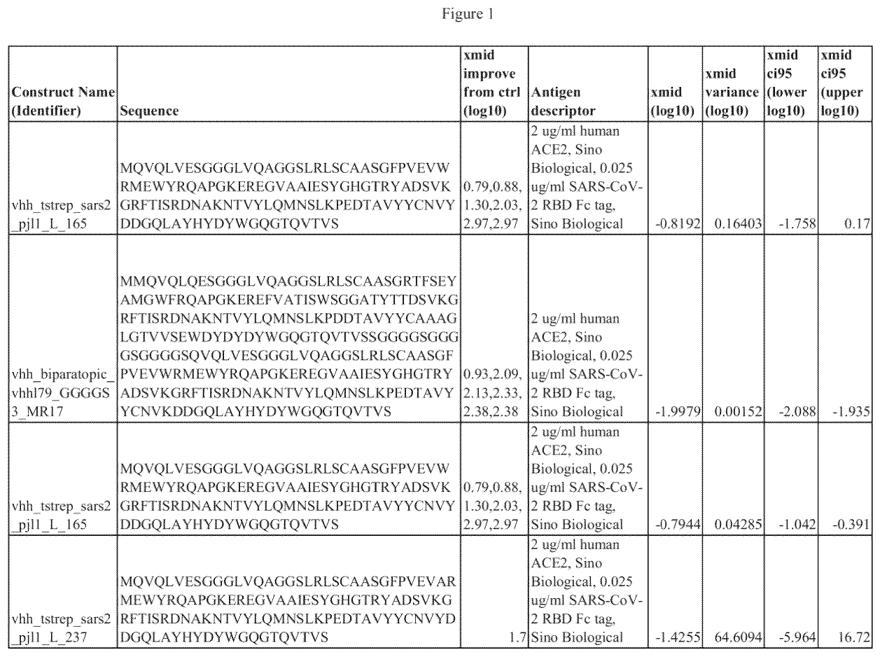

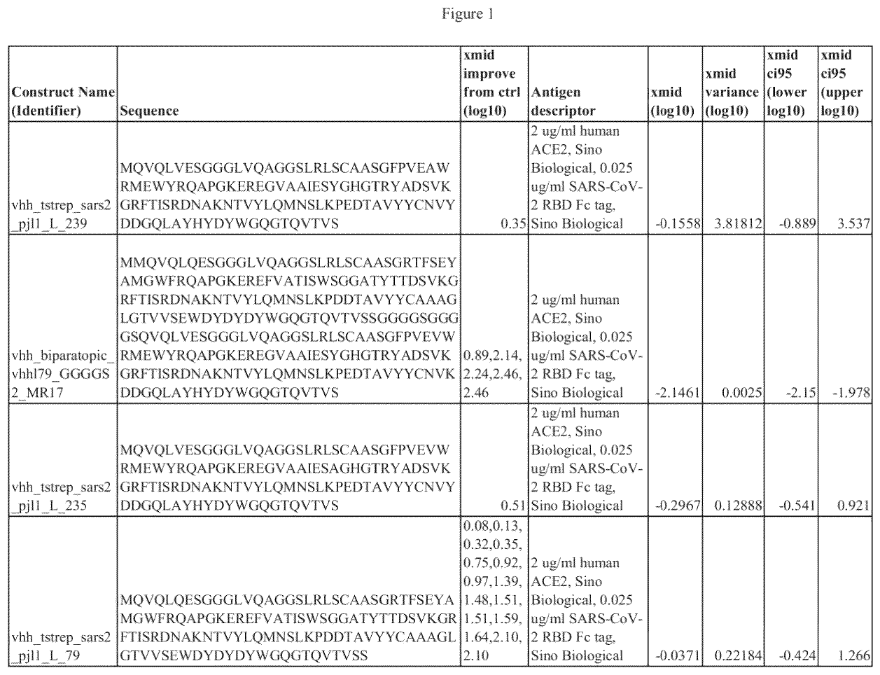

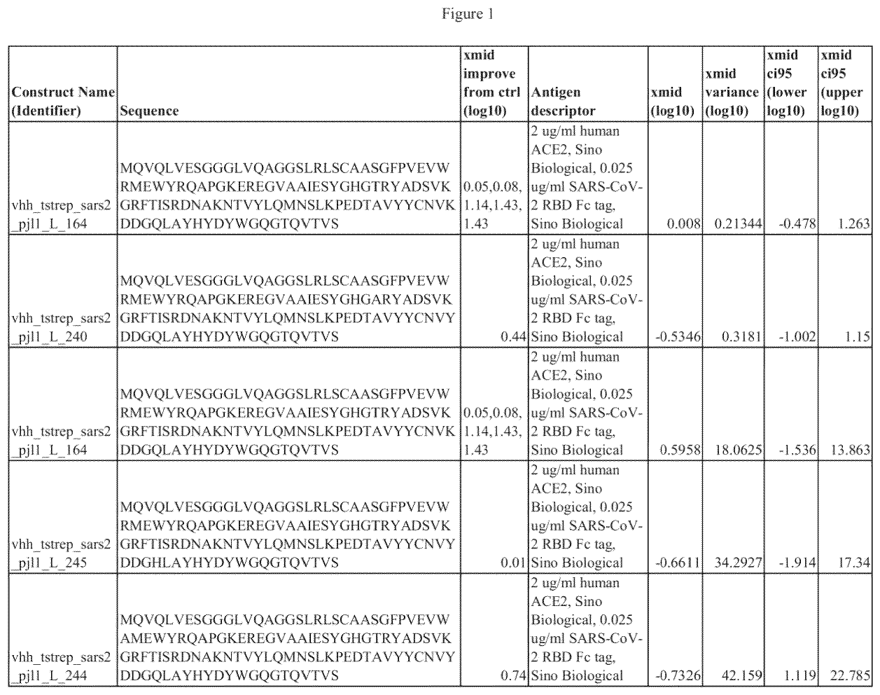

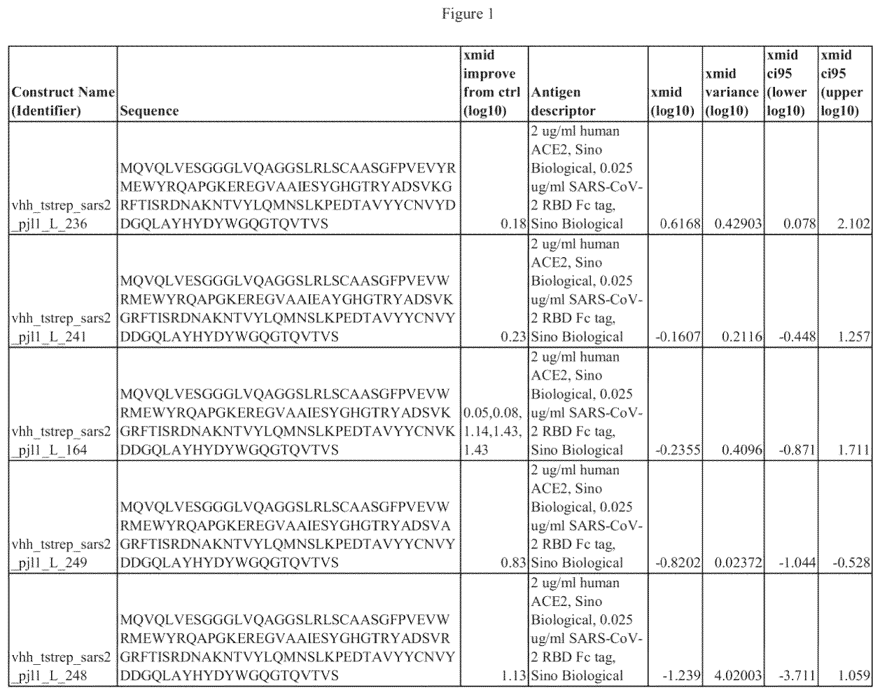

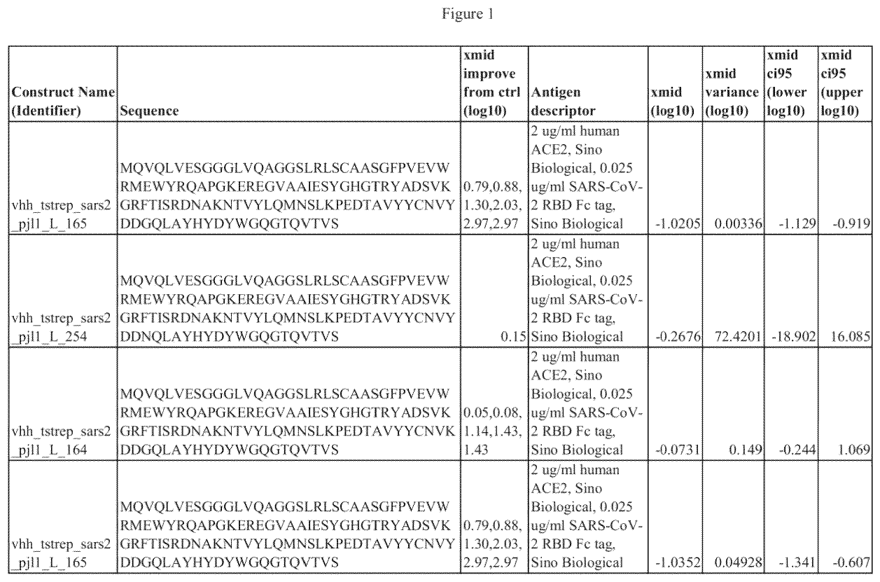

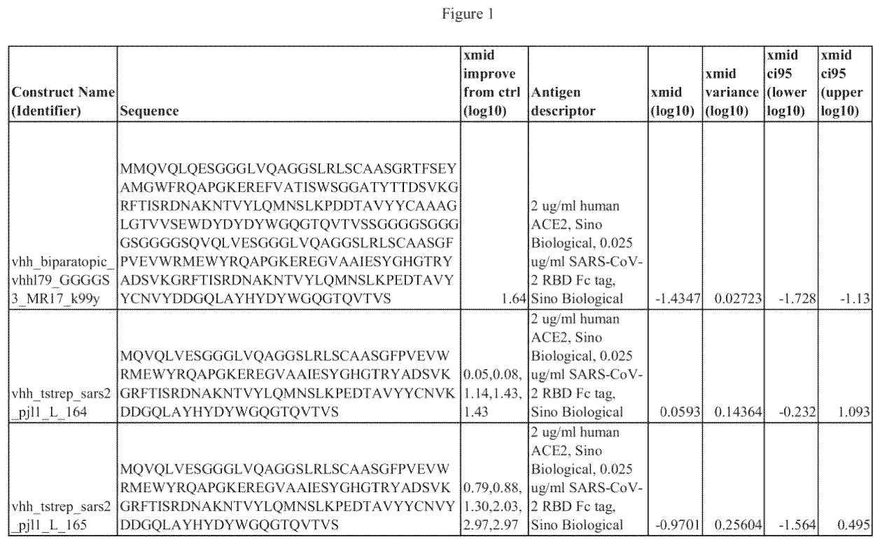

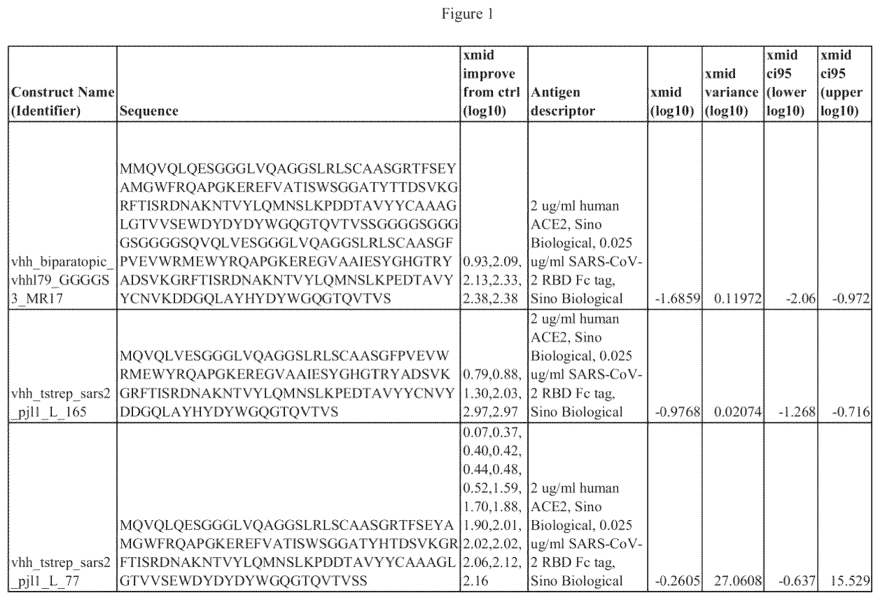

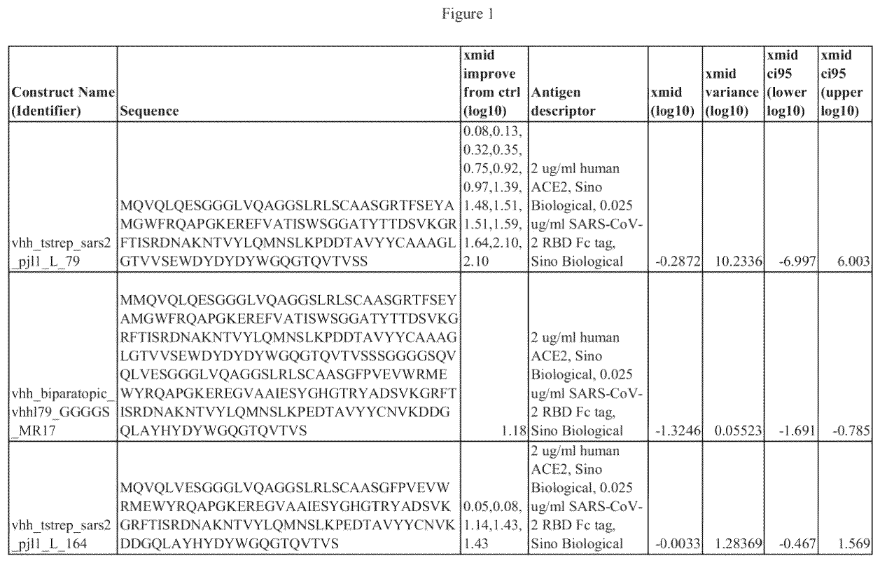

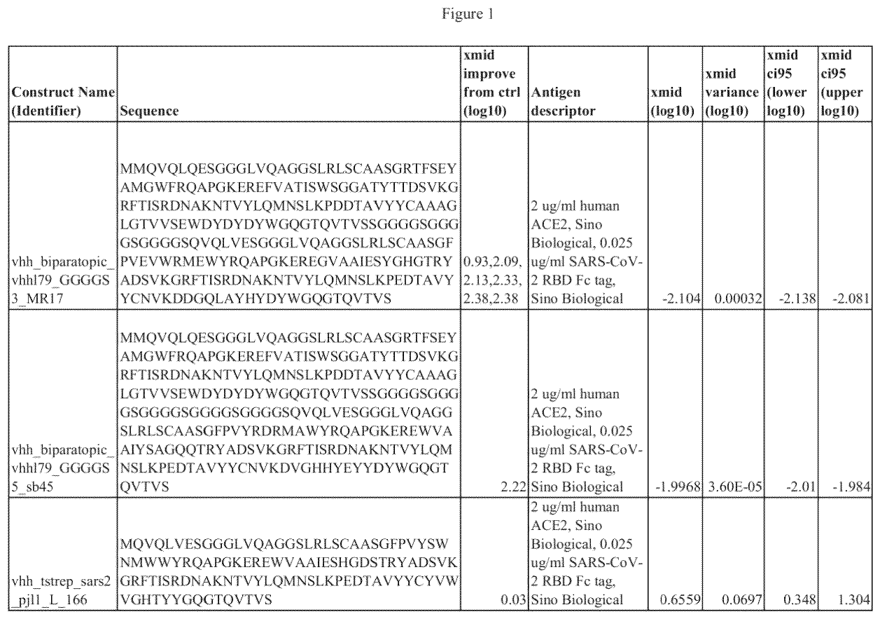

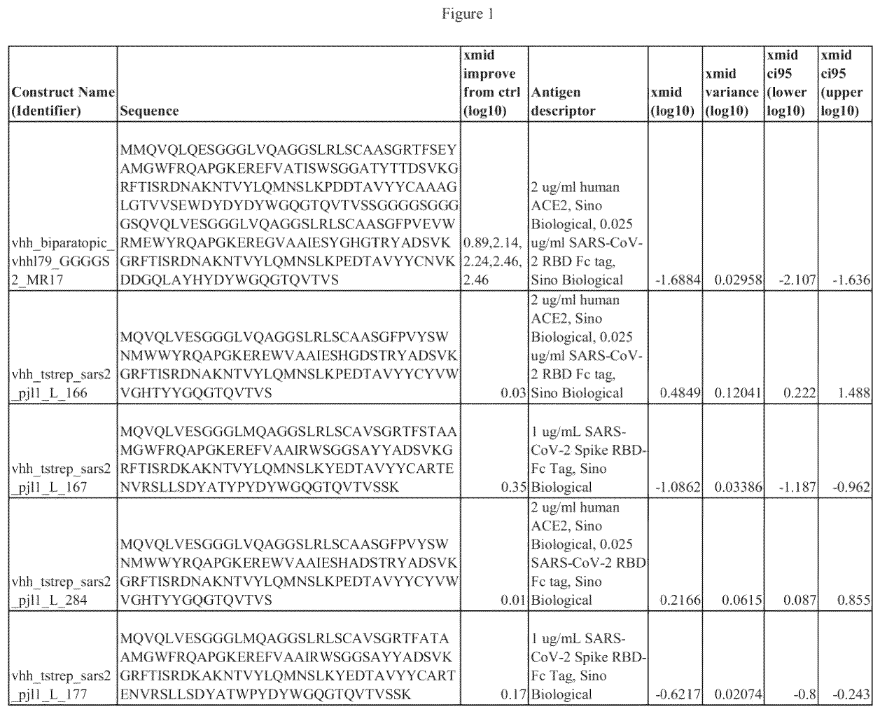

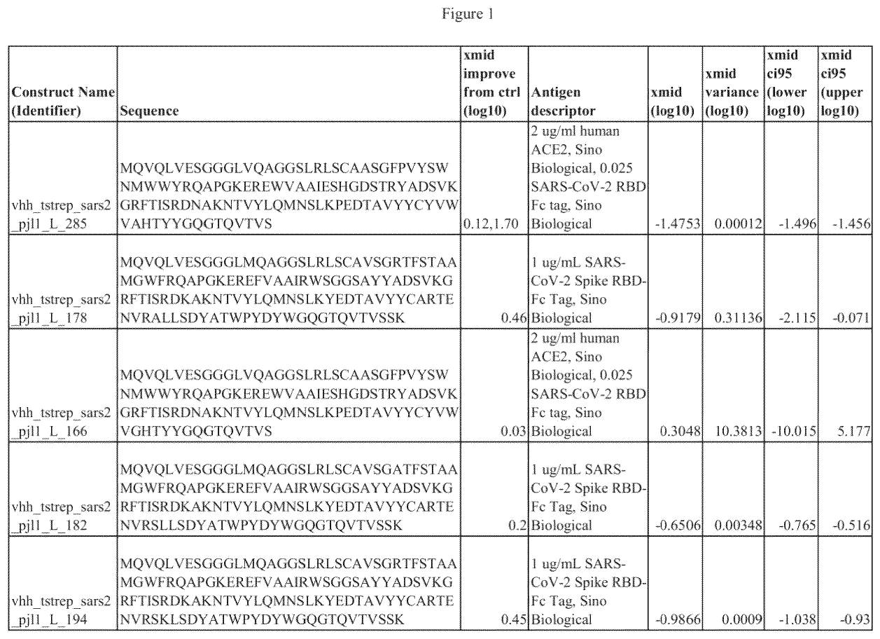

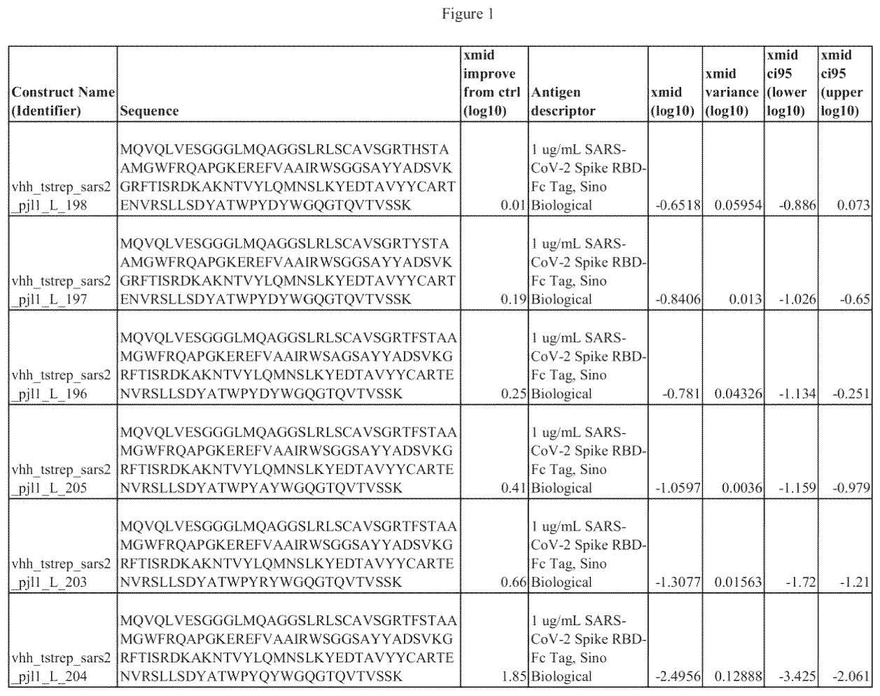

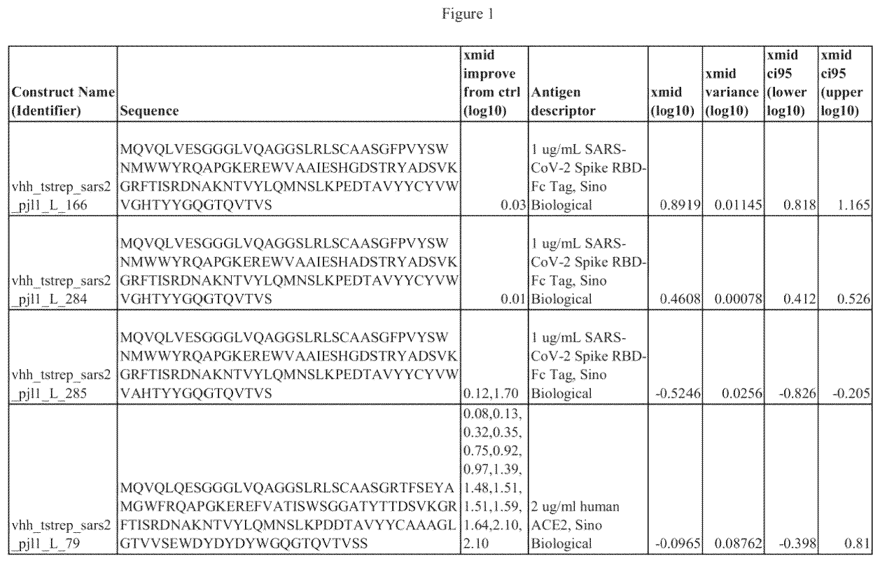

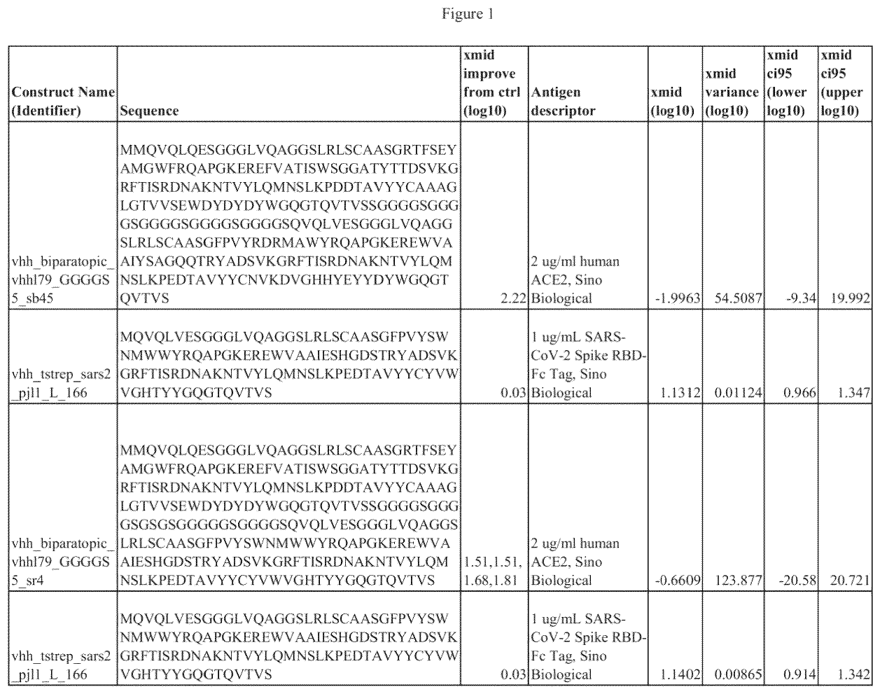

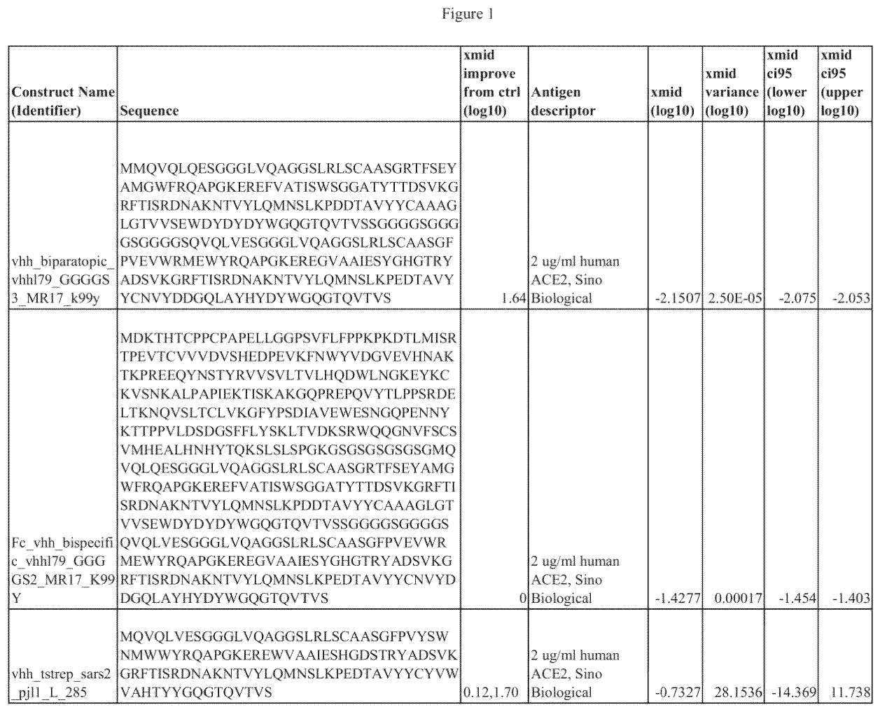

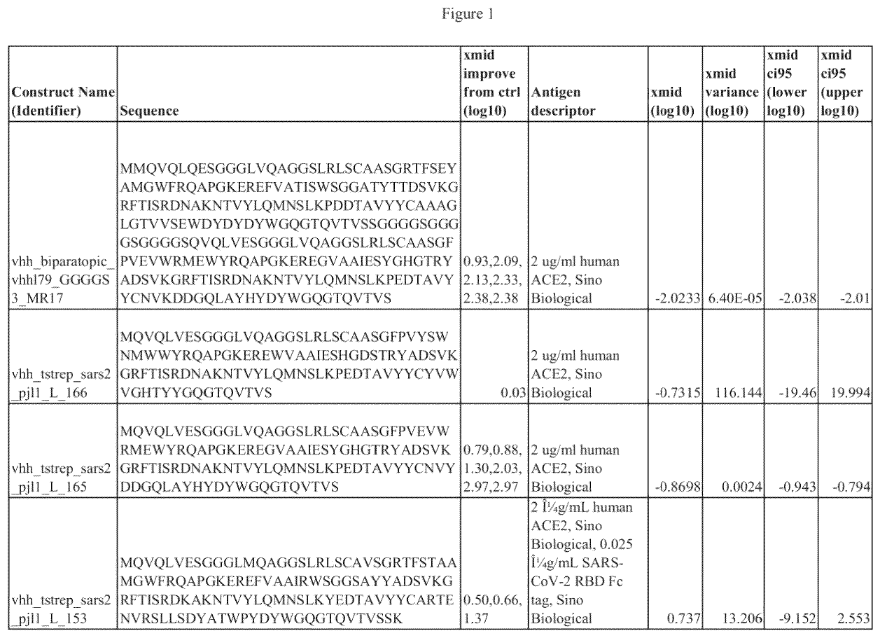

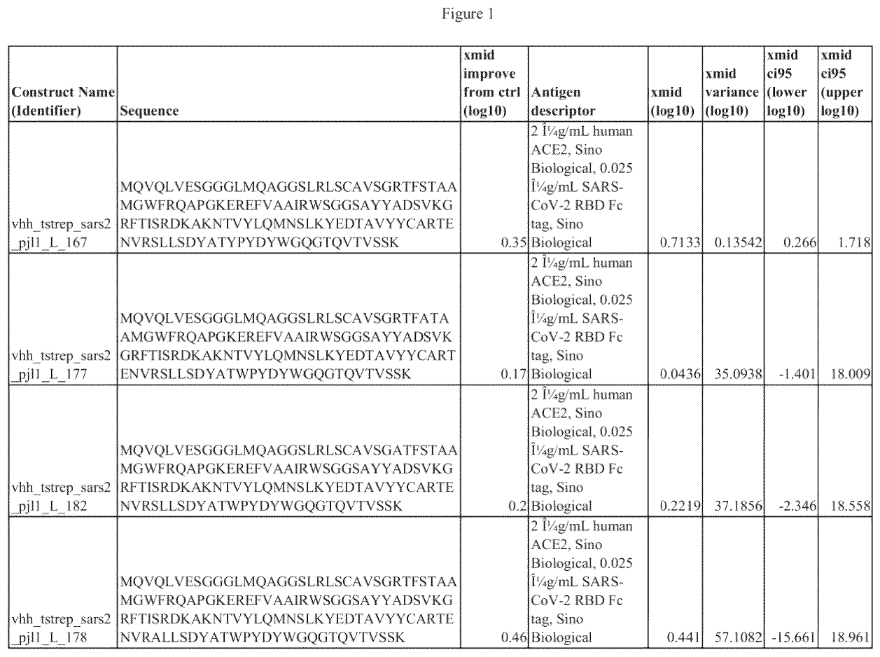

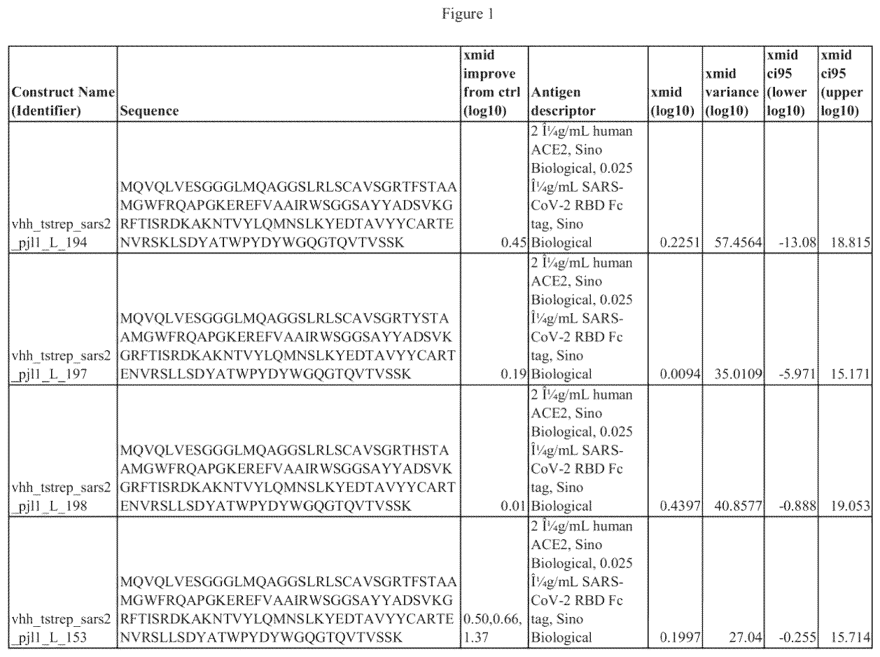

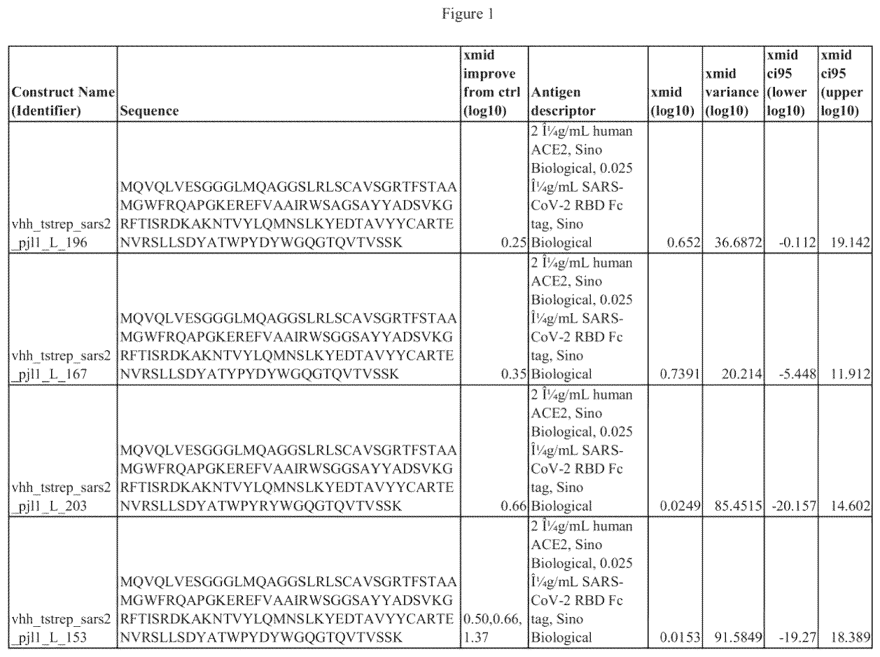

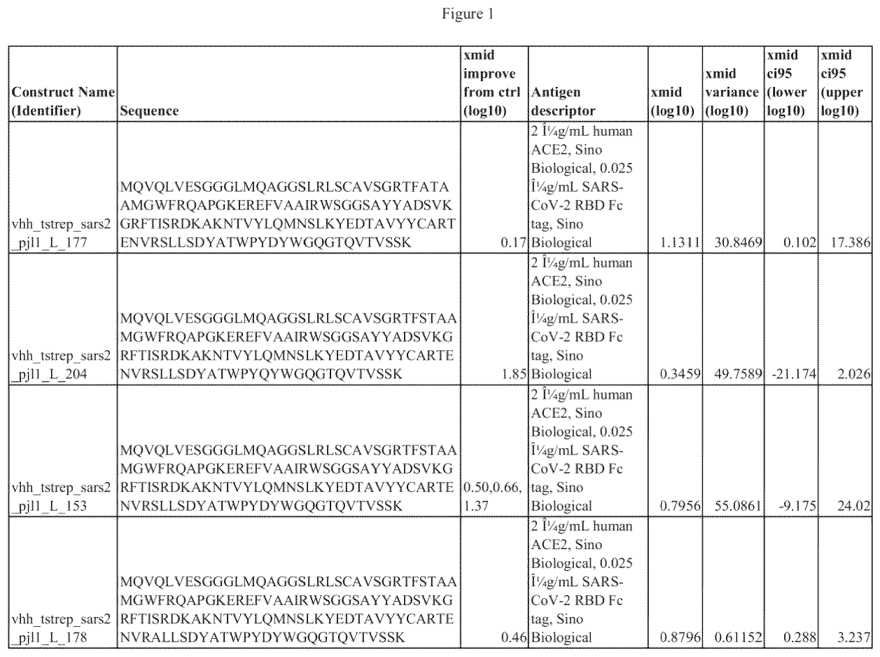

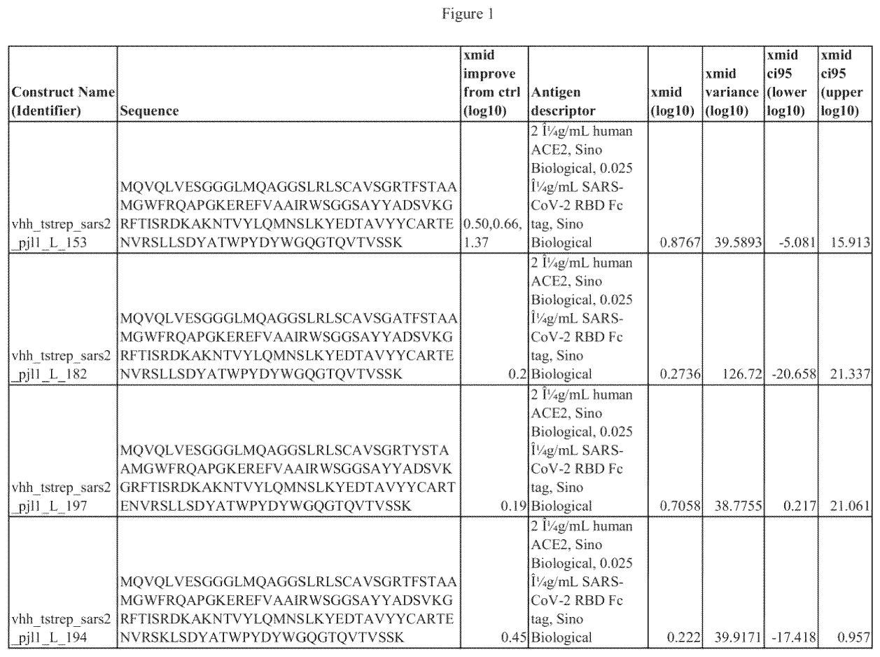

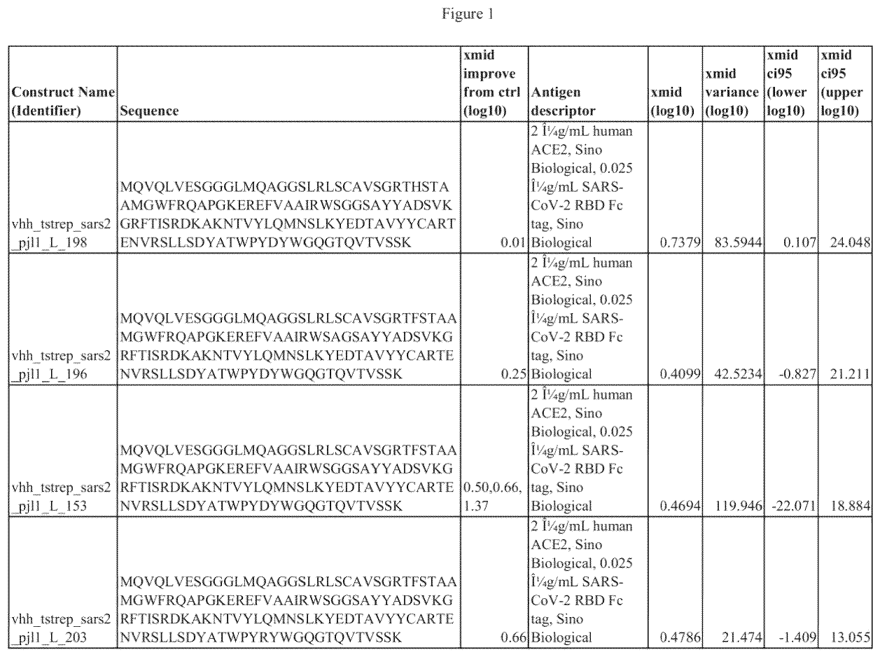

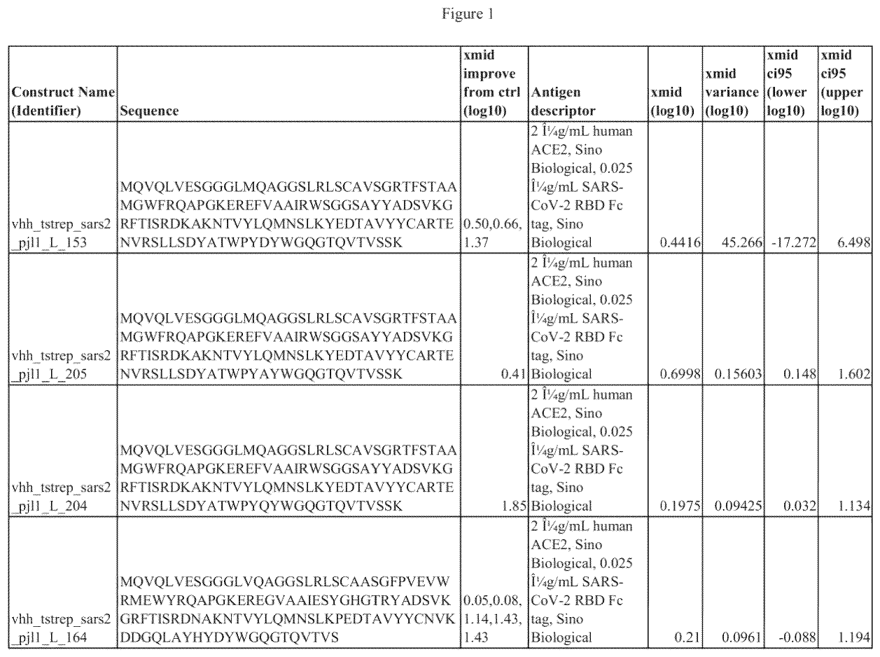

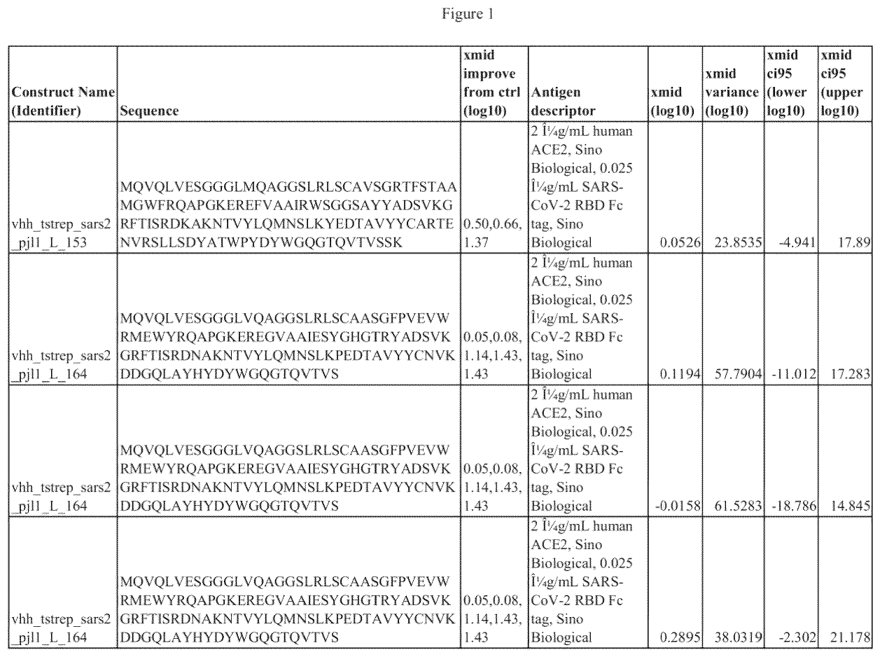

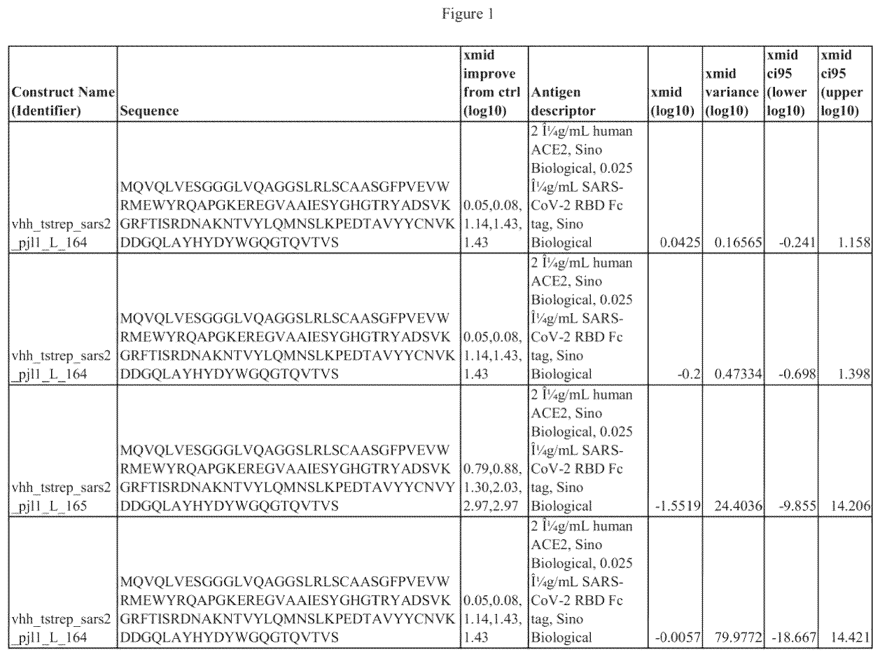

A number of single-chain polypeptides were generated that include either a single antigen-binding domain that bind specifically to the glycoprotein of SARS-CoV-2 or two antigen-binding domains (e.g., the same or different antigen-binding domains) that bind specifically to the glycoprotein of SARS-CoV2 (see, FIG. 1). The ability of each construct to bind SARS-CoV-2 glycoprotein was assessed using the enzyme-linked immunosorbent assay and/or the ACE2-competitive binding assay described below.

### Methods

### Enzyme-Linked Immunosorbent Assay

Briefly, SARS-CoV-2 RBD (SinoBiological, Cat. No. 40592-V02H) was coated onto 96 well plates overnight at 5 pg/mL optical density. Wells were blocked with 1% BSA in TBS-T for 1 hour at room temperature, and serial dilutions of purified polypeptides (e.g., VHH antibodies), ranging from 5 pg/mL to 0.0028 pg/mL, were prepared in TBS-T, added across the plate, and incubated for 1 hour at room temperature. Plates were washed with TBS-T three times, and HRP-conjugated rabbit anti-VHH antibody (GenScript, Cat. No. A01861) was added at 1:5000 dilution in blocking buffer. Plates were again washed as above, and BioFX TMB One Component HRP Substrate (Surmodics, Cat. No. TMBW-0100-01) was added for detection. Color development was stopped with BioFX stop solution (Surmodics, Cat. No. LSTP-0100-001), and color was read out on a Promega GloMax plate reader at 450 nm. Binding curves and EC₅₀ estimates were prepared using a proprietary analysis tool fitting a Bayesian logistic model to the concentration/absorbance data.

### ACE2-Competitive Binding Assay

Recombinant ACE2 (SinoBiological, Cat. No. 10108-H08B) was coated onto plates at 2 μg/mL, and plates were blocked with 1% BSA in TBS-T for 1 hour at room temperature. Separately, serial dilutions of polypeptides (e.g., VHH antibodies) were prepared, ranging from 5 μg/mL to 0.0028 μg/mL, in blocking buffer spiked with 0.025 μg/mL SARS-CoV-2 RBD (SinoBiological, Cat. No. 40592-V02H). These dilutions were incubated for 1 hour at room temperature and then added to the blocked, ACE2-coated plates for 30 minutes at room temperature. Plates were then washed three times with TBS-T. For detecting ACE2-bound RBD-Fc, HRP-conjugated goat anti-human IgG (gamma specific, Jackson ImmunoResearch, Cat. No. 109-036-008) was added to the wells at 1:5000 dilution in blocking buffer and incubated for 30 minutes at room temperature. Plates were washed for a second time following incubation. Color was developed and binding competition was analyzed as above.

### Results

The constructs in FIG. 1 demonstrate that each tested construct has SARS-CoV spike glycoprotein binding activity, SARS-CoV-2 spike glycoprotein-binding activity and/or ACE2-competitive binding activity. The “xmid improve from ctrl (log 10)” value indicates the estimated EC₅₀ in log 10 space for each sample relative to the EC₅₀ for a control. For each row in FIG. 1, the antigen(s) to which binding activity is being measured is indicated in the “antigen descriptor” column. FIG. 1 discloses SEQ ID NOS 222, 220, 226, 221, 217, 232, 223, 218, 222, 224, 226, 225, 232, 216, 217, 219, 219, 220, 218, 221, 223, 216, 225, 226, 224, 216, 217, 222, 232, 220, 218, 221, 219, 223-225, 222, 218, 226, 232, 221, 219, 216, 220, 217, 223, 226, 222, 232, 225, 218, 224, 219, 216, 217, 224, 221, 223, 220, 225, 232, 223, 223, 232-236, 232, 232, 237-241, 232, 242, 232, 234, 232, 237, 236, 233, 239, 232, 235, 240, 241, 238, 232, 242, 232, 232, 236, 235, 233, 239, 238, 240, 234, 232, 237, 232, 242, 232, 241, 232, 234, 237, 233, 236, 235, 232, 241, 240, 239, 238, 232, 242, 232, 243, 244, 243, 244, 244, 243, 244, 243, 245-258, 236, 236, 259-264, 236, 265-271, 236, 272-274, 248, 245, 275, 246, 236, 249, 276, 247, 254, 251, 255-257, 263, 250, 253, 260, 261, 258, 266, 262, 252, 264, 236, 259, 267, 265, 269, 268, 236, 271, 276, 275, 273, 236, 247, 248, 272, 245, 270, 250, 274, 236, 249, 255, 254, 259, 252, 251, 246, 256, 260, 263, 258, 267, 261, 236, 257, 262, 266, 253, 269, 265, 264, 268, 236, 275, 236, 273, 272, 277, 278, 276, 271, 279, 270, 280, 281, 237, 274, 244, 282, 283, 243, 284, 285, 237, 286, 287, 279, 278, 282, 284, 244, 280, 277, 243, 285, 281, 283, 287, 237, 277, 281, 286, 243, 287, 237, 284, 280, 279, 244, 282, 237, 287, 278, 278, 279, 277, 237, 285, 237, 283, 286, 284, 244, 280, 282, 285, 243, 283, 281, 286, 288, 289, 236, 290-293, 237, 294, 252, 295-297, 237, 298-302, 237, 303-305, 252, 252, 252, 306, 303, 307, 298, 299, 308, 237, 309-311, 283, 312, 282, 313-318, 283, 282, 319-321, 283, 285, 283, 317, 308, 286, 313, 237, 282, 318, 282, 309, 310, 312, 311, 282, 316, 314, 283, 319, 282, 283, 321, 282, 283, 285, 283, 236, 237, 320, 282, 285, 322, 287, 286, 287, 323-327, 287, 328-335, 287, 324, 326, 237, 322, 287, 278, 287, 321, 336, 326, 285, 287, 283, 243, 323, 325, 328, 327, 329, 331, 330, 243, 332, 323, 334, 243, 325, 335, 243, 327, 243, 328, 331, 329, 330, 332, 243, 334, 243, 333, 335, 282, 243, 282, 282, 282, 282, 282, 283, and 282, respectively, in order of appearance.

### Example 2: Affinity and Optimization of VHH-72

### Affinity Studies

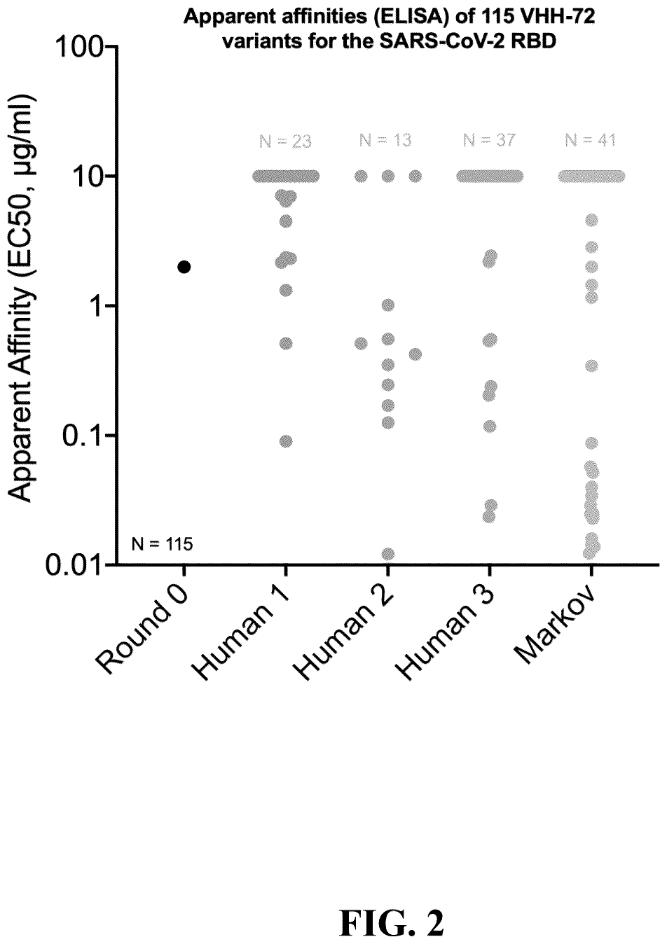

VHH-72 was subjected to successive rounds of affinity and functional optimization. VHH-72 expresses in CFPS with post-purification yields >0.1 mg/ml from a standard 100 μl PURExpress reaction containing 25-50 ng of template DNA. Purified VHH-72 demonstrated an appropriate gel-shift under reducing compared to non-reducing conditions, consistent with the proper formation of the single expected internal disulfide bond during cell-free expression. By ELISA, VHH-72 bound to the SARS-CoV-2 RBD with an EC₅₀ of approximately 2 μg/ml. (FIG. 2, Round 0)

### Optimization Studies

Optimization of VHH-72 for RBD binding and ACE2 competition was conducted by mutating each residue sequentially to the small, neutral residue alanine in individual CDR residues; native alanine residues were mutated to glycine (FIG. 2, Human 1). Only CDR residues (variants) within the 5 Å interface with the RBD (PDB: 6WAQ) were selected for optimization, which included 24 of 45 CDR residues.

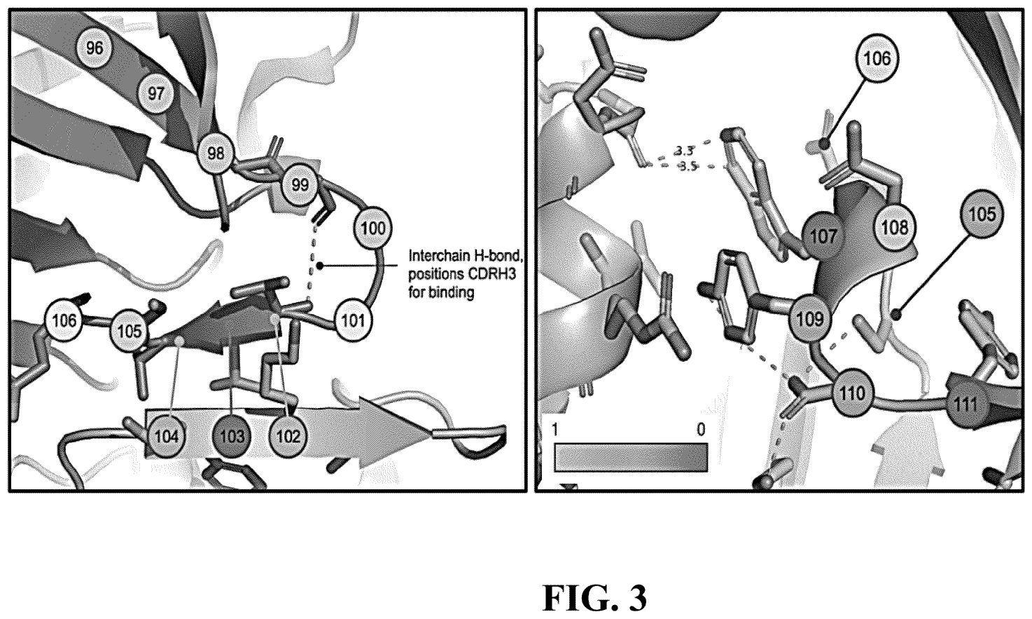

Of these 24 variants, one variant was unable to be synthesized by IDT, and the other 23 variants were successfully expressed and purified and compared by ELISA for RBD binding. Of the 23 expressed neutral variants, 16 displayed reduced binding, 4 displayed similar binding, and 3 displayed at least 1.5-fold increased binding to the RBD as compared to the wildtype antibody (FIG. 2, Human 1; FIG. 3). VHH-72 (S57A), a CDR2 variant, displayed a 22-fold increase in RBD affinity (EC₅₀=0.09 μg/ml), and VHH-72(D61A), also a CDR2 variant, displayed 4-fold increased RBD binding (EC₅₀=0.51 μg/ml). S57 and D61 are the native germline CDR2 residues. No CDR3 neutral variants enhanced binding, though several had similar affinity to the wildtype VHH (FIG. 2, Human 1).

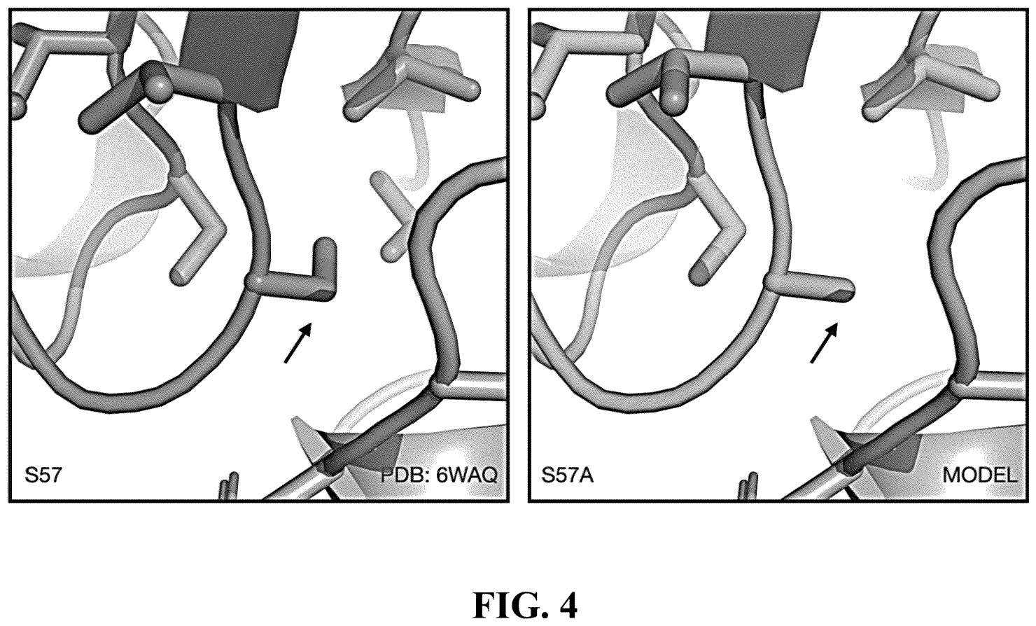

Given S57A and D61A significantly enhanced RBD binding, we next performed a library screen of sidechain geometry and chemistry at these positions, exploring the consequences of Ser, Glu, Gln, Lys, The, Tyr, and Gly. Besides Gly and Tyr, only in the case of SARS-CoV-2, and Ala as described above, all nonsynonymous mutants at S57 were deleterious demonstrating that S57 is not simply an unfavorable germline residue at this position (Table 1). VHH-72(S57S), a silent recodonisation of the wildtype residue, demonstrated equivalent RBD binding compared to the parental construct (Table 1). VHH-72 (S57Y) bound the SARS-CoV-2 RBD with roughly 4-fold increased apparent affinity, but this variant did not bind the SARS-CoV-1 RBD (EC50>10 μg/ml). Molecular Modeling demonstrates that the substitution to alanine may relieve a polar mismatch—S57 presents a polar sidechain to a relatively hydrophobic pocket on the RBD—and may allow CDR2 to relax into a stabilizing interaction (FIG. 4). These molecular models suggested VHH-72(S57T) may be further stabilizing, but this mutant abrogated binding to both SARS-CoV-1 and SARS-CoV-2 RBDs. Asp 61 was more tolerant to mutation than Ser 57. Variants expressing Lys and Tyr substitutions at codon 61 displayed similar affinity to the alanine mutant, whereas Gln and Ser substitutions enhanced binding by roughly 3-fold (D61Q EC₅₀: 0.13 μg/ml, D61S EC₅₀: 0.17 μg/ml). A single variant expressing both S57A and D61A displayed more than 160-fold improved binding (EC₅₀ 0.012 μg/ml; lowest point in Human Round 2, FIG. 2) compared to the wildtype VHH-72, 2-fold greater than the geometric product of the individual mutations, suggesting a synergistic effect of both mutations (combinatorial mutations) at the VHH:RBD interface.

TABLE 1
Rel. EC50 - ELISA

| | SARS1 | SARS2 |
|---|---|---|
| WT | 1 | 1 |
| S57S | 1 | 1 |
| S57E | n.b. | n.b. |
| S57K | n.b. | n.b. |
| S57Q | n.b. | n.b. |
| S57Y | n.b. | 0.28 |
| S57T | n.b. | n.b. |
| S57A | 0.43 | 0.05 |
| S57G | 0.30 | 0.09 |

### Example 3: Combinatorial Mutations of VHH-72 (S57A)

A combination of structure-guided and electrochemical library mutagenesis approaches were used to generate and study combinatorial mutations. Additional amino acid sidechains were then explored at twelve positions associated with increased or moderately (less than 5-fold) decreased binding in the neutral residue scan on the backbone of VHH-72(S57A). Specifically, the residues Ser, Thr, Glu, Gln, Lys, Arg, His, Val, and Tyr were sampled at these locations where structural modeling indicated such a mutation would not obviously disrupt the antibody peptide backbone structure (e.g. kinking) or secondary folding, resulting in total of 37 additional CDR variants (FIG. 2, Human Round 3).

The majority of these mutations reduced RBD binding. Of the 37 expressed variants, two constructs, expressing VHH-72 (S57A, Y60H) and VHH-72 (S57A, Y60T), displayed approximately 3.5-fold further-increased apparent RBD affinity (EC₅₀=0.029 μg/ml, EC₅₀=0.024 μg/ml respectively), and four constructs displayed similar or slightly binding to VHH-72(S57A). The remaining 31 constructs displayed dramatically reduced binding, indicating significant rearrangements in the interacting surface or CDR loops.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

Sequence Appendix

| SEQ | | |
|---|---|---|
| ID NO: | Identifier | Sequence |
| SEQ ID | L_7 CDR1 | GRTFSEYAMG |
| NO: 1 | | |
| SEQ ID | L_7 CDR2 | A-X₁-X₂-S-X₃-X₄-G-X₅-X₆-X₇-X₈-X₉-X₁₀-X₁₁-X₁₂-V-X₁₃-X₁₄ |
| NO: 2 | | |
| SEQ ID | L_7 CDR3 | A-X₁₅-A-X₁₆-X₁₇-X₁₈-T-V-V-S-X₁₉-X₂₀-X₂₁-Y-D-Y-D-Y |
| NO: 3 | | |
| SEQ ID | L_7 CDR2 | TISWSGGATYHTDTVKG |
| NO: 4 | | |
| SEQ ID | L_7 CDR2 | TISWSGGATYHTDSVKG |
| NO: 5 | | |
| SEQ ID | L_7 CDR2 | SISWSGGATYHTDSVKG |
| NO: 6 | | |
| SEQ ID | L_7 CDR2 | TISWSGGATYHTWSVKG |
| NO: 7 | | |
| SEQ ID | L_7 CDR2 | TISWSGGATGHTDSVKG |
| NO: 8 | | |
| SEQ ID | L_7 CDR2 | TESWSGGATYHTDSVKG |
| NO: 9 | | |
| SEQ ID | L_7 CDR2 | TISWSGGATYHTDSVDG |
| NO: 10 | | |
| SEQ ID | L_7 CDR2 | TISWSGWATYHTDSVKG |
| NO: 11 | | |
| SEQ ID | L_7 CDR2 | TISWIGGATYHTDSVKG |
| NO: 12 | | |
| SEQ ID | L_7 CDR2 | TISWSGGGTYHTDSVKG |
| NO: 13 | | |
| SEQ ID | L_7 CDR2 | TISWSGGASYHTDSVKG |
| NO: 14 | | |
| SEQ ID | L_7 CDR2 | TISWSGGAAYHTDSVKG |
| NO: 15 | | |
| SEQ ID | L_7 CDR2 | TISPSGGATYHTDSVKG |
| NO: 16 | | |
| SEQ ID | L_7 CDR2 | TISWSGGATYHTTSVKG |
| NO: 17 | | |
| SEQ ID | L_7 CDR2 | TISWSGGATYHTDSVKA |
| NO: 18 | | |
| SEQ ID | L_7 CDR2 | TISWGGGATYHTDSVKG |
| NO: 19 | | |
| SEQ ID | L_7 CDR2 | TSSWSGGATYHTDSVKG |
| NO: 20 | | |
| SEQ ID | L_7 CDR2 | TISLSGGATYHTDSVKG |
| NO: 21 | | |
| SEQ ID | L_7 CDR2 | TISWSGGATYHTDAVKG |
| NO: 22 | | |
| SEQ ID | L_7 CDR2 | TISWSGGATYHTASVKG |
| NO: 23 | | |
| SEQ ID | L_7 CDR2 | TISWSGGATYHYDSVKG |
| NO: 24 | | |
| SEQ ID | L_7 CDR2 | TISWSGGAGYHTDSVKG |
| NO: 25 | | |
| SEQ ID | L_7 CDR2 | TISGGWSGGATYHTDSVKG |
| NO: 26 | | |
| SEQ ID | L_7 CDR2 | TISGWSGGATYHTDSVKG |
| NO: 27 | | |
| SEQ ID | L_7 CDR2 | TISWSGGSTYYTRSVKG |
| NO: 28 | | |
| SEQ ID | L_7 CDR2 | TISWSGGSTYTTRSVKG |
| NO: 29 | | |
| SEQ ID | L_7 CDR2 | TISWSGGSTYTTDSVKG |
| NO: 30 | | |
| SEQ ID | L_7 CDR2 | TISWSGGSTYHTRSVKG |
| NO: 31 | | |
| SEQ ID | L_7 CDR2 | TISWSGGSTYHTDSVKG |
| NO: 32 | | |
| SEQ ID | L_7 CDR2 | TISWSGGGTYYTWSVKG |
| NO: 33 | | |
| SEQ ID | L_7 CDR2 | TISWSGGGTYYTRSVKG |
| NO: 34 | | |
| SEQ ID | L_7 CDR2 | TISWSGGGTYTTRSVKG |
| NO: 35 | | |
| SEQ ID | L_7 CDR2 | TISWSGGGTYHTRSVKG |
| NO: 36 | | |
| SEQ ID | L_7 CDR2 | TISWSGGATYYTQSVKG |
| NO: 37 | | |
| SEQ ID | L_7 CDR2 | TISWSGGATYYTDSVKG |
| NO: 38 | | |
| SEQ ID | L_7 CDR2 | TISWSGGGTYTTDSVKG |
| NO: 39 | | |
| SEQ ID | L_7 CDR2 | TISWSGGATYQTDSVKG |
| NO: 40 | | |
| SEQ ID | L_7 CDR2 | TISWSGGATYSTDSVKG |
| NO: 41 | | |
| SEQ ID | L_7 CDR2 | TISWSGGATYETDSVKG |
| NO: 42 | | |
| SEQ ID | L_7 CDR2 | TISWSGGATYTTDSVKG |
| NO: 43 | | |
| SEQ ID | L_7 CDR2 | TISWSGGATYYTRSVKG |
| NO: 44 | | |
| SEQ ID | L_7 CDR3 | AAAGLGTVVSEWDYDYDY |
| NO: 45 | | |
| SEQ ID | L_7 CDR3 | AAAGLGTVVSEWDDYDY |
| NO: 46 | | |
| SEQ ID | L_7 CDR3 | APAGLGTVVSEWDYDYDY |
| NO: 47 | | |
| SEQ ID | L_7 CDR3 | AAADLGTVVSEWDYDYDY |
| NO: 48 | | |
| SEQ ID | L_7 CDR3 | AAAGLGTVVSEADYDYDY |
| NO: 49 | | |
| SEQ ID | L_7 CDR3 | AAAGLRTVVSEWDYDYDY |
| NO: 50 | | |
| SEQ ID | L_7 CDR3 | AYAGLGTVVSEWDYDYDY |
| NO: 51 | | |
| SEQ ID | L_7 CDR3 | AAAGLGTVVSEWDYDYDS |
| NO: 52 | | |
| SEQ ID | L_7 CDR3 | AAAGLWTVVSEWDYDYDY |
| NO: 53 | | |
| SEQ ID | L_7 CDR3 | AAAGLGTVVSAWDYDYDY |
| NO: 54 | | |
| SEQ ID | L_7 CDR3 | AAAGLGTVVSEWDYDDY |
| NO: 55 | | |
| SEQ ID | L_7 CDR3 | AAAGQGTVVSEWDYDYDY |
| NO: 56 | | |
| SEQ ID | L_7 CDR3 | AAAGLETVVSEWDYDYDY |
| NO: 57 | | |
| SEQ ID | L_7 CDR3 | AAARLGTVVSEWDYDYDY |
| NO: 58 | | |
| SEQ ID | L_7 CDR3 | AAAELGTVVSEWDYDYDY |
| NO: 59 | | |
| SEQ ID | L_7 CDR3 | AAAGLGTVVSEWAYDYDA |
| NO: 60 | | |
| SEQ ID | Framework region | MQVQLQESGGGLVQAGGSLRLSCAASG |
| NO: 61 | 1 | |
| SEQ ID | Framework region | MQVQLQESGGGLVQAGGSLRLSCAASGSG |
| NO: 62 | 1 | |
| SEQ ID | vhh_tstrep_sars2_ | MQVQLQESGGGLVQAGGSLRLSCAASGRTFSEYAMG |
| NO: 63 | pjl1_L_112 | WFRQAPGKEREFVATISWSGGATYHTDTVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 64 | pjl1_L_113 | WFRQAPGKEREFVATISWSGGATYHTDSVKGRFTISRD |
| | | NAKNTVYLQMNSLKPDDTAVYYCAAAGLGTVVSEWD |
| | | FDYDYWGQGTQVTVSSS |
| SEQ ID | vhh_tstrep_sars2_ | MQVQLQESGGGLVQAGGSLRLSCAASGRTFSEYAMG |
| NO: 65 | pjl1_L_114 | WFRQAPGKEREFVATISWSGGATYHTDSVKGRFTISRD |
| | | NAKNTVYLQMNSLKPDDTAVYYCAPAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 66 | pjl1_L_115 | WFRQAPGKEREFVATISWSGGATYHTDSVKGRFTISRD |
| | | NAKNTVYLQMNSLKPDDTAVYYCAAADLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 67 | pjl1_L_116 | WFRQAPGKEREFVASISWSGGATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 68 | pjl1_L_117 | WFRQAPGKEREFVATISWSGGATYHTWSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 69 | pjl1_L_118 | WFRQAPGKEREFVATISWSGGATGHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 70 | pjl1_L_119 | WFRQAPGKEREFVATESWSGGATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 71 | pjl1_L_120 | WFRQAPGKEREFVATISWSGGATYHTDSVDGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 72 | pjl1_L_121 | WFRQAPGKEREFVATISWSGWATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 73 | pjl1_L_122 | WFRQAPGKEREFVATISWSGGATYHTDSVKGRFTISRD |
| | | NAKNTVYLQMNSLKPDDTAVYYCAAAGLGTVVSEAD |
| | | YDYDYWGQGTQVTVSSS |
| SEQ ID | vhh_tstrep_sars2_ | MQVQLQESGGGLVQAGGSLRLSCAASGRTFSEYAMG |
| NO: 74 | pjl1_L_123 | WFRQAPGKEREFVATISWIGGATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 75 | pjl1_L_124 | WFRQAPGKEREFVATISWSGGGTYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 76 | pjl1_L_125 | WFRQAPGKEREFVATISWSGGATYHTDSVKGRFTISRD |
| | | NAKNTVYLQMNSLKPDDTAVYYCAAAGLRTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 77 | pjl1_L_126 | WFRQAPGKEREFVATISWSGGASYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 78 | pjl1_L_127 | WFRQAPGKEREFVATISWSGGAAYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 79 | pjl1_L_128 | WFRQAPGKEREFVATISPSGGATYHTDSVKGRFTISRDN |
| | | AKNTVYLQMNSLKPDDTAVYYCAAAGLGTVVSEWDY |
| | | DYDYWGQGTQVTVSSS |
| SEQ ID | vhh_tstrep_sars2_ | MQVQLQESGGGLVQAGGSLRLSCAASGRTFSEYAMG |
| NO: 80 | pjl1_L_129 | WFRQAPGKEREFVATISWSGGATYHTTSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 81 | pjl1_L_130 | WFRQAPGKEREFVATISWSGGATYHTDSVKARFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 82 | pjl1_L_131 | WFRQAPGKEREFVATISWSGGATYHTDSVKGRFTISRD |
| | | NAKNTVYLQMNSLKPDDTAVYYCAYAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 83 | pjl1_L_132 | WFRQAPGKEREFVATISWGGGATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 84 | pjl1_L_133 | WFRQAPGKEREFVATISWSGGATYHTDSVKGRFTISRD |
| | | NAKNTVYLQMNSLKPDDTAVYYCAAAGLGTVVSEWD |
| | | YDYDSWGQGTQVTVSSS |
| SEQ ID | vhh_tstrep_sars2_ | MQVQLQESGGGLVQAGGSLRLSCAASGRTFSEYAMG |
| NO: 85 | pjl1_L_135 | WFRQAPGKEREFVATISWSGGATYHTDSVKGRFTISRD |
| | | NAKNTVYLQMNSLKPDDTAVYYCAAAGLWTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 86 | pjl1_L_137 | WFRQAPGKEREFVATISWSGGATYHTDSVKGRFTISRD |
| | | NAKNTVYLQMNSLKPDDTAVYYCAAAGLGTVVSAWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 87 | pjl1_L_139 | WFRQAPGKEREFVATISWSGGATYHTDSVKGRFTISRD |
| | | NAKNTVYLQMNSLKPDDTAVYYCAAAGLGTVVSEWD |
| | | YDFDYWGQGTQVTVSSS |
| SEQ ID | vhh_tstrep_sars2_ | MQVQLQESGGGLVQAGGSLRLSCAASGRTFSEYAMG |
| NO: 88 | pjl1_L_144 | WFRQAPGKEREFVATSSWSGGATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 89 | pjl1_L_145 | WFRQAPGKEREFVATISLSGGATYHTDSVKGRFTISRDN |
| | | AKNTVYLQMNSLKPDDTAVYYCAAAGLGTVVSEWDY |
| | | DYDYWGQGTQVTVSSS |
| SEQ ID | vhh_tstrep_sars2_ | MQVQLQESGGGLVQAGGSLRLSCAASGRTFSEYAMG |
| NO: 90 | pjl1_L_146 | WFRQAPGKEREFVATISWSGGATYHTDAVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 91 | pjl1_L_147 | WFRQAPGKEREFVATISWSGGATYHTDSVKGRFTISRD |
| | | NAKNTVYLQMNSLKPDDTAVYYCAAAGQGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 92 | pjl1_L_148 | WFRQAPGKEREFVATISWSGGATYHTASVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 93 | pjl1_L_149 | WFRQAPGKEREFVATISWSGGATYHYDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 94 | pjl1_L_150 | WFRQAPGKEREFVATISWSGGAGYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 95 | pjl1_L_206 | WFRQAPGKEREFVATISWSGGATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: 96 | pjl1_L_207 | WFRQAPGKEREFVATISGGWSGGATYHTDSVKGRFTIS |
| | | RDNAKNTVYLQMNSLKPDDTAVYYCAAAGLGTVVSE |
| | | WDYDYDYWGQGTQVTVSSS |
| SEQ ID | vhh_tstrep_sars2_ | MQVQLQESGGGLVQAGGSLRLSCAASGRTFSEYAMG |
| NO: 97 | pjl1_L_208 | WFRQAPGKEREFVATISGWSGGATYHTDSVKGRFTISR |
| | | DNAKNTVYLQMNSLKPDDTAVYYCAAAGLGTVVSEW |
| | | DYDYDYWGQGTQVTVSSS |
| SEQ ID | vhh_tstrep_sars2_ | MQVQLQESGGGLVQAGGSLRLSCAASGSGGRTFSEYA |
| NO: 98 | pjl1_L_209 | MGWFRQAPGKEREFVATISWSGGATYHTDSVKGRFTIS |
| | | RDNAKNTVYLQMNSLKPDDTAVYYCAAAGLGTVVSE |
| | | WDYDYDYWGQGTQVTVSSS |
| SEQ ID | vhh_tstrep_sars2_ | MQVQLQESGGGLVQAGGSLRLSCAASGGRTFSEYAMG |
| NO: 99 | pjl1_L_211 | WFRQAPGKEREFVATISWSGGATYHTDSVKGRFTISRD |
| | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_212 | WFRQAPGKEREFVATISWSGGSTYYTRSVKGRFTISRD |
| 100 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_213 | WFRQAPGKEREFVATISWSGGSTYTTRSVKGRFTISRDN |
| 101 | | AKNTVYLQMNSLKPDDTAVYYCAAAGLETVVSEWDY |
| | | DYDYWGQGTQVTVSSS |
| SEQ ID | vhh_tstrep_sars2_ | MQVQLQESGGGLVQAGGSLRLSCAASGRTFSEYAMG |
| NO: | pjl1_L_214 | WFRQAPGKEREFVATISWSGGSTYTTRSVKGRFTISRDN |
| 102 | | AKNTVYLQMNSLKPDDTAVYYCAAAGLGTVVSEWDY |
| | | DYDYWGQGTQVTVSSS |
| SEQ ID | vhh_tstrep_sars2_ | MQVQLQESGGGLVQAGGSLRLSCAASGRTFSEYAMG |
| NO: | pjl1_L_215 | WFRQAPGKEREFVATISWSGGSTYTTDSVKGRFTISRD |
| 103 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_216 | WFRQAPGKEREFVATISWSGGSTYHTRSVKGRFTISRD |
| 104 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_217 | WFRQAPGKEREFVATISWSGGSTYHTRSVKGRFTISRD |
| 105 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_218 | WFRQAPGKEREFVATISWSGGSTYHTDSVKGRFTISRD |
| 106 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_219 | WFRQAPGKEREFVATISWSGGGTYYTWSVKGRFTISRD |
| 107 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_220 | WFRQAPGKEREFVATISWSGGGTYYTRSVKGRFTISRD |
| 108 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_222 | WFRQAPGKEREFVATISWSGGGTYTTRSVKGRFTISRD |
| 109 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_224 | WFRQAPGKEREFVATISWSGGGTYHTRSVKGRFTISRD |
| 110 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_225 | WFRQAPGKEREFVATISWSGGATYYTQSVKGRFTISRD |
| 111 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_229 | WFRQAPGKEREFVATISWSGGGTYTTRSVKGRFTISRD |
| 112 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_23 | WFRQAPGKEREFVATISWSGGATYYTDSVKGRFTISRD |
| 113 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_230 | WFRQAPGKEREFVATISWSGGGTYTTDSVKGRFTISRD |
| 114 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_231 | WFRQAPGKEREFVATISWSGGGTYTTDSVKGRFTISRD |
| 115 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_232 | WFRQAPGKEREFVATISWSGGGTYHTRSVKGRFTISRD |
| 116 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_234 | WFRQAPGKEREFVATISWSGGGTYHTDSVKGRFTISRD |
| 117 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_74 | WFRQAPGKEREFVATISWSGGATYQTDSVKGRFTISRD |
| 118 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_75 | WFRQAPGKEREFVATISWSGGATYSTDSVKGRFTISRD |
| 119 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_76 | WFRQAPGKEREFVATISWSGGATYETDSVKGRFTISRD |
| 120 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_79 | WFRQAPGKEREFVATISWSGGATYTTDSVKGRFTISRD |
| 121 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_80 | WFRQAPGKEREFVATISWSGGATYYTRSVKGRFTISRD |
| 122 | | NAKNTVYLQMNSLKPDDTAVYYCAAAGLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_81 | WFRQAPGKEREFVATISWSGGATYYTDSVKGRFTISRD |
| 123 | | NAKNTVYLQMNSLKPDDTAVYYCAAARLGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_82 | WFRQAPGKEREFVATISWSGGATYYTDSVKGRFTISRD |
| 124 | | NAKNTVYLQMNSLKPDDTAVYYCAAAELGTVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_85 | WFRQAPGKEREFVATISWSGGATYYTDSVKGRFTISRD |
| 125 | | NAKNTVYLQMNSLKPDDTAVYYCAAAGLETVVSEWD |
| | | YDYDYWGQGTQVTVSSS |
| SEQ ID | vhh_tstrep_sars2_ | MQVQLQESGGGLVQAGGSLRLSCAASGRTFSEYAMG |
| NO: | pjl1_L_99 | WFRQAPGKEREFVATISWSGGATYYTDSVKGRFTISRD |
| 126 | | NAKNTVYLQMNSLKPDDTAVYYCAAAGLGTVVSEWA |
| | | YDYDAWGQGTQVTVSSS |
| SEQ ID | vhh_tstrep_sars2_ | GFPVYSWN |
| NO: | pjl1_L_166 CDR1 | |
| 127 | | |
| SEQ ID | vhh_tstrep_sars2_ | I-E-S-H-X1-D-S-T |
| NO: | pjl1_L_166 CDR2 | |
| 128 | | |
| SEQ ID | vhh_tstrep_sars2_ | Y-V-W-V-X2-H-T-Y-Y-G-Q |
| NO: | pjl1_L_166 CDR3 | |
| 129 | | |
| SEQ ID | vhh_tstrep_sars2_ | IESHGDST |
| NO: | pjl1_L_166 CDR2 | |
| 130 | | |
| SEQ ID | vhh_tstrep_sars2_ | IESHADST |
| NO: | pjl1_L_166 CDR2 | |
| 231 | | |
| SEQ ID | vhh_tstrep_sars2_ | YVWVGHTYYGQ |
| NO: | pjl1_L_166 CDR3 | |
| 132 | | |
| SEQ ID | vhh_tstrep_sars2_ | YVWVAHTYYGQ |
| NO: | pjl1_L_166 CDR3 | |
| 133 | | |
| SEQ ID | vhh_tstrep_sars2_ | MQVQLVESGGGLVQAGGSLRLSCAASGFPVYSWNMW |
| NO: | pjl1_L_166 Full | WYRQAPGKEREWVAAIESHGDSTRYADSVKGRFTISRD |
| 134 | | NAKNTVYLQMNSLKPEDTAVYYCYVWVGHTYYGQGT |
| | | QVTVSS |
| SEQ ID | vhh_tstrep_sars2_ | MQVQLVESGGGLVQAGGSLRLSCAASGFPVYSWNMW |
| NO: | pjl1_L_284 (166 | WYRQAPGKEREWVAAIESHADSTRYADSVKGRFTISRD |
| 135 | Full) | NAKNTVYLQMNSLKPEDTAVYYCYVWVGHTYYGQGT |
| | | QVTVSS |
| SEQ ID | vhh_tstrep_sars2_ | MQVQLVESGGGLVQAGGSLRLSCAASGFPVYSWNMW |
| NO: | pjl1_L_285_(166 | WYRQAPGKEREWVAAIESHGDSTRYADSVKGRFTISRD |
| 136 | Full) | NAKNTVYLQMNSLKPEDTAVYYCYVWVAHTYYGQGT |
| | | QVTVSS |
| SEQ ID | vhh_tstrep_sars_ | G-X₁-T-X₂-S-T-A-A |
| NO: | pjl1_L_153 CDR1 | |
| 137 | | |
| SEQ ID | vhh_tstrep_sars_ | I-R-W-S-X₃-G-S-A |
| NO: | pjl1_L_153 CDR2 | |
| 138 | | |
| SEQ ID | vhh_tstrep_sars_ | A-R-T-E-N-V-R-X₄-X₅-L-S-D-Y-A-T-X₆-P-Y-X₇-Y |
| NO: | pjl1_L_153 CDR3 | |
| 139 | | |
| SEQ ID | vhh_tstrep_sars_ | GRTFSTAA |
| NO: | pjl1_L_153 CDR1 | |
| 140 | | |
| SEQ ID | vhh_tstrep_sars_ | GRTFATAA |
| NO: | pjl1_L_153 CDR1 | |
| 141 | | |
| SEQ ID | vhh_tstrep_sars_ | GATFSTAA |
| NO: | pjl1_L_153 CDR1 | |
| 142 | | |
| SEQ ID | vhh_tstrep_sars_ | GRTYSTAA |
| NO: | pjl1_L_153 CDR1 | |
| 143 | | |
| SEQ ID | vhh_tstrep_sars_ | GRTHSTAA |
| NO: | pjl1_L_153 CDR1 | |
| 144 | | |
| SEQ ID | vhh_tstrep_sars_ | IRWSGGSA |
| NO: | pjl1_L_153 CDR2 | |
| 145 | | |
| SEQ ID | vhh_tstrep_sars_ | IRWSAGSA |
| NO: | pjl1_L_153 CDR2 | |
| 146 | | |
| SEQ ID | vhh_tstrep_sars_ | ARTENVRSLLSDYATWPYDY |
| NO: | pjl1_L_153 CDR3 | |
| 147 | | |
| SEQ ID | vhh_tstrep_sars_ | ARTENVRSLLSDYATYPYDY |
| NO: | pjl1_L_153 CDR3 | |
| 148 | | |
| SEQ ID | vhh_tstrep_sars_ | ARTENVRALLSDYATWPYDY |
| NO: | pjl1_L_153 CDR3 | |
| 149 | | |
| SEQ ID | vhh_tstrep_sars_ | ARTENVRSKLSDYATWPYDY |
| NO: | pjl1_L_153 CDR3 | |
| 150 | | |
| SEQ ID | vhh_tstrep_sars_ | ARTENVRSLLSDYATWPYRY |
| NO: | pjl1_L_153 CDR3 | |
| 151 | | |
| SEQ ID | vhh_tstrep_sars_ | ARTENVRSLLSDYATWPYQY |
| NO: | pjl1_L_153 CDR3 | |
| 152 | | |
| SEQ ID | vhh_tstrep_sars_ | ARTENVRSLLSDYATWPYAY |
| NO: | pjl1_L_153 CDR3 | |
| 153 | | |
| SEQ ID | vhh_tstrep_sars2_ | MQVQLVESGGGLMQAGGSLRLSCAVSGRTFSTAAMG |
| NO: | pjl1_L_153 | WFRQAPGKEREFVAAIRWSGGSAYYADSVKGRFTISRD |
| 154 | (153_Full) | KAKNTVYLQMNSLKYEDTAVYYCARTENVRSLLSDYA |
| | | TWPYDYWGQGTQVTVSSKS |
| SEQ ID | vhh_tstrep_sars2_ | MQVQLVESGGGLMQAGGSLRLSCAVSGRTFSTAAMG |
| NO: | pjl1_L_167 | WFRQAPGKEREFVAAIRWSGGSAYYADSVKGRFTISRD |
| 155 | (153_Full) | KAKNTVYLQMNSLKYEDTAVYYCARTENVRSLLSDYA |
| | | TYPYDYWGQGTQVTVSSKS |
| SEQ ID | vhh_tstrep_sars2_ | MQVQLVESGGGLMQAGGSLRLSCAVSGRTFATAAMG |
| NO: | pjl1_L_177 | WFRQAPGKEREFVAAIRWSGGSAYYADSVKGRFTISRD |
| 156 | (153_Full) | KAKNTVYLQMNSLKYEDTAVYYCARTENVRSLLSDYA |
| | | TWPYDYWGQGTQVTVSSKS |
| SEQ ID | vhh_tstrep_sars2_ | MQVQLVESGGGLMQAGGSLRLSCAVSGRTFSTAAMG |
| NO: | pjl1_L_178 | WFRQAPGKEREFVAAIRWSGGSAYYADSVKGRFTISRD |
| 157 | (153_Full) | KAKNTVYLQMNSLKYEDTAVYYCARTENVRALLSDY |
| | | ATWPYDYWGQGTQVTVSSKS |
| SEQ ID | vhh_tstrep_sars2_ | MQVQLVESGGGLMQAGGSLRLSCAVSGATFSTAAMG |
| NO: | pjl1_L_182 | WFRQAPGKEREFVAAIRWSGGSAYYADSVKGRFTISRD |
| 158 | (153_Full) | KAKNTVYLQMNSLKYEDTAVYYCARTENVRSLLSDYA |
| | | TWPYDYWGQGTQVTVSSKS |
| SEQ ID | vhh_tstrep_sars2_ | MQVQLVESGGGLMQAGGSLRLSCAVSGRTFSTAAMG |
| NO: | pjl1_L_194 | WFRQAPGKEREFVAAIRWSGGSAYYADSVKGRFTISRD |
| 159 | (153_Full) | KAKNTVYLQMNSLKYEDTAVYYCARTENVRSKLSDY |
| | | ATWPYDYWGQGTQVTVSSKS |
| SEQ ID | vhh_tstrep_sars2_ | MQVQLVESGGGLMQAGGSLRLSCAVSGRTFSTAAMG |
| NO: | pjl1_L_196 | WFRQAPGKEREFVAAIRWSAGSAYYADSVKGRFTISRD |
| 160 | (153_Full) | KAKNTVYLQMNSLKYEDTAVYYCARTENVRSLLSDYA |
| | | TWPYDYWGQGTQVTVSSKS |
| SEQ ID | vhh_tstrep_sars2_ | MQVQLVESGGGLMQAGGSLRLSCAVSGRTYSTAAMG |
| NO: | pjl1_L_197 | WFRQAPGKEREFVAAIRWSGGSAYYADSVKGRFTISRD |
| 161 | (153_Full) | KAKNTVYLQMNSLKYEDTAVYYCARTENVRSLLSDYA |
| | | TWPYDYWGQGTQVTVSSKS |
| SEQ ID | vhh_tstrep_sars2_ | MQVQLVESGGGLMQAGGSLRLSCAVSGRTHSTAAMG |
| NO: | pjl1_L_198 | WFRQAPGKEREFVAAIRWSGGSAYYADSVKGRFTISRD |
| 162 | (153_Full) | KAKNTVYLQMNSLKYEDTAVYYCARTENVRSLLSDYA |
| | | TWPYDYWGQGTQVTVSSKS |
| SEQ ID | vhh_tstrep_sars2_ | MQVQLVESGGGLMQAGGSLRLSCAVSGRTFSTAAMG |
| NO: | pjl1_L_203 | WFRQAPGKEREFVAAIRWSGGSAYYADSVKGRFTISRD |
| 163 | (153_Full) | KAKNTVYLQMNSLKYEDTAVYYCARTENVRSLLSDYA |
| | | TWPYRYWGQGTQVTVSSKS |
| SEQ ID | vhh_tstrep_sars2_ | MQVQLVESGGGLMQAGGSLRLSCAVSGRTFSTAAMG |
| NO: | pjl1_L_204 | WFRQAPGKEREFVAAIRWSGGSAYYADSVKGRFTISRD |
| 164 | (153_Full) | KAKNTVYLQMNSLKYEDTAVYYCARTENVRSLLSDYA |
| | | TWPYQYWGQGTQVTVSSKS |
| SEQ ID | vhh_tstrep_sars2_ | MQVQLVESGGGLMQAGGSLRLSCAVSGRTFSTAAMG |
| NO: | pjl1_L_205 | WFRQAPGKEREFVAAIRWSGGSAYYADSVKGRFTISRD |
| 165 | (153_Full) | KAKNTVYLQMNSLKYEDTAVYYCARTENVRSLLSDYA |
| | | TWPYAYWGQGTQVTVSSKS |
| SEQ ID | vhh_tstrep_sars2_ | G-F-P-V-E-X₁-X₂-X₃ |
| NO: | pjl1_L_164 CDR1 | |
| 166 | | |
| SEQ ID | vhh_tstrep_sars2_ | I-E-X₄-X₅-G-H-G-X₆ |
| NO: | pjl1_L_164 CDR2 | |
| 167 | | |
| SEQ ID | vhh_tstrep_sars2_ | N-V-X₇-D-D-X₈-X₉-L-A-Y-H-Y-D-Y |
| NO: | pjl1_L_164 CDR3 | |
| 168 | | |
| SEQ ID | vhh_tstrep_sars2_ | GFPVEVWR |
| NO: | pjl1_L_164 CDR1 | |
| 169 | | |
| SEQ ID | vhh_tstrep_sars2_ | GFPVEVYR |
| NO: | pjl1_L_164 CDR1 | |
| 170 | | |
| SEQ ID | vhh_tstrep_sars2_ | GFPVEVAR |
| NO: | pjl1_L_164 CDR1 | |
| 171 | | |
| SEQ ID | vhh_tstrep_sars2_ | GFPVEAWR |
| NO: | pjl1_L_164 CDR1 | |
| 172 | | |
| SEQ ID | vhh_tstrep_sars2_ | GFPVEVWA |
| NO: | pjl1_L_164 CDR1 | |
| 173 | | |
| SEQ ID | vhh_tstrep_sars2_ | IESYGHGT |
| NO: | pjl1_L_164 CDR2 | |
| 174 | | |
| SEQ ID | vhh_tstrep_sars2_ | IESAGHGT |
| NO: | pjl1_L_164 CDR2 | |
| 175 | | |
| SEQ ID | vhh_tstrep_sars2_ | IESYGHGA |
| NO: | pjl1_L_164 CDR2 | |
| 176 | | |
| SEQ ID | vhh_tstrep_sars2_ | IEAYGHGT |
| NO: | pjl1_L_164 CDR2 | |
| 177 | | |
| SEQ ID | vhh_tstrep_sars2_ | NVKDDGQLAYHYDY |
| NO: | pjl1_L_164 CDR3 | |
| 178 | | |
| SEQ ID | vhh_tstrep_sars2_ | NVYDDGQLAYHYDY |
| NO: | pjl1_L_164 CDR3 | |
| 179 | | |
| SEQ ID | vhh_tstrep_sars2_ | NVYDDGHLAYHYDY |
| NO: | pjl1_L_164 CDR3 | |
| 180 | | |
| SEQ ID | vhh_tstrep_sars2_ | NVYDDNQLAYHYDY |
| NO: | pjl1_L_164 CDR3 | |
| 181 | | |
| SEQ ID | Framekwork | RYADSVKGRFTISRDNAKNTVYLQMNSLKPEDTAVYY |
| NO: | region 3 | C |
| 182 | | |
| SEQ ID | Framework region | RYADSVRGRFTISRDNAKNTVYLQMNSLKPEDTAVYY |
| NO: | 3 | C |
| 183 | | |
| SEQ ID | Framework region | RYADSVAGRFTISRDNAKNTVYLQMNSLKPEDTAVYY |
| NO: | 3 | C |
| 184 | | |
| SEQ ID | vhh_tstrep_sars2_ | MQVQLVESGGGLVQAGGSLRLSCAASGFPVEVWRME |
| NO: | pjl1_L_164 | WYRQAPGKEREGVAAIESYGHGTRYADSVKGRFTISRD |
| 185 | (164_Full) | NAKNTVYLQMNSLKPEDTAVYYCNVKDDGQLAYHYD |
| | | YWGQGTQVTVSS |
| SEQ ID | vhh_tstrep_sars2_ | MQVQLVESGGGLVQAGGSLRLSCAASGFPVEVWRME |
| NO: | pjl1_L_165 | WYRQAPGKEREGVAAIESYGHGTRYADSVKGRFTISRD |
| 186 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEVWRME |
| NO: | pjl1_L_235 | WYRQAPGKEREGVAAIESAGHGTRYADSVKGRFTISRD |
| 187 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEVYRME |
| NO: | pjl1_L_236 | WYRQAPGKEREGVAAIESYGHGTRYADSVKGRFTISRD |
| 188 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEVARME |
| NO: | pjl1_L_237 | WYRQAPGKEREGVAAIESYGHGTRYADSVKGRFTISRD |
| 189 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEAWRME |
| NO: | pjl1_L_239 | WYRQAPGKEREGVAAIESYGHGTRYADSVKGRFTISRD |
| 190 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEVWRME |
| NO: | pjl1_L_240 | WYRQAPGKEREGVAAIESYGHGARYADSVKGRFTISR |
| 191 | (164_Full) | DNAKNTVYLQMNSLKPEDTAVYYCNVYDDGQLAYHY |
| | | DYWGQGTQVTVSS |
| SEQ ID | vhh_tstrep_sars2_ | MQVQLVESGGGLVQAGGSLRLSCAASGFPVEVWRME |
| NO: | pjl1_L_241 | WYRQAPGKEREGVAAIEAYGHGTRYADSVKGRFTISR |
| 192 | (164_Full) | DNAKNTVYLQMNSLKPEDTAVYYCNVYDDGQLAYHY |
| | | DYWGQGTQVTVSS |
| SEQ ID | vhh_tstrep_sars2_ | MQVQLVESGGGLVQAGGSLRLSCAASGFPVEVWAME |
| NO: | pjl1_L_244 | WYRQAPGKEREGVAAIESYGHGTRYADSVKGRFTISRD |
| 193 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEVWRME |
| NO: | pjl1_L_245 | WYRQAPGKEREGVAAIESYGHGTRYADSVKGRFTISRD |
| 194 | (164_Full) | NAKNTVYLQMNSLKPEDTAVYYCNVYDDGHLAYHYD |
| | | YWGQGTQVTVSS |
| SEQ ID | vhh_tstrep_sars2_ | MQVQLVESGGGLVQAGGSLRLSCAASGFPVEVWRME |
| NO: | pjl1_L_248 | WYRQAPGKEREGVAAIESYGHGTRYADSVRGRFTISRD |
| 195 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEVWRME |
| NO: | pjl1_L_249 | WYRQAPGKEREGVAAIESYGHGTRYADSVAGRFTISRD |
| 196 | (164_Full) | NAKNTVYLQMNSLKPEDTAVYYCNVYDDGQLAYHYD |
| | | YWGQGTQVTVSS |
| SEQ ID | vhh_tstrep_sars2_ | MQVQLVESGGGLVQAGGSLRLSCAASGFPVEVWRME |
| NO: | pjl1_L_254 | WYRQAPGKEREGVAAIESYGHGTRYADSVKGRFTISRD |
| 197 | (164_Full) | NAKNTVYLQMNSLKPEDTAVYYCNVYDDNQLAYHYD |
| | | YWGQGTQVTVSS |
| SEQ ID | vhh_biparatopic_ | MMQVQLQESGGGLVQAGGSLRLSCAASGRTFSEYAMG |
| NO: | vhhl79_GGGGS2_ | WFRQAPGKEREFVATISWSGGATYTTDSVKGRFTISRD |
| 198 | MR17 | NAKNTVYLQMNSLKPDDTAVYYCAAAGLGTVVSEWD |
| | | YDYDYWGQGTQVTVSSGGGGSGGGGSQVQLVESGGG |
| | | LVQAGGSLRLSCAASGFPVEVWRMEWYRQAPGKEREG |
| | | VAAIESYGHGTRYADSVKGRFTISRDNAKNTVYLQMNS |
| | | LKPEDTAVYYCNVKDDGQLAYHYDYWGQGTQVTVSS |
| SEQ ID | vhh_biparatopic_ | MMQVQLQESGGGLVQAGGSLRLSCAASGRTFSEYAMG |
| NO: | vhhl79_GGGGS3_ | WFRQAPGKEREFVATISWSGGATYTTDSVKGRFTISRD |
| 199 | MR17 | NAKNTVYLQMNSLKPDDTAVYYCAAAGLGTVVSEWD |
| | | YDYDYWGQGTQVTVSSQVQLVESGGGLVQAGGSLRLS |
| | | CAASGFPVEVWRMEWYRQAPGKEREGVAAIESYGHGT |
| | | RYADSVKGRFTISRDNAKNTVYLQMNSLKPEDTAVYY |
| | | CNVKDDGQLAYHYDYWGQGTQVTVSS |
| SEQ ID | vhh_biparatopic_ | MMQVQLQESGGGLVQAGGSLRLSCAASGRTFSEYAMG |
| NO: | vhhl79_GGGGS3_ | WFRQAPGKEREFVATISWSGGATYTTDSVKGRFTISRD |
| 200 | MR17_k99y | NAKNTVYLQMNSLKPDDTAVYYCAAAGLGTVVSEWD |
| | | YDYDYWGQGTQVTVSSQVQLVESGGGLVQAGGSLRLS |
| | | CAASGFPVEVWRMEWYRQAPGKEREGVAAIESYGHGT |
| | | RYADSVKGRFTISRDNAKNTVYLQMNSLKPEDTAVYY |
| | | CNVYDDGQLAYHYDYWGQGTQVTVSS |
| SEQ ID | vhh_biparatopic_ | MMQVQLQESGGGLVQAGGSLRLSCAASGRTFSEYAMG |
| NO: | vhhl79_GGGGS3_ | WFRQAPGKEREFVATISWSGGATYTTDSVKGRFTISRD |
| 201 | oxnb1 | NAKNTVYLQMNSLKPDDTAVYYCAAAGLGTVVSEWD |
| | | YDYDYWGQGTQVTVSSQVQLVESGGGLMQAGGSLRL |
| | | SCAVSGRTFSTAAMGWFRQAPGKEREFVAAIRWSGGS |
| | | AYYADSVKGRFTISRDKAKNTVYLQMNSLKYEDTAVY |
| | | YCARTENVRSLLSDYATWPYDYWGQGTQVTVSSKS |
| SEQ ID | vhh_biparatopic_ | MMQVQLQESGGGLVQAGGSLRLSCAASGRTFSEYAMG |
| NO: | vhhl79_GGGGS4_ | WFRQAPGKEREFVATISWSGGATYTTDSVKGRFTISRD |
| 202 | oxnb1 | NAKNTVYLQMNSLKPDDTAVYYCAAAGLGTVVSEWD |
| | | YDYDYWGQGTQVTVSSQVQLVESGGGLMQAGGSLRL |
| | | SCAVSGRTFSTAAMGWFRQAPGKEREFVAAIRWSGGS |
| | | AYYADSVKGRFTISRDKAKNTVYLQMNSLKYEDTAVY |
| | | YCARTENVRSLLSDYATWPYDYWGQGTQVTVSSKS |
| SEQ ID | vhh_biparatopic_ | MMQVQLQESGGGLVQAGGSLRLSCAASGRTFSEYAMG |
| NO: | vhhl79_GGGGS4_ | WFRQAPGKEREFVATISWSGGATYTTDSVKGRFTISRD |
| 203 | sr4 | NAKNTVYLQMNSLKPDDTAVYYCAAAGLGTVVSEWD |
| | | YDYDYWGQGTQVTVSSQVQLVESGGGLVQAGGSLRLS |
| | | CAASGFPVYSWNMWWYRQAPGKEREWVAAIESHGDS |
| | | TRYADSVKGRFTISRDNAKNTVYLQMNSLKPEDTAVY |
| | | YCYVWVGHTYYGQGTQVTVSS |
| SEQ ID | vhh_biparatopic_ | MMQVQLQESGGGLVQAGGSLRLSCAASGRTFSEYAMG |
| NO: | vhhl79_GGGGS4_ | WFRQAPGKEREFVATISWSGGATYTTDSVKGRFTISRD |
| 204 | ty1 | NAKNTVYLQMNSLKPDDTAVYYCAAAGLGTVVSEWD |
| | | YDYDYWGQGTQVTVSSQVQLVETGGGLVQPGGSLRLS |
| | | CAASGFTFSSVYMNWVRQAPGKGPEWVSRISPNSGNIG |
| | | YTDSVKGRFTISRDNAKNTLYLQMNNLKPEDTALYYC |
| | | AIGLNLSSSSVRGQGTQVTVSSS |
| SEQ ID | vhh_biparatopic_ | MMQVQLQESGGGLVQAGGSLRLSCAASGRTFSEYAMG |
| NO: | vhhl79_GGGGS5_ | WFRQAPGKEREFVATISWSGGATYTTDSVKGRFTISRD |
| 205 | sb45 | NAKNTVYLQMNSLKPDDTAVYYCAAAGLGTVVSEWD |
| | | YDYDYWGQGTQVTVSSQVQLVESGGGLVQAGGSLRLS |
| | | CAASGFPVYRDRMAWYRQAPGKEREWVAAIYSAGQQ |
| | | TRYADSVKGRFTISRDNAKNTVYLQMNSLKPEDTAVY |
| | | YCNVKDVGHHYEYYDYWGQGTQVTVSS |
| SEQ ID | vhh_biparatopic_ | MMQVQLQESGGGLVQAGGSLRLSCAASGRTFSEYAMG |
| NO: | vhhl79_GGGGS_ | WFRQAPGKEREFVATISWSGGATYTTDSVKGRFTISRD |
| 206 | MR17 | NAKNTVYLQMNSLKPDDTAVYYCAAAGLGTVVSEWD |
| | | YDYDYWGQGTQVTVSSSGGGGSQVQLVESGGGLVQA |
| | | GGSLRLSCAASGFPVEVWRMEWYRQAPGKEREGVAAI |
| | | ESYGHGTRYADSVKGRFTISRDNAKNTVYLQMNSLKPE |
| | | DTAVYYCNVKDDGQLAYHYDYWGQGTQVTVSS |
| SEQ ID | Linker | GGGGS |
| NO: | | |
| 207 | | |
| SEQ ID | Linker | GGGGSGGGGS |
| NO: | | |
| 208 | | |
| SEQ ID | Linker | GGGGSGGGGSGGGGS |
| NO: | | |
| 209 | | |
| SEQ ID | Linker | GGGGSGGGGSGGGGSGGGGS |
| NO: | | |
| 210 | | |
| SEQ ID | Linker | GGGS |
| NO: | | |
| 211 | | |
| SEQ ID | Linker | GGSG |
| NO: | | |
| 212 | | |
| SEQ ID | vhh_biparatopic_ | MMQVQLQESGGGLVQAGGSLRLSCAASGRTFSEYAMG |
| NO: | vhhl79 | WFRQAPGKEREFVATISWSGGATYTTDSVKGRFTISRD |
| 213 | | NAKNTVYLQMNSLKPDDTAVYYCAAAGLGTVVSEWD |
| | | YDYDYWGQGTQVTVSS |
| SEQ ID | vhh_biparatopic_ | QVQLVETGGGLVQPGGSLRLSCAASGFTFSSVYMNWV |
| NO: | ty1 | RQAPGKGPEWVSRISPNSGNIGYTDSVKGRFTISRDNAK |
| 214 | | NTLYLQMNNLKPEDTALYYCAIGLNLSSSSVRGQGTQV |
| | | TVSSS |
| SEQ ID | vhh_biparatopic_ | QVQLVESGGGLVQAGGSLRLSCAASGFPVYRDRMAW |
| NO: | sb45 | YRQAPGKEREWVAAIYSAGQQTRYADSVKGRFTISRD |
| 215 | | NAKNTVYLQMNSLKPEDTAVYYCNVKDVGHHYEYYD |
| | | YWGQGTQVTVSS |
| SEQ ID | vhh_tstrep_sars2_ | MQVQLQESGGGLVQAGGSLRLSCAASGRTFSEYAMG |
| NO: | pjl1_L_22 | WFRQAPGKEREFVATISASGGSTYYTDSVKGRFTISRDN |
| 216 | | AKNTVYLQMNSLKPDDTAVYYCAAAGLGTVVSEWDY |
| | | DYDYWGQGTQVTVSS |
| SEQ ID | vhh_tstrep_sars2_ | MQVQLQESGGGLVQAGGSLRLSCAASGRTFSEYAMG |
| NO: | pjl1_L_24 | WFRQAPGKEREFVATISWSGGSTAYTDSVKGRFTISRD |
| 217 | | NAKNTVYLQMNSLKPDDTAVYYCAAAGLGTVVSEWD |
| | | YDYDYWGQGTQVTVSS |
| SEQ ID | vhh_tstrep_sars2_ | MQVQLQESGGGLVQAGGSLRLSCAASGRTFSEYAMG |
| NO: | pjl1_L_27 | WFRQAPGKEREFVATISWSGGSTYYTDSVKGRFTISRD |
| 218 | | NAKNTVYLQMNSLKPDDTAVYYCAAAGLATVVSEWD |
| | | YDYDYWGQGTQVTVSS |
| SEQ ID | vhh_tstrep_sars2_ | MQVQLQESGGGLVQAGGSLRLSCAASGRTFSEYAMG |
| NO: | pil1_L_32 | WFRQAPGKEREFVATISWSGGSTYYTDSVKGRFTISRD |
| 219 | | NAKNTVYLQMNSLKPDDTAVYYCAAAGLGTVVSAWD |
| | | YDYDYWGQGTQVTVSS |
| SEQ ID | vhh_tstrep_sars2_ | MQVQLQESGGGLVQAGGSLRLSCAASGRTFSEYAMG |
| NO: | pjl1_L_35 | WFRQAPGKEREFVATISWSGGSTYYTDSVKGRFTISRD |
| 220 | | NAKNTVYLQMNSLKPDDTAVYYCAGAGLGTVVSEWD |
| | | YDYDYWGQGTQVTVSS |
| SEQ ID | vhh_tstrep_sars2_ | MQVQLQESGGGLVQAGGSLRLSCAASGRTFSEYAMG |
| NO: | pjl1_L_38 | WFRQAPGKEREFVATISWSGGSTYYTDSVKGRFTISRD |
| 221 | | NAKNTVYLQMNSLKPDDTAVYYCAAAGLGTVVSEWA |
| | | YDYDYWGQGTQVTVSS |
| SEQ ID | vhh_tstrep_sars2_ | MQVQLQESGGGLVQAGGSLRLSCAASGRTFSEYAMG |
| NO: | pjl1_L_4 | WFRQAPGKEREFVATISWSGGSTYYTDSVKGRFTISRD |
| 222 | | NAKNTVYLQMNSLKPDDTAVYYCAAGGLGTVVSEWD |
| | | YDYDYWGQGTQVTVSS |
| SEQ ID | vhh_tstrep_sars2_ | MQVQLQESGGGLVQAGGSLRLSCAASGRTFSEYAMG |
| NO: | pjl1_L_40 | WFRQAPGKEREFVATISWSGGSTYYTASVKGRFTISRD |
| 223 | | NAKNTVYLQMNSLKPDDTAVYYCAAAGLGTVVSEWD |
| | | YDYDYWGQGTQVTVSS |
| SEQ ID | vhh_tstrep_sars2_ | MQVQLQESGGGLVQAGGSLRLSCAASGRTFSEYAMG |
| NO: | pjl1_L_46 | WFRQAPGKEREFVATISWSGGSTYYTDSVKGRFTISRD |
| 224 | | NAKNTVYLQMNSLKPDDTAVYYCAAAGLGTVVSEAD |
| | | YDYDYWGQGTQVTVSS |
| SEQ ID | vhh_tstrep_sars2_ | MQVQLQESGGGLVQAGGSLRLSCAASGRTFSEYAMG |
| NO: | pil1_L_47 | WFRQAPGKEREFVATISWSGGSTYYTDSVKGRFTISRD |
| 225 | | NAKNTVYLQMNSLKPDDTAVYYCAAAGLGTVVSEWD |
| | | YDADYWGQGTQVTVSS |
| SEQ ID | vhh_tstrep_sars2_ | MQVQLQESGGGLVQAGGSLRLSCAASGRTFSEYAMG |
| NO: | pjl1_L_1 | WFRQAPGKEREFVATISWSGGSTYYTDSVKGRFTISRD |
| 226 | | NAKNTVYLQMNSLKPDDTAVYYCGAAGLGTVVSEWD |
| | | YDYDYWGQGTQVTVSS |

1. An antibody that specifically binds SARS-CoV-2 receptor binding domain (RBD) comprising:
a) a heavy chain complementarity determining region 1 (VH-CDR1) comprising an amino acid sequence GRTFSEYA (SEQ ID NO: 227), or an amino acid sequence that has 1, 2, or 3 amino acid substitutions, additions, or deletions relative to SEQ ID NO: 227;
b) a heavy chain complementarity determining region 2 (VH-CDR2) comprising an amino acid sequence TISWSGGATYYTDSVKG (SEQ ID NO: 38), an amino acid sequence TISWSGGSTYYTASVKG (SEQ ID NO: 228), an amino acid sequence TISWSGGATYYTASVKG (SEQ ID NO: 229); or an amino acid sequence TISWSGGGTYYTDSVKG (SEQ ID NO: 230); and
c) a heavy chain complementarity determining region 3 (VH-CDR3) comprising an amino acid sequence AAAGLGTVVSEWDYDYDY (SEQ ID NO: 45), or an amino acid sequence that has 1, 2, or 3 amino acid substitutions, additions, or deletions relative to SEQ ID NO: 45.

2. The antibody of claim 1, wherein the VH-CDR1 comprises an amino acid sequence GX₁TX₂X₃X₄X₅A, wherein X₁ is R or A, X₂ is F, Y or H, X₃ is S or A, X₄ is T or E, and X₅ is Y or A.

3. The antibody of claim 2, wherein the VH-CDR1 comprises an amino acid sequence GRTFSTAA (SEQ ID NO: 140), GRTFATAA (SEQ ID NO: 141), GATFSTAA (SEQ ID NO: 142), GRTYSTAA (SEQ ID NO: 143), or GRTHSTAA (SEQ ID NO: 144).

4. The antibody of claim 1, wherein the VH-CDR1 comprises an amino acid sequence GRTFSEYA (SEQ ID NO: 227).

5. The antibody of claim 1, wherein the VH-CDR2 comprises an amino acid sequence TISWSGGATYYTDSVKG (SEQ ID NO: 38).

6. The antibody of claim 1, wherein the VH-CDR2 comprises an amino acid sequence TISWSGGSTYYTASVKG (SEQ ID NO: 228).

7. The antibody of claim 1, wherein the VH-CDR3 comprises an amino acid sequence AX₆AX₇X₈X₉TVVSX₁₀X₁₁X₁₂YDYDX₁₃, wherein X₆ is Y, P or A, X₇ is G, D, or R, X₈ is L or Q, X₉ is G, R, W, or E, X₁₀ is E or A, X₁₁ is W or A, X₁₂ is D or A, and X₁₃ is Y, S, or A.

8. The antibody of claim 7, wherein the VH-CDR3 comprises an amino acid sequence AAAGLGTVVSEWDYDYDY (SEQ ID NO: 45), APAGLGTVVSEWDYDYDY (SEQ ID NO: 47), AAADLGTVVSEWDYDYDY (SEQ ID NO: 48), AAAGLGTVVSEADYDYDY (SEQ ID NO: 49), AAAGLRTVVSEWDYDYDY (SEQ ID NO: 50), AYAGLGTVVSEWDYDYDY (SEQ ID NO: 51), AAAGLGTVVSEWDYDYDS (SEQ ID NO: 52), AAAGLWTVVSEWDYDYDY (SEQ ID NO: 53), AAAGLGTVVSAWDYDYDY (SEQ ID NO: 54), AAAGQGTVVSEWDYDYDY (SEQ ID NO: 56), AAAGLETVVSEWDYDYDY (SEQ ID NO: 57), AAARLGTVVSEWDYDYDY (SEQ ID NO: 58), AAAGLETVVSEWDYDYDY (SEQ ID NO: 59); or AAAGLGTVVSEWAYDYDA (SEQ ID NO: 60).

9. The antibody of claim 8, wherein the VH-CDR3 comprises an amino acid sequence AAAGLGTVVSEWDYDYDY (SEQ ID NO: 45).

10. The antibody of claim 1, wherein the VH-CDR1 comprises an amino acid sequence GRTFSEYA (SEQ ID NO: 227), wherein the VH-CDR2 comprises an amino acid sequence TISWSGGATYYTDSVKG (SEQ ID NO: 38), and wherein the VH-CDR3 comprises an amino acid sequence AAAGLGTVVSEWDYDYDY (SEQ ID NO: 45).

11. The antibody of claim 1, wherein the VH-CDR1 comprises an amino acid sequence GRTFSEYA (SEQ ID NO: 227), wherein the VH-CDR2 comprises an amino acid sequence TISWSGGSTYYTASVKG (SEQ ID NO: 228), and wherein the VH-CDR3 comprises an amino acid sequence AAAGLGTVVSEWDYDYDY (SEQ ID NO: 45).

12. The antibody of claim 1, wherein the VH-CDR1 comprises an amino acid sequence GRTFSEYA (SEQ ID NO: 227), wherein the VH-CDR2 comprises an amino acid sequence TISWSGGATYYTASVKG (SEQ ID NO: 229), and wherein the VH-CDR3 comprises an amino acid sequence AAAGLGTVVSEWDYDYDY (SEQ ID NO: 45).

13. The antibody of claim 1, wherein the VH-CDR1 comprises an amino acid sequence GRTFSEYA (SEQ ID NO: 227), wherein the VH-CDR2 comprises an amino acid sequence TISWSGGATYYTDSVKG (SEQ ID NO: 38), and wherein the VH-CDR3 comprises an amino acid sequence AAAGQGTVVSEWDYDYDY (SEQ ID NO: 56).

14. The antibody of claim 1, wherein the VH-CDR1 comprises an amino acid sequence GRTFSEYA (SEQ ID NO: 227), wherein the VH-CDR2 comprises an amino acid sequence TISWSGGGTYYTDSVKG (SEQ ID NO: 230), and wherein the VH-CDR3 comprises an amino acid sequence AAAGQGTVVSEWDYDYDY (SEQ ID NO: 56).

15. The antibody of claim 1, wherein the antibody is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain fragment variable (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or an antigen-binding fragments thereof.

16. The antibody of claim 1, wherein the antibody is a bispecific antibody.

17. The antibody of claim 1, wherein the antibody is a VHH antibody.

18. The antibody of claim 1, wherein the antibody is humanized.

19. The antibody of claim 1, wherein the antibody specifically binds to the SARS-CoV-2 RBD with an EC₅₀ of less than about 1 μg/ml.

20. The antibody of claim 1, wherein the antibody specifically binds to the SARS-CoV-2 RBD with an EC₅₀ of less than about 0.1 μg/ml.

21.-31. (canceled)


SEQUENCE LISTING (338 sequences)

SEQ ID NO: 1 - protein, 10 aa, synthetic construct
  REGION 1..10: note = source = /note="Description of Artificial Sequence: Synthetic peptide"
GRTFSEYAMG

SEQ ID NO: 2 - protein, 20 aa, synthetic construct
  VARIANT 2: note = /replace="Ser"
  VARIANT 3: note = /replace="Glu" or "Ser"
  VARIANT 5..7: note = SITE - /note="This region may encompass one of the following sequences: 'W' or 'L' or 'P' or 'GW' or 'GL' or 'GP' or 'GGW' or 'GGL' or 'GGP'"
  VARIANT 8: note = /replace="Gly" or "Ile"
  VARIANT 10: note = /replace="Trp"
  VARIANT 11: note = /replace="Ala" or "Gly"
  VARIANT 12: note = /replace="Ala" or "Gly" or "Ser"
  VARIANT 13: note = /replace="Gly"
  VARIANT 14: note = /replace="Glu" or "His" or "Gln" or "Ser" or "Thr"
  VARIANT 15: note = /replace="Tyr"
  VARIANT 16: note = /replace="Ala" or "Gln" or "Arg" or "Thr" or "Trp"
  VARIANT 17: note = /replace="Thr" or "Ala"
  VARIANT 19: note = /replace="Asp"
  VARIANT 20: note = /replace="Ala"
  REGION 1..20: note = SITE - /note="Variant residues given in the sequence have no preference with respect to those in the annotations for variant positions"
  REGION 1..20: note = source = /note="Description of Artificial Sequence: Synthetic peptide"
  REGION 1..20: note = source = /note="See specification as filed for detailed description of substitutions and preferred embodiments"
ATISXXXSGGSTYYTDSVKG

SEQ ID NO: 3 - protein, 18 aa, synthetic construct
  VARIANT 2: note = /replace="Pro" or "Tyr"
  VARIANT 4: note = /replace="Asp" or "Glu" or "Arg"
  VARIANT 5: note = /replace="Gln"
  VARIANT 6: note = /replace="Glu" or "Arg" or "Trp"
  VARIANT 11: note = /replace="Ala"
  VARIANT 12: note = /replace="Ala"
  VARIANT 13: note = /replace="Ala"
  REGION 1..18: note = SITE - /note="Variant residues given in the sequence have no preference with respect to those in the annotations for variant positions"
  REGION 1..18: note = source = /note="Description of Artificial Sequence: Synthetic peptide"
AAAGLGTVVSEWDYDYDY

SEQ ID NO: 4 - protein, 17 aa, synthetic construct
  REGION 1..17: note = source = /note="Description of Artificial Sequence: Synthetic peptide"
TISWSGGATYHTDTVKG

SEQ ID NO: 5 - protein, 17 aa, synthetic construct
  REGION 1..17: note = source = /note="Description of Artificial Sequence: Synthetic peptide"
TISWSGGATYHTDSVKG

SEQ ID NO: 6 - protein, 17 aa, synthetic construct
  REGION 1..17: note = source = /note="Description of Artificial Sequence: Synthetic peptide"
SISWSGGATYHTDSVKG

SEQ ID NO: 7 - protein, 17 aa, synthetic construct
  REGION 1..17: note = source = /note="Description of Artificial Sequence: Synthetic peptide"
TISWSGGATYHTWSVKG

SEQ ID NO: 8 - protein, 17 aa, synthetic construct
  REGION 1..17: note = source = /note="Description of Artificial Sequence: Synthetic peptide"
TISWSGGATGHTDSVKG

SEQ ID NO: 9 - protein, 17 aa, synthetic construct
  REGION 1..17: note = source = /note="Description of Artificial Sequence: Synthetic peptide"
TESWSGGATYHTDSVKG

SEQ ID NO: 10 - protein, 17 aa, synthetic construct
  REGION 1..17: note = source = /note="Description of Artificial Sequence: Synthetic peptide"
TISWSGGATYHTDSVDG

SEQ ID NO: 11 - protein, 17 aa, synthetic construct
  REGION 1..17: note = source = /note="Description of Artificial Sequence: Synthetic peptide"
TISWSGWATYHTDSVKG

SEQ ID NO: 12 - protein, 17 aa, synthetic construct
  REGION 1..17: note = source = /note="Description of Artificial Sequence: Synthetic peptide"
TISWIGGATYHTDSVKG

SEQ ID NO: 13 - protein, 17 aa, synthetic construct
  REGION 1..17: note = source = /note="Description of Artificial Sequence: Synthetic peptide"
TISWSGGGTYHTDSVKG

SEQ ID NO: 14 - protein, 17 aa, synthetic construct
  REGION 1..17: note = source = /note="Description of Artificial Sequence: Synthetic peptide"
TISWSGGASYHTDSVKG

SEQ ID NO: 15 - protein, 17 aa, synthetic construct
  REGION 1..17: note = source = /note="Description of Artificial Sequence: Synthetic peptide"
TISWSGGAAYHTDSVKG

SEQ ID NO: 16 - protein, 17 aa, synthetic construct
  REGION 1..17: note = source = /note="Description of Artificial Sequence: Synthetic peptide"
TISPSGGATYHTDSVKG

SEQ ID NO: 17 - protein, 17 aa, synthetic construct
  REGION 1..17: note = source = /note="Description of Artificial Sequence: Synthetic peptide"
TISWSGGATYHTTSVKG

SEQ ID NO: 18 - protein, 17 aa, synthetic construct
  REGION 1..17: note = source = /note="Description of Artificial Sequence: Synthetic peptide"
TISWSGGATYHTDSVKA

SEQ ID NO: 19 - protein, 17 aa, synthetic construct
  REGION 1..17: note = source = /note="Description of Artificial Sequence: Synthetic peptide"
TISWGGGATYHTDSVKG

SEQ ID NO: 20 - protein, 17 aa, synthetic construct
  REGION 1..17: note = source = /note="Description of Artificial Sequence: Synthetic peptide"
TSSWSGGATYHTDSVKG

SEQ ID NO: 21 - protein, 17 aa, synthetic construct
  REGION 1..17: note = source = /note="Description of Artificial Sequence: Synthetic peptide"
TISLSGGATYHTDSVKG

SEQ ID NO: 22 - protein, 17 aa, synthetic construct
  REGION 1..17: note = source = /note="Description of Artificial Sequence: Synthetic peptide"
TISWSGGATYHTDAVKG

SEQ ID NO: 23 - protein, 17 aa, synthetic construct
  REGION 1..17: note = source = /note="Description of Artificial Sequence: Synthetic peptide"
TISWSGGATYHTASVKG

SEQ ID NO: 24 - protein, 17 aa, synthetic construct
  REGION 1..17: note = source = /note="Description of Artificial Sequence: Synthetic peptide"
TISWSGGATYHYDSVKG

SEQ ID NO: 25 - protein, 17 aa, synthetic construct
  REGION 1..17: note = source = /note="Description of Artificial Sequence: Synthetic peptide"
TISWSGGAGYHTDSVKG

SEQ ID NO: 26 - protein, 19 aa, synthetic construct
  REGION 1..19: note = source = /note="Description of Artificial Sequence: Synthetic peptide"
TISGGWSGGATYHTDSVKG

SEQ ID NO: 27 - protein, 18 aa, synthetic construct
  REGION 1..18: note = source = /note="Description of Artificial Sequence: Synthetic peptide"
TISGWSGGATYHTDSVKG

SEQ ID NO: 28 - protein, 17 aa, synthetic construct
  REGION 1..17: note = source = /note="Description of Artificial Sequence: Synthetic peptide"
TISWSGGSTYYTRSVKG

SEQ ID NO: 29 - protein, 17 aa, synthetic construct
  REGION 1..17: note = source = /note="Description of Artificial Sequence: Synthetic peptide"
TISWSGGSTYTTRSVKG

SEQ ID NO: 30 - protein, 17 aa, synthetic construct
  REGION 1..17: note = source = /note="Description of Artificial Sequence: Synthetic peptide"
TISWSGGSTYTTDSVKG

SEQ ID NO: 31 - protein, 17 aa, synthetic construct
  REGION 1..17: note = source = /note="Description of Artificial Sequence: Synthetic peptide"
TISWSGGSTYHTRSVKG

SEQ ID NO: 32 - protein, 17 aa, synthetic construct
  REGION 1..17: note = source = /note="Description of Artificial Sequence: Synthetic peptide"
TISWSGGSTYHTDSVKG

SEQ ID NO: 33 - protein, 17 aa, synthetic construct
  REGION 1..17: note = source = /note="Description of Artificial Sequence: Synthetic peptide"
TISWSGGGTYYTWSVKG

SEQ ID NO: 34 - protein, 17 aa, synthetic construct
  REGION 1..17: note = source = /note="Description of Artificial Sequence: Synthetic peptide"
TISWSGGGTYYTRSVKG

SEQ ID NO: 35 - protein, 17 aa, synthetic construct
  REGION 1..17: note = source = /note="Description of Artificial Sequence: Synthetic peptide"
TISWSGGGTYTTRSVKG

SEQ ID NO: 36 - protein, 17 aa, synthetic construct
  REGION 1..17: note = source = /note="Description of Artificial Sequence: Synthetic peptide"
TISWSGGGTYHTRSVKG

SEQ ID NO: 37 - protein, 17 aa, synthetic construct
  REGION 1..17: note = source = /note="Description of Artificial Sequence: Synthetic peptide"
TISWSGGATYYTQSVKG

SEQ ID NO: 38 - protein, 17 aa, synthetic construct
  REGION 1..17: note = source = /note="Description of Artificial Sequence: Synthetic peptide"
TISWSGGATYYTDSVKG

SEQ ID NO: 39 - protein, 17 aa, synthetic construct
  REGION 1..17: note = source = /note="Description of Artificial Sequence: Synthetic peptide"
TISWSGGGTYTTDSVKG

SEQ ID NO: 40 - protein, 17 aa, synthetic construct
  REGION 1..17: note = source = /note="Description of Artificial Sequence: Synthetic peptide"
TISWSGGATYQTDSVKG

SEQ ID NO: 41 - protein, 17 aa, synthetic construct
  REGION 1..17: note = source = /note="Description of Artificial Sequence: Synthetic peptide"
TISWSGGATYSTDSVKG

SEQ ID NO: 42 - protein, 17 aa, synthetic construct
  REGION 1..17: note = source = /note="Description of Artificial Sequence: Synthetic peptide"
TISWSGGATYETDSVKG

SEQ ID NO: 43 - protein, 17 aa, synthetic construct
  REGION 1..17: note = source = /note="Description of Artificial Sequence: Synthetic peptide"
TISWSGGATYTTDSVKG

SEQ ID NO: 44 - protein, 17 aa, synthetic construct
  REGION 1..17: note = source = /note="Description of Artificial Sequence: Synthetic peptide"
TISWSGGATYYTRSVKG

SEQ ID NO: 45 - protein, 18 aa, synthetic construct
  REGION 1..18: note = source = /note="Description of Artificial Sequence: Synthetic peptide"
AAAGLGTVVSEWDYDYDY

SEQ ID NO: 46 - protein, 17 aa, synthetic construct
  REGION 1..17: note = source = /note="Description of Artificial Sequence: Synthetic peptide"
AAAGLGTVVSEWDDYDY

SEQ ID NO: 47 - protein, 18 aa, synthetic construct
  REGION 1..18: note = source = /note="Description of Artificial Sequence: Synthetic peptide"
APAGLGTVVSEWDYDYDY

SEQ ID NO: 48 - protein, 18 aa, synthetic construct
  REGION 1..18: note = source = /note="Description of Artificial Sequence: Synthetic peptide"
AAADLGTVVSEWDYDYDY

SEQ ID NO: 49 - protein, 18 aa, synthetic construct
  REGION 1..18: note = source = /note="Description of Artificial Sequence: Synthetic peptide"
AAAGLGTVVSEADYDYDY

SEQ ID NO: 50 - protein, 18 aa, synthetic construct
  REGION 1..18: note = source = /note="Description of Artificial Sequence: Synthetic peptide"
AAAGLRTVVSEWDYDYDY

SEQ ID NO: 51 - protein, 18 aa, synthetic construct
  REGION 1..18: note = source = /note="Description of Artificial Sequence: Synthetic peptide"
AYAGLGTVVSEWDYDYDY

SEQ ID NO: 52 - protein, 18 aa, synthetic construct
  REGION 1..18: note = source = /note="Description of Artificial Sequence: Synthetic peptide"
AAAGLGTVVSEWDYDYDS

SEQ ID NO: 53 - protein, 18 aa, synthetic construct
  REGION 1..18: note = source = /note="Description of Artificial Sequence: Synthetic peptide"
AAAGLWTVVSEWDYDYDY

SEQ ID NO: 54 - protein, 18 aa, synthetic construct
  REGION 1..18: note = source = /note="Description of Artificial Sequence: Synthetic peptide"
AAAGLGTVVSAWDYDYDY

SEQ ID NO: 55 - protein, 17 aa, synthetic construct
  REGION 1..17: note = source = /note="Description of Artificial Sequence: Synthetic peptide"
AAAGLGTVVSEWDYDDY

SEQ ID NO: 56 - protein, 18 aa, synthetic construct
  REGION 1..18: note = source = /note="Description of Artificial Sequence: Synthetic peptide"
AAAGQGTVVSEWDYDYDY

SEQ ID NO: 57 - protein, 18 aa, synthetic construct
  REGION 1..18: note = source = /note="Description of Artificial Sequence: Synthetic peptide"
AAAGLETVVSEWDYDYDY

SEQ ID NO: 58 - protein, 18 aa, synthetic construct
  REGION 1..18: note = source = /note="Description of Artificial Sequence: Synthetic peptide"
AAARLGTVVSEWDYDYDY

SEQ ID NO: 59 - protein, 18 aa, synthetic construct
  REGION 1..18: note = source = /note="Description of Artificial Sequence: Synthetic peptide"
AAAELGTVVSEWDYDYDY

SEQ ID NO: 60 - protein, 18 aa, synthetic construct
  REGION 1..18: note = source = /note="Description of Artificial Sequence: Synthetic peptide"
AAAGLGTVVSEWAYDYDA

SEQ ID NO: 61 - protein, 27 aa, synthetic construct
  REGION 1..27: note = source = /note="Description of Artificial Sequence: Synthetic peptide"
MQVQLQESGGGLVQAGGSLRLSCAASG

SEQ ID NO: 62 - protein, 29 aa, synthetic construct
  REGION 1..29: note = source = /note="Description of Artificial Sequence: Synthetic peptide"
MQVQLQESGGGLVQAGGSLRLSCAASGSG

SEQ ID NO: 63 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDTVKGRFTISRDNAKNTVYLQMNSLKPDDTAVYYCAAAGLGTVVSEWDYDYDYWGQGTQVTVSSS

SEQ ID NO: 64 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDFDYDYWGQGTQVTVSSS

SEQ ID NO: 65 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAPAGLGTVVSEWDYDYDYWGQGTQVTVSSS

SEQ ID NO: 66 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAADLGTVVSEWDYDYDYWGQGTQVTVSSS

SEQ ID NO: 67 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVASISWSGGATYHTDSVKGRFTISRDNAKNTVYLQMNSLKPDDTAVYYCAAAGLGTVVSEWDYDYDYWGQGTQVTVSSS

SEQ ID NO: 68 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTWSVKGRFTISRDNAKNTVYLQMNSLKPDDTAVYYCAAAGLGTVVSEWDYDYDYWGQGTQVTVSSS

SEQ ID NO: 69 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATGHTDSVKGRFTISRDNAKNTVYLQMNSLKPDDTAVYYCAAAGLGTVVSEWDYDYDYWGQGTQVTVSSS

SEQ ID NO: 70 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATESWSGGATYHTDSVKGRFTISRDNAKNTVYLQMNSLKPDDTAVYYCAAAGLGTVVSEWDYDYDYWGQGTQVTVSSS

SEQ ID NO: 71 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVDGRFTISRDNAKNTVYLQMNSLKPDDTAVYYCAAAGLGTVVSEWDYDYDYWGQGTQVTVSSS

SEQ ID NO: 72 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGWATYHTDSVKGRFTISRDNAKNTVYLQMNSLKPDDTAVYYCAAAGLGTVVSEWDYDYDYWGQGTQVTVSSS

SEQ ID NO: 73 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEADYDYDYWGQGTQVTVSSS

SEQ ID NO: 74 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWIGGATYHTDSVKGRFTISRDNAKNTVYLQMNSLKPDDTAVYYCAAAGLGTVVSEWDYDYDYWGQGTQVTVSSS

SEQ ID NO: 75 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYHTDSVKGRFTISRDNAKNTVYLQMNSLKPDDTAVYYCAAAGLGTVVSEWDYDYDYWGQGTQVTVSSS

SEQ ID NO: 76 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLRTVVSEWDYDYDYWGQGTQVTVSSS

SEQ ID NO: 77 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGASYHTDSVKGRFTISRDNAKNTVYLQMNSLKPDDTAVYYCAAAGLGTVVSEWDYDYDYWGQGTQVTVSSS

SEQ ID NO: 78 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGAAYHTDSVKGRFTISRDNAKNTVYLQMNSLKPDDTAVYYCAAAGLGTVVSEWDYDYDYWGQGTQVTVSSS

SEQ ID NO: 79 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISPSGGATYHTDSVKGRFTISRDNAKNTVYLQMNSLKPDDTAVYYCAAAGLGTVVSEWDYDYDYWGQGTQVTVSSS

SEQ ID NO: 80 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTTSVKGRFTISRDNAKNTVYLQMNSLKPDDTAVYYCAAAGLGTVVSEWDYDYDYWGQGTQVTVSSS

SEQ ID NO: 81 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKARFTISRDNAKNTVYLQMNSLKPDDTAVYYCAAAGLGTVVSEWDYDYDYWGQGTQVTVSSS

SEQ ID NO: 82 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAYAGLGTVVSEWDYDYDYWGQGTQVTVSSS

SEQ ID NO: 83 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWGGGATYHTDSVKGRFTISRDNAKNTVYLQMNSLKPDDTAVYYCAAAGLGTVVSEWDYDYDYWGQGTQVTVSSS

SEQ ID NO: 84 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDYDYDSWGQGTQVTVSSS

SEQ ID NO: 85 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLWTVVSEWDYDYDYWGQGTQVTVSSS

SEQ ID NO: 86 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSAWDYDYDYWGQGTQVTVSSS

SEQ ID NO: 87 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDYDFDYWGQGTQVTVSSS

SEQ ID NO: 88 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATSSWSGGATYHTDSVKGRFTISRDNAKNTVYLQMNSLKPDDTAVYYCAAAGLGTVVSEWDYDYDYWGQGTQVTVSSS

SEQ ID NO: 89 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISLSGGATYHTDSVKGRFTISRDNAKNTVYLQMNSLKPDDTAVYYCAAAGLGTVVSEWDYDYDYWGQGTQVTVSSS

SEQ ID NO: 90 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDAVKGRFTISRDNAKNTVYLQMNSLKPDDTAVYYCAAAGLGTVVSEWDYDYDYWGQGTQVTVSSS

SEQ ID NO: 91 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGQGTVVSEWDYDYDYWGQGTQVTVSSS

SEQ ID NO: 92 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTASVKGRFTISRDNAKNTVYLQMNSLKPDDTAVYYCAAAGLGTVVSEWDYDYDYWGQGTQVTVSSS

SEQ ID NO: 93 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHYDSVKGRFTISRDNAKNTVYLQMNSLKPDDTAVYYCAAAGLGTVVSEWDYDYDYWGQGTQVTVSSS

SEQ ID NO: 94 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGAGYHTDSVKGRFTISRDNAKNTVYLQMNSLKPDDTAVYYCAAAGLGTVVSEWDYDYDYWGQGTQVTVSSS

SEQ ID NO: 95 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDYDYDYWGQGTQVTVSSS

SEQ ID NO: 96 - protein, 129 aa, synthetic construct
  REGION 1..129: note = source = /note="Description of Artificial Sequence: Synthetic polypeptide"
MQVQLQESGGGLVQAGGSLRLSCAASGRTFSEYAMGWFRQAPGKEREFVATISGGWSGGATYHTDSVKGRFTISRDNAKNTVYLQMNSLKPDDTAVYYCAAAGLGTVVSEWDYDYDYWGQGTQVTVSSS

SEQ ID NO: 97 - protein, 128 aa, synthetic construct
  REGION 1..128: note = source = /note="Description of Artificial Sequence: Synthetic polypeptide"
MQVQLQESGGGLVQAGGSLRLSCAASGRTFSEYAMGWFRQAPGKEREFVATISGWSGGATYHTDSVKGRFTISRDNAKNTVYLQMNSLKPDDTAVYYCAAAGLGTVVSEWDYDYDYWGQGTQVTVSSS

SEQ ID NO: 98 - protein, 130 aa, synthetic construct
  REGION 1..130: note = source = /note="Description of Artificial Sequence: Synthetic polypeptide"
MQVQLQESGGGLVQAGGSLRLSCAASGSGGRTFSEYAMGWFRQAPGKEREFVATISWSGGATYHTDSVKGRFTISRDNAKNTVYLQMNSLKPDDTAVYYCAAAGLGTVVSEWDYDYDYWGQGTQVTVSSS

SEQ ID NO: 99 - protein, 128 aa, synthetic construct
  REGION 1..128: note = source = /note="Description of Artificial Sequence: Synthetic polypeptide"
MQVQLQESGGGLVQAGGSLRLSCAASGGRTFSEYAMGWFRQAPGKEREFVATISWSGGATYHTDSVKGRFTISRDNAKNTVYLQMNSLKPDDTAVYYCAAAGLGTVVSEWDYDYDYWGQGTQVTVSSS

SEQ ID NO: 100 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGSTYYTRSVKGRFTISRDNAKNTVYLQMNSLKPDDTAVYYCAAAGLETVVSEWDYDYDYWGQGTQVTVSSS

SEQ ID NO: 101 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGSTYTTRSVKGRFTISRDNAKNTVYLQMNSLKPDDTAVYYCAAAGLETVVSEWDYDYDYWGQGTQVTVSSS

SEQ ID NO: 102 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGSTYTTRSVKGRFTISRDNAKNTVYLQMNSLKPDDTAVYYCAAAGLGTVVSEWDYDYDYWGQGTQVTVSSS

SEQ ID NO: 103 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGSTYTTDSVKGRFTISRDNAKNTVYLQMNSLKPDDTAVYYCAAAGLETVVSEWDYDYDYWGQGTQVTVSSS

SEQ ID NO: 104 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGSTYHTRSVKGRFTISRDNAKNTVYLQMNSLKPDDTAVYYCAAAGLGTVVSEWDYDYDYWGQGTQVTVSSS

SEQ ID NO: 105 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGSTYHTRSVKGRFTISRDNAKNTVYLQMNSLKPDDTAVYYCAAAGLETVVSEWDYDYDYWGQGTQVTVSSS

SEQ ID NO: 106 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGSTYHTDSVKGRFTISRDNAKNTVYLQMNSLKPDDTAVYYCAAAGLETVVSEWDYDYDYWGQGTQVTVSSS

SEQ ID NO: 107 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYYTWSVKGRFTISRDNAKNTVYLQMNSLKPDDTAVYYCAAAGLETVVSEWDYDYDYWGQGTQVTVSSS

SEQ ID NO: 108 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYYTRSVKGRFTISRDNAKNTVYLQMNSLKPDDTAVYYCAAAGLETVVSEWDYDYDYWGQGTQVTVSSS

SEQ ID NO: 109 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYTTRSVKGRFTISRDNAKNTVYLQMNSLKPDDTAVYYCAAAGLETVVSEWDYDYDYWGQGTQVTVSSS

SEQ ID NO: 110 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYHTRSVKGRFTISRDNAKNTVYLQMNSLKPDDTAVYYCAAAGLGTVVSEWDYDYDYWGQGTQVTVSSS

SEQ ID NO: 111 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYYTQSVKGRFTISRDNAKNTVYLQMNSLKPDDTAVYYCAAAGLGTVVSEWDYDYDYWGQGTQVTVSSS

SEQ ID NO: 112 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYTTRSVKGRFTISRDNAKNTVYLQMNSLKPDDTAVYYCAAAGLGTVVSEWDYDYDYWGQGTQVTVSSS

SEQ ID NO: 113 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLGTVVSEWDYDYDYWGQGTQVTVSSS

SEQ ID NO: 114 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYTTDSVKGRFTISRDNAKNTVYLQMNSLKPDDTAVYYCAAAGLGTVVSEWDYDYDYWGQGTQVTVSSS

SEQ ID NO: 115 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYTTDSVKGRFTISRDNAKNTVYLQMNSLKPDDTAVYYCAAAGLETVVSEWDYDYDYWGQGTQVTVSSS

SEQ ID NO: 116 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYHTRSVKGRFTISRDNAKNTVYLQMNSLKPDDTAVYYCAAAGLETVVSEWDYDYDYWGQGTQVTVSSS

SEQ ID NO: 117 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGGTYHTDSVKGRFTISRDNAKNTVYLQMNSLKPDDTAVYYCAAAGLETVVSEWDYDYDYWGQGTQVTVSSS

SEQ ID NO: 118 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYQTDSVKGRFTISRDNAKNTVYLQMNSLKPDDTAVYYCAAAGLGTVVSEWDYDYDYWGQGTQVTVSSS

SEQ ID NO: 119 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYSTDSVKGRFTISRDNAKNTVYLQMNSLKPDDTAVYYCAAAGLGTVVSEWDYDYDYWGQGTQVTVSSS

SEQ ID NO: 120 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYETDSVKGRFTISRDNAKNTVYLQMNSLKPDDTAVYYCAAAGLGTVVSEWDYDYDYWGQGTQVTVSSS

SEQ ID NO: 121 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSS

SEQ ID NO: 122 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYYTRSVKGRFTISRDNAKNTVYLQMNSLKPDDTAVYYCAAAGLGTVVSEWDYDYDYWGQGTQVTVSSS

SEQ ID NO: 123 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAARLGTVVSEWDYDYDYWGQGTQVTVSSS

SEQ ID NO: 124 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAELGTVVSEWDYDYDYWGQGTQVTVSSS

SEQ ID NO: 125 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLETVVSEWDYDYDYWGQGTQVTVSSS

SEQ ID NO: 126 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLGTVVSEWAYDYDAWGQGTQVTVSSS

SEQ ID NO: 127 - protein, 8 aa, synthetic construct
  REGION 1..8: note = source = /note="Description of Artificial Sequence: Synthetic peptide"
GFPVYSWN

SEQ ID NO: 128 - protein, 8 aa, synthetic construct
  VARIANT 5: note = /replace="Ala"
  REGION 1..8: note = SITE - /note="Variant residues given in the sequence have no preference with respect to those in the annotations for variant positions"
  REGION 1..8: note = source = /note="Description of Artificial Sequence: Synthetic peptide"
IESHGDST

SEQ ID NO: 129 - protein, 11 aa, synthetic construct
  VARIANT 5: note = /replace="Ala"
  REGION 1..11: note = SITE - /note="Variant residues given in the sequence have no preference with respect to those in the annotations for variant positions"
  REGION 1..11: note = source = /note="Description of Artificial Sequence: Synthetic peptide"
YVWVGHTYYGQ

SEQ ID NO: 130 - protein, 8 aa, synthetic construct
  REGION 1..8: note = source = /note="Description of Artificial Sequence: Synthetic peptide"
IESHGDST

SEQ ID NO: 131 - protein, 8 aa, synthetic construct
  REGION 1..8: note = source = /note="Description of Artificial Sequence: Synthetic peptide"
IESYAHGT

SEQ ID NO: 132 - protein, 11 aa, synthetic construct
  REGION 1..11: note = source = /note="Description of Artificial Sequence: Synthetic peptide"
YVWVGHTYYGQ

SEQ ID NO: 133 - protein, 11 aa, synthetic construct
  REGION 1..11: note = source = /note="Description of Artificial Sequence: Synthetic peptide"
YVWVAHTYYGQ

SEQ ID NO: 134 - protein, 116 aa, synthetic construct
  REGION 1..116: note = source = /note="Description of Artificial Sequence: Synthetic polypeptide"
MQVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVGHTYYGQGTQVTVSS

SEQ ID NO: 135 - protein, 116 aa, synthetic construct
  REGION 1..116: note = source = /note="Description of Artificial Sequence: Synthetic polypeptide"
MQVQLVESGGGLVQAGGSLRLSCAASGFPVYSWNMWWYRQAPGKEREWVAAIESHADSTRYADSVKGRFTISRDNAKNTVYLQMNSLKPEDTAVYYCYVWVGHTYYGQGTQVTVSS

SEQ ID NO: 136 - protein, 116 aa, synthetic construct
  REGION 1..116: note = source = /note="Description of Artificial Sequence: Synthetic polypeptide"
MQVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVAHTYYGQGTQVTVSS

SEQ ID NO: 137 - protein, 8 aa, synthetic construct
  VARIANT 2: note = /replace="Ala"
  VARIANT 4: note = /replace="His" or "Tyr"
  REGION 1..8: note = SITE - /note="Variant residues given in the sequence have no preference with respect to those in the annotations for variant positions"
  REGION 1..8: note = source = /note="Description of Artificial Sequence: Synthetic peptide"
GRTFSTAA

SEQ ID NO: 138 - protein, 8 aa, synthetic construct
  VARIANT 5: note = /replace="Ala"
  REGION 1..8: note = SITE - /note="Variant residues given in the sequence have no preference with respect to those in the annotations for variant positions"
  REGION 1..8: note = source = /note="Description of Artificial Sequence: Synthetic peptide"
IRWSGGSA

SEQ ID NO: 139 - protein, 20 aa, synthetic construct
  VARIANT 8: note = /replace="Ala"
  VARIANT 9: note = /replace="Lys"
  VARIANT 16: note = /replace="Tyr"
  VARIANT 19: note = /replace="Gln" or "Arg"
  REGION 1..20: note = SITE - /note="Variant residues given in the sequence have no preference with respect to those in the annotations for variant positions"
  REGION 1..20: note = source = /note="Description of Artificial Sequence: Synthetic peptide"
ARTENVRSLLSDYATWPYAY

SEQ ID NO: 140 - protein, 8 aa, synthetic construct
  REGION 1..8: note = source = /note="Description of Artificial Sequence: Synthetic peptide"
GRTFSTAA

SEQ ID NO: 141 - protein, 8 aa, synthetic construct
  REGION 1..8: note = source = /note="Description of Artificial Sequence: Synthetic peptide"
GRTFATAA

SEQ ID NO: 142 - protein, 8 aa, synthetic construct
  REGION 1..8: note = source = /note="Description of Artificial Sequence: Synthetic peptide"
GATFSTAA

SEQ ID NO: 143 - protein, 8 aa, synthetic construct
  REGION 1..8: note = source = /note="Description of Artificial Sequence: Synthetic peptide"
GRTYSTAA

SEQ ID NO: 144 - protein, 8 aa, synthetic construct
  REGION 1..8: note = source = /note="Description of Artificial Sequence: Synthetic peptide"
GRTHSTAA

SEQ ID NO: 145 - protein, 8 aa, synthetic construct
  REGION 1..8: note = source = /note="Description of Artificial Sequence: Synthetic peptide"
IRWSGGSA

SEQ ID NO: 146 - protein, 8 aa, synthetic construct
  REGION 1..8: note = source = /note="Description of Artificial Sequence: Synthetic peptide"
IRWSAGSA

SEQ ID NO: 147 - protein, 20 aa, synthetic construct
  REGION 1..20: note = source = /note="Description of Artificial Sequence: Synthetic peptide"
ARTENVRSLLSDYATWPYDY

SEQ ID NO: 148 - protein, 20 aa, synthetic construct
  REGION 1..20: note = source = /note="Description of Artificial Sequence: Synthetic peptide"
ARTENVRSLLSDYATYPYDY

SEQ ID NO: 149 - protein, 20 aa, synthetic construct
  REGION 1..20: note = source = /note="Description of Artificial Sequence: Synthetic peptide"
ARTENVRALLSDYATWPYDY

SEQ ID NO: 150 - protein, 20 aa, synthetic construct
  REGION 1..20: note = source = /note="Description of Artificial Sequence: Synthetic peptide"
ARTENVRSKLSDYATWPYDY

SEQ ID NO: 151 - protein, 20 aa, synthetic construct
  REGION 1..20: note = source = /note="Description of Artificial Sequence: Synthetic peptide"
ARTENVRSLLSDYATWPYRY

SEQ ID NO: 152 - protein, 20 aa, synthetic construct
  REGION 1..20: note = source = /note="Description of Artificial Sequence: Synthetic peptide"
ARTENVRSLLSDYATWPYQY

SEQ ID NO: 153 - protein, 20 aa, synthetic construct
  REGION 1..20: note = source = /note="Description of Artificial Sequence: Synthetic peptide"
ARTENVRSLLSDYATWPYAY

SEQ ID NO: 154 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSKS

SEQ ID NO: 155 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATYPYDYWGQGTQVTVSSKS

SEQ ID NO: 156 - protein, 130 aa, synthetic construct
  REGION 1..130: note = source = /note="Description of Artificial Sequence: Synthetic polypeptide"
MQVQLVESGGGLMQAGGSLRLSCAVSGRTFATAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSKS

SEQ ID NO: 157 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRALLSDYATWPYDYWGQGTQVTVSSKS

SEQ ID NO: 158 - protein, 130 aa, synthetic construct
  REGION 1..130: note = source = /note="Description of Artificial Sequence: Synthetic polypeptide"
MQVQLVESGGGLMQAGGSLRLSCAVSGATFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSKS

SEQ ID NO: 159 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSKLSDYATWPYDYWGQGTQVTVSSKS

SEQ ID NO: 160 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSAGSAYYADSVKGRFTISRDKAKNTVYLQMNSLKYEDTAVYYCARTENVRSLLSDYATWPYDYWGQGTQVTVSSKS

SEQ ID NO: 161 - protein, 130 aa, synthetic construct
  REGION 1..130: note = source = /note="Description of Artificial Sequence: Synthetic polypeptide"
MQVQLVESGGGLMQAGGSLRLSCAVSGRTYSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSKS

SEQ ID NO: 162 - protein, 130 aa, synthetic construct
  REGION 1..130: note = source = /note="Description of Artificial Sequence: Synthetic polypeptide"
MQVQLVESGGGLMQAGGSLRLSCAVSGRTHSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSKS

SEQ ID NO: 163 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYRYWGQGTQVTVSSKS

SEQ ID NO: 164 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYQYWGQGTQVTVSSKS

SEQ ID NO: 165 - protein, 130 aa, synthetic construct
  REGION 1..130: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYAYWGQGTQVTVSSKS

SEQ ID NO: 166 - protein, 8 aa, synthetic construct
  VARIANT 6: note = /replace="Ala"
  VARIANT 7: note = /replace="Tyr" or "Ala"
  VARIANT 8: note = /replace="Ala"
  REGION 1..8: note = SITE - /note="Variant residues given in the sequence have no preference with respect to those in the annotations for variant positions"
  REGION 1..8: note = source = /note="Description of Artificial Sequence: Synthetic peptide"
GFPVEVWR

SEQ ID NO: 167 - protein, 8 aa, synthetic construct
  VARIANT 3: note = /replace="Ala"
  VARIANT 4: note = /replace="Ala"
  VARIANT 8: note = /replace="Ala"
  REGION 1..8: note = SITE - /note="Variant residues given in the sequence have no preference with respect to those in the annotations for variant positions"
  REGION 1..8: note = source = /note="Description of Artificial Sequence: Synthetic peptide"
IEGYGHGT

SEQ ID NO: 168 - protein, 14 aa, synthetic construct
  VARIANT 3: note = /replace="Ala"
  VARIANT 6: note = /replace="Asn"
  VARIANT 7: note = /replace="His"
  REGION 1..14: note = SITE - /note="Variant residues given in the sequence have no preference with respect to those in the annotations for variant positions"
  REGION 1..14: note = source = /note="Description of Artificial Sequence: Synthetic peptide"
NVGDDGQLAYHYDY

SEQ ID NO: 169 - protein, 8 aa, synthetic construct
  REGION 1..8: note = source = /note="Description of Artificial Sequence: Synthetic peptide"
GFPVEVWR

SEQ ID NO: 170 - protein, 8 aa, synthetic construct
  REGION 1..8: note = source = /note="Description of Artificial Sequence: Synthetic peptide"
GFPVEVYR

SEQ ID NO: 171 - protein, 8 aa, synthetic construct
  REGION 1..8: note = source = /note="Description of Artificial Sequence: Synthetic peptide"
GFPVEVAR

SEQ ID NO: 172 - protein, 8 aa, synthetic construct
  REGION 1..8: note = source = /note="Description of Artificial Sequence: Synthetic peptide"
GFPVEAWR

SEQ ID NO: 173 - protein, 8 aa, synthetic construct
  REGION 1..8: note = source = /note="Description of Artificial Sequence: Synthetic peptide"
GFPVEVWA

SEQ ID NO: 174 - protein, 8 aa, synthetic construct
  REGION 1..8: note = source = /note="Description of Artificial Sequence: Synthetic peptide"
IESYGHGT

SEQ ID NO: 175 - protein, 8 aa, synthetic construct
  REGION 1..8: note = source = /note="Description of Artificial Sequence: Synthetic peptide"
IESAGHGT

SEQ ID NO: 176 - protein, 8 aa, synthetic construct
  REGION 1..8: note = source = /note="Description of Artificial Sequence: Synthetic peptide"
IESYGHGA

SEQ ID NO: 177 - protein, 8 aa, synthetic construct
  REGION 1..8: note = source = /note="Description of Artificial Sequence: Synthetic peptide"
IEAYGHGT

SEQ ID NO: 178 - protein, 14 aa, synthetic construct
  REGION 1..14: note = source = /note="Description of Artificial Sequence: Synthetic peptide"
NVKDDGQLAYHYDY

SEQ ID NO: 179 - protein, 14 aa, synthetic construct
  REGION 1..14: note = source = /note="Description of Artificial Sequence: Synthetic peptide"
NVYDDGQLAYHYDY

SEQ ID NO: 180 - protein, 14 aa, synthetic construct
  REGION 1..14: note = source = /note="Description of Artificial Sequence: Synthetic peptide"
NVYDDGHLAYHYDY

SEQ ID NO: 181 - protein, 14 aa, synthetic construct
  REGION 1..14: note = source = /note="Description of Artificial Sequence: Synthetic peptide"
NVYDDNQLAYHYDY

SEQ ID NO: 182 - protein, 38 aa, synthetic construct
  REGION 1..38: note = source = /note="Description of Artificial Sequence: Synthetic polypeptide"
RYADSVKGRFTISRDNAKNTVYLQMNSLKPEDTAVYYC

SEQ ID NO: 183 - protein, 38 aa, synthetic construct
  REGION 1..38: note = source = /note="Description of Artificial Sequence: Synthetic polypeptide"
RYADSVRGRFTISRDNAKNTVYLQMNSLKPEDTAVYYC

SEQ ID NO: 184 - protein, 38 aa, synthetic construct
  REGION 1..38: note = source = /note="Description of Artificial Sequence: Synthetic polypeptide"
RYADSVAGRFTISRDNAKNTVYLQMNSLKPEDTAVYYC

SEQ ID NO: 185 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVSS

SEQ ID NO: 186 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGQLAYHYDYWGQGTQVTVSS

SEQ ID NO: 187 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESAGHGTRYADSVKGRFTISRDNAKNTVYLQMNSLKPEDTAVYYCNVYDDGQLAYHYDYWGQGTQVTVSS

SEQ ID NO: 188 - protein, 122 aa, synthetic construct
  REGION 1..122: note = source = /note="Description of Artificial Sequence: Synthetic polypeptide"
MQVQLVESGGGLVQAGGSLRLSCAASGFPVEVYRMEWYRQAPGKEREGVAAIESYGHGTRYADSVKGRFTISRDNAKNTVYLQMNSLKPEDTAVYYCNVYDDGQLAYHYDYWGQGTQVTVSS

SEQ ID NO: 189 - protein, 122 aa, synthetic construct
  REGION 1..122: note = source = /note="Description of Artificial Sequence: Synthetic polypeptide"
MQVQLVESGGGLVQAGGSLRLSCAASGFPVEVARMEWYRQAPGKEREGVAAIESYGHGTRYADSVKGRFTISRDNAKNTVYLQMNSLKPEDTAVYYCNVYDDGQLAYHYDYWGQGTQVTVSS

SEQ ID NO: 190 - protein, 122 aa, synthetic construct
  REGION 1..122: note = source = /note="Description of Artificial Sequence: Synthetic polypeptide"
MQVQLVESGGGLVQAGGSLRLSCAASGFPVEAWRMEWYRQAPGKEREGVAAIESYGHGTRYADSVKGRFTISRDNAKNTVYLQMNSLKPEDTAVYYCNVYDDGQLAYHYDYWGQGTQVTVSS

SEQ ID NO: 191 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESYGHGARYADSVKGRFTISRDNAKNTVYLQMNSLKPEDTAVYYCNVYDDGQLAYHYDYWGQGTQVTVSS

SEQ ID NO: 192 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIEAYGHGTRYADSVKGRFTISRDNAKNTVYLQMNSLKPEDTAVYYCNVYDDGQLAYHYDYWGQGTQVTVSS

SEQ ID NO: 193 - protein, 122 aa, synthetic construct
  REGION 1..122: note = source = /note="Description of Artificial Sequence: Synthetic polypeptide"
MQVQLVESGGGLVQAGGSLRLSCAASGFPVEVWAMEWYRQAPGKEREGVAAIESYGHGTRYADSVKGRFTISRDNAKNTVYLQMNSLKPEDTAVYYCNVYDDGQLAYHYDYWGQGTQVTVSS

SEQ ID NO: 194 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGHLAYHYDYWGQGTQVTVSS

SEQ ID NO: 195 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVRGRFTISRDNAKNTVYLQMNSLKPEDTAVYYCNVYDDGQLAYHYDYWGQGTQVTVSS

SEQ ID NO: 196 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVAGRFTISRDNAKNTVYLQMNSLKPEDTAVYYCNVYDDGQLAYHYDYWGQGTQVTVSS

SEQ ID NO: 197 - protein, 122 aa, synthetic construct
  REGION 1..122: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDNQLAYHYDYWGQGTQVTVSS

SEQ ID NO: 198 - protein, 258 aa, synthetic construct
  REGION 1..258: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVSS

SEQ ID NO: 199 - protein, 248 aa, synthetic construct
  REGION 1..248: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVSS

SEQ ID NO: 200 - protein, 248 aa, synthetic construct
  REGION 1..248: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGQLAYHYDYWGQGTQVTVSS

SEQ ID NO: 201 - protein, 256 aa, synthetic construct
  REGION 1..256: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSKS

SEQ ID NO: 202 - protein, 256 aa, synthetic construct; identical to SEQ ID NO: 201
  REGION 1..256: note = source = /note="Description of Artificial Sequence: Synthetic polypeptide"

SEQ ID NO: 203 - protein, 242 aa, synthetic construct
  REGION 1..242: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSQVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVGHTYYGQGTQVTVSS

SEQ ID NO: 204 - protein, 246 aa, synthetic construct
  REGION 1..246: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSS

SEQ ID NO: 205 - protein, 248 aa, synthetic construct
  REGION 1..248: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSQVQLVESGGGLVQAGGSLRLSCAASGFPVYRDRMAWYRQAPGKEREWVAAIYSAGQQTRYADSVKGRFTISRDNAKNTVYLQMNSLKPEDTAVYYCNVKDVGHHYEYYDYWGQGTQVTVSS

SEQ ID NO: 206 - protein, 254 aa, synthetic construct
  REGION 1..254: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSSGGGGSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVSS

SEQ ID NO: 207 - protein, 5 aa, synthetic construct
  REGION 1..5: note = source = /note="Description of Artificial Sequence: Synthetic peptide"
GGGGS

SEQ ID NO: 208 - protein, 10 aa, synthetic construct
  REGION 1..10: note = source = /note="Description of Artificial Sequence: Synthetic peptide"
GGGGSGGGGS

SEQ ID NO: 209 - protein, 15 aa, synthetic construct
  REGION 1..15: note = source = /note="Description of Artificial Sequence: Synthetic peptide"
GGGGSGGGGSGGGGS

SEQ ID NO: 210 - protein, 20 aa, synthetic construct
  REGION 1..20: note = source = /note="Description of Artificial Sequence: Synthetic peptide"
GGGGSGGGGSGGGGSGGGGS

SEQ ID NO: 211 - protein, 4 aa, synthetic construct
  REGION 1..4: note = source = /note="Description of Artificial Sequence: Synthetic peptide"
GGGS

SEQ ID NO: 212 - protein, 4 aa, synthetic construct
  REGION 1..4: note = source = /note="Description of Artificial Sequence: Synthetic peptide"
GGSG

SEQ ID NO: 213 - protein, 127 aa, synthetic construct
  REGION 1..127: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSS

SEQ ID NO: 214 - protein, 119 aa, synthetic construct
  REGION 1..119: note = source = /note="Description of Artificial Sequence: Synthetic polypeptide"
QVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSS

SEQ ID NO: 215 - protein, 121 aa, synthetic construct
  REGION 1..121: note = source = /note="Description of Artificial Sequence: Synthetic polypeptide"
QVQLVESGGGLVQAGGSLRLSCAASGFPVYRDRMAWYRQAPGKEREWVAAIYSAGQQTRYADSVKGRFTISRDNAKNTVYLQMNSLKPEDTAVYYCNVKDVGHHYEYYDYWGQGTQVTVSS

SEQ ID NO: 216 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISASGGSTYYTDSVKGRFTISRDNAKNTVYLQMNSLKPDDTAVYYCAAAGLGTVVSEWDYDYDYWGQGTQVTVSS

SEQ ID NO: 217 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTAYTDSVKGRFTISRDNAKNTVYLQMNSLKPDDTAVYYCAAAGLGTVVSEWDYDYDYWGQGTQVTVSS

SEQ ID NO: 218 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLATVVSEWDYDYDYWGQGTQVTVSS

SEQ ID NO: 219 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSAWDYDYDYWGQGTQVTVSS

SEQ ID NO: 220 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAGAGLGTVVSEWDYDYDYWGQGTQVTVSS

SEQ ID NO: 221 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWAYDYDYWGQGTQVTVSS

SEQ ID NO: 222 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAGGLGTVVSEWDYDYDYWGQGTQVTVSS

SEQ ID NO: 223 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTASVKGRFTISRDNAKNTVYLQMNSLKPDDTAVYYCAAAGLGTVVSEWDYDYDYWGQGTQVTVSS

SEQ ID NO: 224 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEADYDYDYWGQGTQVTVSS

SEQ ID NO: 225 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDADYWGQGTQVTVSS

SEQ ID NO: 226 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCGAAGLGTVVSEWDYDYDYWGQGTQVTVSS

SEQ ID NO: 227 - protein, 8 aa, synthetic construct
  REGION 1..8: note = source = /note="Description of Artificial Sequence: Synthetic peptide"
GRTFSEYA

SEQ ID NO: 228 - protein, 17 aa, synthetic construct
  REGION 1..17: note = source = /note="Description of Artificial Sequence: Synthetic peptide"
TISWSGGSTYYTASVKG

SEQ ID NO: 229 - protein, 17 aa, synthetic construct
  REGION 1..17: note = source = /note="Description of Artificial Sequence: Synthetic peptide"
TISWSGGATYYTASVKG

SEQ ID NO: 230 - protein, 17 aa, synthetic construct
  REGION 1..17: note = source = /note="Description of Artificial Sequence: Synthetic peptide"
TISWSGGGTYYTDSVKG

SEQ ID NO: 231 - protein, 8 aa, synthetic construct
  REGION 1..8: note = source = /note="Description of Artificial Sequence: Synthetic peptide"
IESHADST

SEQ ID NO: 232 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLGTVVSEWDYDYDYWGQGTQVTVSS

SEQ ID NO: 233 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYSTDSVKGRFTISRDNAKNTVYLQMNSLKPDDTAVYYCAAAGLGTVVSEWDYDYDYWGQGTQVTVSS

SEQ ID NO: 234 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYQTDSVKGRFTISRDNAKNTVYLQMNSLKPDDTAVYYCAAAGLGTVVSEWDYDYDYWGQGTQVTVSS

SEQ ID NO: 235 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYYTRSVKGRFTISRDNAKNTVYLQMNSLKPDDTAVYYCAAAGLGTVVSEWDYDYDYWGQGTQVTVSS

SEQ ID NO: 236 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDYDYDYWGQGTQVTVSS

SEQ ID NO: 237 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSS

SEQ ID NO: 238 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAARLGTVVSEWDYDYDYWGQGTQVTVSS

SEQ ID NO: 239 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYETDSVKGRFTISRDNAKNTVYLQMNSLKPDDTAVYYCAAAGLGTVVSEWDYDYDYWGQGTQVTVSS

SEQ ID NO: 240 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAELGTVVSEWDYDYDYWGQGTQVTVSS

SEQ ID NO: 241 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLETVVSEWDYDYDYWGQGTQVTVSS

SEQ ID NO: 242 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYYTDSVKGRFTISRDNAKNTVYLQMNSLKPDDTAVYYCAAAGLGTVVSEWAYDYDAWGQGTQVTVSS

SEQ ID NO: 243 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 244 - protein, 119 aa, synthetic construct
  REGION 1..119: note = source = /note="Description of Artificial Sequence: Synthetic polypeptide"
MQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 245 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAPAGLGTVVSEWDYDYDYWGQGTQVTVSS

SEQ ID NO: 246 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDFDYDYWGQGTQVTVSS

SEQ ID NO: 247 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAADLGTVVSEWDYDYDYWGQGTQVTVSS

SEQ ID NO: 248 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDTVKGRFTISRDNAKNTVYLQMNSLKPDDTAVYYCAAAGLGTVVSEWDYDYDYWGQGTQVTVSS

SEQ ID NO: 249 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVASISWSGGATYHTDSVKGRFTISRDNAKNTVYLQMNSLKPDDTAVYYCAAAGLGTVVSEWDYDYDYWGQGTQVTVSS

SEQ ID NO: 250 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTWSVKGRFTISRDNAKNTVYLQMNSLKPDDTAVYYCAAAGLGTVVSEWDYDYDYWGQGTQVTVSS

SEQ ID NO: 251 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATGHTDSVKGRFTISRDNAKNTVYLQMNSLKPDDTAVYYCAAAGLGTVVSEWDYDYDYWGQGTQVTVSS

SEQ ID NO: 252 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYHTDSVKGRFTISRDNAKNTVYLQMNSLKPDDTAVYYCAAAGLGTVVSEWDYDYDYWGQGTQVTVSS

SEQ ID NO: 253 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWIGGATYHTDSVKGRFTISRDNAKNTVYLQMNSLKPDDTAVYYCAAAGLGTVVSEWDYDYDYWGQGTQVTVSS

SEQ ID NO: 254 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVDGRFTISRDNAKNTVYLQMNSLKPDDTAVYYCAAAGLGTVVSEWDYDYDYWGQGTQVTVSS

SEQ ID NO: 255 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATESWSGGATYHTDSVKGRFTISRDNAKNTVYLQMNSLKPDDTAVYYCAAAGLGTVVSEWDYDYDYWGQGTQVTVSS

SEQ ID NO: 256 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGWATYHTDSVKGRFTISRDNAKNTVYLQMNSLKPDDTAVYYCAAAGLGTVVSEWDYDYDYWGQGTQVTVSS

SEQ ID NO: 257 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTTSVKGRFTISRDNAKNTVYLQMNSLKPDDTAVYYCAAAGLGTVVSEWDYDYDYWGQGTQVTVSS

SEQ ID NO: 258 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISPSGGATYHTDSVKGRFTISRDNAKNTVYLQMNSLKPDDTAVYYCAAAGLGTVVSEWDYDYDYWGQGTQVTVSS

SEQ ID NO: 259 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLRTVVSEWDYDYDYWGQGTQVTVSS

SEQ ID NO: 260 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGASYHTDSVKGRFTISRDNAKNTVYLQMNSLKPDDTAVYYCAAAGLGTVVSEWDYDYDYWGQGTQVTVSS

SEQ ID NO: 261 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKARFTISRDNAKNTVYLQMNSLKPDDTAVYYCAAAGLGTVVSEWDYDYDYWGQGTQVTVSS

SEQ ID NO: 262 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAYAGLGTVVSEWDYDYDYWGQGTQVTVSS

SEQ ID NO: 263 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGAAYHTDSVKGRFTISRDNAKNTVYLQMNSLKPDDTAVYYCAAAGLGTVVSEWDYDYDYWGQGTQVTVSS

SEQ ID NO: 264 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDYDYDSWGQGTQVTVSS

SEQ ID NO: 265 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWGGGATYHTDSVKGRFTISRDNAKNTVYLQMNSLKPDDTAVYYCAAAGLGTVVSEWDYDYDYWGQGTQVTVSS

SEQ ID NO: 266 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLWTVVSEWDYDYDYWGQGTQVTVSS

SEQ ID NO: 267 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEADYDYDYWGQGTQVTVSS

SEQ ID NO: 268 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSEWDYDFDYWGQGTQVTVSS

SEQ ID NO: 269 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGLGTVVSAWDYDYDYWGQGTQVTVSS

SEQ ID NO: 270 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGAGYHTDSVKGRFTISRDNAKNTVYLQMNSLKPDDTAVYYCAAAGLGTVVSEWDYDYDYWGQGTQVTVSS

SEQ ID NO: 271 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATSSWSGGATYHTDSVKGRFTISRDNAKNTVYLQMNSLKPDDTAVYYCAAAGLGTVVSEWDYDYDYWGQGTQVTVSS

SEQ ID NO: 272 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISLSGGATYHTDSVKGRFTISRDNAKNTVYLQMNSLKPDDTAVYYCAAAGLGTVVSEWDYDYDYWGQGTQVTVSS

SEQ ID NO: 273 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHYDSVKGRFTISRDNAKNTVYLQMNSLKPDDTAVYYCAAAGLGTVVSEWDYDYDYWGQGTQVTVSS

SEQ ID NO: 274 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDSVKGRFTISRDNAKNTVYLQMNSLKPDDTAVYYCAAAGQGTVVSEWDYDYDYWGQGTQVTVSS

SEQ ID NO: 275 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTDAVKGRFTISRDNAKNTVYLQMNSLKPDDTAVYYCAAAGLGTVVSEWDYDYDYWGQGTQVTVSS

SEQ ID NO: 276 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYHTASVKGRFTISRDNAKNTVYLQMNSLKPDDTAVYYCAAAGLGTVVSEWDYDYDYWGQGTQVTVSS

SEQ ID NO: 277 - protein, 261 aa, synthetic construct
  REGION 1..261: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGGGGSGGGGSQVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVGHTYYGQGTQVTVS

SEQ ID NO: 278 - protein, 266 aa, synthetic construct
  REGION 1..266: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGSGSGGGGGSGGGGSQVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVGHTYYGQGTQVTVS

SEQ ID NO: 279 - protein, 275 aa, synthetic construct
  REGION 1..275: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGGGGSGGGGSQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 280 - protein, 270 aa, synthetic construct
  REGION 1..270: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGGGGSQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 281 - protein, 265 aa, synthetic construct
  REGION 1..265: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGGGGSGGGGSQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 282 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVS

SEQ ID NO: 283 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGQLAYHYDYWGQGTQVTVS

SEQ ID NO: 284 - protein, 270 aa, synthetic construct
  REGION 1..270: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGSGSGGGGGSGGGGSQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 285 - protein, 262 aa, synthetic construct
  REGION 1..262: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGGGGSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVS

SEQ ID NO: 286 - protein, 257 aa, synthetic construct
  REGION 1..257: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVS

SEQ ID NO: 287 - protein, 115 aa, synthetic construct
  REGION 1..115: note = source = /note="Description of Artificial Sequence: Synthetic polypeptide"
MQVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVGHTYYGQGTQVTVS

SEQ ID NO: 288 - protein, 128 aa, synthetic construct
  REGION 1..128: note = source = /note="Description of Artificial Sequence: Synthetic polypeptide"
MQVQLQESGGGLVQAGGSLRLSCAASGRTFSEYAMGWFRQAPGKEREFVATISGGWSGGATYHTDSVKGRFTISRDNAKNTVYLQMNSLKPDDTAVYYCAAAGLGTVVSEWDYDYDYWGQGTQVTVSS

SEQ ID NO: 289 - protein, 129 aa, synthetic construct
  REGION 1..129: note = source = /note="Description of Artificial Sequence: Synthetic polypeptide"
MQVQLQESGGGLVQAGGSLRLSCAASGSGGRTFSEYAMGWFRQAPGKEREFVATISWSGGATYHTDSVKGRFTISRDNAKNTVYLQMNSLKPDDTAVYYCAAAGLGTVVSEWDYDYDYWGQGTQVTVSS

SEQ ID NO: 290 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYYTRSVKGRFTISRDNAKNTVYLQMNSLKPDDTAVYYCAAAGLETVVSEWDYDYDYWGQGTQVTVSS

SEQ ID NO: 291 - protein, 127 aa, synthetic construct
  REGION 1..127: note = source = /note="Description of Artificial Sequence: Synthetic polypeptide"
MQVQLQESGGGLVQAGGSLRLSCAASGGRTFSEYAMGWFRQAPGKEREFVATISWSGGATYHTDSVKGRFTISRDNAKNTVYLQMNSLKPDDTAVYYCAAAGLGTVVSEWDYDYDYWGQGTQVTVSS

SEQ ID NO: 292 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYTTRSVKGRFTISRDNAKNTVYLQMNSLKPDDTAVYYCAAAGLETVVSEWDYDYDYWGQGTQVTVSS

SEQ ID NO: 293 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYHTRSVKGRFTISRDNAKNTVYLQMNSLKPDDTAVYYCAAAGLGTVVSEWDYDYDYWGQGTQVTVSS

SEQ ID NO: 294 - protein, 127 aa, synthetic construct
  REGION 1..127: note = source = /note="Description of Artificial Sequence: Synthetic polypeptide"
MQVQLQESGGGLVQAGGSLRLSCAASGRTFSEYAMGWFRQAPGKEREFVATISGWSGGATYHTDSVKGRFTISRDNAKNTVYLQMNSLKPDDTAVYYCAAAGLGTVVSEWDYDYDYWGQGTQVTVSS

SEQ ID NO: 295 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYTTRSVKGRFTISRDNAKNTVYLQMNSLKPDDTAVYYCAAAGLGTVVSEWDYDYDYWGQGTQVTVSS

SEQ ID NO: 296 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYYTWSVKGRFTISRDNAKNTVYLQMNSLKPDDTAVYYCAAAGLETVVSEWDYDYDYWGQGTQVTVSS

SEQ ID NO: 297 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYHTRSVKGRFTISRDNAKNTVYLQMNSLKPDDTAVYYCAAAGLETVVSEWDYDYDYWGQGTQVTVSS

SEQ ID NO: 298 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYHTRSVKGRFTISRDNAKNTVYLQMNSLKPDDTAVYYCAAAGLGTVVSEWDYDYDYWGQGTQVTVSS

SEQ ID NO: 299 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYTTRSVKGRFTISRDNAKNTVYLQMNSLKPDDTAVYYCAAAGLETVVSEWDYDYDYWGQGTQVTVSS

SEQ ID NO: 300 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYHTDSVKGRFTISRDNAKNTVYLQMNSLKPDDTAVYYCAAAGLETVVSEWDYDYDYWGQGTQVTVSS

SEQ ID NO: 301 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYTTDSVKGRFTISRDNAKNTVYLQMNSLKPDDTAVYYCAAAGLETVVSEWDYDYDYWGQGTQVTVSS

SEQ ID NO: 302 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGSTYTTDSVKGRFTISRDNAKNTVYLQMNSLKPDDTAVYYCAAAGLETVVSEWDYDYDYWGQGTQVTVSS

SEQ ID NO: 303 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYYTRSVKGRFTISRDNAKNTVYLQMNSLKPDDTAVYYCAAAGLETVVSEWDYDYDYWGQGTQVTVSS

SEQ ID NO: 304 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYHTRSVKGRFTISRDNAKNTVYLQMNSLKPDDTAVYYCAAAGLETVVSEWDYDYDYWGQGTQVTVSS

SEQ ID NO: 305 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGATYYTQSVKGRFTISRDNAKNTVYLQMNSLKPDDTAVYYCAAAGLGTVVSEWDYDYDYWGQGTQVTVSS

SEQ ID NO: 306 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYTTDSVKGRFTISRDNAKNTVYLQMNSLKPDDTAVYYCAAAGLGTVVSEWDYDYDYWGQGTQVTVSS

SEQ ID NO: 307 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYHTDSVKGRFTISRDNAKNTVYLQMNSLKPDDTAVYYCAAAGLETVVSEWDYDYDYWGQGTQVTVSS

SEQ ID NO: 308 - protein, 121 aa, synthetic construct
  REGION 1..121: note = source = /note="Description of Artificial Sequence: Synthetic polypeptide"
MQVQLVESGGGLVQAGGSLRLSCAASGFPVEAWRMEWYRQAPGKEREGVAAIESYGHGTRYADSVKGRFTISRDNAKNTVYLQMNSLKPEDTAVYYCNVYDDGQLAYHYDYWGQGTQVTVS

SEQ ID NO: 309 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGHLAYHYDYWGQGTQVTVS

SEQ ID NO: 310 - protein, 121 aa, synthetic construct
  REGION 1..121: note = source = /note="Description of Artificial Sequence: Synthetic polypeptide"
MQVQLVESGGGLVQAGGSLRLSCAASGFPVEVWAMEWYRQAPGKEREGVAAIESYGHGTRYADSVKGRFTISRDNAKNTVYLQMNSLKPEDTAVYYCNVYDDGQLAYHYDYWGQGTQVTVS

SEQ ID NO: 311 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIEAYGHGTRYADSVKGRFTISRDNAKNTVYLQMNSLKPEDTAVYYCNVYDDGQLAYHYDYWGQGTQVTVS

SEQ ID NO: 312 - protein, 121 aa, synthetic construct
  REGION 1..121: note = source = /note="Description of Artificial Sequence: Synthetic polypeptide"
MQVQLVESGGGLVQAGGSLRLSCAASGFPVEVYRMEWYRQAPGKEREGVAAIESYGHGTRYADSVKGRFTISRDNAKNTVYLQMNSLKPEDTAVYYCNVYDDGQLAYHYDYWGQGTQVTVS

SEQ ID NO: 313 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESAGHGTRYADSVKGRFTISRDNAKNTVYLQMNSLKPEDTAVYYCNVYDDGQLAYHYDYWGQGTQVTVS

SEQ ID NO: 314 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVRGRFTISRDNAKNTVYLQMNSLKPEDTAVYYCNVYDDGQLAYHYDYWGQGTQVTVS

SEQ ID NO: 315 - protein, 126 aa, synthetic construct
  REGION 1..126: note = source = /note="Description of Artificial Sequence: Synthetic polypeptide"
MQVQLQESGGGLVQAGGSLRLSCAASGRTFSEYAMGWFRQAPGKEREFVATISWSGGGTYTTRSVKGRFTISRDNAKNTVYLQMNSLKPDDTAVYYCAAAGLGTVVSEWDYDYDYWGQGTQVTVSS

SEQ ID NO: 316 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVAGRFTISRDNAKNTVYLQMNSLKPEDTAVYYCNVYDDGQLAYHYDYWGQGTQVTVS

SEQ ID NO: 317 - protein, 121 aa, synthetic construct
  REGION 1..121: note = source = /note="Description of Artificial Sequence: Synthetic polypeptide"
MQVQLVESGGGLVQAGGSLRLSCAASGFPVEVARMEWYRQAPGKEREGVAAIESYGHGTRYADSVKGRFTISRDNAKNTVYLQMNSLKPEDTAVYYCNVYDDGQLAYHYDYWGQGTQVTVS

SEQ ID NO: 318 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESYGHGARYADSVKGRFTISRDNAKNTVYLQMNSLKPEDTAVYYCNVYDDGQLAYHYDYWGQGTQVTVS

SEQ ID NO: 319 - protein, 121 aa, synthetic construct
  REGION 1..121: note = source = /note="Description of Artificial Sequence: Synthetic polypeptide"
MQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDNQLAYHYDYWGQGTQVTVS

SEQ ID NO: 320 - protein, 253 aa, synthetic construct
  REGION 1..253: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSSGGGGSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVKDDGQLAYHYDYWGQGTQVTVS

SEQ ID NO: 321 - protein, 262 aa, synthetic construct
  REGION 1..262: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGGGGSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGQLAYHYDYWGQGTQVTVS

SEQ ID NO: 322 - protein, 272 aa, synthetic construct
  REGION 1..272: note = source = /note="Description of Artificial Sequence: Synthetic polypeptide"
MMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSGGGGSGGGGSGGGGSQVQLVESGGGLVQAGGSLRLSCAASGFPVYRDRMAWYRQAPGKEREWVAAIYSAGQQTRYADSVKGRFTISRDNAKNTVYLQMNSLKPEDTAVYYCNVKDVGHHYEYYDYWGQGTQVTVS

SEQ ID NO: 323 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATYPYDYWGQGTQVTVSSK

SEQ ID NO: 324 - protein, 115 aa, synthetic construct
  REGION 1..115: note = source = /note="Description of Artificial Sequence: Synthetic polypeptide"
MQVQLVESGGGLVQAGGSLRLSCAASGFPVYSWNMWWYRQAPGKEREWVAAIESHADSTRYADSVKGRFTISRDNAKNTVYLQMNSLKPEDTAVYYCYVWVGHTYYGQGTQVTVS

SEQ ID NO: 325 - protein, 129 aa, synthetic construct
  REGION 1..129: note = source = /note="Description of Artificial Sequence: Synthetic polypeptide"
MQVQLVESGGGLMQAGGSLRLSCAVSGRTFATAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 326 - protein, 115 aa, synthetic construct
  REGION 1..115: note = source = /note="Description of Artificial Sequence: Synthetic polypeptide"
MQVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVAHTYYGQGTQVTVS

SEQ ID NO: 327 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRALLSDYATWPYDYWGQGTQVTVSSK

SEQ ID NO: 328 - protein, 129 aa, synthetic construct
  REGION 1..129: note = source = /note="Description of Artificial Sequence: Synthetic polypeptide"
MQVQLVESGGGLMQAGGSLRLSCAVSGATFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 329 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSKLSDYATWPYDYWGQGTQVTVSSK

SEQ ID NO: 330 - protein, 129 aa, synthetic construct
  REGION 1..129: note = source = /note="Description of Artificial Sequence: Synthetic polypeptide"
MQVQLVESGGGLMQAGGSLRLSCAVSGRTHSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 331 - protein, 129 aa, synthetic construct
  REGION 1..129: note = source = /note="Description of Artificial Sequence: Synthetic polypeptide"
MQVQLVESGGGLMQAGGSLRLSCAVSGRTYSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 332 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSAGSAYYADSVKGRFTISRDKAKNTVYLQMNSLKYEDTAVYYCARTENVRSLLSDYATWPYDYWGQGTQVTVSSK

SEQ ID NO: 333 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYAYWGQGTQVTVSSK

SEQ ID NO: 334 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYRYWGQGTQVTVSSK

SEQ ID NO: 335 - protein, 129 aa, synthetic construct
  REGION 1..129: note = source = /note="Description of Artificial Sequence: Synthetic polypeptide"
MQVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYQYWGQGTQVTVSSK

SEQ ID NO: 336 - protein, 495 aa, synthetic construct
  REGION 1..495: note = source = /note="Description of Artificial Sequence: Synthetic polypeptide"
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGMQVQLQESGGGLVQAGGSLRLSCAASGRTFSEYAMGWFRQAPGKEREFVATISWSGGATYTTDSVKGRFTISRDNAKNTVYLQMNSLKPDDTAVYYCAAAGLGTVVSEWDYDYDYWGQGTQVTVSSGGGGSGGGGSQVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGQLAYHYDYWGQGTQVTVS

SEQ ID NO: 337 - protein, 8 aa, synthetic construct
  VARIANT 2: note = /replace="Ala"
  VARIANT 4: note = /replace="Tyr" or "His"
  VARIANT 5: note = /replace="Ala"
  VARIANT 6: note = /replace="Glu"
  VARIANT 7: note = /replace="Ala"
  REGION 1..8: note = SITE - /note="Variant residues given in the sequence have no preference with respect to those in the annotations for variant positions"
  REGION 1..8: note = source = /note="Description of Artificial Sequence: Synthetic peptide"
GRTFSTYA

SEQ ID NO: 338 - protein, 18 aa, synthetic construct
  VARIANT 2: note = /replace="Pro" or "Ala"
  VARIANT 4: note = /replace="Asp" or "Arg"
  VARIANT 5: note = /replace="Gln"
  VARIANT 6: note = /replace="Arg" or "Trp" or "Glu"
  VARIANT 11: note = /replace="Ala"
  VARIANT 12: note = /replace="Ala"
  VARIANT 13: note = /replace="Ala"
  VARIANT 18: note = /replace="Ser" or "Ala"
  REGION 1..18: note = SITE - /note="Variant residues given in the sequence have no preference with respect to those in the annotations for variant positions"
  REGION 1..18: note = source = /note="Description of Artificial Sequence: Synthetic peptide"
AYAGLGTVVSEWDYDYDY